(19) United States
(12) Patent Application Publication
(10) Pub. No.: US 2023/0263779 A1
(43) Pub. Date: Aug. 24, 2023

(54) METHODS OF TREATING SOLID TUMORS USING NANOPARTICLE MTOR INHIBITOR COMBINATION THERAPY

(71) Applicant: Abraxis BioScience, LLC, Summit, NJ (US)

(72) Inventor: Neil P. DESAI, Pacific Palisades, CA (US)

(21) Appl. No.: 17/850,806

(22) Filed: Jun. 27, 2022

Related U.S. Application Data
(63) Continuation of application No. 15/737,943, filed on Dec. 19, 2017, now abandoned.
(60) Provisional application No. 62/186,325, filed on Jun. 29, 2015.

(51) Int. Cl. A61K 31/436; A61K 47/42; A61K 9/00; A61K 9/51; A61K 45/06; A61K 31/454; A61K 31/505; A61K 38/15; A61K 31/4045; A61K 31/44; A61K 31/506; A61K 31/18; A61P 35/00

(52) U.S. Cl. CPC A61K 31/436; A61K 47/42; A61K 9/0019; A61K 9/5169; A61K 45/06; A61K 31/454; A61K 31/505; A61K 38/15; A61K 31/4045; A61K 31/44; A61K 31/506; A61K 31/18; A61P 35/00; A61K 2039/555

(57) ABSTRACT

The present invention relates to methods and compositions for the treatment of a solid tumor by administering compositions comprising nanoparticles that comprise an mTOR inhibitor (such as a limus drug, e.g., sirolimus or a derivative thereof) and an albumin in combination with compositions comprising a second therapeutic agent.

### CROSS-REFERENCE TO RELATED APPLICATIONS

This application claims priority to U.S. Provisional Application No. 62/186,325, filed on Jun. 29, 2015, which is hereby incorporated by reference in its entirety.

### FIELD OF THE INVENTION

This invention pertains to methods and compositions for the treatment of a solid tumor by administering compositions comprising nanoparticles that comprise an mTOR inhibitor (such as a limus drug, e.g., sirolimus or a derivative thereof) and an albumin in combination with a second therapeutic agent.

### BACKGROUND OF THE INVENTION

The mammalian target of rapamycin (mTOR) is a conserved serine/threonine kinase that serves as a central hub of signaling in the cell to integrate intracellular and extracellular signals and to regulate cellular growth and homeostasis. Activation of the mTOR pathway is associated with cell proliferation and survival, while inhibition of mTOR signaling leads to inflammation and cell death. Dysregulation of the mTOR signaling pathway has been implicated in an increasing number of human diseases, including cancer and autoimmune disorders. Consequently, mTOR inhibitors have found wide applications in treating diverse pathological conditions such as solid tumors, hematological malignancies, organ transplantation, restenosis, and rheumatoid arthritis.

Sirolimus (INN/USAN), also known as rapamycin, is an immunosuppressant drug used rejection in organ transplantation; it is especially useful in kidney transplants. Sirolimus-eluting stents were approved in the United States to treat coronary restenosis. Additionally, sirolimus has been demonstrated as an effective inhibitor of tumor growth in various cell lines and animal models. Other limus drugs, such as analogs of sirolimus, have been designed to improve the pharmacokinetic and pharmacodynamic properties of sirolimus. For example, Temsirolimus was approved in the United States and Europe for the treatment of renal cell carcinoma. Everolimus was approved in the U.S. for treatment of advanced breast cancer, pancreatic neuroendocrine tumors, advanced renal cell carcinoma, and subependymal giant cell astrocytoma (SEGA) associated with Tuberous Sclerosis. The mode of action of sirolimus is to bind the cytosolic protein FK-binding protein 12 (FKBP12), and the sirolimus-FKBP12 complex in turn inhibits the mTOR pathway by directly binding to the mTOR Complex 1 (mTORC1).

Albumin-based nanoparticle compositions have been developed as a drug delivery system for delivering substantially water insoluble drugs. See, for example, U.S. Pat. Nos. 5,916,596; 6,506,405; 6,749,868, and 6,537,579, 7,820,788, and 7,923,536. Abraxane®, an albumin stabilized nanoparticle formulation of paclitaxel, was approved in the United States in 2005 and subsequently in various other countries for treating metastatic breast cancer. It was recently approved for treating non-small cell lung cancer in the United States, and has also shown therapeutic efficacy in various clinical trials for treating difficult-to-treat cancers such as bladder cancer and melanoma. Albumin derived from human blood has been used for the manufacture of Abraxane® as well as various other albumin-based nanoparticle compositions.

The disclosures of all publications, patents, patent applications and published patent applications referred to herein are hereby incorporated herein by reference in their entirety.

### BRIEF SUMMARY OF THE INVENTION

The present invention provides methods of treating a solid tumor (such as bladder cancer, renal cell carcinoma, or melanoma) in an individual, comprising administering to the individual a) an effective amount of a composition comprising nanoparticles comprising an mTOR inhibitor (such as a limus drug, e.g., sirolimus or a derivative thereof) and an albumin; and b) an effective amount of a second therapeutic agent. In some embodiments, the mTOR inhibitor nanoparticle composition (such as sirolimus/albumin nanoparticle composition) and the second therapeutic agent act synergistically to inhibit cell proliferation. In some embodiments, the mTOR inhibitor is a limus drug. In some embodiments, the mTOR inhibitor is sirolimus or a derivative thereof. In some embodiments, the mTOR inhibitor is sirolimus. In some embodiments, the albumin is human albumin (such as human serum albumin). In some embodiments, the nanoparticles comprise sirolimus or a derivative thereof associated (e.g., coated) with albumin. In some embodiments, the nanoparticles comprise sirolimus or a derivative thereof coated with albumin. In some embodiments, the average particle size of the nanoparticles in the mTOR inhibitor nanoparticle composition (such as sirolimus/albumin nanoparticle composition) is no greater than about 150 nm (such as no greater than about 120 nm). In some embodiments, the average particle size of the nanoparticles in the mTOR inhibitor nanoparticle composition (such as sirolimus/albumin nanoparticle composition) is no more than about 120 nm. In some embodiments, the nanoparticles in the mTOR inhibitor nanoparticle composition (such as sirolimus/albumin nanoparticle composition) are sterile filterable. In some embodiments, the mTOR inhibitor nanoparticle composition comprises the albumin stabilized nanoparticle formulation of sirolimus (nab-sirolimus, a formulation of sirolimus stabilized by human albumin USP, where the weight ratio of human albumin and sirolimus is about 8:1 to about 9:1). In some embodiments, the mTOR inhibitor nanoparticle composition is nab-sirolimus. In some embodiments, the mTOR inhibitor nanoparticle composition is administered intravenously, intraarterially, intraperitoneally, intravesicularlly, subcutaneously, intrathecally, intrapulmonarily, intramuscularly, intratracheally, intraocularly, transdermally, orally, or by inhalation. In some embodiments, the mTOR inhibitor nanoparticle composition is administered intravenously. In some embodiments, the mTOR inhibitor nanoparticle composition is administered subcutaneously. In some embodiments, the individual is a human.

In some embodiments, according to any of the methods described above, the second therapeutic agent is selected from the group consisting of an immunomodulator (such as an immunostimulator or an immune checkpoint inhibitor), a histone deacetylase inhibitor, a kinase inhibitor (such as a tyrosine kinase inhibitor), and a cancer vaccine (such as a vaccine prepared using tumor cells or at least one tumor-associated antigen). In some embodiments, the second therapeutic agent is an immunomodulator. In some embodiments, the immunomodulator is an IMiDs® compound (small molecule immunomodulator, such as lenalidomide or pomalidomide). In some embodiments, the second therapeutic agent is an immunomodulator that stimulates the immune system (hereinafter also referred to as an “immunostimulator”). In some embodiments, the immunomodulator is an agonistic antibody that targets an activating receptor (including co-stimulatory receptors) on an immune cell (such as a T cell). In some embodiments, the immunomodulator is an immune checkpoint inhibitor. In some embodiments, the immune checkpoint inhibitor is an antagonistic antibody that targets an immune checkpoint protein. In some embodiments, the second therapeutic agent is an immunomodulator selected from the group consisting of pomalidomide and lenalidomide. In some embodiments, the immunomodulator is small molecule or antibody-based IDO inhibitor. In some embodiments, the second therapeutic agent is a histone deacetylase inhibitor. In some embodiments, the histone deacetylase inhibitor is selected from the group consisting of romidepsin, panobinostat, ricolinostat, and belinostat. In some embodiments, the second therapeutic agent is a kinase inhibitor. In some embodiments, the kinase inhibitor is selected from the group consisting of nilotinib and sorafenib. In some embodiments, the second therapeutic agent is a cancer vaccine. In some embodiments, the cancer vaccine is a vaccine prepared from a tumor cell or a vaccine prepared from at least one tumor-associated antigen.

In some embodiments, according to any of the methods described above, the solid tumor is selected from the group consisting of bladder cancer, renal cell carcinoma, and melanoma. In some embodiments, the solid tumor is a relapsed solid tumor. In some embodiments, the solid tumor is refractory to a standard therapy for the solid tumor.

In some embodiments, according to any of the methods described above, the solid tumor is bladder cancer, and the second therapeutic agent is selected from the group consisting of an immunomodulator (such as an immunostimulator or an immune checkpoint inhibitor), a histone deacetylase inhibitor, a kinase inhibitor (such as a tyrosine kinase inhibitor), and a cancer vaccine. In some embodiments, the solid tumor is renal cell carcinoma, and the second therapeutic agent is selected from the group consisting of an immunomodulator (such as an immunostimulator or an immune checkpoint inhibitor), a histone deacetylase inhibitor, a kinase inhibitor (such as a tyrosine kinase inhibitor), and a cancer vaccine. In some embodiments, the solid tumor is melanoma, and the second therapeutic agent is selected from the group consisting of an immunomodulator (such as an immunostimulator or an immune checkpoint inhibitor), a histone deacetylase inhibitor, a kinase inhibitor (such as a tyrosine kinase inhibitor), and a cancer vaccine.

In some embodiments, according to any of the methods described above, the mTOR inhibitor nanoparticle composition (such as sirolimus/albumin nanoparticle composition) and the second therapeutic agent are administered simultaneously. In other embodiments, the mTOR inhibitor nanoparticle composition (such as sirolimus/albumin nanoparticle composition) and the second therapeutic agent are not administered simultaneously. In some embodiments, the mTOR inhibitor nanoparticle composition (such as sirolimus/albumin nanoparticle composition) and the second therapeutic agent are administered sequentially.

In some embodiments, according to any of the methods described above, the mTOR inhibitor nanoparticle composition (such as sirolimus/albumin nanoparticle composition) and the second therapeutic agent are present in amounts that produce a synergistic effect in the treatment of a solid tumor (such as bladder cancer, renal cell carcinoma, or melanoma) in an individual in need thereof.

In some embodiments, according to any of the methods described above, the method is carried out in a neoadjuvant setting. In some embodiments, the method is carried out in an adjuvant setting.

In some embodiments, according to any of the methods described above, the solid tumor is refractory to a standard therapy or recurrent after the standard therapy. In some embodiments, the treatment is first line treatment. In some embodiments, the treatment is second line treatment.

In some embodiments, according to any of the methods described above, the individual has progressed from an earlier therapy for a solid tumor. In some embodiments, the individual is refractory to an earlier therapy for a solid tumor. In some embodiments, the individual has recurrent solid tumor.

In some embodiments, according to any of the methods described above, the amount of the nanoparticles in the mTOR inhibitor nanoparticle composition (such as sirolimus/albumin nanoparticle composition) is about 10 mg/m² to about 200 mg/m² (such as about any of 10, 20, 30, 45, 75, 100, 150, or 200 mg/m², including any range between these values). In some embodiments, the amount of the nanoparticles in the mTOR inhibitor nanoparticle composition (such as sirolimus/albumin nanoparticle composition) is about 45 mg/m². In some embodiments, the amount of the nanoparticles in the mTOR inhibitor nanoparticle composition (such as sirolimus/albumin nanoparticle composition) is about 75 mg/m². In some embodiments, the amount of the nanoparticles in the mTOR inhibitor nanoparticle composition (such as sirolimus/albumin nanoparticle composition) is about 100 mg/m². In some embodiments, the mTOR inhibitor nanoparticle composition (such as sirolimus/albumin nanoparticle composition) is administered weekly (such as 3 out of 4 weeks). In some embodiments, the mTOR inhibitor nanoparticle composition (such as sirolimus/albumin nanoparticle composition) is administered at least twice (such as at least 2, 3, or 4 times) in a 28-day cycle for at least one (such at least 2, 3, 4, 5, 6, 7, 8, 9, 10, or more) cycle. In some embodiments, the mTOR inhibitor nanoparticle composition (such as sirolimus/albumin nanoparticle composition) is administered at least twice (such as at least 2, 3, or 4 times) at weekly intervals in a 28-day cycle (such as on days 1, 8, and 15 of the 28-day cycle) for at least one (such at least 2, 3, 4, 5, 6, 7, 8, 9, 10, or more) cycle. In some embodiments, the mTOR inhibitor nanoparticle composition (such as sirolimus/albumin nanoparticle composition) is administered three times in a 28-day cycle (such as on days 1, 8, and 15 of the 28-day cycle) for at least one (such at least 2, 3, 4, 5, 6, 7, 8, 9, 10, or more) cycle.

Also provided are methods of treating a solid tumor according to any of the methods described above, wherein the treatment is based on the level of at least one biomarker. In some embodiments, the method further comprises selecting the individual for treatment based on the presence of at least one mTOR-activating aberration. In some embodiments, the mTOR-activating aberration comprises a mutation in an mTOR-associated gene. In some embodiments, the mTOR-activating aberration is in at least one mTOR-associated gene selected from the group consisting of protein kinase B (PKB/Akt), fins-like tyrosine kinase 3 internal tandem duplication (FLT-3ITD), mechanistic target of rapamycin (mTOR), phosphoinositide 3-kinase (PI3K), TSC1, TSC2, RHEB, STK11, NF1, NF2, Kirsten rat sarcoma viral oncogene homolog (KRAS), neuroblastoma RAS viral (v-ras) oncogene homolog (NRAS) and PTEN. In some embodiments, the treatment is based on the presence of at least one genetic variant in a gene selected from the group consisting of drug metabolism genes, cancer genes, and drug target genes.

In some embodiments, according to any of the methods described above, the method further comprises selecting the individual for treatment based on the presence of at least one biomarker indicative of favorable response to treatment with an immunomodulator. In some embodiments, the at least one biomarker comprises a mutation in an immunomodulator-associated gene.

In some embodiments, according to any of the methods described above, the method further comprises selecting the individual for treatment based on the presence of at least one biomarker indicative of favorable response to treatment with a histone deacetylase inhibitor (HDACi). In some embodiments, the at least one biomarker comprises a mutation in an HDACi-associated gene.

In some embodiments, according to any of the methods described above, the method further comprises selecting the individual for treatment based on the presence of at least one biomarker indicative of favorable response to treatment with a kinase inhibitor. In some embodiments, the at least one biomarker comprises a mutation in a kinase inhibitor-associated gene.

In some embodiments, according to any of the methods described above, the method further comprises selecting the individual for treatment based on the presence of at least one biomarker indicative of favorable response to treatment with a cancer vaccine. In some embodiments, the at least one biomarker comprises a tumor-associated antigen (TAA) expressed in tumor cells in the individual, such as an aberrantly expressed protein or a neo-antigen.

The methods described herein can be used for any one or more of the following purposes: alleviating one or more symptoms of a solid tumor, delaying progressing of a solid tumor, shrinking tumor size in a solid tumor patient, inhibiting solid tumor growth, prolonging overall survival, prolonging disease-free survival, prolonging time to tumor progression, preventing or delaying metastasis, reducing (such as eradicating) preexisting metastasis, reducing incidence or burden of preexisting metastasis, and preventing recurrence of solid tumor.

### BRIEF DESCRIPTION OF THE DRAWINGS

FIG. 1 shows the experimental design schema for a Phase I clinical study in pediatric patients of ABI-009 as a single agent and in combination with temozolomide and irinotecan.

### DETAILED DESCRIPTION OF THE INVENTION

The present invention provides methods and compositions for treating a solid tumor (such as bladder cancer, renal cell carcinoma, or melanoma) in an individual by administering to the individual a composition comprising nanoparticles comprising an mTOR inhibitor (such as a limus drug, e.g., sirolimus or a derivative thereof) and an albumin (hereinafter also referred to as an “mTOR inhibitor nanoparticle composition”) in conjunction with a second therapeutic agent. The second therapeutic agent may be an immunomodulator (such as an immunostimulator or an immune checkpoint inhibitor), a histone deacetylase inhibitor, a kinase inhibitor (such as a tyrosine kinase inhibitor), or a cancer vaccine (such as a vaccine prepared from a tumor cell or a vaccine prepared from at least one tumor-associated antigen).

The present application thus provides methods of combination therapy. It is to be understood by a person of ordinary skill in the art that the combination therapy methods described herein requires that one agent or composition be administered in conjunction with another agent.

Also provided are compositions (such as pharmaceutical compositions), kits, and unit dosages useful for the methods described herein.

### Definitions

As used herein “nab” stands for nanoparticle albumin-bound, and “nab-sirolimus” is an albumin stabilized nanoparticle formulation of sirolimus. nab-sirolimus is also known as nab-rapamycin, which has been previously described. See, for example, WO2008109163A1, WO2014151853, WO2008137148A2, and WO2012149451A1, each of which is incorporated herein by reference in their entirety.

As used herein, “treatment” or “treating” is an approach for obtaining beneficial or desired results including clinical results. For purposes of this invention, beneficial or desired clinical results include, but are not limited to, one or more of the following: alleviating one or more symptoms resulting from the disease, diminishing the extent of the disease, stabilizing the disease (e.g., preventing or delaying the worsening of the disease), preventing or delaying the spread (e.g., metastasis) of the disease, preventing or delaying the recurrence of the disease, reducing recurrence rate of the disease, delay or slowing the progression of the disease, ameliorating the disease state, providing a remission (partial or total) of the disease, decreasing the dose of one or more other medications required to treat the disease, delaying the progression of the disease, increasing the quality of life, and/or prolonging survival. In some embodiments, the treatment reduces the severity of one or more symptoms associated with cancer by at least about any of 10%, 20%, 30%, 40%, 50%, 60%, 70%, 80%, 90%, 95% or 100% compared to the corresponding symptom in the same subject prior to treatment or compared to the corresponding symptom in other subjects not receiving the treatment. Also encompassed by “treatment” is a reduction of pathological consequence of cancer. The methods of the invention contemplate any one or more of these aspects of treatment.

The terms “recurrence,” “relapse” or “relapsed” refers to the return of a cancer or disease after clinical assessment of the disappearance of disease. A diagnosis of distant metastasis or local recurrence can be considered a relapse.

The term “refractory” or “resistant” refers to a cancer or disease that has not responded to treatment.

As used herein, an “at risk” individual is an individual who is at risk of developing cancer. An individual “at risk” may or may not have detectable disease, and may or may not have displayed detectable disease prior to the treatment methods described herein. “At risk” denotes that an individual has one or more so-called risk factors, which are measurable parameters that correlate with development of cancer, which are described herein. An individual having one or more of these risk factors has a higher probability of developing cancer than an individual without these risk factor(s).

“Adjuvant setting” refers to a clinical setting in which an individual has had a history of cancer, and generally (but not necessarily) been responsive to therapy, which includes, but is not limited to, surgery (e.g., surgery resection), radiotherapy, and chemotherapy. However, because of their history of cancer, these individuals are considered at risk of development of the disease. Treatment or administration in the “adjuvant setting” refers to a subsequent mode of treatment. The degree of risk (e.g., when an individual in the adjuvant setting is considered as “high risk” or “low risk”) depends upon several factors, most usually the extent of disease when first treated.

“Neoadjuvant setting” refers to a clinical setting in which the method is carried out before the primary/definitive therapy.

As used herein, “delaying” the development of cancer means to defer, hinder, slow, retard, stabilize, and/or postpone development of the disease. This delay can be of varying lengths of time, depending on the history of the disease and/or individual being treated. As is evident to one skilled in the art, a sufficient or significant delay can, in effect, encompass prevention, in that the individual does not develop the disease. A method that “delays” development of cancer is a method that reduces probability of disease development in a given time frame and/or reduces the extent of the disease in a given time frame, when compared to not using the method. Such comparisons are typically based on clinical studies, using a statistically significant number of subjects. Cancer development can be detectable using standard methods, including, but not limited to, computerized axial tomography (CAT scan), Magnetic Resonance Imaging (MRI), ultrasound, clotting tests, arteriography, biopsy, urine cytology, and cystoscopy. Development may also refer to cancer progression that may be initially undetectable and includes occurrence, recurrence, and onset.

The term “effective amount” used herein refers to an amount of a compound or composition sufficient to treat a specified disorder, condition or disease such as ameliorate, palliate, lessen, and/or delay one or more of its symptoms. In reference to cancer, an effective amount comprises an amount sufficient to cause a tumor to shrink and/or to decrease the growth rate of the tumor (such as to suppress tumor growth) or to prevent or delay other unwanted cell proliferation in cancer. In some embodiments, an effective amount is an amount sufficient to delay development of cancer. In some embodiments, an effective amount is an amount sufficient to prevent or delay recurrence. In some embodiments, an effective amount is an amount sufficient to reduce recurrence rate in the individual. An effective amount can be administered in one or more administrations. The effective amount of the drug or composition may: (i) reduce the number of cancer cells; (ii) reduce tumor size; (iii) inhibit, retard, slow to some extent and preferably stop cancer cell infiltration into peripheral organs; (iv) inhibit (i.e., slow to some extent and preferably stop) tumor metastasis; (v) inhibit tumor growth; (vi) prevent or delay occurrence and/or recurrence of tumor; (vii) reduce recurrence rate of tumor, and/or (viii) relieve to some extent one or more of the symptoms associated with the cancer.

As is understood in the art, an “effective amount” may be in one or more doses, i.e., a single dose or multiple doses may be required to achieve the desired treatment endpoint. An effective amount may be considered in the context of administering one or more therapeutic agents, and a nanoparticle composition (e.g., a composition including sirolimus and an albumin) may be considered to be given in an effective amount if, in conjunction with one or more other agents, a desirable or beneficial result may be or is achieved. The components (e.g., the first and second therapies) in a combination therapy of the invention may be administered sequentially, simultaneously, or concurrently using the same or different routes of administration for each component. Thus, an effective amount of a combination therapy includes an amount of the first therapy and an amount of the second therapy that when administered sequentially, simultaneously, or concurrently produces a desired outcome.

“In conjunction with” or “in combination with” refers to administration of one treatment modality in addition to another treatment modality, such as administration of a nanoparticle composition described herein in addition to administration of the other agent to the same individual under the same treatment plan. As such, “in conjunction with” or “in combination with” refers to administration of one treatment modality before, during or after delivery of the other treatment modality to the individual.

The term “simultaneous administration,” as used herein, means that a first therapy and second therapy in a combination therapy are administered with a time separation of no more than about 15 minutes, such as no more than about any of 10, 5, or 1 minutes. When the first and second therapies are administered simultaneously, the first and second therapies may be contained in the same composition (e.g., a composition comprising both a first and second therapy) or in separate compositions (e.g., a first therapy is contained in one composition and a second therapy is contained in another composition).

As used herein, the term “sequential administration” means that the first therapy and second therapy in a combination therapy are administered with a time separation of more than about 15 minutes, such as more than about any of 20, 30, 40, 50, 60, or more minutes. Either the first therapy or the second therapy may be administered first. The first and second therapies are contained in separate compositions, which may be contained in the same or different packages or kits.

As used herein, the term “concurrent administration” means that the administration of the first therapy and that of a second therapy in a combination therapy overlap with each other.

As used herein, “specific”, “specificity”, or “selective” or “selectivity” as used when describing a compound as an inhibitor, means that the compound preferably interacts with (e.g., binds to, modulates, and inhibits) a particular target (e.g., a protein and an enzyme) than a non-target. For example, the compound has a higher affinity, a higher avidity, a higher binding coefficient, or a lower dissociation coefficient for a particular target. The specificity or selectivity of a compound for a particular target can be measured, determined, or assessed by using various methods well known in the art. For example, the specificity or selectivity can be measured, determined, or assessed by measuring the IC₅₀ of a compound for a target. A compound is specific or selective for a target when the IC₅₀ of the compound for the target is 2-fold, 4-fold, 6-fold, 8-fold, 10-fold, 20-fold, 50-fold, 100-fold, 500-fold, 1000-fold, or more lower than the IC₅₀ of the same compound for a non-target. For example, the IC₅₀ of a histone deacetylase inhibitor of the present invention for HDACs is 2-fold, 4-fold, 6-fold, 8-fold, 10-fold, 20-fold, 50-fold, 100-fold, 500-fold, 1000-fold, or more lower than the IC₅₀ of the same histone deacetylase inhibitor for non-HDACs. For example, the IC₅₀ of a histone deacetylase inhibitor of the present invention for class-I HDACs is 2-fold, 4-fold, 6-fold, 8-fold, 10-fold, 20-fold, 50-fold, 100-fold, 500-fold, 1000-fold, or more lower than the IC₅₀ of the same histone deacetylase inhibitor for other HDACs (e.g., class-II HDACs). For example, the IC₅₀ of a histone deacetylase inhibitor of the present invention for HDAC3 is 2-fold, 4-fold, 6-fold, 8-fold, 10-fold, 20-fold, 50-fold, 100-fold, 500-fold, 1000-fold, or more lower than the IC₅₀ of the same histone deacetylase inhibitor for other HDACs (e.g., HDAC1, 2, or 6). IC₅₀ can be determined by commonly known methods in the art.

As used herein, by “pharmaceutically acceptable” or “pharmacologically compatible” is meant a material that is not biologically or otherwise undesirable, e.g., the material may be incorporated into a pharmaceutical composition administered to a patient without causing any significant undesirable biological effects or interacting in a deleterious manner with any of the other components of the composition in which it is contained. Pharmaceutically acceptable carriers or excipients have preferably met the required standards of toxicological and manufacturing testing and/or are included on the Inactive Ingredient Guide prepared by the U.S. Food and Drug administration.

It is understood that embodiments of the invention described herein include “consisting” and/or “consisting essentially of” embodiments.

Reference to “about” a value or parameter herein includes (and describes) variations that are directed to that value or parameter per se. For example, description referring to “about X” includes description of “X”.

As used herein, reference to “not” a value or parameter generally means and describes “other than” a value or parameter. For example, the method is not used to treat cancer of type X means the method is used to treat cancer of types other than X.

As used herein and in the appended claims, the singular forms “a,” “or,” and “the” include plural referents unless the context clearly dictates otherwise.

### Methods of Treating a Solid Tumor

The present invention provides methods of treating a solid tumor (such as bladder cancer, renal cell carcinoma, or melanoma) in an individual (such as a human) comprising administering to the individual a) an effective amount of a composition comprising nanoparticles comprising an mTOR inhibitor (such as a limus drug, e.g., sirolimus or a derivative thereof) and an albumin; and b) an effective amount of a second therapeutic agent. In some embodiments, the solid tumor includes, but is not limited to, sarcomas and carcinomas such as fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, Kaposi's sarcoma, soft tissue sarcoma, uterine sacronomasynovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilm's tumor, cervical cancer, testicular tumor, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, menangioma, melanoma, neuroblastoma, and retinoblastoma.

In some embodiments, there is provided a method of treating a solid tumor (such as bladder cancer, renal cell carcinoma, or melanoma) in an individual (such as a human) comprising administering to the individual a) an effective amount of a composition comprising nanoparticles comprising an mTOR inhibitor (such as a limus drug, e.g., sirolimus or a derivative thereof) and an albumin, wherein the mTOR inhibitor in the nanoparticles is associated (e.g., coated) with the albumin; and b) an effective amount of a second therapeutic agent. In some embodiments, the method comprises administering to the individual a) an effective amount of a composition comprising nanoparticles comprising an mTOR inhibitor (such as a limus drug, e.g., sirolimus or a derivative thereof) and an albumin, wherein the nanoparticles have an average particle size of no greater than about 150 nm (such as no greater than about 120 nm); and b) an effective amount of a second therapeutic agent. In some embodiments, the method comprises administering to the individual a) an effective amount of a composition comprising nanoparticles comprising an mTOR inhibitor (such as a limus drug, e.g., sirolimus or a derivative thereof) and an albumin, wherein the nanoparticles comprise the mTOR inhibitor associated (e.g., coated) with albumin, and wherein the nanoparticles have an average particle size of no greater than about 150 nm (such as no greater than about 120 nm); and b) an effective amount of a second therapeutic agent. In some embodiments, the method comprises administering to the individual a) an effective amount of a composition comprising nanoparticles comprising an mTOR inhibitor (such as a limus drug, e.g., sirolimus or a derivative thereof) and an albumin, wherein the nanoparticles comprise the mTOR inhibitor associated (e.g., coated) with the albumin, wherein the nanoparticles have an average particle size of no greater than about 150 nm (such as no greater than about 120 nm, for example about 100 nm), and wherein the weight ratio of albumin and the mTOR inhibitor in the mTOR inhibitor nanoparticle composition is about 9:1 or less (such as about 9:1 or about 8:1); and b) an effective amount of a second therapeutic agent. In some embodiments, the method further comprises administering to the individual at least one therapeutic agent used in a standard combination therapy with the second therapeutic agent. In some embodiments, the mTOR inhibitor is a limus drug. In some embodiments, the mTOR inhibitor is sirolimus or a derivative thereof. In some embodiments, the mTOR inhibitor nanoparticle composition comprises nab-sirolimus. In some embodiments, the mTOR inhibitor nanoparticle composition is nab-sirolimus. In some embodiments, the second therapeutic agent is selected from the group consisting of an immunomodulator (such as an immunostimulator or an immune checkpoint inhibitor), a histone deacetylase inhibitor, a kinase inhibitor (such as a tyrosine kinase inhibitor), and a cancer vaccine (such as a vaccine prepared using tumor cells or at least one tumor-associated antigen). In some embodiments, the second therapeutic agent is an immunomodulator (such as an immunostimulator or an immune checkpoint inhibitor). In some embodiments, the immunomodulator is an immunostimulator that directly stimulates the immune system of an individual. In some embodiments, the immunomodulator is an agonistic antibody that targets an activating receptor on an immune cell (such as a T cell). In some embodiments, the immunomodulator is an immune checkpoint inhibitor. In some embodiments, the immune checkpoint inhibitor is an antagonistic antibody that targets an immune checkpoint protein. In some embodiments, the immunomodulator is an IMiDs® compound (small molecule immunomodulator, such as lenalidomide or pomalidomide). In some embodiments, the immunomodulator is small molecule or antibody-based IDO inhibitor. In some embodiments, the second therapeutic agent is a histone deacetylase inhibitor. In some embodiments, the histone deacetylase inhibitor is specific to only one HDAC. In some embodiments, the histone deacetylase inhibitor is specific to only one class of HDAC. In some embodiments, the histone deacetylase inhibitor is specific to two or more HDACs or two or more classes of HDACs. In some embodiments, the histone deacetylase inhibitor is specific to class I and II HDACs. In some embodiments, the histone deacetylase inhibitor is specific to class III HDACs. In some embodiments, the histone deacetylase inhibitor is selected from the group consisting of romidepsin, panobinostat, ricolinostat, and belinostat. In some embodiments, the second therapeutic agent is a kinase inhibitor, such as a tyrosine kinase inhibitor. In some embodiments, the kinase inhibitor is a serine/threonine kinase inhibitor. In some embodiments, the kinase inhibitor is a Raf kinase inhibitor. In some embodiments, the kinase inhibitor inhibits more than one class of kinase (e.g., an inhibitor of more than one of a tyrosine kinase, a Raf kinase, and a serine/threonine kinase). In some embodiments, the kinase inhibitor is selected from the group consisting of erlotinib, imatinib, lapatinib, nilotinib, sorafenib, and sunitinib. In some embodiments, the second therapeutic agent is a cancer vaccine, such as a vaccine prepared using tumor cells or at least one tumor-associated antigen. In some embodiments, the second therapeutic agent and the nanoparticle composition are administered sequentially. In some embodiments, the second therapeutic agent and the nanoparticle composition are administered simultaneously. In some embodiments, the second therapeutic agent and the nanoparticle composition are administered concurrently. In some embodiments, the solid tumor is selected from the group consisting of bladder cancer, renal cell carcinoma, and melanoma. In some embodiments, the solid tumor is a relapsed or refractory solid tumor.

In some embodiments, there is provided a method of treating a solid tumor (such as bladder cancer, renal cell carcinoma, or melanoma) in an individual (such as a human) comprising administering to the individual a) an effective amount of a composition comprising nanoparticles comprising an mTOR inhibitor (such as a limus drug, e.g., sirolimus or a derivative thereof) and an albumin; and b) an effective amount of a second therapeutic agent, wherein the nanoparticle composition and the second therapeutic agent are administered concurrently. In some embodiments, the administrations of the nanoparticle composition and the second therapeutic agent are initiated at about the same time (for example, within any one of 1, 2, 3, 4, 5, 6, or 7 days). In some embodiments, the administrations of the nanoparticle composition and the second therapeutic agent are terminated at about the same time (for example, within any one of 1, 2, 3, 4, 5, 6, or 7 days). In some embodiments, the administration of the second therapeutic agent continues (for example for about any one of 0.5, 1, 2, 3, 4, 5, 6, 7, 8, 9, 10, 11, or 12 months) after the termination of the administration of the nanoparticle composition. In some embodiments, the administration of the second therapeutic agent is initiated after (for example after about any one of 0.5, 1, 2, 3, 4, 5, 6, 7, 8, 9, 10, 11, or 12 months) the initiation of the administration of the nanoparticle composition. In some embodiments, the administrations of the nanoparticle composition and the second therapeutic agent are initiated and terminated at about the same time. In some embodiments, the administrations of the nanoparticle composition and the second therapeutic agent are initiated at about the same time and the administration of the second therapeutic agent continues (for example for about any one of 0.5, 1, 2, 3, 4, 5, 6, 7, 8, 9, 10, 11, or 12 months) after the termination of the administration of the nanoparticle composition. In some embodiments, the administration of the nanoparticle composition and the second therapeutic agent stop at about the same time and the administration of the second therapeutic agent is initiated after (for example after about any one of 0.5, 1, 2, 3, 4, 5, 6, 7, 8, 9, 10, 11, or 12 months) the initiation of the administration of the nanoparticle composition. In some embodiments, the administration of the nanoparticle composition and the second therapeutic agent stop at about the same time and the administration of the nanoparticle composition is initiated after (for example after about any one of 0.5, 1, 2, 3, 4, 5, 6, 7, 8, 9, 10, 11, or 12 months) the initiation of the administration of the second therapeutic agent.

“mTOR inhibitor” used herein refers to inhibitors of mTOR. mTOR is a serine/threonine-specific protein kinase downstream of the phosphatidylinositol 3-kinase (PI3K)/Akt (protein kinase B) pathway, and a key regulator of cell survival, proliferation, stress, and metabolism. mTOR pathway dysregulation has been found in many human carcinomas, and mTOR inhibition produced substantial inhibitory effects on tumor progression. In some embodiments, the mTOR inhibitor is an mTOR kinase inhibitor. mTOR inhibitors described herein include, but are not limited to, BEZ235 (NVP-BEZ235), everolimus (also known as RAD001, Zortress, Certican, and Afinitor), rapamycin (also known as sirolimus or Rapamune), AZD8055, temsirolimus (also known as CCI-779 and Torisel), CC-115, CC-223, PI-103, Ku-0063794, INK 128, AZD2014, NVP-BGT226, PF-04691502, CH5132799, GDC-0980 (RG7422), Torin 1, WAY-600, WYE-125132, WYE-687, GSK2126458, PF-05212384 (PKI-587), PP-121, OSI-027, Palomid 529, PP242, XL765, GSK1059615, WYE-354, and ridaforolimus (also known as deforolimus).

In some embodiments, the mTOR inhibitor is a limus drug, which includes sirolimus and its analogs. Examples of limus drugs include, but are not limited to, temsirolimus (CCI-779), everolimus (RAD001), ridaforolimus (AP-23573), deforolimus (MK-8669), zotarolimus (ABT-578), pimecrolimus, and tacrolimus (FK-506). In some embodiments, the limus drug is selected from the group consisting of temsirolimus (CCI-779), everolimus (RAD001), ridaforolimus (AP-23573), deforolimus (MK-8669), zotarolimus (ABT-578), pimecrolimus, and tacrolimus (FK-506). In some embodiments, the mTOR inhibitor is an mTOR kinase inhibitor, such as CC-115 or CC-223.

Thus, for example, in some embodiments, there is provided a method of treating a solid tumor (such as bladder cancer, renal cell carcinoma, or melanoma) in an individual (such as a human) comprising administering to the individual a) an effective amount of a composition comprising nanoparticles comprising an mTOR inhibitor (such as a limus drug, e.g., sirolimus or a derivative thereof) and an albumin, wherein the mTOR inhibitor is selected from the group consisting of BEZ235 (NVP-BEZ235), everolimus (also known as RAD001, Zortress, Certican, and Afinitor), rapamycin (also known as sirolimus or Rapamune), AZD8055, temsirolimus (also known as CCI-779 and Torisel), CC-115, CC-223, PI-103, Ku-0063794, INK 128, AZD2014, NVP-BGT226, PF-04691502, CH5132799, GDC-0980 (RG7422), Torin 1, WAY-600, WYE-125132, WYE-687, GSK2126458, PF-05212384 (PKI-587), PP-121, OSI-027, Palomid 529, PP242, XL765, GSK1059615, WYE-354, and ridaforolimus (also known as deforolimus); and b) an effective amount of a second therapeutic agent.

In some embodiments, there is provided a method of treating a solid tumor (such as bladder cancer, renal cell carcinoma, or melanoma) in an individual (such as a human) comprising administering to the individual a) an effective amount of a composition comprising nanoparticles comprising an mTOR inhibitor (such as a limus drug, e.g., sirolimus or a derivative thereof) and an albumin, wherein the mTOR inhibitor is a limus drug selected from the group consisting of temsirolimus (CCI-779), everolimus (RAD001), ridaforolimus (AP-23573), deforolimus (MK-8669), zotarolimus (ABT-578), pimecrolimus, and tacrolimus (FK-506); and b) an effective amount of a second therapeutic agent.

In some embodiments, the second therapeutic agent is an immunomodulator. In some embodiments, the immunomodulator is an immunostimulator. In some embodiments, the immunostimulator directly stimulates the immune system of an individual. In some embodiments, the immunomodulator is an IMiDs® compound (Celgene). IMiDs® compounds are proprietary small molecule, orally available compounds that modulate the immune system and other biological targets through multiple mechanisms of action; IMiDs® compounds include lenalidomide and pomalidomide. In some embodiments, the immunomodulator is small molecule or antibody-based IDO inhibitor. In some embodiments, the immunomodulator is selected from the group consisting of a cytokine, a chemokine, a stem cell growth factor, a lymphotoxin, an hematopoietic factor, a colony stimulating factor (CSF), erythropoietin, thrombopoietin, tumor necrosis factor-alpha (TNF), TNF-beta, granulocyte-colony stimulating factor (G-CSF), granulocyte macrophage-colony stimulating factor (GM-CSF), interferon-alpha, interferon-beta, interferon-gamma, interferon-lambda, stem cell growth factor designated “S1 factor”, human growth hormone, N-methionyl human growth hormone, bovine growth hormone, parathyroid hormone, thyroxine, insulin, proinsulin, relaxin, prorelaxin, follicle stimulating hormone (FSH), thyroid stimulating hormone (TSH), luteinizing hormone (LH), hepatic growth factor, prostaglandin, fibroblast growth factor, prolactin, placental lactogen, OB protein, mullerian-inhibiting substance, mouse gonadotropin-associated peptide, inhibin, activin, vascular endothelial growth factor, integrin, NGF-beta, platelet-growth factor, TGF-alpha, TGF-beta, insulin-like growth factor-I, insulin-like growth factor-II, macrophage-CSF (M-CSF), IL-1, IL-1a, IL-2, IL-3, IL-4, IL-5, IL-6, IL-7, IL-8, IL-9, IL-10, IL-11, IL-12, IL-13, IL-14, IL-15, IL-16, IL-17, IL-18, IL-21, IL-25, LIF, FLT-3, angiostatin, thrombospondin, endostatin, lymphotoxin, thalidomide, lenalidomide, and pomalidomide. In some embodiments, the immunomodulator is lenalidomide. In some embodiments, the immunomodulator is pomalidomide. In some embodiments, the immunomodulator is an agonistic antibody that targets an activating receptor (including co-stimulatory receptors) on an immune cell (such as a T cell). In some embodiments, the immunomodulator is an agonistic antibody selected from the group consisting of anti-CD28, anti-OX40 (such as MED16469), anti-GITR (such as TRX518), anti-4-1BB (such as BMS-663513 and PF-05082566), anti-ICOS (such as JTX-2011, Jounce Therapeutics), anti-CD27 (such as Varlilumab and hCD27.15), anti-CD40 (such as CP870,893), and anti-HVEM. In some embodiments, the immunomodulator is an immune checkpoint inhibitor. In some embodiments, the immune checkpoint inhibitor is an antagonistic antibody that targets an immune checkpoint protein. In some embodiments, the immunomodulator is an antagonistic antibody selected from the group consisting of anti-CTLA4 (such as Ipilimumab and Tremelimumab), anti-PD-1 (such as Nivolumab, Pidilizumab, and Pembrolizumab), anti-PD-L1 (such as MPDL3280A, BMS-936559, MED14736, and Avelumab), anti-PD-L2, anti-LAG3 (such as BMS-986016 or C9B7W), anti-B7-1, anti-B7-H3 (such as MGA271), anti-B7-H4, anti-TIM3, anti-BTLA, anti-VISTA, anti-KIR (such as Lirilumab and IPH2101), anti-A2aR, anti-CD52 (such as alemtuzumab), anti-IL-10, anti-FasL (such as disclosed in U.S. Pat. No. 9,255,150), anti-IL-35, and anti-TGF-0 (such as Fresolumimab).

Thus, for example, in some embodiments, there is provided a method of treating a solid tumor (such as bladder cancer, renal cell carcinoma, or melanoma) in an individual (such as a human) comprising administering to the individual a) an effective amount of a composition comprising nanoparticles comprising an mTOR inhibitor (such as a limus drug, e.g., sirolimus or a derivative thereof) and an albumin; and b) an effective amount of an immunomodulator. In some embodiments, there is provided a method of treating a solid tumor (such as bladder cancer, renal cell carcinoma, or melanoma) in an individual (such as a human) comprising administering to the individual a) an effective amount of a composition comprising nanoparticles comprising an mTOR inhibitor (such as a limus drug, e.g., sirolimus or a derivative thereof) and an albumin; and b) an effective amount of an immunostimulator. In some embodiments, there is provided a method of treating a solid tumor (such as bladder cancer, renal cell carcinoma, or melanoma) in an individual (such as a human) comprising administering to the individual a) an effective amount of a composition comprising nanoparticles comprising an mTOR inhibitor (such as a limus drug, e.g., sirolimus or a derivative thereof) and an albumin; and b) an effective amount of an immunostimulator that directly stimulates the immune system of an individual. In some embodiments, there is provided a method of treating a solid tumor (such as bladder cancer, renal cell carcinoma, or melanoma) in an individual (such as a human) comprising administering to the individual a) an effective amount of a composition comprising nanoparticles comprising an mTOR inhibitor (such as a limus drug, e.g., sirolimus or a derivative thereof) and an albumin; and b) an effective amount of an IMiDs® compound (small molecule immunomodulator, such as lenalidomide or pomalidomide). In some embodiments, there is provided a method of treating a solid tumor (such as bladder cancer, renal cell carcinoma, or melanoma) in an individual (such as a human) comprising administering to the individual a) an effective amount of a composition comprising nanoparticles comprising an mTOR inhibitor (such as a limus drug, e.g., sirolimus or a derivative thereof) and an albumin; and b) an effective amount of a small molecule or antibody-based IDO inhibitor.

In some embodiments, there is provided a method of treating a solid tumor (such as bladder cancer, renal cell carcinoma, or melanoma) in an individual (such as a human) comprising administering to the individual a) an effective amount of a composition comprising nanoparticles comprising an mTOR inhibitor (such as a limus drug, e.g., sirolimus or a derivative thereof) and an albumin; and b) an effective amount of an immunomodulator (such as an immunostimulator) selected from the group consisting of a cytokine, a chemokine, a stem cell growth factor, a lymphotoxin, an hematopoietic factor, a colony stimulating factor (CSF), erythropoietin, thrombopoietin, tumor necrosis factor-alpha (TNF), TNF-beta, granulocyte-colony stimulating factor (G-CSF), granulocyte macrophage-colony stimulating factor (GM-CSF), interferon-alpha, interferon-beta, interferon-gamma, interferon-lambda, stem cell growth factor designated “S1 factor”, human growth hormone, N-methionyl human growth hormone, bovine growth hormone, parathyroid hormone, thyroxine, insulin, proinsulin, relaxin, prorelaxin, follicle stimulating hormone (FSH), thyroid stimulating hormone (TSH), luteinizing hormone (LH), hepatic growth factor, prostaglandin, fibroblast growth factor, prolactin, placental lactogen, OB protein, mullerian-inhibiting substance, mouse gonadotropin-associated peptide, inhibin, activin, vascular endothelial growth factor, integrin, NGF-beta, platelet-growth factor, TGF-alpha, TGF-beta, insulin-like growth factor-I, insulin-like growth factor-II, macrophage-CSF (M-CSF), IL-1, IL-1a, IL-2, IL-3, IL-4, IL-5, IL-6, IL-7, IL-8, IL-9, IL-10, IL-11, IL-12, IL-13, IL-14, IL-15, IL-16, IL-17, IL-18, IL-21, IL-25, LIF, FLT-3, angiostatin, thrombospondin, endostatin, lymphotoxin, thalidomide, lenalidomide, and pomalidomide. In some embodiments, the immunomodulator is lenalidomide. In some embodiments, the immunomodulator is pomalidomide.

In some embodiments, there is provided a method of treating a solid tumor (such as bladder cancer, renal cell carcinoma, or melanoma) in an individual (such as a human) comprising administering to the individual a) an effective amount of a composition comprising nanoparticles comprising an mTOR inhibitor (such as a limus drug, e.g., sirolimus or a derivative thereof) and an albumin; and b) an effective amount of an agonist of an activating receptor (including co-stimulatory receptors) on an immune cell (such as a T cell). In some embodiments, the agonist of an activating receptor (including co-stimulatory receptors) on an immune cell (such as a T cell) is an agonistic antibody selected from the group consisting of anti-CD28, anti-OX40 (such as MED16469), anti-ICOS (such as JTX-2011, Jounce Therapeutics), anti-GITR (such as TRX518), anti-4-1BB (such as BMS-663513 and PF-05082566), anti-CD27 (such as Varlilumab and hCD27.15), anti-CD40 (such as CP870,893), and anti-HVEM.

In some embodiments, there is provided a method of treating a solid tumor (such as bladder cancer, renal cell carcinoma, or melanoma) in an individual (such as a human) comprising administering to the individual a) an effective amount of a composition comprising nanoparticles comprising an mTOR inhibitor (such as a limus drug, e.g., sirolimus or a derivative thereof) and an albumin; and b) an effective amount of an immune checkpoint inhibitor. In some embodiments, the immune checkpoint inhibitor is an antagonistic antibody that targets an immune checkpoint protein. In some embodiments, the immune checkpoint inhibitor is an antagonistic antibody selected from the group consisting of anti-CTLA4 (such as Ipilimumab and Tremelimumab), anti-PD-1 (such as Nivolumab, Pidilizumab, and Pembrolizumab), anti-PD-L1 (such as MPDL3280A, BMS-936559, MED14736, and Avelumab), anti-PD-L2, anti-LAG3 (such as BMS-986016 or C9B7W), anti-B7-1, anti-B7-H3 (such as MGA271), anti-B7-H4, anti-TIM3, anti-BTLA, anti-VISTA, anti-KIR (such as Lirilumab and IPH2101), anti-A2aR, anti-CD52 (such as alemtuzumab), anti-IL-10, anti-FasL, anti-IL-35, and anti-TGF-0 (such as Fresolumimab).

In some embodiments, the second therapeutic agent is a histone deacetylase inhibitor. In some embodiments, the histone deacetylase inhibitor is specific to only one HDAC. In some embodiments, the histone deacetylase inhibitor is specific to only one class of HDAC. In some embodiments, the histone deacetylase inhibitor is specific to two or more HDACs or two or more classes of HDACs. In some embodiments, the histone deacetylase inhibitor is specific to class I and II HDACs. In some embodiments, the histone deacetylase inhibitor is specific to class III HDACs. In some embodiments, the histone deacetylase inhibitor is selected from the group consisting of vorinostat (SAHA), panobinostat (LBH589), belinostat (PXD101, CAS 414864-00-9), tacedinaline (N-acetyldinaline, CI-994), givinostat (gavinostat, ITF2357), FRM-0334 (EVP-0334), resveratrol (SRT501), CUDC-101, quisinostat (JNJ-26481585), abexinostat (PCI-24781), dacinostat (LAQ824, NVP-LAQ824), valproic acid, 4-(dimethylamino)N-[6-(hydroxyamino)-6-oxohexyl]-benzamide (HDAC1 inhibitor), 4-Iodo suberoylanilide hydroxamic acid (HDAC1 and HDAC6 inhibitor), romidepsin (a cyclic tetrapeptide with HDAC inhibitory activity primarily towards class-I HDACs), 1-naphthohydroxamic acid (HDAC1 and HDAC6 inhibitor), HDAC inhibitors based on amino-benzamide biasing elements (e.g., mocetinostat (MGCD103) and entinostat (MS275), which are highly selective for HDAC1, 2 and 3), AN-9 (CAS 122110-53-6), APHA Compound 8 (CAS 676599-90-9), apicidin (CAS 183506-66-3), BML-210 (CAS 537034-17-6), salermide (CAS 1105698-15-4), suberoyl bis-hydroxamic acid (CAS 38937-66-5) (HDAC1 and HDAC3 inhibitor), butyrylhydroxamic acid (CAS 4312-91-8), CAY10603 (CAS 1045792-66-2) (HDAC6 inhibitor), CBHA (CAS 174664-65-4), ricolinostat (ACY1215, rocilinostat), trichostatin-A, WT-161, tubacin, and Merck60. In some embodiments, the second therapeutic agent is the histone deacetylase inhibitor romidepsin.

Thus, for example, in some embodiments, there is provided a method of treating a solid tumor (such as bladder cancer, renal cell carcinoma, or melanoma) in an individual (such as a human) comprising administering to the individual a) an effective amount of a composition comprising nanoparticles comprising an mTOR inhibitor (such as a limus drug, e.g., sirolimus or a derivative thereof) and an albumin; and b) an effective amount of a histone deacetylase inhibitor. In some embodiments, the histone deacetylase inhibitor is specific to only one HDAC. In some embodiments, the histone deacetylase inhibitor is specific to only one class of HDAC. In some embodiments, the histone deacetylase inhibitor is specific to two or more HDACs or two or more classes of HDACs. In some embodiments, the histone deacetylase inhibitor is specific to class I and II HDACs. In some embodiments, the histone deacetylase inhibitor is specific to class III HDACs. In some embodiments, the histone deacetylase inhibitor is a hydroxamic acid, including, but not limited to, vorinostat (suberoylanilide hydroxamic acid or “SAHA”), trichostatin A (“TSA”), LBH589 (panobinostat), PXD101 (belinostat), oxamflatin, tubacin, seriptaid, NVP-LAQ824, cinnamic acid hydroxamic acid (CBHA), CBHA derivatives, and ITF2357. In some embodiments, the histone deacetylase inhibitor is a benzamide, including, but not limited to, mocetinostat (MGCD0103), benzamide M344, BML-210, entinostat (SNDX-275 or MS-275), pimelic diphenylamide 4b, pimelic diphenylamide 106, MS-994, CI-994 (acetyldinaline, PD 123654, and 4-acetylamino-N-(Uaminophenyl)-benzamide). In some embodiments, the histone deacetylase inhibitor is romidepsin.

In some embodiments, the second therapeutic agent is a kinase inhibitor, such as a tyrosine kinase inhibitor. In some embodiments, the kinase inhibitor is a serine/threonine kinase inhibitor. In some embodiments, the kinase inhibitor is a Raf kinase inhibitor. In some embodiments, the kinase inhibitor inhibits more than one class of kinase (e.g., an inhibitor of more than one of a tyrosine kinase, a Raf kinase, and a serine/threonine kinase). In some embodiments, the kinase inhibitor is selected from the group consisting of apatinib, cabozantinib, canertinib, crenolanib, crizotinib, dasatinib, erlotinib, foretinib, fostamatinib, ibrutinib, idelalisib, imatinib, lapatinib, linifanib, motesanib, mubritinib, nilotinib, nintedanib, radotinib, sorafenib, sunitinib, vatalanib, and vemurafenib. In some embodiments, the second therapeutic agent is the kinase inhibitor nilotinib. In some embodiments, the second therapeutic agent is the kinase inhibitor sorafenib.

Thus, for example, in some embodiments, there is provided a method of treating a solid tumor (such as bladder cancer, renal cell carcinoma, or melanoma) in an individual (such as a human) comprising administering to the individual a) an effective amount of a composition comprising nanoparticles comprising an mTOR inhibitor (such as a limus drug, e.g., sirolimus or a derivative thereof) and an albumin; and b) an effective amount of a kinase inhibitor. In some embodiments, the kinase inhibitor is a tyrosine kinase inhibitor. In some embodiments, the kinase inhibitor is a serine/threonine kinase inhibitor. In some embodiments, the kinase inhibitor is a Raf kinase inhibitor. In some embodiments, the kinase inhibitor inhibits more than one class of kinase (e.g., an inhibitor of more than one of a tyrosine kinase, a Raf kinase, and a serine/threonine kinase). In some embodiments, the kinase inhibitor is selected from the group consisting of apatinib, cabozantinib, canertinib, crenolanib, crizotinib, dasatinib, erlotinib, foretinib, fostamatinib, ibrutinib, idelalisib, imatinib, lapatinib, linifanib, motesanib, mubritinib, nilotinib, nintedanib, radotinib, sorafenib, sunitinib, vatalanib, and vemurafenib. In some embodiments, the kinase inhibitor is nilotinib. In some embodiments, the kinase inhibitor is sorafenib.

In some embodiments, the second therapeutic agent is a cancer vaccine, such as a vaccine prepared using tumor cells or at least one tumor-associated antigen. In some embodiments, the cancer vaccine is a vaccine prepared using autologous tumor cells. In some embodiments, the cancer vaccine is a vaccine prepared using allogeneic tumor cells. In some embodiments, the cancer vaccine is a vaccine prepared using at least one tumor-associated antigen (TAA).

Thus, for example, in some embodiments, there is provided a method of treating a solid tumor (such as bladder cancer, renal cell carcinoma, or melanoma) in an individual (such as a human) comprising administering to the individual a) an effective amount of a composition comprising nanoparticles comprising an mTOR inhibitor (such as a limus drug, e.g., sirolimus or a derivative thereof) and an albumin; and b) an effective amount of a cancer vaccine. In some embodiments, the cancer vaccine is a vaccine prepared using autologous tumor cells. In some embodiments, the cancer vaccine is a vaccine prepared using allogeneic tumor cells. In some embodiments, the cancer vaccine is a vaccine prepared using at least one tumor-associated antigen (TAA).

Reference to a second therapeutic agent herein applies to the second therapeutic agent or its derivatives and accordingly the invention contemplates and includes either of these embodiments (second therapeutic agent; second therapeutic agent or derivative(s) thereof). “Derivatives” or “analogs” of an agent or other chemical moiety include, but are not limited to, compounds that are structurally similar to the agent or moiety or are in the same general chemical class as the agent or moiety. In some embodiments, the derivative or analog of the second therapeutic agent or moiety retains similar chemical and/or physical property (including, for example, functionality) of the second therapeutic agent or moiety.

In some embodiments, according to any of the methods described herein, the method further comprises administering to the individual one or more additional therapeutic agents used in a standard combination therapy with the second therapeutic agent. Thus, in some embodiments, there is provided a method of treating a solid tumor (such as bladder cancer, renal cell carcinoma, or melanoma) in an individual (such as a human) comprising administering to the individual a) an effective amount of a composition comprising nanoparticles comprising an mTOR inhibitor (such as a limus drug, e.g., sirolimus or a derivative thereof) and an albumin; b) an effective amount of a second therapeutic agent; and c) an effective amount of at least one therapeutic agent used in a standard combination therapy with the second therapeutic agent.

The methods provided herein can be used to treat an individual (e.g., human) who has been diagnosed with or is suspected of having a solid tumor. In some embodiments, the individual is a human. In some embodiments, the individual is a clinical patient, a clinical trial volunteer, an experimental animal, etc. In some embodiments, the individual is younger than about 60 years old (including for example younger than about any of 50, 40, 30, 25, 20, 15, or 10 years old). In some embodiments, the individual is older than about 60 years old (including for example older than about any of 70, 80, 90, or 100 years old). In some embodiments, the individual is diagnosed with or genetically prone to one or more of the diseases or disorders described herein (such as bladder cancer, renal cell carcinoma, or melanoma). In some embodiments, the individual has one or more risk factors associated with one or more diseases or disorders described herein.

Cancer treatments can be evaluated, for example, by tumor regression, tumor weight or size shrinkage, time to progression, duration of survival, progression free survival, overall response rate, duration of response, quality of life, protein expression and/or activity. Approaches to determining efficacy of the therapy can be employed, including for example, measurement of response through radiological imaging.

In some embodiments, the efficacy of treatment is measured as the percentage tumor growth inhibition (% TGI), calculated using the equation 100−(T/C×100), where T is the mean relative tumor volume of the treated tumor, and C is the mean relative tumor volume of a non-treated tumor. In some embodiments, the % TGI is about 10%, about 20%, about 30%, about 40%, about 50%, about 60%, about 70%, about 80%, about 90%, about 91%, about 92%, about 93%, about 94%, about 95%, or more than 95%.

### Bladder Cancer

In some embodiments, there is provided a method of treating bladder cancer (such as non-muscle invasive bladder cancer, e.g., BCG-refractory NMIBC) in an individual (such as a human) comprising administering to the individual a) an effective amount of a composition comprising nanoparticles comprising an mTOR inhibitor (such as a limus drug, e.g., sirolimus or a derivative thereof) and an albumin; and b) an effective amount of a second therapeutic agent. In some embodiments, the method comprises administering to the individual a) an effective amount of a composition comprising nanoparticles comprising an mTOR inhibitor (such as a limus drug, e.g., sirolimus or a derivative thereof) and an albumin, wherein the mTOR inhibitor in the nanoparticles is associated (e.g., coated) with the albumin; and b) an effective amount of a second therapeutic agent. In some embodiments, the method comprises administering to the individual a) an effective amount of a composition comprising nanoparticles comprising an mTOR inhibitor (such as a limus drug, e.g., sirolimus or a derivative thereof) and an albumin, wherein the nanoparticles have an average particle size of no greater than about 150 nm (such as no greater than about 120 nm); and b) an effective amount of a second therapeutic agent. In some embodiments, the method comprises administering to the individual a) an effective amount of a composition comprising nanoparticles comprising an mTOR inhibitor (such as a limus drug, e.g., sirolimus or a derivative thereof) and an albumin, wherein the nanoparticles comprise the mTOR inhibitor associated (e.g., coated) with albumin, wherein the nanoparticles have an average particle size of no greater than about 150 nm (such as no greater than about 120 nm); and b) an effective amount of a second therapeutic agent. In some embodiments, the method comprises administering to the individual a) an effective amount of a composition comprising nanoparticles comprising an mTOR inhibitor (such as a limus drug, e.g., sirolimus or a derivative thereof) and an albumin, wherein the nanoparticles comprise the mTOR inhibitor associated (e.g., coated) with the albumin, wherein the nanoparticles have an average particle size of no greater than about 150 nm (such as no greater than about 120 nm, for example about 100 nm), wherein the weight ratio of albumin and the mTOR inhibitor in the mTOR inhibitor nanoparticle composition is about 9:1 or less (such as about 9:1 or about 8:1); and b) an effective amount of a second therapeutic agent. In some embodiments, the method further comprises administering to the individual at least one therapeutic agent used in a standard combination therapy with the second therapeutic agent. In some embodiments, the mTOR inhibitor is a limus drug. In some embodiments, the mTOR inhibitor is sirolimus or a derivative thereof. In some embodiments, the mTOR inhibitor nanoparticle composition comprises nab-sirolimus. In some embodiments, the mTOR inhibitor nanoparticle composition is nab-sirolimus. In some embodiments, the second therapeutic agent is selected from the group consisting of an immunomodulator (such as an immunostimulator or an immune checkpoint inhibitor), a histone deacetylase inhibitor, a kinase inhibitor (such as a tyrosine kinase inhibitor), and a cancer vaccine (such as a vaccine prepared using tumor cells or at least one tumor-associated antigen). In some embodiments, the second therapeutic agent is an immunomodulator. In some embodiments, the immunomodulator is an immunostimulator that directly stimulates the immune system of an individual. In some embodiments, the immunomodulator is an agonistic antibody that targets an activating receptor on an immune cell (such as a T cell). In some embodiments, the immunomodulator is an immune checkpoint inhibitor. In some embodiments, the immune checkpoint inhibitor is an antagonistic antibody that targets an immune checkpoint protein. In some embodiments, the immunomodulator is an IMiDs® compound (small molecule immunomodulator, such as lenalidomide or pomalidomide). In some embodiments, the immunomodulator is lenalidomide. In some embodiments, the immunomodulator is pomalidomide. In some embodiments, the immunomodulator is small molecule or antibody-based IDO inhibitor. In some embodiments, the second therapeutic agent is a histone deacetylase inhibitor. In some embodiments, the histone deacetylase inhibitor is specific to only one HDAC. In some embodiments, the histone deacetylase inhibitor is specific to only one class of HDAC. In some embodiments, the histone deacetylase inhibitor is specific to two or more HDACs or two or more classes of HDACs. In some embodiments, the histone deacetylase inhibitor is specific to class I and II HDACs. In some embodiments, the histone deacetylase inhibitor is specific to class III HDACs. In some embodiments, the histone deacetylase inhibitor is selected from the group consisting of romidepsin, panobinostat, ricolinostat, and belinostat. In some embodiments, the histone deacetylase inhibitor is romidepsin. In some embodiments, the second therapeutic agent is a kinase inhibitor, such as a tyrosine kinase inhibitor. In some embodiments, the kinase inhibitor is a serine/threonine kinase inhibitor. In some embodiments, the kinase inhibitor is a Raf kinase inhibitor. In some embodiments, the kinase inhibitor inhibits more than one class of kinase (e.g., an inhibitor of more than one of a tyrosine kinase, a Raf kinase, and a serine/threonine kinase). In some embodiments, the kinase inhibitor is selected from the group consisting of erlotinib, imatinib, lapatinib, nilotinib, sorafenib, and sunitinib. In some embodiments, the kinase inhibitor is sorafenib. In some embodiments, the kinase inhibitor is nilotinib. In some embodiments, the second therapeutic agent is a cancer vaccine, such as a vaccine prepared using tumor cells or at least one tumor-associated antigen. In some embodiments, the second therapeutic agent and the nanoparticle composition are administered sequentially. In some embodiments, the second therapeutic agent and the nanoparticle composition are administered simultaneously. In some embodiments, the second therapeutic agent and the nanoparticle composition are administered concurrently.

In some embodiments, the bladder cancer is a low grade bladder cancer. In some embodiments, the bladder cancer is a high grade bladder cancer. In some embodiments, the bladder cancer is invasive. In some embodiments, the bladder cancer is non-invasive. In some embodiments, the bladder cancer is non-muscle invasive.

In some embodiments, the bladder cancer is transitional cell carcinoma or urothelial carcinoma (such as metastatic urothelial carcinoma), including, but not limited to, papillary tumors and flat carcinomas. In some embodiments, the bladder cancer is metastatic urothelial carcinoma. In some embodiments, the bladder cancer is urothelial carcinoma of the bladder. In some embodiments, the bladder cancer is urothelial carcinoma of the ureter. In some embodiments, the bladder cancer is urothelial carcinoma of the urethra. In some embodiments, the bladder cancer is urothelial carcinoma of the renal pelvis.

In some embodiments, the bladder cancer is squamous cell carcinoma. In some embodiments, the bladder cancer is non-squamous cell carcinoma. In some embodiments, the bladder cancer is adenocarcinoma. In some embodiments, the bladder cancer is small cell carcinoma.

In some embodiments, the bladder cancer is early stage bladder cancer, non-metastatic bladder cancer, non-invasive bladder cancer, non-muscle-invasive bladder cancer, primary bladder cancer, advanced bladder cancer, locally advanced bladder cancer (such as unresectable locally advanced bladder cancer), metastatic bladder cancer, or bladder cancer in remission. In some embodiments, the bladder cancer is localized resectable, localized unresectable, or unresectable. In some embodiments, the bladder cancer is a high grade, non-muscle-invasive cancer that has been refractory to standard intra-bladder infusion (intravesicular) therapy.

The methods provided herein can be used to treat an individual (e.g., human) who has been diagnosed with or is suspected of having bladder cancer. In some embodiments, the individual has undergone a tumor resection. In some embodiments, the individual has refused surgery. In some embodiments, the individual is medically inoperable. In some embodiments, the individual is at a clinical stage of Ta, Tis, T1, T2, T3a, T3b, or T4 bladder cancer. In some embodiments, the individual is at a clinical stage of Tis, CIS, Ta, or T1.

In some embodiments, the individual is a human who exhibits one or more symptoms associated with bladder cancer. In some embodiments, the individual is at an early stage of bladder cancer. In some embodiments, the individual is at an advanced stage of bladder cancer. In some of embodiments, the individual is genetically or otherwise predisposed (e.g., having a risk factor) to developing bladder cancer. Individuals at risk for bladder cancer include, e.g., those having relatives who have experienced bladder cancer, and those whose risk is determined by analysis of genetic or biochemical markers. In some embodiments, the individual is positive for SPARC expression (for example based on IHC standard). In some embodiments, the individual is negative for SPARC expression. In some embodiments, the individual has a mutation in FGFR2. In some embodiments, the individual has a mutation in p53. In some embodiments, the individual has a mutation in MIB-1. In some embodiments, the individual has a mutation in one or more of FEZ1/LZTS1, PTEN, CDKN2A/MTS1/P6, CDKN2B/INK4B/P15, TSC1, DBCCR1, HRAS1, ERBB2, or NF1. In some embodiments, the individual has mutation in both p53 and PTEN.

In some embodiments, the individual has been previously treated for bladder cancer (also referred to as the “prior therapy”). In some embodiments, individual has been previously treated with a standard therapy for bladder cancer. In some embodiments, the prior standard therapy is treatment with BCG. In some embodiments, the prior standard therapy is treatment with mitomycin C. In some embodiments, the prior standard therapy is treatment with interferon (such as interferon-α). In some embodiments, the individual has bladder cancer in remission, progressive bladder cancer, or recurrent bladder cancer. In some embodiments, the individual is resistant to treatment of bladder cancer with other agents (such as platinum-based agents, BCG, mitomycin C, and/or interferon). In some embodiments, the individual is initially responsive to treatment of bladder cancer with other agents (such as platinum-based agents, or BCG) but has progressed after treatment.

In some embodiments, the individual has recurrent bladder cancer (such as a bladder cancer at the clinical stage of Ta, Tis, T1, T2, T3a, T3b, or T4) after a prior therapy (such as prior standard therapy, for example treatment with BCG). For example, the individual may be initially responsive to the treatment with the prior therapy, but develops bladder cancer after about any of about 2, 3, 4, 5, 6, 7, 8, 9, 10, 11, 12, 24, 36, 48, or 60 months upon the cessation of the prior therapy.

In some embodiments, the individual is refractory to a prior therapy (such as prior standard therapy, for example treatment with BCG).

In some embodiments, the individual has progressed on the prior therapy (such as prior standard therapy, for example treatment with BCG) at the time of treatment. For example, the individual has progressed within any of about 1, 2, 3, 4, 5, 6, 7, 8, 9, 10, 11, or 12 months upon treatment with the prior therapy.

In some embodiments, the individual is resistant to the prior therapy (such as prior standard therapy, for example treatment with BCG).

In some embodiments, the individual is unsuitable to continue with the prior therapy (such as prior standard therapy, for example treatment with BCG), for example due to failure to respond and/or due to toxicity.

In some embodiments, the individual is non-responsive to the prior therapy (such as prior standard therapy, for example treatment with BCG).

In some embodiments, the individual is partially responsive to the prior therapy (such as prior standard therapy, for example treatment with BCG), or exhibits a less desirable degree of responsiveness.

In some embodiments, there is provided a method of treating bladder cancer (such as non-muscle invasive bladder cancer, e.g., BCG-refractory NMIBC) in an individual (such as a human) comprising administering to the individual a) an effective amount of a composition comprising nanoparticles comprising an mTOR inhibitor (such as a limus drug, e.g., sirolimus or a derivative thereof) and an albumin; and b) an effective amount of an immunomodulator (such as lenalidomide, pomalidomide, or an immune checkpoint inhibitor). In some embodiments, the method comprises administering to the individual a) an effective amount of a composition comprising nanoparticles comprising an mTOR inhibitor (such as a limus drug, e.g., sirolimus or a derivative thereof) and an albumin, wherein the mTOR inhibitor in the nanoparticles is associated (e.g., coated) with the albumin; and b) an effective amount of an immunomodulator (such as lenalidomide, pomalidomide, or an immune checkpoint inhibitor). In some embodiments, the method comprises administering to the individual a) an effective amount of a composition comprising nanoparticles comprising an mTOR inhibitor (such as a limus drug, e.g., sirolimus or a derivative thereof) and an albumin, wherein the nanoparticles have an average particle size of no greater than about 150 nm (such as no greater than about 120 nm); and b) an effective amount of an immunomodulator (such as lenalidomide, pomalidomide, or an immune checkpoint inhibitor). In some embodiments, the method comprises administering to the individual a) an effective amount of a composition comprising nanoparticles comprising an mTOR inhibitor (such as a limus drug, e.g., sirolimus or a derivative thereof) and an albumin, wherein the nanoparticles comprise the mTOR inhibitor associated (e.g., coated) with albumin, wherein the nanoparticles have an average particle size of no greater than about 150 nm (such as no greater than about 120 nm); and b) an effective amount of an immunomodulator (such as lenalidomide, pomalidomide, or an immune checkpoint inhibitor). In some embodiments, the method comprises administering to the individual a) an effective amount of a composition comprising nanoparticles comprising an mTOR inhibitor (such as a limus drug, e.g., sirolimus or a derivative thereof) and an albumin, wherein the nanoparticles comprise the mTOR inhibitor associated (e.g., coated) with the albumin, wherein the nanoparticles have an average particle size of no greater than about 150 nm (such as no greater than about 120 nm, for example about 100 nm), wherein the weight ratio of albumin and the mTOR inhibitor in the mTOR inhibitor nanoparticle composition is about 9:1 or less (such as about 9:1 or about 8:1); and b) an effective amount of an immunomodulator (such as lenalidomide, pomalidomide, or an immune checkpoint inhibitor). In some embodiments, the method further comprises administering to the individual at least one therapeutic agent used in a standard combination therapy with the immunomodulator. In some embodiments, the mTOR inhibitor is a limus drug. In some embodiments, the mTOR inhibitor is sirolimus or a derivative thereof. In some embodiments, the mTOR inhibitor nanoparticle composition comprises nab-sirolimus. In some embodiments, the mTOR inhibitor nanoparticle composition is nab-sirolimus. In some embodiments, the immunomodulator is an immunostimulator that directly stimulates the immune system of an individual. In some embodiments, the immunomodulator is an agonistic antibody that targets an activating receptor on an immune cell (such as a T cell). In some embodiments, the immunomodulator is an immune checkpoint inhibitor. In some embodiments, the immune checkpoint inhibitor is an antagonistic antibody that targets an immune checkpoint protein. In some embodiments, the immunomodulator is an IMiDs® compound (small molecule immunomodulator, such as lenalidomide or pomalidomide). In some embodiments, the immunomodulator is lenalidomide. In some embodiments, the immunomodulator is pomalidomide. In some embodiments, the immunomodulator is small molecule or antibody-based IDO inhibitor. In some embodiments, the bladder cancer is recurrent bladder cancer. In some embodiments, the bladder cancer is refractory to one or more drugs used in a standard therapy for bladder cancer, such as, but not limited to, platinum-based agents, BCG, mitomycin C, and/or interferon.

In some embodiments, there is provided a method of treating bladder cancer (such as non-muscle invasive bladder cancer, e.g., BCG-refractory NMIBC) in an individual (such as a human) comprising administering to the individual a) an effective amount of a composition comprising nanoparticles comprising sirolimus or a derivative thereof and an albumin; and b) an effective amount of an immunomodulator (such as an immunostimulator, e.g., pomalidomide). In some embodiments, the method comprises administering to the individual a) an effective amount of a composition comprising nanoparticles comprising sirolimus or a derivative thereof and an albumin, wherein the sirolimus or derivative thereof in the nanoparticles is associated (e.g., coated) with the albumin; and b) an effective amount of an immunomodulator (such as an immunostimulator, e.g., pomalidomide). In some embodiments, the method comprises administering to the individual a) an effective amount of a composition comprising nanoparticles comprising sirolimus or a derivative thereof and an albumin, wherein the nanoparticles have an average particle size of no greater than about 150 nm (such as no greater than about 120 nm); and b) an effective amount of an immunomodulator (such as an immunostimulator, e.g., pomalidomide). In some embodiments, the method comprises administering to the individual a) an effective amount of a composition comprising nanoparticles comprising sirolimus or a derivative thereof and an albumin, wherein the nanoparticles comprise the sirolimus or derivative thereof associated (e.g., coated) with albumin, wherein the nanoparticles have an average particle size of no greater than about 150 nm (such as no greater than about 120 nm); and b) an effective amount of an immunomodulator (such as an immunostimulator, e.g., pomalidomide). In some embodiments, the method comprises administering to the individual a) an effective amount of a composition comprising nanoparticles comprising sirolimus or a derivative thereof and albumin, wherein the nanoparticles comprise the sirolimus or derivative thereof associated (e.g., coated) with the albumin, wherein the nanoparticles have an average particle size of no greater than about 150 nm (such as no greater than about 120 nm, for example about 100 nm), wherein the weight ratio of albumin and sirolimus or a derivative thereof in the sirolimus nanoparticle composition is about 9:1 or less (such as about 9:1 or about 8:1); and b) an effective amount of an immunomodulator (such as an immunostimulator, e.g., pomalidomide). In some embodiments, the method further comprises administering to the individual at least one therapeutic agent used in a standard combination therapy with the immunomodulator. In some embodiments, the sirolimus or derivative thereof is sirolimus. In some embodiments, the sirolimus nanoparticle composition comprises nab-sirolimus. In some embodiments, the sirolimus nanoparticle composition is nab-sirolimus. In some embodiments, the immunomodulator is an immunostimulator that directly stimulates the immune system of an individual. In some embodiments, the immunomodulator is an agonistic antibody that targets an activating receptor on an immune cell (such as a T cell). In some embodiments, the immunomodulator is an immune checkpoint inhibitor. In some embodiments, the immune checkpoint inhibitor is an antagonistic antibody that targets an immune checkpoint protein. In some embodiments, the immunomodulator is an IMiDs® compound (small molecule immunomodulator, such as lenalidomide or pomalidomide). In some embodiments, the immunomodulator is lenalidomide. In some embodiments, the immunomodulator is pomalidomide. In some embodiments, the immunomodulator is small molecule or antibody-based IDO inhibitor. In some embodiments, the bladder cancer is recurrent bladder cancer. In some embodiments, the bladder cancer is refractory to one or more drugs used in a standard therapy for bladder cancer, such as, but not limited to, platinum-based agents, BCG, mitomycin C, and/or interferon.

In some embodiments, there is provided a method of treating bladder cancer (such as non-muscle invasive bladder cancer, e.g., BCG-refractory NMIBC) in an individual (such as a human) comprising administering to the individual a) an effective amount of a composition comprising nanoparticles comprising an mTOR inhibitor (such as a limus drug, e.g., sirolimus or a derivative thereof) and an albumin; and b) an effective amount of a histone deacetylase inhibitor (such as romidepsin). In some embodiments, the method comprises administering to the individual a) an effective amount of a composition comprising nanoparticles comprising an mTOR inhibitor (such as a limus drug, e.g., sirolimus or a derivative thereof) and an albumin, wherein the mTOR inhibitor in the nanoparticles is associated (e.g., coated) with the albumin; and b) an effective amount of a histone deacetylase inhibitor (such as romidepsin). In some embodiments, the method comprises administering to the individual a) an effective amount of a composition comprising nanoparticles comprising an mTOR inhibitor (such as a limus drug, e.g., sirolimus or a derivative thereof) and an albumin, wherein the nanoparticles have an average particle size of no greater than about 150 nm (such as no greater than about 120 nm); and b) an effective amount of a histone deacetylase inhibitor (such as romidepsin). In some embodiments, the method comprises administering to the individual a) an effective amount of a composition comprising nanoparticles comprising an mTOR inhibitor (such as a limus drug, e.g., sirolimus or a derivative thereof) and an albumin, wherein the nanoparticles comprise the mTOR inhibitor associated (e.g., coated) with albumin, wherein the nanoparticles have an average particle size of no greater than about 150 nm (such as no greater than about 120 nm); and b) an effective amount of a histone deacetylase inhibitor (such as romidepsin). In some embodiments, the method comprises administering to the individual a) an effective amount of a composition comprising nanoparticles comprising an mTOR inhibitor (such as a limus drug, e.g., sirolimus or a derivative thereof) and an albumin, wherein the nanoparticles comprise the mTOR inhibitor associated (e.g., coated) with the albumin, wherein the nanoparticles have an average particle size of no greater than about 150 nm (such as no greater than about 120 nm, for example about 100 nm), wherein the weight ratio of albumin and the mTOR inhibitor in the mTOR inhibitor nanoparticle composition is about 9:1 or less (such as about 9:1 or about 8:1); and b) an effective amount of a histone deacetylase inhibitor (such as romidepsin). In some embodiments, the method further comprises administering to the individual at least one therapeutic agent used in a standard combination therapy with the histone deacetylase inhibitor. In some embodiments, the mTOR inhibitor is a limus drug. In some embodiments, the mTOR inhibitor is sirolimus or a derivative thereof. In some embodiments, the mTOR inhibitor nanoparticle composition comprises nab-sirolimus. In some embodiments, the mTOR inhibitor nanoparticle composition is nab-sirolimus. In some embodiments, the histone deacetylase inhibitor is selected from the group consisting of romidepsin, panobinostat, ricolinostat, and belinostat. In some embodiments, the histone deacetylase inhibitor is romidepsin. In some embodiments, the bladder cancer is recurrent bladder cancer. In some embodiments, the bladder cancer is refractory to one or more drugs used in a standard therapy for bladder cancer, such as, but not limited to, platinum-based agents, BCG, mitomycin C, and/or interferon.

In some embodiments, there is provided a method of treating bladder cancer (such as non-muscle invasive bladder cancer, e.g., BCG-refractory NMIBC) in an individual (such as a human) comprising administering to the individual a) an effective amount of a composition comprising nanoparticles comprising sirolimus or a derivative thereof and an albumin; and b) an effective amount of a histone deacetylase inhibitor (such as romidepsin). In some embodiments, the method comprises administering to the individual a) an effective amount of a composition comprising nanoparticles comprising sirolimus or a derivative thereof and an albumin, wherein the sirolimus or derivative thereof in the nanoparticles is associated (e.g., coated) with the albumin; and b) an effective amount of a histone deacetylase inhibitor (such as romidepsin). In some embodiments, the method comprises administering to the individual a) an effective amount of a composition comprising nanoparticles comprising sirolimus or a derivative thereof and an albumin, wherein the nanoparticles have an average particle size of no greater than about 150 nm (such as no greater than about 120 nm); and b) an effective amount of a histone deacetylase inhibitor (such as romidepsin). In some embodiments, the method comprises administering to the individual a) an effective amount of a composition comprising nanoparticles comprising sirolimus or a derivative thereof and an albumin, wherein the nanoparticles comprise the sirolimus or derivative thereof associated (e.g., coated) with albumin, wherein the nanoparticles have an average particle size of no greater than about 150 nm (such as no greater than about 120 nm); and b) an effective amount of a histone deacetylase inhibitor (such as romidepsin). In some embodiments, the method comprises administering to the individual a) an effective amount of a composition comprising nanoparticles comprising sirolimus or a derivative thereof and albumin, wherein the nanoparticles comprise the sirolimus or derivative thereof associated (e.g., coated) with the albumin, wherein the nanoparticles have an average particle size of no greater than about 150 nm (such as no greater than about 120 nm, for example about 100 nm), wherein the weight ratio of albumin and sirolimus or a derivative thereof in the sirolimus nanoparticle composition is about 9:1 or less (such as about 9:1 or about 8:1); and b) an effective amount of a histone deacetylase inhibitor (such as romidepsin). In some embodiments, the method further comprises administering to the individual at least one therapeutic agent used in a standard combination therapy with the histone deacetylase inhibitor. In some embodiments, the sirolimus or derivative thereof is sirolimus. In some embodiments, the sirolimus nanoparticle composition comprises nab-sirolimus. In some embodiments, the sirolimus nanoparticle composition is nab-sirolimus. In some embodiments, the histone deacetylase inhibitor is selected from the group consisting of romidepsin, panobinostat, ricolinostat, and belinostat. In some embodiments, the histone deacetylase inhibitor is romidepsin. In some embodiments, the bladder cancer is recurrent bladder cancer. In some embodiments, the bladder cancer is refractory to one or more drugs used in a standard therapy for bladder cancer, such as, but not limited to, platinum-based agents, BCG, mitomycin C, and/or interferon.

In some embodiments, there is provided a method of treating bladder cancer (such as non-muscle invasive bladder cancer, e.g., BCG-refractory NMIBC) in an individual (such as a human) comprising administering to the individual a) an effective amount of a composition comprising nanoparticles comprising an mTOR inhibitor (such as a limus drug, e.g., sirolimus or a derivative thereof) and an albumin; and b) an effective amount of a kinase inhibitor (such as a tyrosine kinase inhibitor, e.g., nilotinib or sorafenib). In some embodiments, the method comprises administering to the individual a) an effective amount of a composition comprising nanoparticles comprising an mTOR inhibitor (such as a limus drug, e.g., sirolimus or a derivative thereof) and an albumin, wherein the mTOR inhibitor in the nanoparticles is associated (e.g., coated) with the albumin; and b) an effective amount of a kinase inhibitor (such as a tyrosine kinase inhibitor, e.g., nilotinib or sorafenib). In some embodiments, the method comprises administering to the individual a) an effective amount of a composition comprising nanoparticles comprising an mTOR inhibitor (such as a limus drug, e.g., sirolimus or a derivative thereof) and an albumin, wherein the nanoparticles have an average particle size of no greater than about 150 nm (such as no greater than about 120 nm); and b) an effective amount of a kinase inhibitor (such as a tyrosine kinase inhibitor, e.g., nilotinib or sorafenib). In some embodiments, the method comprises administering to the individual a) an effective amount of a composition comprising nanoparticles comprising an mTOR inhibitor (such as a limus drug, e.g., sirolimus or a derivative thereof) and an albumin, wherein the nanoparticles comprise the mTOR inhibitor associated (e.g., coated) with albumin, wherein the nanoparticles have an average particle size of no greater than about 150 nm (such as no greater than about 120 nm); and b) an effective amount of a kinase inhibitor (such as a tyrosine kinase inhibitor, e.g., nilotinib or sorafenib). In some embodiments, the method comprises administering to the individual a) an effective amount of a composition comprising nanoparticles comprising an mTOR inhibitor (such as a limus drug, e.g., sirolimus or a derivative thereof) and an albumin, wherein the nanoparticles comprise the mTOR inhibitor associated (e.g., coated) with the albumin, wherein the nanoparticles have an average particle size of no greater than about 150 nm (such as no greater than about 120 nm, for example about 100 nm), wherein the weight ratio of albumin and the mTOR inhibitor in the mTOR inhibitor nanoparticle composition is about 9:1 or less (such as about 9:1 or about 8:1); and b) an effective amount of a kinase inhibitor (such as a tyrosine kinase inhibitor, e.g., nilotinib or sorafenib). In some embodiments, the method further comprises administering to the individual at least one therapeutic agent used in a standard combination therapy with the kinase inhibitor. In some embodiments, the mTOR inhibitor is a limus drug. In some embodiments, the mTOR inhibitor is sirolimus or a derivative thereof. In some embodiments, the mTOR inhibitor nanoparticle composition comprises nab-sirolimus. In some embodiments, the mTOR inhibitor nanoparticle composition is nab-sirolimus. In some embodiments, the kinase inhibitor is a tyrosine kinase inhibitor. In some embodiments, the kinase inhibitor is a serine/threonine kinase inhibitor. In some embodiments, the kinase inhibitor is a Raf kinase inhibitor. In some embodiments, the kinase inhibitor inhibits more than one class of kinase (e.g., an inhibitor of more than one of a tyrosine kinase, a Raf kinase, and a serine/threonine kinase). In some embodiments, the kinase inhibitor is selected from the group consisting of erlotinib, imatinib, lapatinib, nilotinib, sorafenib, and sunitinib. In some embodiments, the kinase inhibitor is nilotinib. In some embodiments, the bladder cancer is recurrent bladder cancer. In some embodiments, the bladder cancer is refractory to one or more drugs used in a standard therapy for bladder cancer, such as, but not limited to, platinum-based agents, BCG, mitomycin C, and/or interferon.

In some embodiments, there is provided a method of treating bladder cancer (such as non-muscle invasive bladder cancer, e.g., BCG-refractory NMIBC) in an individual (such as a human) comprising administering to the individual a) an effective amount of a composition comprising nanoparticles comprising sirolimus or a derivative thereof and an albumin; and b) an effective amount of a kinase inhibitor (such as a tyrosine kinase inhibitor, e.g., nilotinib or sorafenib). In some embodiments, the method comprises administering to the individual a) an effective amount of a composition comprising nanoparticles comprising sirolimus or a derivative thereof and an albumin, wherein the sirolimus or derivative thereof in the nanoparticles is associated (e.g., coated) with the albumin; and b) an effective amount of a kinase inhibitor (such as a tyrosine kinase inhibitor, e.g., nilotinib or sorafenib). In some embodiments, the method comprises administering to the individual a) an effective amount of a composition comprising nanoparticles comprising sirolimus or a derivative thereof and an albumin, wherein the nanoparticles have an average particle size of no greater than about 150 nm (such as no greater than about 120 nm); and b) an effective amount of a kinase inhibitor (such as a tyrosine kinase inhibitor, e.g., nilotinib or sorafenib). In some embodiments, the method comprises administering to the individual a) an effective amount of a composition comprising nanoparticles comprising sirolimus or a derivative thereof and an albumin, wherein the nanoparticles comprise the sirolimus or derivative thereof associated (e.g., coated) with albumin, wherein the nanoparticles have an average particle size of no greater than about 150 nm (such as no greater than about 120 nm); and b) an effective amount of a kinase inhibitor (such as a tyrosine kinase inhibitor, e.g., nilotinib or sorafenib). In some embodiments, the method comprises administering to the individual a) an effective amount of a composition comprising nanoparticles comprising sirolimus or a derivative thereof and an albumin, wherein the nanoparticles comprise the sirolimus or derivative thereof associated (e.g., coated) with the albumin, wherein the nanoparticles have an average particle size of no greater than about 150 nm (such as no greater than about 120 nm, for example about 100 nm), wherein the weight ratio of albumin and the sirolimus or derivative thereof in the sirolimus nanoparticle composition is about 9:1 or less (such as about 9:1 or about 8:1); and b) an effective amount of a kinase inhibitor (such as a tyrosine kinase inhibitor, e.g., nilotinib or sorafenib). In some embodiments, the method further comprises administering to the individual at least one therapeutic agent used in a standard combination therapy with the kinase inhibitor. In some embodiments, the sirolimus or derivative thereof is sirolimus. In some embodiments, the sirolimus nanoparticle composition comprises nab-sirolimus. In some embodiments, the sirolimus nanoparticle composition is nab-sirolimus. In some embodiments, the kinase inhibitor is a tyrosine kinase inhibitor. In some embodiments, the kinase inhibitor is a serine/threonine kinase inhibitor. In some embodiments, the kinase inhibitor is a Raf kinase inhibitor. In some embodiments, the kinase inhibitor inhibits more than one class of kinase (e.g., an inhibitor of more than one of a tyrosine kinase, a Raf kinase, and a serine/threonine kinase). In some embodiments, the kinase inhibitor is selected from the group consisting of erlotinib, imatinib, lapatinib, nilotinib, sorafenib, and sunitinib. In some embodiments, the kinase inhibitor is nilotinib. In some embodiments, the bladder cancer is recurrent bladder cancer. In some embodiments, the bladder cancer is refractory to one or more drugs used in a standard therapy for bladder cancer, such as, but not limited to, platinum-based agents, BCG, mitomycin C, and/or interferon.

In some embodiments, there is provided a method of treating bladder cancer (such as non-muscle invasive bladder cancer, e.g., BCG-refractory NMIBC) in an individual (such as a human) comprising administering to the individual a) an effective amount of a composition comprising nanoparticles comprising an mTOR inhibitor (such as a limus drug, e.g., sirolimus or a derivative thereof) and an albumin; and b) an effective amount of a cancer vaccine. In some embodiments, the method comprises administering to the individual a) an effective amount of a composition comprising nanoparticles comprising an mTOR inhibitor (such as a limus drug, e.g., sirolimus or a derivative thereof) and an albumin, wherein the mTOR inhibitor in the nanoparticles is associated (e.g., coated) with the albumin; and b) an effective amount of a cancer vaccine. In some embodiments, the method comprises administering to the individual a) an effective amount of a composition comprising nanoparticles comprising an mTOR inhibitor (such as a limus drug, e.g., sirolimus or a derivative thereof) and an albumin, wherein the nanoparticles have an average particle size of no greater than about 150 nm (such as no greater than about 120 nm); and b) an effective amount of a cancer vaccine. In some embodiments, the method comprises administering to the individual a) an effective amount of a composition comprising nanoparticles comprising an mTOR inhibitor (such as a limus drug, e.g., sirolimus or a derivative thereof) and an albumin, wherein the nanoparticles comprise the mTOR inhibitor associated (e.g., coated) with albumin, wherein the nanoparticles have an average particle size of no greater than about 150 nm (such as no greater than about 120 nm); and b) an effective amount of a cancer vaccine. In some embodiments, the method comprises administering to the individual a) an effective amount of a composition comprising nanoparticles comprising an mTOR inhibitor (such as a limus drug, e.g., sirolimus or a derivative thereof) and an albumin, wherein the nanoparticles comprise the mTOR inhibitor associated (e.g., coated) with the albumin, wherein the nanoparticles have an average particle size of no greater than about 150 nm (such as no greater than about 120 nm, for example about 100 nm), wherein the weight ratio of albumin and the mTOR inhibitor in the mTOR inhibitor nanoparticle composition is about 9:1 or less (such as about 9:1 or about 8:1); and b) an effective amount of a cancer vaccine. In some embodiments, the method further comprises administering to the individual at least one therapeutic agent used in a standard combination therapy with the cancer vaccine. In some embodiments, the mTOR inhibitor is a limus drug. In some embodiments, the mTOR inhibitor is sirolimus or a derivative thereof. In some embodiments, the mTOR inhibitor nanoparticle composition comprises nab-sirolimus. In some embodiments, the mTOR inhibitor nanoparticle composition is nab-sirolimus. In some embodiments, the cancer vaccine is a vaccine prepared using autologous tumor cells. In some embodiments, the cancer vaccine is a vaccine prepared using allogeneic tumor cells. In some embodiments, the bladder cancer is recurrent bladder cancer. In some embodiments, the bladder cancer is refractory to one or more drugs used in a standard therapy for bladder cancer, such as, but not limited to, platinum-based agents, BCG, mitomycin C, and/or interferon.

In some embodiments, there is provided a method of treating bladder cancer (such as non-muscle invasive bladder cancer, e.g., BCG-refractory NMIBC) in an individual (such as a human) comprising administering to the individual a) an effective amount of a composition comprising nanoparticles comprising sirolimus or a derivative thereof and an albumin; and b) an effective amount of a cancer vaccine. In some embodiments, the method comprises administering to the individual a) an effective amount of a composition comprising nanoparticles comprising sirolimus or a derivative thereof and an albumin, wherein the sirolimus or derivative thereof in the nanoparticles is associated (e.g., coated) with the albumin; and b) an effective amount of a cancer vaccine. In some embodiments, the method comprises administering to the individual a) an effective amount of a composition comprising nanoparticles comprising sirolimus or a derivative thereof and an albumin, wherein the nanoparticles have an average particle size of no greater than about 150 nm (such as no greater than about 120 nm); and b) an effective amount of a cancer vaccine. In some embodiments, the method comprises administering to the individual a) an effective amount of a composition comprising nanoparticles comprising sirolimus or a derivative thereof and an albumin, wherein the nanoparticles comprise the sirolimus or derivative thereof associated (e.g., coated) with albumin, wherein the nanoparticles have an average particle size of no greater than about 150 nm (such as no greater than about 120 nm); and b) an effective amount of a cancer vaccine. In some embodiments, the method comprises administering to the individual a) an effective amount of a composition comprising nanoparticles comprising sirolimus or a derivative thereof and an albumin, wherein the nanoparticles comprise the sirolimus or derivative thereof associated (e.g., coated) with the albumin, wherein the nanoparticles have an average particle size of no greater than about 150 nm (such as no greater than about 120 nm, for example about 100 nm), wherein the weight ratio of albumin and the sirolimus or derivative thereof in the sirolimus nanoparticle composition is about 9:1 or less (such as about 9:1 or about 8:1); and b) an effective amount of a cancer vaccine. In some embodiments, the method further comprises administering to the individual at least one therapeutic agent used in a standard combination therapy with the cancer vaccine. In some embodiments, the sirolimus or derivative thereof is sirolimus. In some embodiments, the sirolimus nanoparticle composition comprises nab-sirolimus. In some embodiments, the sirolimus nanoparticle composition is nab-sirolimus. In some embodiments, the cancer vaccine is a vaccine prepared using autologous tumor cells. In some embodiments, the cancer vaccine is a vaccine prepared using allogeneic tumor cells. In some embodiments, the bladder cancer is recurrent bladder cancer. In some embodiments, the bladder cancer is refractory to one or more drugs used in a standard therapy for bladder cancer, such as, but not limited to, platinum-based agents, BCG, mitomycin C, and/or interferon.

In some embodiments, there is provided a method of treating bladder cancer (such as non-muscle invasive bladder cancer, e.g., BCG-refractory NMIBC) in an individual (such as a human) comprising administering to the individual a) an effective amount of a composition comprising nanoparticles comprising sirolimus or a derivative thereof and an albumin; and b) an effective amount of a second therapeutic agent selected from the group consisting of platinum-based agents, BCG, mitomycin C, and interferon. In some embodiments, the method comprises administering to the individual a) an effective amount of a composition comprising nanoparticles comprising sirolimus or a derivative thereof and an albumin, wherein the sirolimus or derivative thereof in the nanoparticles is associated (e.g., coated) with the albumin; and b) an effective amount of a second therapeutic agent selected from the group consisting of platinum-based agents, BCG, mitomycin C, and interferon. In some embodiments, the method comprises administering to the individual a) an effective amount of a composition comprising nanoparticles comprising sirolimus or a derivative thereof and an albumin, wherein the nanoparticles have an average particle size of no greater than about 150 nm (such as no greater than about 120 nm); and b) an effective amount of a second therapeutic agent selected from the group consisting of platinum-based agents, BCG, mitomycin C, and interferon. In some embodiments, the method comprises administering to the individual a) an effective amount of a composition comprising nanoparticles comprising sirolimus or a derivative thereof and an albumin, wherein the nanoparticles comprise the sirolimus or derivative thereof associated (e.g., coated) with albumin, wherein the nanoparticles have an average particle size of no greater than about 150 nm (such as no greater than about 120 nm); and b) an effective amount of a second therapeutic agent selected from the group consisting of platinum-based agents, BCG, mitomycin C, and interferon. In some embodiments, the method comprises administering to the individual a) an effective amount of a composition comprising nanoparticles comprising sirolimus or a derivative thereof and an albumin, wherein the nanoparticles comprise the sirolimus or derivative thereof associated (e.g., coated) with the albumin, wherein the nanoparticles have an average particle size of no greater than about 150 nm (such as no greater than about 120 nm, for example about 100 nm), wherein the weight ratio of albumin and the sirolimus or derivative thereof in the sirolimus nanoparticle composition is about 9:1 or less (such as about 9:1 or about 8:1); and b) an effective amount of a second therapeutic agent selected from the group consisting of platinum-based agents, BCG, mitomycin C, and interferon. In some embodiments, the method further comprises administering to the individual at least one therapeutic agent used in a standard combination therapy with the second therapeutic agent. In some embodiments, the sirolimus or derivative thereof is sirolimus. In some embodiments, the sirolimus nanoparticle composition comprises nab-sirolimus. In some embodiments, the sirolimus nanoparticle composition is nab-sirolimus. In some embodiments, the bladder cancer is recurrent bladder cancer. In some embodiments, the bladder cancer is refractory to one or more drugs used in a standard therapy for bladder cancer, such as, but not limited to, platinum-based agents, BCG, mitomycin C, and/or interferon.

In some embodiments, according to any of the methods of treating bladder cancer (such as non-muscle invasive bladder cancer, e.g., BCG-refractory NMIBC) in an individual described herein, the individual is a human who exhibits one or more symptoms associated with bladder cancer. In some embodiments, the individual is at an early stage of bladder cancer. In some embodiments, the individual is at an advanced stage of bladder cancer. In some of embodiments, the individual is genetically or otherwise predisposed (e.g., having a risk factor) to developing bladder cancer. Individuals at risk for bladder cancer include, e.g., those having relatives who have experienced bladder cancer, and those whose risk is determined by analysis of genetic or biochemical markers. In some embodiments, the individual may be a human who has a gene, genetic mutation, or polymorphism associated with bladder cancer (e.g., HRAS, KRAS2, RB1, or FGFR3) or has one or more extra copies of a gene associated with bladder cancer. In some embodiments, the individual has a ras or PTEN mutation. In some embodiments, the cancer cells are dependent on an mTOR pathway to translate one or more mRNAs. In some embodiments, the cancer cells are not capable of synthesizing mRNAs by an mTOR-independent pathway. In some embodiments, the cancer cells have decreased or no PTEN activity or have decreased or no expression of PTEN compared to non-cancerous cells. In some embodiments, the individual has at least one tumor biomarker selected from the group consisting of elevated PI3K activity, elevated mTOR activity, presence of FLT-3ITD, elevated AKT activity, elevated KRAS activity, and elevated NRAS activity. In some embodiments, the individual has a variation in at least one gene selected from the group consisting of drug metabolism genes, cancer genes, and drug target genes.

### Renal Cell Carcinoma

In some embodiments, there is provided a method of treating renal cell carcinoma in an individual (such as a human) comprising administering to the individual a) an effective amount of a composition comprising nanoparticles comprising an mTOR inhibitor (such as a limus drug, e.g., sirolimus or a derivative thereof) and an albumin; and b) an effective amount of a second therapeutic agent. In some embodiments, the method comprises administering to the individual a) an effective amount of a composition comprising nanoparticles comprising an mTOR inhibitor (such as a limus drug, e.g., sirolimus or a derivative thereof) and an albumin, wherein the mTOR inhibitor in the nanoparticles is associated (e.g., coated) with the albumin; and b) an effective amount of a second therapeutic agent. In some embodiments, the method comprises administering to the individual a) an effective amount of a composition comprising nanoparticles comprising an mTOR inhibitor (such as a limus drug, e.g., sirolimus or a derivative thereof) and an albumin, wherein the nanoparticles have an average particle size of no greater than about 150 nm (such as no greater than about 120 nm); and b) an effective amount of a second therapeutic agent. In some embodiments, the method comprises administering to the individual a) an effective amount of a composition comprising nanoparticles comprising an mTOR inhibitor (such as a limus drug, e.g., sirolimus or a derivative thereof) and an albumin, wherein the nanoparticles comprise the mTOR inhibitor associated (e.g., coated) with albumin, wherein the nanoparticles have an average particle size of no greater than about 150 nm (such as no greater than about 120 nm); and b) an effective amount of a second therapeutic agent. In some embodiments, the method comprises administering to the individual a) an effective amount of a composition comprising nanoparticles comprising an mTOR inhibitor (such as a limus drug, e.g., sirolimus or a derivative thereof) and an albumin, wherein the nanoparticles comprise the mTOR inhibitor associated (e.g., coated) with the albumin, wherein the nanoparticles have an average particle size of no greater than about 150 nm (such as no greater than about 120 nm, for example about 100 nm), wherein the weight ratio of albumin and the mTOR inhibitor in the mTOR inhibitor nanoparticle composition is about 9:1 or less (such as about 9:1 or about 8:1); and b) an effective amount of a second therapeutic agent. In some embodiments, the method further comprises administering to the individual at least one therapeutic agent used in a standard combination therapy with the second therapeutic agent. In some embodiments, the mTOR inhibitor is a limus drug. In some embodiments, the mTOR inhibitor is sirolimus or a derivative thereof. In some embodiments, the mTOR inhibitor nanoparticle composition comprises nab-sirolimus. In some embodiments, the mTOR inhibitor nanoparticle composition is nab-sirolimus. In some embodiments, the second therapeutic agent is selected from the group consisting of an immunomodulator (such as an immunostimulator or an immune checkpoint inhibitor), a histone deacetylase inhibitor, a kinase inhibitor (such as a tyrosine kinase inhibitor), and a cancer vaccine (such as a vaccine prepared using tumor cells or at least one tumor-associated antigen). In some embodiments, the second therapeutic agent is an immunomodulator. In some embodiments, the immunomodulator is an immunostimulator that directly stimulates the immune system of an individual. In some embodiments, the immunomodulator is an agonistic antibody that targets an activating receptor on an immune cell (such as a T cell). In some embodiments, the immunomodulator is an immune checkpoint inhibitor. In some embodiments, the immune checkpoint inhibitor is an antagonistic antibody that targets an immune checkpoint protein. In some embodiments, the immunomodulator is an IMiDs® compound (small molecule immunomodulator, such as lenalidomide or pomalidomide). In some embodiments, the immunomodulator is lenalidomide. In some embodiments, the immunomodulator is pomalidomide. In some embodiments, the immunomodulator is small molecule or antibody-based IDO inhibitor. In some embodiments, the second therapeutic agent is a histone deacetylase inhibitor. In some embodiments, the histone deacetylase inhibitor is specific to only one HDAC. In some embodiments, the histone deacetylase inhibitor is specific to only one class of HDAC. In some embodiments, the histone deacetylase inhibitor is specific to two or more HDACs or two or more classes of HDACs. In some embodiments, the histone deacetylase inhibitor is specific to class I and II HDACs. In some embodiments, the histone deacetylase inhibitor is specific to class III HDACs. In some embodiments, the histone deacetylase inhibitor is selected from the group consisting of romidepsin, panobinostat, ricolinostat, and belinostat. In some embodiments, the histone deacetylase inhibitor is romidepsin. In some embodiments, the second therapeutic agent is a kinase inhibitor, such as a tyrosine kinase inhibitor. In some embodiments, the kinase inhibitor is a serine/threonine kinase inhibitor. In some embodiments, the kinase inhibitor is a Raf kinase inhibitor. In some embodiments, the kinase inhibitor inhibits more than one class of kinase (e.g., an inhibitor of more than one of a tyrosine kinase, a Raf kinase, and a serine/threonine kinase). In some embodiments, the kinase inhibitor is selected from the group consisting of erlotinib, imatinib, lapatinib, nilotinib, sorafenib, and sunitinib. In some embodiments, the kinase inhibitor is sorafenib. In some embodiments, the kinase inhibitor is nilotinib. In some embodiments, the second therapeutic agent is a cancer vaccine, such as a vaccine prepared using tumor cells or at least one tumor-associated antigen. In some embodiments, the second therapeutic agent and the nanoparticle composition are administered sequentially. In some embodiments, the second therapeutic agent and the nanoparticle composition are administered simultaneously. In some embodiments, the second therapeutic agent and the nanoparticle composition are administered concurrently.

In some embodiments, the renal cell carcinoma (also called kidney cancer, renal adenocarcinoma, or hypernephroma) is an adenocarcinoma. In some embodiments, the renal cell carcinoma is a clear cell renal cell carcinoma, papillary renal cell carcinoma (also called chromophilic renal cell carcinoma), chromophobe renal cell carcinoma, collecting duct renal cell carcinoma, granular renal cell carcinoma, mixed granular renal cell carcinoma, renal angiomyolipomas, or spindle renal cell carcinoma. In some embodiments, the individual may be a human who has a gene, genetic mutation, or polymorphism associated with renal cell carcinoma (e.g., VHL, TSC1, TSC2, CUL2, MSH2, MLH1, INK4a/ARF, MET, TGF-α, TGF-β1, IGF-I, IGF-IR, AKT, and/or PTEN) or has one or more extra copies of a gene associated with renal cell carcinoma. In some embodiments, the renal cell carcinoma is associated with (1) von Hippel-Lindau (VHL) syndrome, (2) hereditary papillary renal carcinoma (HPRC), (3) familial renal oncocytoma (FRO) associated with Birt-Hogg-Dube syndrome (BHDS), or (4) hereditary renal carcinoma (HRC). There are provided methods of treating renal cell carcinoma at any of the four stages, I, II, III, or IV, according to the American Joint Committee on Cancer (AJCC) staging groups. In some embodiments, the renal cell carcinoma is stage IV renal cell carcinoma.

In some embodiments, there is provided a method of treating renal cell carcinoma in an individual (such as a human) comprising administering to the individual a) an effective amount of a composition comprising nanoparticles comprising an mTOR inhibitor (such as a limus drug, e.g., sirolimus or a derivative thereof) and an albumin; and b) an effective amount of an immunomodulator (such as lenalidomide, pomalidomide, or an immune checkpoint inhibitor). In some embodiments, the method comprises administering to the individual a) an effective amount of a composition comprising nanoparticles comprising an mTOR inhibitor (such as a limus drug, e.g., sirolimus or a derivative thereof) and an albumin, wherein the mTOR inhibitor in the nanoparticles is associated (e.g., coated) with the albumin; and b) an effective amount of an immunomodulator (such as lenalidomide, pomalidomide, or an immune checkpoint inhibitor). In some embodiments, the method comprises administering to the individual a) an effective amount of a composition comprising nanoparticles comprising an mTOR inhibitor (such as a limus drug, e.g., sirolimus or a derivative thereof) and an albumin, wherein the nanoparticles have an average particle size of no greater than about 150 nm (such as no greater than about 120 nm); and b) an effective amount of an immunomodulator (such as lenalidomide, pomalidomide, or an immune checkpoint inhibitor). In some embodiments, the method comprises administering to the individual a) an effective amount of a composition comprising nanoparticles comprising an mTOR inhibitor (such as a limus drug, e.g., sirolimus or a derivative thereof) and an albumin, wherein the nanoparticles comprise the mTOR inhibitor associated (e.g., coated) with albumin, wherein the nanoparticles have an average particle size of no greater than about 150 nm (such as no greater than about 120 nm); and b) an effective amount of an immunomodulator (such as lenalidomide, pomalidomide, or an immune checkpoint inhibitor). In some embodiments, the method comprises administering to the individual a) an effective amount of a composition comprising nanoparticles comprising an mTOR inhibitor (such as a limus drug, e.g., sirolimus or a derivative thereof) and an albumin, wherein the nanoparticles comprise the mTOR inhibitor associated (e.g., coated) with the albumin, wherein the nanoparticles have an average particle size of no greater than about 150 nm (such as no greater than about 120 nm, for example about 100 nm), wherein the weight ratio of albumin and the mTOR inhibitor in the mTOR inhibitor nanoparticle composition is about 9:1 or less (such as about 9:1 or about 8:1); and b) an effective amount of an immunomodulator (such as lenalidomide, pomalidomide, or an immune checkpoint inhibitor). In some embodiments, the method further comprises administering to the individual at least one therapeutic agent used in a standard combination therapy with the immunomodulator. In some embodiments, the mTOR inhibitor is a limus drug. In some embodiments, the mTOR inhibitor is sirolimus or a derivative thereof. In some embodiments, the mTOR inhibitor nanoparticle composition comprises nab-sirolimus. In some embodiments, the mTOR inhibitor nanoparticle composition is nab-sirolimus. In some embodiments, the immunomodulator is an immunostimulator that directly stimulates the immune system of an individual. In some embodiments, the immunomodulator is an agonistic antibody that targets an activating receptor on an immune cell (such as a T cell). In some embodiments, the immunomodulator is an immune checkpoint inhibitor. In some embodiments, the immune checkpoint inhibitor is an antagonistic antibody that targets an immune checkpoint protein. In some embodiments, the immunomodulator is an IMiDs® compound (small molecule immunomodulator, such as lenalidomide or pomalidomide). In some embodiments, the immunomodulator is lenalidomide. In some embodiments, the immunomodulator is pomalidomide. In some embodiments, the immunomodulator is small molecule or antibody-based IDO inhibitor. In some embodiments, the renal cell carcinoma is recurrent renal cell carcinoma. In some embodiments, the renal cell carcinoma is refractory to one or more drugs used in a standard therapy for renal cell carcinoma, such as, but not limited to, Afinitor (everolimus), temsirolimus, aldesleukin, Avastin (bevacizumab), axitinib, sorafenib, sunitinib, and/or Votrient (pazopanib hydrochloride).

In some embodiments, there is provided a method of treating renal cell carcinoma in an individual (such as a human) comprising administering to the individual a) an effective amount of a composition comprising nanoparticles comprising sirolimus or a derivative thereof and an albumin; and b) an effective amount of an immunomodulator (such as an immunostimulator, e.g., pomalidomide). In some embodiments, the method comprises administering to the individual a) an effective amount of a composition comprising nanoparticles comprising sirolimus or a derivative thereof and an albumin, wherein the sirolimus or derivative thereof in the nanoparticles is associated (e.g., coated) with the albumin; and b) an effective amount of an immunomodulator (such as an immunostimulator, e.g., pomalidomide). In some embodiments, the method comprises administering to the individual a) an effective amount of a composition comprising nanoparticles comprising sirolimus or a derivative thereof and an albumin, wherein the nanoparticles have an average particle size of no greater than about 150 nm (such as no greater than about 120 nm); and b) an effective amount of an immunomodulator (such as an immunostimulator, e.g., pomalidomide). In some embodiments, the method comprises administering to the individual a) an effective amount of a composition comprising nanoparticles comprising sirolimus or a derivative thereof and an albumin, wherein the nanoparticles comprise the sirolimus or derivative thereof associated (e.g., coated) with albumin, wherein the nanoparticles have an average particle size of no greater than about 150 nm (such as no greater than about 120 nm); and b) an effective amount of an immunomodulator (such as an immunostimulator, e.g., pomalidomide). In some embodiments, the method comprises administering to the individual a) an effective amount of a composition comprising nanoparticles comprising sirolimus or a derivative thereof and albumin, wherein the nanoparticles comprise the sirolimus or derivative thereof associated (e.g., coated) with the albumin, wherein the nanoparticles have an average particle size of no greater than about 150 nm (such as no greater than about 120 nm, for example about 100 nm), wherein the weight ratio of albumin and sirolimus or a derivative thereof in the sirolimus nanoparticle composition is about 9:1 or less (such as about 9:1 or about 8:1); and b) an effective amount of an immunomodulator (such as an immunostimulator, e.g., pomalidomide). In some embodiments, the method further comprises administering to the individual at least one therapeutic agent used in a standard combination therapy with the immunomodulator. In some embodiments, the sirolimus or derivative thereof is sirolimus. In some embodiments, the sirolimus nanoparticle composition comprises nab-sirolimus. In some embodiments, the sirolimus nanoparticle composition is nab-sirolimus. In some embodiments, the immunomodulator is an immunostimulator that directly stimulates the immune system of an individual. In some embodiments, the immunomodulator is an agonistic antibody that targets an activating receptor on an immune cell (such as a T cell). In some embodiments, the immunomodulator is an immune checkpoint inhibitor. In some embodiments, the immune checkpoint inhibitor is an antagonistic antibody that targets an immune checkpoint protein. In some embodiments, the immunomodulator is an IMiDs® compound (small molecule immunomodulator, such as lenalidomide or pomalidomide). In some embodiments, the immunomodulator is lenalidomide. In some embodiments, the immunomodulator is pomalidomide. In some embodiments, the immunomodulator is small molecule or antibody-based IDO inhibitor. In some embodiments, the renal cell carcinoma is recurrent renal cell carcinoma. In some embodiments, the renal cell carcinoma is refractory to one or more drugs used in a standard therapy for renal cell carcinoma, such as, but not limited to, Afinitor (everolimus), temsirolimus, aldesleukin, Avastin (bevacizumab), axitinib, sorafenib, sunitinib, and/or Votrient (pazopanib hydrochloride).

In some embodiments, there is provided a method of treating renal cell carcinoma in an individual (such as a human) comprising administering to the individual a) an effective amount of a composition comprising nanoparticles comprising an mTOR inhibitor (such as a limus drug, e.g., sirolimus or a derivative thereof) and an albumin; and b) an effective amount of a histone deacetylase inhibitor (such as romidepsin). In some embodiments, the method comprises administering to the individual a) an effective amount of a composition comprising nanoparticles comprising an mTOR inhibitor (such as a limus drug, e.g., sirolimus or a derivative thereof) and an albumin, wherein the mTOR inhibitor in the nanoparticles is associated (e.g., coated) with the albumin; and b) an effective amount of a histone deacetylase inhibitor (such as romidepsin). In some embodiments, the method comprises administering to the individual a) an effective amount of a composition comprising nanoparticles comprising an mTOR inhibitor (such as a limus drug, e.g., sirolimus or a derivative thereof) and an albumin, wherein the nanoparticles have an average particle size of no greater than about 150 nm (such as no greater than about 120 nm); and b) an effective amount of a histone deacetylase inhibitor (such as romidepsin). In some embodiments, the method comprises administering to the individual a) an effective amount of a composition comprising nanoparticles comprising an mTOR inhibitor (such as a limus drug, e.g., sirolimus or a derivative thereof) and an albumin, wherein the nanoparticles comprise the mTOR inhibitor associated (e.g., coated) with albumin, wherein the nanoparticles have an average particle size of no greater than about 150 nm (such as no greater than about 120 nm); and b) an effective amount of a histone deacetylase inhibitor (such as romidepsin). In some embodiments, the method comprises administering to the individual a) an effective amount of a composition comprising nanoparticles comprising an mTOR inhibitor (such as a limus drug, e.g., sirolimus or a derivative thereof) and an albumin, wherein the nanoparticles comprise the mTOR inhibitor associated (e.g., coated) with the albumin, wherein the nanoparticles have an average particle size of no greater than about 150 nm (such as no greater than about 120 nm, for example about 100 nm), wherein the weight ratio of albumin and the mTOR inhibitor in the mTOR inhibitor nanoparticle composition is about 9:1 or less (such as about 9:1 or about 8:1); and b) an effective amount of a histone deacetylase inhibitor (such as romidepsin). In some embodiments, the method further comprises administering to the individual at least one therapeutic agent used in a standard combination therapy with the histone deacetylase inhibitor.

In some embodiments, the mTOR inhibitor is a limus drug. In some embodiments, the mTOR inhibitor is sirolimus or a derivative thereof. In some embodiments, the mTOR inhibitor nanoparticle composition comprises nab-sirolimus. In some embodiments, the mTOR inhibitor nanoparticle composition is nab-sirolimus. In some embodiments, the histone deacetylase inhibitor is selected from the group consisting of romidepsin, panobinostat, ricolinostat, and belinostat. In some embodiments, the histone deacetylase inhibitor is romidepsin. In some embodiments, the renal cell carcinoma is recurrent renal cell carcinoma. In some embodiments, the renal cell carcinoma is refractory to one or more drugs used in a standard therapy for renal cell carcinoma, such as, but not limited to, Afinitor (everolimus), temsirolimus, aldesleukin, Avastin (bevacizumab), axitinib, sorafenib, sunitinib, and/or Votrient (pazopanib hydrochloride).

In some embodiments, there is provided a method of treating renal cell carcinoma in an individual (such as a human) comprising administering to the individual a) an effective amount of a composition comprising nanoparticles comprising sirolimus or a derivative thereof and an albumin; and b) an effective amount of a histone deacetylase inhibitor (such as romidepsin). In some embodiments, the method comprises administering to the individual a) an effective amount of a composition comprising nanoparticles comprising sirolimus or a derivative thereof and an albumin, wherein the sirolimus or derivative thereof in the nanoparticles is associated (e.g., coated) with the albumin; and b) an effective amount of a histone deacetylase inhibitor (such as romidepsin). In some embodiments, the method comprises administering to the individual a) an effective amount of a composition comprising nanoparticles comprising sirolimus or a derivative thereof and an albumin, wherein the nanoparticles have an average particle size of no greater than about 150 nm (such as no greater than about 120 nm); and b) an effective amount of a histone deacetylase inhibitor (such as romidepsin). In some embodiments, the method comprises administering to the individual a) an effective amount of a composition comprising nanoparticles comprising sirolimus or a derivative thereof and an albumin, wherein the nanoparticles comprise the sirolimus or derivative thereof associated (e.g., coated) with albumin, wherein the nanoparticles have an average particle size of no greater than about 150 nm (such as no greater than about 120 nm); and b) an effective amount of a histone deacetylase inhibitor (such as romidepsin). In some embodiments, the method comprises administering to the individual a) an effective amount of a composition comprising nanoparticles comprising sirolimus or a derivative thereof and albumin, wherein the nanoparticles comprise the sirolimus or derivative thereof associated (e.g., coated) with the albumin, wherein the nanoparticles have an average particle size of no greater than about 150 nm (such as no greater than about 120 nm, for example about 100 nm), wherein the weight ratio of albumin and sirolimus or a derivative thereof in the sirolimus nanoparticle composition is about 9:1 or less (such as about 9:1 or about 8:1); and b) an effective amount of a histone deacetylase inhibitor (such as romidepsin). In some embodiments, the method further comprises administering to the individual at least one therapeutic agent used in a standard combination therapy with the histone deacetylase inhibitor. In some embodiments, the sirolimus or derivative thereof is sirolimus. In some embodiments, the sirolimus nanoparticle composition comprises nab-sirolimus. In some embodiments, the sirolimus nanoparticle composition is nab-sirolimus. In some embodiments, the histone deacetylase inhibitor is selected from the group consisting of romidepsin, panobinostat, ricolinostat, and belinostat. In some embodiments, the histone deacetylase inhibitor is romidepsin. In some embodiments, the renal cell carcinoma is recurrent renal cell carcinoma. In some embodiments, the renal cell carcinoma is refractory to one or more drugs used in a standard therapy for renal cell carcinoma, such as, but not limited to, Afinitor (everolimus), temsirolimus, aldesleukin, Avastin (bevacizumab), axitinib, sorafenib, sunitinib, and/or Votrient (pazopanib hydrochloride).

In some embodiments, there is provided a method of treating renal cell carcinoma in an individual (such as a human) comprising administering to the individual a) an effective amount of a composition comprising nanoparticles comprising an mTOR inhibitor (such as a limus drug, e.g., sirolimus or a derivative thereof) and an albumin; and b) an effective amount of a kinase inhibitor (such as a tyrosine kinase inhibitor, e.g., nilotinib or sorafenib). In some embodiments, the method comprises administering to the individual a) an effective amount of a composition comprising nanoparticles comprising an mTOR inhibitor (such as a limus drug, e.g., sirolimus or a derivative thereof) and an albumin, wherein the mTOR inhibitor in the nanoparticles is associated (e.g., coated) with the albumin; and b) an effective amount of a kinase inhibitor (such as a tyrosine kinase inhibitor, e.g., nilotinib or sorafenib). In some embodiments, the method comprises administering to the individual a) an effective amount of a composition comprising nanoparticles comprising an mTOR inhibitor (such as a limus drug, e.g., sirolimus or a derivative thereof) and an albumin, wherein the nanoparticles have an average particle size of no greater than about 150 nm (such as no greater than about 120 nm); and b) an effective amount of a kinase inhibitor (such as a tyrosine kinase inhibitor, e.g., nilotinib or sorafenib). In some embodiments, the method comprises administering to the individual a) an effective amount of a composition comprising nanoparticles comprising an mTOR inhibitor (such as a limus drug, e.g., sirolimus or a derivative thereof) and an albumin, wherein the nanoparticles comprise the mTOR inhibitor associated (e.g., coated) with albumin, wherein the nanoparticles have an average particle size of no greater than about 150 nm (such as no greater than about 120 nm); and b) an effective amount of a kinase inhibitor (such as a tyrosine kinase inhibitor, e.g., nilotinib or sorafenib). In some embodiments, the method comprises administering to the individual a) an effective amount of a composition comprising nanoparticles comprising an mTOR inhibitor (such as a limus drug, e.g., sirolimus or a derivative thereof) and an albumin, wherein the nanoparticles comprise the mTOR inhibitor associated (e.g., coated) with the albumin, wherein the nanoparticles have an average particle size of no greater than about 150 nm (such as no greater than about 120 nm, for example about 100 nm), wherein the weight ratio of albumin and the mTOR inhibitor in the mTOR inhibitor nanoparticle composition is about 9:1 or less (such as about 9:1 or about 8:1); and b) an effective amount of a kinase inhibitor (such as a tyrosine kinase inhibitor, e.g., nilotinib or sorafenib). In some embodiments, the method further comprises administering to the individual at least one therapeutic agent used in a standard combination therapy with the kinase inhibitor. In some embodiments, the mTOR inhibitor is a limus drug. In some embodiments, the mTOR inhibitor is sirolimus or a derivative thereof. In some embodiments, the mTOR inhibitor nanoparticle composition comprises nab-sirolimus. In some embodiments, the mTOR inhibitor nanoparticle composition is nab-sirolimus. In some embodiments, the kinase inhibitor is a tyrosine kinase inhibitor. In some embodiments, the kinase inhibitor is a serine/threonine kinase inhibitor. In some embodiments, the kinase inhibitor is a Raf kinase inhibitor. In some embodiments, the kinase inhibitor inhibits more than one class of kinase (e.g., an inhibitor of more than one of a tyrosine kinase, a Raf kinase, and a serine/threonine kinase). In some embodiments, the kinase inhibitor is selected from the group consisting of erlotinib, imatinib, lapatinib, nilotinib, sorafenib, and sunitinib. In some embodiments, the kinase inhibitor is nilotinib. In some embodiments, the renal cell carcinoma is recurrent renal cell carcinoma. In some embodiments, the renal cell carcinoma is refractory to one or more drugs used in a standard therapy for renal cell carcinoma, such as, but not limited to, Afinitor (everolimus), temsirolimus, aldesleukin, Avastin (bevacizumab), axitinib, sorafenib, sunitinib, and/or Votrient (pazopanib hydrochloride).

In some embodiments, there is provided a method of treating renal cell carcinoma in an individual (such as a human) comprising administering to the individual a) an effective amount of a composition comprising nanoparticles comprising sirolimus or a derivative thereof and an albumin; and b) an effective amount of a kinase inhibitor (such as a tyrosine kinase inhibitor, e.g., nilotinib or sorafenib). In some embodiments, the method comprises administering to the individual a) an effective amount of a composition comprising nanoparticles comprising sirolimus or a derivative thereof and an albumin, wherein the sirolimus or derivative thereof in the nanoparticles is associated (e.g., coated) with the albumin; and b) an effective amount of a kinase inhibitor (such as a tyrosine kinase inhibitor, e.g., nilotinib or sorafenib). In some embodiments, the method comprises administering to the individual a) an effective amount of a composition comprising nanoparticles comprising sirolimus or a derivative thereof and an albumin, wherein the nanoparticles have an average particle size of no greater than about 150 nm (such as no greater than about 120 nm); and b) an effective amount of a kinase inhibitor (such as a tyrosine kinase inhibitor, e.g., nilotinib or sorafenib). In some embodiments, the method comprises administering to the individual a) an effective amount of a composition comprising nanoparticles comprising sirolimus or a derivative thereof and an albumin, wherein the nanoparticles comprise the sirolimus or derivative thereof associated (e.g., coated) with albumin, wherein the nanoparticles have an average particle size of no greater than about 150 nm (such as no greater than about 120 nm); and b) an effective amount of a kinase inhibitor (such as a tyrosine kinase inhibitor, e.g., nilotinib or sorafenib). In some embodiments, the method comprises administering to the individual a) an effective amount of a composition comprising nanoparticles comprising sirolimus or a derivative thereof and an albumin, wherein the nanoparticles comprise the sirolimus or derivative thereof associated (e.g., coated) with the albumin, wherein the nanoparticles have an average particle size of no greater than about 150 nm (such as no greater than about 120 nm, for example about 100 nm), wherein the weight ratio of albumin and the sirolimus or derivative thereof in the sirolimus nanoparticle composition is about 9:1 or less (such as about 9:1 or about 8:1); and b) an effective amount of a kinase inhibitor (such as a tyrosine kinase inhibitor, e.g., nilotinib or sorafenib). In some embodiments, the method further comprises administering to the individual at least one therapeutic agent used in a standard combination therapy with the kinase inhibitor. In some embodiments, the sirolimus or derivative thereof is sirolimus. In some embodiments, the sirolimus nanoparticle composition comprises nab-sirolimus. In some embodiments, the sirolimus nanoparticle composition is nab-sirolimus. In some embodiments, the kinase inhibitor is a tyrosine kinase inhibitor. In some embodiments, the kinase inhibitor is a serine/threonine kinase inhibitor. In some embodiments, the kinase inhibitor is a Raf kinase inhibitor. In some embodiments, the kinase inhibitor inhibits more than one class of kinase (e.g., an inhibitor of more than one of a tyrosine kinase, a Raf kinase, and a serine/threonine kinase). In some embodiments, the kinase inhibitor is selected from the group consisting of erlotinib, imatinib, lapatinib, nilotinib, sorafenib, and sunitinib. In some embodiments, the kinase inhibitor is nilotinib. In some embodiments, the renal cell carcinoma is recurrent renal cell carcinoma. In some embodiments, the renal cell carcinoma is refractory to one or more drugs used in a standard therapy for renal cell carcinoma, such as, but not limited to, Afinitor (everolimus), temsirolimus, aldesleukin, Avastin (bevacizumab), axitinib, sorafenib, sunitinib, and/or Votrient (pazopanib hydrochloride).

In some embodiments, there is provided a method of treating renal cell carcinoma in an individual (such as a human) comprising administering to the individual a) an effective amount of a composition comprising nanoparticles comprising an mTOR inhibitor (such as a limus drug, e.g., sirolimus or a derivative thereof) and an albumin; and b) an effective amount of a cancer vaccine. In some embodiments, the method comprises administering to the individual a) an effective amount of a composition comprising nanoparticles comprising an mTOR inhibitor (such as a limus drug, e.g., sirolimus or a derivative thereof) and an albumin, wherein the mTOR inhibitor in the nanoparticles is associated (e.g., coated) with the albumin; and b) an effective amount of a cancer vaccine. In some embodiments, the method comprises administering to the individual a) an effective amount of a composition comprising nanoparticles comprising an mTOR inhibitor (such as a limus drug, e.g., sirolimus or a derivative thereof) and an albumin, wherein the nanoparticles have an average particle size of no greater than about 150 nm (such as no greater than about 120 nm); and b) an effective amount of a cancer vaccine. In some embodiments, the method comprises administering to the individual a) an effective amount of a composition comprising nanoparticles comprising an mTOR inhibitor (such as a limus drug, e.g., sirolimus or a derivative thereof) and an albumin, wherein the nanoparticles comprise the mTOR inhibitor associated (e.g., coated) with albumin, wherein the nanoparticles have an average particle size of no greater than about 150 nm (such as no greater than about 120 nm); and b) an effective amount of a cancer vaccine. In some embodiments, the method comprises administering to the individual a) an effective amount of a composition comprising nanoparticles comprising an mTOR inhibitor (such as a limus drug, e.g., sirolimus or a derivative thereof) and an albumin, wherein the nanoparticles comprise the mTOR inhibitor associated (e.g., coated) with the albumin, wherein the nanoparticles have an average particle size of no greater than about 150 nm (such as no greater than about 120 nm, for example about 100 nm), wherein the weight ratio of albumin and the mTOR inhibitor in the mTOR inhibitor nanoparticle composition is about 9:1 or less (such as about 9:1 or about 8:1); and b) an effective amount of a cancer vaccine. In some embodiments, the method further comprises administering to the individual at least one therapeutic agent used in a standard combination therapy with the cancer vaccine. In some embodiments, the mTOR inhibitor is a limus drug. In some embodiments, the mTOR inhibitor is sirolimus or a derivative thereof. In some embodiments, the mTOR inhibitor nanoparticle composition comprises nab-sirolimus. In some embodiments, the mTOR inhibitor nanoparticle composition is nab-sirolimus. In some embodiments, the cancer vaccine is a vaccine prepared using autologous tumor cells. In some embodiments, the cancer vaccine is a vaccine prepared using allogeneic tumor cells. In some embodiments, the renal cell carcinoma is recurrent renal cell carcinoma. In some embodiments, the renal cell carcinoma is refractory to one or more drugs used in a standard therapy for renal cell carcinoma, such as, but not limited to, Afinitor (everolimus), temsirolimus, aldesleukin, Avastin (bevacizumab), axitinib, sorafenib, sunitinib, and/or Votrient (pazopanib hydrochloride).

In some embodiments, there is provided a method of treating renal cell carcinoma in an individual (such as a human) comprising administering to the individual a) an effective amount of a composition comprising nanoparticles comprising sirolimus or a derivative thereof and an albumin; and b) an effective amount of a cancer vaccine. In some embodiments, the method comprises administering to the individual a) an effective amount of a composition comprising nanoparticles comprising sirolimus or a derivative thereof and an albumin, wherein the sirolimus or derivative thereof in the nanoparticles is associated (e.g., coated) with the albumin; and b) an effective amount of a cancer vaccine. In some embodiments, the method comprises administering to the individual a) an effective amount of a composition comprising nanoparticles comprising sirolimus or a derivative thereof and an albumin, wherein the nanoparticles have an average particle size of no greater than about 150 nm (such as no greater than about 120 nm); and b) an effective amount of a cancer vaccine. In some embodiments, the method comprises administering to the individual a) an effective amount of a composition comprising nanoparticles comprising sirolimus or a derivative thereof and an albumin, wherein the nanoparticles comprise the sirolimus or derivative thereof associated (e.g., coated) with albumin, wherein the nanoparticles have an average particle size of no greater than about 150 nm (such as no greater than about 120 nm); and b) an effective amount of a cancer vaccine. In some embodiments, the method comprises administering to the individual a) an effective amount of a composition comprising nanoparticles comprising sirolimus or a derivative thereof and an albumin, wherein the nanoparticles comprise the sirolimus or derivative thereof associated (e.g., coated) with the albumin, wherein the nanoparticles have an average particle size of no greater than about 150 nm (such as no greater than about 120 nm, for example about 100 nm), wherein the weight ratio of albumin and the sirolimus or derivative thereof in the sirolimus nanoparticle composition is about 9:1 or less (such as about 9:1 or about 8:1); and b) an effective amount of a cancer vaccine. In some embodiments, the method further comprises administering to the individual at least one therapeutic agent used in a standard combination therapy with the cancer vaccine. In some embodiments, the sirolimus or derivative thereof is sirolimus. In some embodiments, the sirolimus nanoparticle composition comprises nab-sirolimus. In some embodiments, the sirolimus nanoparticle composition is nab-sirolimus. In some embodiments, the cancer vaccine is a vaccine prepared using autologous tumor cells. In some embodiments, the cancer vaccine is a vaccine prepared using allogeneic tumor cells. In some embodiments, the renal cell carcinoma is recurrent renal cell carcinoma. In some embodiments, the renal cell carcinoma is refractory to one or more drugs used in a standard therapy for renal cell carcinoma, such as, but not limited to, Afinitor (everolimus), temsirolimus, aldesleukin, Avastin (bevacizumab), axitinib, sorafenib, sunitinib, and/or Votrient (pazopanib hydrochloride).

In some embodiments, there is provided a method of treating renal cell carcinoma in an individual (such as a human) comprising administering to the individual a) an effective amount of a composition comprising nanoparticles comprising sirolimus or a derivative thereof and an albumin; and b) an effective amount of a second therapeutic agent selected from the group consisting of Afinitor (everolimus), temsirolimus, aldesleukin, Avastin (bevacizumab), axitinib, sorafenib, sunitinib, and Votrient (pazopanib hydrochloride). In some embodiments, the method comprises administering to the individual a) an effective amount of a composition comprising nanoparticles comprising sirolimus or a derivative thereof and an albumin, wherein the sirolimus or derivative thereof in the nanoparticles is associated (e.g., coated) with the albumin; and b) an effective amount of a second therapeutic agent selected from the group consisting of Afinitor (everolimus), temsirolimus, aldesleukin, Avastin (bevacizumab), axitinib, sorafenib, sunitinib, and Votrient (pazopanib hydrochloride). In some embodiments, the method comprises administering to the individual a) an effective amount of a composition comprising nanoparticles comprising sirolimus or a derivative thereof and an albumin, wherein the nanoparticles have an average particle size of no greater than about 150 nm (such as no greater than about 120 nm); and b) an effective amount of a second therapeutic agent selected from the group consisting of Afinitor (everolimus), temsirolimus, aldesleukin, Avastin (bevacizumab), axitinib, sorafenib, sunitinib, and Votrient (pazopanib hydrochloride). In some embodiments, the method comprises administering to the individual a) an effective amount of a composition comprising nanoparticles comprising sirolimus or a derivative thereof and an albumin, wherein the nanoparticles comprise the sirolimus or derivative thereof associated (e.g., coated) with albumin, wherein the nanoparticles have an average particle size of no greater than about 150 nm (such as no greater than about 120 nm); and b) an effective amount of a second therapeutic agent selected from the group consisting of Afinitor (everolimus), temsirolimus, aldesleukin, Avastin (bevacizumab), axitinib, sorafenib, sunitinib, and Votrient (pazopanib hydrochloride). In some embodiments, the method comprises administering to the individual a) an effective amount of a composition comprising nanoparticles comprising sirolimus or a derivative thereof and an albumin, wherein the nanoparticles comprise the sirolimus or derivative thereof associated (e.g., coated) with the albumin, wherein the nanoparticles have an average particle size of no greater than about 150 nm (such as no greater than about 120 nm, for example about 100 nm), wherein the weight ratio of albumin and the sirolimus or derivative thereof in the sirolimus nanoparticle composition is about 9:1 or less (such as about 9:1 or about 8:1); and b) an effective amount of a second therapeutic agent selected from the group consisting of Afinitor (everolimus), temsirolimus, aldesleukin, Avastin (bevacizumab), axitinib, sorafenib, sunitinib, and Votrient (pazopanib hydrochloride). In some embodiments, the method further comprises administering to the individual at least one therapeutic agent used in a standard combination therapy with the second therapeutic agent. In some embodiments, the sirolimus or derivative thereof is sirolimus. In some embodiments, the sirolimus nanoparticle composition comprises nab-sirolimus. In some embodiments, the sirolimus nanoparticle composition is nab-sirolimus. In some embodiments, the renal cell carcinoma is recurrent renal cell carcinoma. In some embodiments, the renal cell carcinoma is refractory to one or more drugs used in a standard therapy for renal cell carcinoma, such as, but not limited to, Afinitor (everolimus), temsirolimus, aldesleukin, Avastin (bevacizumab), axitinib, sorafenib, sunitinib, and/or Votrient (pazopanib hydrochloride).

In some embodiments, according to any of the methods of treating renal cell carcinoma in an individual described herein, the individual is a human who exhibits one or more symptoms associated with renal cell carcinoma. In some embodiments, the individual is at an early stage of renal cell carcinoma. In some embodiments, the individual is at an advanced stage of renal cell carcinoma. In some of embodiments, the individual is genetically or otherwise predisposed (e.g., having a risk factor) to developing renal cell carcinoma. Individuals at risk for renal cell carcinoma include, e.g., those having relatives who have experienced renal cell carcinoma, and those whose risk is determined by analysis of genetic or biochemical markers. In some embodiments, the individual may be a human who has a gene, genetic mutation, or polymorphism associated with renal cell carcinoma (e.g., VHL, TSC1, TSC2, CUL2, MSH2, MLH1, INK4a/ARF, MET, TGF-α, TGF-β1, IGF-I, IGF-IR, AKT, and/or PTEN) or has one or more extra copies of a gene associated with renal cell carcinoma. In some embodiments, the individual has a ras or PTEN mutation. In some embodiments, the cancer cells are dependent on an mTOR pathway to translate one or more mRNAs. In some embodiments, the cancer cells are not capable of synthesizing mRNAs by an mTOR-independent pathway. In some embodiments, the cancer cells have decreased or no PTEN activity or have decreased or no expression of PTEN compared to non-cancerous cells. In some embodiments, the individual has at least one tumor biomarker selected from the group consisting of elevated PI3K activity, elevated mTOR activity, presence of FLT-3ITD, elevated AKT activity, elevated KRAS activity, and elevated NRAS activity. In some embodiments, the individual has a variation in at least one gene selected from the group consisting of drug metabolism genes, cancer genes, and drug target genes.

### Melanoma

In some embodiments, there is provided a method of treating melanoma in an individual (such as a human) comprising administering to the individual a) an effective amount of a composition comprising nanoparticles comprising an mTOR inhibitor (such as a limus drug, e.g., sirolimus or a derivative thereof) and an albumin; and b) an effective amount of a second therapeutic agent. In some embodiments, the method comprises administering to the individual a) an effective amount of a composition comprising nanoparticles comprising an mTOR inhibitor (such as a limus drug, e.g., sirolimus or a derivative thereof) and an albumin, wherein the mTOR inhibitor in the nanoparticles is associated (e.g., coated) with the albumin; and b) an effective amount of a second therapeutic agent. In some embodiments, the method comprises administering to the individual a) an effective amount of a composition comprising nanoparticles comprising an mTOR inhibitor (such as a limus drug, e.g., sirolimus or a derivative thereof) and an albumin, wherein the nanoparticles have an average particle size of no greater than about 150 nm (such as no greater than about 120 nm); and b) an effective amount of a second therapeutic agent. In some embodiments, the method comprises administering to the individual a) an effective amount of a composition comprising nanoparticles comprising an mTOR inhibitor (such as a limus drug, e.g., sirolimus or a derivative thereof) and an albumin, wherein the nanoparticles comprise the mTOR inhibitor associated (e.g., coated) with albumin, wherein the nanoparticles have an average particle size of no greater than about 150 nm (such as no greater than about 120 nm); and b) an effective amount of a second therapeutic agent. In some embodiments, the method comprises administering to the individual a) an effective amount of a composition comprising nanoparticles comprising an mTOR inhibitor (such as a limus drug, e.g., sirolimus or a derivative thereof) and an albumin, wherein the nanoparticles comprise the mTOR inhibitor associated (e.g., coated) with the albumin, wherein the nanoparticles have an average particle size of no greater than about 150 nm (such as no greater than about 120 nm, for example about 100 nm), wherein the weight ratio of albumin and the mTOR inhibitor in the mTOR inhibitor nanoparticle composition is about 9:1 or less (such as about 9:1 or about 8:1); and b) an effective amount of a second therapeutic agent. In some embodiments, the method further comprises administering to the individual at least one therapeutic agent used in a standard combination therapy with the second therapeutic agent. In some embodiments, the mTOR inhibitor is a limus drug. In some embodiments, the mTOR inhibitor is sirolimus or a derivative thereof. In some embodiments, the mTOR inhibitor nanoparticle composition comprises nab-sirolimus. In some embodiments, the mTOR inhibitor nanoparticle composition is nab-sirolimus. In some embodiments, the second therapeutic agent is selected from the group consisting of an immunomodulator (such as an immunostimulator or an immune checkpoint inhibitor), a histone deacetylase inhibitor, a kinase inhibitor (such as a tyrosine kinase inhibitor), and a cancer vaccine (such as a vaccine prepared using tumor cells or at least one tumor-associated antigen). In some embodiments, the second therapeutic agent is an immunomodulator. In some embodiments, the immunomodulator is an immunostimulator that directly stimulates the immune system of an individual. In some embodiments, the immunomodulator is an agonistic antibody that targets an activating receptor on an immune cell (such as a T cell). In some embodiments, the immunomodulator is an immune checkpoint inhibitor. In some embodiments, the immune checkpoint inhibitor is an antagonistic antibody that targets an immune checkpoint protein. In some embodiments, the immunomodulator is an IMiDs® compound (small molecule immunomodulator, such as lenalidomide or pomalidomide). In some embodiments, the immunomodulator is lenalidomide. In some embodiments, the immunomodulator is pomalidomide. In some embodiments, the immunomodulator is small molecule or antibody-based IDO inhibitor. In some embodiments, the second therapeutic agent is a histone deacetylase inhibitor. In some embodiments, the histone deacetylase inhibitor is specific to only one HDAC. In some embodiments, the histone deacetylase inhibitor is specific to only one class of HDAC. In some embodiments, the histone deacetylase inhibitor is specific to two or more HDACs or two or more classes of HDACs. In some embodiments, the histone deacetylase inhibitor is specific to class I and II HDACs. In some embodiments, the histone deacetylase inhibitor is specific to class III HDACs. In some embodiments, the histone deacetylase inhibitor is selected from the group consisting of romidepsin, panobinostat, ricolinostat, and belinostat. In some embodiments, the histone deacetylase inhibitor is romidepsin. In some embodiments, the second therapeutic agent is a kinase inhibitor, such as a tyrosine kinase inhibitor. In some embodiments, the kinase inhibitor is a serine/threonine kinase inhibitor. In some embodiments, the kinase inhibitor is a Raf kinase inhibitor. In some embodiments, the kinase inhibitor inhibits more than one class of kinase (e.g., an inhibitor of more than one of a tyrosine kinase, a Raf kinase, and a serine/threonine kinase). In some embodiments, the kinase inhibitor is selected from the group consisting of erlotinib, imatinib, lapatinib, nilotinib, sorafenib, and sunitinib. In some embodiments, the kinase inhibitor is sorafenib. In some embodiments, the kinase inhibitor is nilotinib. In some embodiments, the second therapeutic agent is a cancer vaccine, such as a vaccine prepared using tumor cells or at least one tumor-associated antigen. In some embodiments, the second therapeutic agent and the nanoparticle composition are administered sequentially. In some embodiments, the second therapeutic agent and the nanoparticle composition are administered simultaneously. In some embodiments, the second therapeutic agent and the nanoparticle composition are administered concurrently.

In some embodiments, the melanoma is superficial spreading melanoma, lentigo maligna melanoma, nodular melanoma, mucosal melanoma, polypoid melanoma, desmoplastic melanoma, amelanotic melanoma, soft-tissue melanoma, or acral lentiginous melanoma. There are provided methods of treating melanoma at any of the four stages, I, II, III, or IV, according to the American Joint Committee on Cancer (AJCC) staging groups. In some embodiments, the melanoma is recurrent.

In some embodiments, there is provided a method of treating melanoma in an individual (such as a human) comprising administering to the individual a) an effective amount of a composition comprising nanoparticles comprising an mTOR inhibitor (such as a limus drug, e.g., sirolimus or a derivative thereof) and an albumin; and b) an effective amount of an immunomodulator (such as lenalidomide, pomalidomide, or an immune checkpoint inhibitor). In some embodiments, the method comprises administering to the individual a) an effective amount of a composition comprising nanoparticles comprising an mTOR inhibitor (such as a limus drug, e.g., sirolimus or a derivative thereof) and an albumin, wherein the mTOR inhibitor in the nanoparticles is associated (e.g., coated) with the albumin; and b) an effective amount of an immunomodulator (such as lenalidomide, pomalidomide, or an immune checkpoint inhibitor). In some embodiments, the method comprises administering to the individual a) an effective amount of a composition comprising nanoparticles comprising an mTOR inhibitor (such as a limus drug, e.g., sirolimus or a derivative thereof) and an albumin, wherein the nanoparticles have an average particle size of no greater than about 150 nm (such as no greater than about 120 nm); and b) an effective amount of an immunomodulator (such as lenalidomide, pomalidomide, or an immune checkpoint inhibitor). In some embodiments, the method comprises administering to the individual a) an effective amount of a composition comprising nanoparticles comprising an mTOR inhibitor (such as a limus drug, e.g., sirolimus or a derivative thereof) and an albumin, wherein the nanoparticles comprise the mTOR inhibitor associated (e.g., coated) with albumin, wherein the nanoparticles have an average particle size of no greater than about 150 nm (such as no greater than about 120 nm); and b) an effective amount of an immunomodulator (such as lenalidomide, pomalidomide, or an immune checkpoint inhibitor). In some embodiments, the method comprises administering to the individual a) an effective amount of a composition comprising nanoparticles comprising an mTOR inhibitor (such as a limus drug, e.g., sirolimus or a derivative thereof) and an albumin, wherein the nanoparticles comprise the mTOR inhibitor associated (e.g., coated) with the albumin, wherein the nanoparticles have an average particle size of no greater than about 150 nm (such as no greater than about 120 nm, for example about 100 nm), wherein the weight ratio of albumin and the mTOR inhibitor in the mTOR inhibitor nanoparticle composition is about 9:1 or less (such as about 9:1 or about 8:1); and b) an effective amount of an immunomodulator (such as lenalidomide, pomalidomide, or an immune checkpoint inhibitor). In some embodiments, the method further comprises administering to the individual at least one therapeutic agent used in a standard combination therapy with the immunomodulator. In some embodiments, the mTOR inhibitor is a limus drug. In some embodiments, the mTOR inhibitor is sirolimus or a derivative thereof. In some embodiments, the mTOR inhibitor nanoparticle composition comprises nab-sirolimus. In some embodiments, the mTOR inhibitor nanoparticle composition is nab-sirolimus. In some embodiments, the immunomodulator is an immunostimulator that directly stimulates the immune system of an individual. In some embodiments, the immunomodulator is an agonistic antibody that targets an activating receptor on an immune cell (such as a T cell). In some embodiments, the immunomodulator is an immune checkpoint inhibitor. In some embodiments, the immune checkpoint inhibitor is an antagonistic antibody that targets an immune checkpoint protein. In some embodiments, the immunomodulator is an IMiDs® compound (small molecule immunomodulator, such as lenalidomide or pomalidomide). In some embodiments, the immunomodulator is lenalidomide. In some embodiments, the immunomodulator is pomalidomide. In some embodiments, the immunomodulator is small molecule or antibody-based IDO inhibitor. In some embodiments, the melanoma is recurrent melanoma. In some embodiments, the melanoma is refractory to one or more drugs used in a standard therapy for melanoma, such as, but not limited to, aldesleukin, dabrafenib, dacarbazine, interferon alfa-2b, ipilimumab, pembrolizumab, trametinib, nivolumab, and/or vemurafenib.

In some embodiments, there is provided a method of treating melanoma in an individual (such as a human) comprising administering to the individual a) an effective amount of a composition comprising nanoparticles comprising sirolimus or a derivative thereof and an albumin; and b) an effective amount of an immunomodulator (such as an immunostimulator, e.g., pomalidomide). In some embodiments, the method comprises administering to the individual a) an effective amount of a composition comprising nanoparticles comprising sirolimus or a derivative thereof and an albumin, wherein the sirolimus or derivative thereof in the nanoparticles is associated (e.g., coated) with the albumin; and b) an effective amount of an immunomodulator (such as an immunostimulator, e.g., pomalidomide). In some embodiments, the method comprises administering to the individual a) an effective amount of a composition comprising nanoparticles comprising sirolimus or a derivative thereof and an albumin, wherein the nanoparticles have an average particle size of no greater than about 150 nm (such as no greater than about 120 nm); and b) an effective amount of an immunomodulator (such as an immunostimulator, e.g., pomalidomide). In some embodiments, the method comprises administering to the individual a) an effective amount of a composition comprising nanoparticles comprising sirolimus or a derivative thereof and an albumin, wherein the nanoparticles comprise the sirolimus or derivative thereof associated (e.g., coated) with albumin, wherein the nanoparticles have an average particle size of no greater than about 150 nm (such as no greater than about 120 nm); and b) an effective amount of an immunomodulator (such as an immunostimulator, e.g., pomalidomide). In some embodiments, the method comprises administering to the individual a) an effective amount of a composition comprising nanoparticles comprising sirolimus or a derivative thereof and albumin, wherein the nanoparticles comprise the sirolimus or derivative thereof associated (e.g., coated) with the albumin, wherein the nanoparticles have an average particle size of no greater than about 150 nm (such as no greater than about 120 nm, for example about 100 nm), wherein the weight ratio of albumin and sirolimus or a derivative thereof in the sirolimus nanoparticle composition is about 9:1 or less (such as about 9:1 or about 8:1); and b) an effective amount of an immunomodulator (such as an immunostimulator, e.g., pomalidomide). In some embodiments, the method further comprises administering to the individual at least one therapeutic agent used in a standard combination therapy with the immunomodulator. In some embodiments, the sirolimus or derivative thereof is sirolimus. In some embodiments, the sirolimus nanoparticle composition comprises nab-sirolimus. In some embodiments, the sirolimus nanoparticle composition is nab-sirolimus. In some embodiments, the immunomodulator is an immunostimulator that directly stimulates the immune system of an individual. In some embodiments, the immunomodulator is an agonistic antibody that targets an activating receptor on an immune cell (such as a T cell). In some embodiments, the immunomodulator is an immune checkpoint inhibitor. In some embodiments, the immune checkpoint inhibitor is an antagonistic antibody that targets an immune checkpoint protein. In some embodiments, the immunomodulator is an IMiDs® compound (small molecule immunomodulator, such as lenalidomide or pomalidomide). In some embodiments, the immunomodulator is lenalidomide. In some embodiments, the immunomodulator is pomalidomide. In some embodiments, the immunomodulator is small molecule or antibody-based IDO inhibitor. In some embodiments, the melanoma is recurrent melanoma. In some embodiments, the melanoma is refractory to one or more drugs used in a standard therapy for melanoma, such as, but not limited to, aldesleukin, dabrafenib, dacarbazine, interferon alfa-2b, ipilimumab, pembrolizumab, trametinib, nivolumab, and/or vemurafenib.

In some embodiments, there is provided a method of treating melanoma in an individual (such as a human) comprising administering to the individual a) an effective amount of a composition comprising nanoparticles comprising an mTOR inhibitor (such as a limus drug, e.g., sirolimus or a derivative thereof) and an albumin; and b) an effective amount of a histone deacetylase inhibitor (such as romidepsin). In some embodiments, the method comprises administering to the individual a) an effective amount of a composition comprising nanoparticles comprising an mTOR inhibitor (such as a limus drug, e.g., sirolimus or a derivative thereof) and an albumin, wherein the mTOR inhibitor in the nanoparticles is associated (e.g., coated) with the albumin; and b) an effective amount of a histone deacetylase inhibitor (such as romidepsin). In some embodiments, the method comprises administering to the individual a) an effective amount of a composition comprising nanoparticles comprising an mTOR inhibitor (such as a limus drug, e.g., sirolimus or a derivative thereof) and an albumin, wherein the nanoparticles have an average particle size of no greater than about 150 nm (such as no greater than about 120 nm); and b) an effective amount of a histone deacetylase inhibitor (such as romidepsin). In some embodiments, the method comprises administering to the individual a) an effective amount of a composition comprising nanoparticles comprising an mTOR inhibitor (such as a limus drug, e.g., sirolimus or a derivative thereof) and an albumin, wherein the nanoparticles comprise the mTOR inhibitor associated (e.g., coated) with albumin, wherein the nanoparticles have an average particle size of no greater than about 150 nm (such as no greater than about 120 nm); and b) an effective amount of a histone deacetylase inhibitor (such as romidepsin). In some embodiments, the method comprises administering to the individual a) an effective amount of a composition comprising nanoparticles comprising an mTOR inhibitor (such as a limus drug, e.g., sirolimus or a derivative thereof) and an albumin, wherein the nanoparticles comprise the mTOR inhibitor associated (e.g., coated) with the albumin, wherein the nanoparticles have an average particle size of no greater than about 150 nm (such as no greater than about 120 nm, for example about 100 nm), wherein the weight ratio of albumin and the mTOR inhibitor in the mTOR inhibitor nanoparticle composition is about 9:1 or less (such as about 9:1 or about 8:1); and b) an effective amount of a histone deacetylase inhibitor (such as romidepsin). In some embodiments, the method further comprises administering to the individual at least one therapeutic agent used in a standard combination therapy with the histone deacetylase inhibitor. In some embodiments, the mTOR inhibitor is a limus drug. In some embodiments, the mTOR inhibitor is sirolimus or a derivative thereof. In some embodiments, the mTOR inhibitor nanoparticle composition comprises nab-sirolimus. In some embodiments, the mTOR inhibitor nanoparticle composition is nab-sirolimus. In some embodiments, the histone deacetylase inhibitor is selected from the group consisting of romidepsin, panobinostat, ricolinostat, and belinostat. In some embodiments, the histone deacetylase inhibitor is romidepsin. In some embodiments, the melanoma is recurrent melanoma. In some embodiments, the melanoma is refractory to one or more drugs used in a standard therapy for melanoma, such as, but not limited to, aldesleukin, dabrafenib, dacarbazine, interferon alfa-2b, ipilimumab, pembrolizumab, trametinib, nivolumab, and/or vemurafenib.

In some embodiments, there is provided a method of treating melanoma in an individual (such as a human) comprising administering to the individual a) an effective amount of a composition comprising nanoparticles comprising sirolimus or a derivative thereof and an albumin; and b) an effective amount of a histone deacetylase inhibitor (such as romidepsin). In some embodiments, the method comprises administering to the individual a) an effective amount of a composition comprising nanoparticles comprising sirolimus or a derivative thereof and an albumin, wherein the sirolimus or derivative thereof in the nanoparticles is associated (e.g., coated) with the albumin; and b) an effective amount of a histone deacetylase inhibitor (such as romidepsin). In some embodiments, the method comprises administering to the individual a) an effective amount of a composition comprising nanoparticles comprising sirolimus or a derivative thereof and an albumin, wherein the nanoparticles have an average particle size of no greater than about 150 nm (such as no greater than about 120 nm); and b) an effective amount of a histone deacetylase inhibitor (such as romidepsin). In some embodiments, the method comprises administering to the individual a) an effective amount of a composition comprising nanoparticles comprising sirolimus or a derivative thereof and an albumin, wherein the nanoparticles comprise the sirolimus or derivative thereof associated (e.g., coated) with albumin, wherein the nanoparticles have an average particle size of no greater than about 150 nm (such as no greater than about 120 nm); and b) an effective amount of a histone deacetylase inhibitor (such as romidepsin). In some embodiments, the method comprises administering to the individual a) an effective amount of a composition comprising nanoparticles comprising sirolimus or a derivative thereof and albumin, wherein the nanoparticles comprise the sirolimus or derivative thereof associated (e.g., coated) with the albumin, wherein the nanoparticles have an average particle size of no greater than about 150 nm (such as no greater than about 120 nm, for example about 100 nm), wherein the weight ratio of albumin and sirolimus or a derivative thereof in the sirolimus nanoparticle composition is about 9:1 or less (such as about 9:1 or about 8:1); and b) an effective amount of a histone deacetylase inhibitor (such as romidepsin). In some embodiments, the method further comprises administering to the individual at least one therapeutic agent used in a standard combination therapy with the histone deacetylase inhibitor. In some embodiments, the sirolimus or derivative thereof is sirolimus. In some embodiments, the sirolimus nanoparticle composition comprises nab-sirolimus. In some embodiments, the sirolimus nanoparticle composition is nab-sirolimus. In some embodiments, the histone deacetylase inhibitor is selected from the group consisting of romidepsin, panobinostat, ricolinostat, and belinostat. In some embodiments, the histone deacetylase inhibitor is romidepsin. In some embodiments, the melanoma is recurrent melanoma. In some embodiments, the melanoma is refractory to one or more drugs used in a standard therapy for melanoma, such as, but not limited to, aldesleukin, dabrafenib, dacarbazine, interferon alfa-2b, ipilimumab, pembrolizumab, trametinib, nivolumab, and/or vemurafenib.

In some embodiments, there is provided a method of treating melanoma in an individual (such as a human) comprising administering to the individual a) an effective amount of a composition comprising nanoparticles comprising an mTOR inhibitor (such as a limus drug, e.g., sirolimus or a derivative thereof) and an albumin; and b) an effective amount of a kinase inhibitor (such as a tyrosine kinase inhibitor, e.g., nilotinib or sorafenib). In some embodiments, the method comprises administering to the individual a) an effective amount of a composition comprising nanoparticles comprising an mTOR inhibitor (such as a limus drug, e.g., sirolimus or a derivative thereof) and an albumin, wherein the mTOR inhibitor in the nanoparticles is associated (e.g., coated) with the albumin; and b) an effective amount of a kinase inhibitor (such as a tyrosine kinase inhibitor, e.g., nilotinib or sorafenib). In some embodiments, the method comprises administering to the individual a) an effective amount of a composition comprising nanoparticles comprising an mTOR inhibitor (such as a limus drug, e.g., sirolimus or a derivative thereof) and an albumin, wherein the nanoparticles have an average particle size of no greater than about 150 nm (such as no greater than about 120 nm); and b) an effective amount of a kinase inhibitor (such as a tyrosine kinase inhibitor, e.g., nilotinib or sorafenib). In some embodiments, the method comprises administering to the individual a) an effective amount of a composition comprising nanoparticles comprising an mTOR inhibitor (such as a limus drug, e.g., sirolimus or a derivative thereof) and an albumin, wherein the nanoparticles comprise the mTOR inhibitor associated (e.g., coated) with albumin, wherein the nanoparticles have an average particle size of no greater than about 150 nm (such as no greater than about 120 nm); and b) an effective amount of a kinase inhibitor (such as a tyrosine kinase inhibitor, e.g., nilotinib or sorafenib). In some embodiments, the method comprises administering to the individual a) an effective amount of a composition comprising nanoparticles comprising an mTOR inhibitor (such as a limus drug, e.g., sirolimus or a derivative thereof) and an albumin, wherein the nanoparticles comprise the mTOR inhibitor associated (e.g., coated) with the albumin, wherein the nanoparticles have an average particle size of no greater than about 150 nm (such as no greater than about 120 nm, for example about 100 nm), wherein the weight ratio of albumin and the mTOR inhibitor in the mTOR inhibitor nanoparticle composition is about 9:1 or less (such as about 9:1 or about 8:1); and b) an effective amount of a kinase inhibitor (such as a tyrosine kinase inhibitor, e.g., nilotinib or sorafenib). In some embodiments, the method further comprises administering to the individual at least one therapeutic agent used in a standard combination therapy with the kinase inhibitor. In some embodiments, the mTOR inhibitor is a limus drug. In some embodiments, the mTOR inhibitor is sirolimus or a derivative thereof. In some embodiments, the mTOR inhibitor nanoparticle composition comprises nab-sirolimus. In some embodiments, the mTOR inhibitor nanoparticle composition is nab-sirolimus. In some embodiments, the kinase inhibitor is a tyrosine kinase inhibitor. In some embodiments, the kinase inhibitor is a serine/threonine kinase inhibitor. In some embodiments, the kinase inhibitor is a Raf kinase inhibitor. In some embodiments, the kinase inhibitor inhibits more than one class of kinase (e.g., an inhibitor of more than one of a tyrosine kinase, a Raf kinase, and a serine/threonine kinase). In some embodiments, the kinase inhibitor is selected from the group consisting of erlotinib, imatinib, lapatinib, nilotinib, sorafenib, and sunitinib. In some embodiments, the kinase inhibitor is nilotinib. In some embodiments, the melanoma is recurrent melanoma. In some embodiments, the melanoma is refractory to one or more drugs used in a standard therapy for melanoma, such as, but not limited to, aldesleukin, dabrafenib, dacarbazine, interferon alfa-2b, ipilimumab, pembrolizumab, trametinib, nivolumab, and/or vemurafenib.

In some embodiments, there is provided a method of treating melanoma in an individual (such as a human) comprising administering to the individual a) an effective amount of a composition comprising nanoparticles comprising sirolimus or a derivative thereof and an albumin; and b) an effective amount of a kinase inhibitor (such as a tyrosine kinase inhibitor, e.g., nilotinib or sorafenib). In some embodiments, the method comprises administering to the individual a) an effective amount of a composition comprising nanoparticles comprising sirolimus or a derivative thereof and an albumin, wherein the sirolimus or derivative thereof in the nanoparticles is associated (e.g., coated) with the albumin; and b) an effective amount of a kinase inhibitor (such as a tyrosine kinase inhibitor, e.g., nilotinib or sorafenib). In some embodiments, the method comprises administering to the individual a) an effective amount of a composition comprising nanoparticles comprising sirolimus or a derivative thereof and an albumin, wherein the nanoparticles have an average particle size of no greater than about 150 nm (such as no greater than about 120 nm); and b) an effective amount of a kinase inhibitor (such as a tyrosine kinase inhibitor, e.g., nilotinib or sorafenib). In some embodiments, the method comprises administering to the individual a) an effective amount of a composition comprising nanoparticles comprising sirolimus or a derivative thereof and an albumin, wherein the nanoparticles comprise the sirolimus or derivative thereof associated (e.g., coated) with albumin, wherein the nanoparticles have an average particle size of no greater than about 150 nm (such as no greater than about 120 nm); and b) an effective amount of a kinase inhibitor (such as a tyrosine kinase inhibitor, e.g., nilotinib or sorafenib). In some embodiments, the method comprises administering to the individual a) an effective amount of a composition comprising nanoparticles comprising sirolimus or a derivative thereof and an albumin, wherein the nanoparticles comprise the sirolimus or derivative thereof associated (e.g., coated) with the albumin, wherein the nanoparticles have an average particle size of no greater than about 150 nm (such as no greater than about 120 nm, for example about 100 nm), wherein the weight ratio of albumin and the sirolimus or derivative thereof in the sirolimus nanoparticle composition is about 9:1 or less (such as about 9:1 or about 8:1); and b) an effective amount of a kinase inhibitor (such as a tyrosine kinase inhibitor, e.g., nilotinib or sorafenib). In some embodiments, the method further comprises administering to the individual at least one therapeutic agent used in a standard combination therapy with the kinase inhibitor. In some embodiments, the sirolimus or derivative thereof is sirolimus. In some embodiments, the sirolimus nanoparticle composition comprises nab-sirolimus. In some embodiments, the sirolimus nanoparticle composition is nab-sirolimus. In some embodiments, the kinase inhibitor is a tyrosine kinase inhibitor. In some embodiments, the kinase inhibitor is a serine/threonine kinase inhibitor. In some embodiments, the kinase inhibitor is a Raf kinase inhibitor. In some embodiments, the kinase inhibitor inhibits more than one class of kinase (e.g., an inhibitor of more than one of a tyrosine kinase, a Raf kinase, and a serine/threonine kinase). In some embodiments, the kinase inhibitor is selected from the group consisting of erlotinib, imatinib, lapatinib, nilotinib, sorafenib, and sunitinib. In some embodiments, the kinase inhibitor is nilotinib. In some embodiments, the melanoma is recurrent melanoma. In some embodiments, the melanoma is refractory to one or more drugs used in a standard therapy for melanoma, such as, but not limited to, aldesleukin, dabrafenib, dacarbazine, interferon alfa-2b, ipilimumab, pembrolizumab, trametinib, nivolumab, and/or vemurafenib.

In some embodiments, there is provided a method of treating melanoma in an individual (such as a human) comprising administering to the individual a) an effective amount of a composition comprising nanoparticles comprising an mTOR inhibitor (such as a limus drug, e.g., sirolimus or a derivative thereof) and an albumin; and b) an effective amount of a cancer vaccine. In some embodiments, the method comprises administering to the individual a) an effective amount of a composition comprising nanoparticles comprising an mTOR inhibitor (such as a limus drug, e.g., sirolimus or a derivative thereof) and an albumin, wherein the mTOR inhibitor in the nanoparticles is associated (e.g., coated) with the albumin; and b) an effective amount of a cancer vaccine. In some embodiments, the method comprises administering to the individual a) an effective amount of a composition comprising nanoparticles comprising an mTOR inhibitor (such as a limus drug, e.g., sirolimus or a derivative thereof) and an albumin, wherein the nanoparticles have an average particle size of no greater than about 150 nm (such as no greater than about 120 nm); and b) an effective amount of a cancer vaccine. In some embodiments, the method comprises administering to the individual a) an effective amount of a composition comprising nanoparticles comprising an mTOR inhibitor (such as a limus drug, e.g., sirolimus or a derivative thereof) and an albumin, wherein the nanoparticles comprise the mTOR inhibitor associated (e.g., coated) with albumin, wherein the nanoparticles have an average particle size of no greater than about 150 nm (such as no greater than about 120 nm); and b) an effective amount of a cancer vaccine. In some embodiments, the method comprises administering to the individual a) an effective amount of a composition comprising nanoparticles comprising an mTOR inhibitor (such as a limus drug, e.g., sirolimus or a derivative thereof) and an albumin, wherein the nanoparticles comprise the mTOR inhibitor associated (e.g., coated) with the albumin, wherein the nanoparticles have an average particle size of no greater than about 150 nm (such as no greater than about 120 nm, for example about 100 nm), wherein the weight ratio of albumin and the mTOR inhibitor in the mTOR inhibitor nanoparticle composition is about 9:1 or less (such as about 9:1 or about 8:1); and b) an effective amount of a cancer vaccine. In some embodiments, the method further comprises administering to the individual at least one therapeutic agent used in a standard combination therapy with the cancer vaccine. In some embodiments, the mTOR inhibitor is a limus drug. In some embodiments, the mTOR inhibitor is sirolimus or a derivative thereof. In some embodiments, the mTOR inhibitor nanoparticle composition comprises nab-sirolimus. In some embodiments, the mTOR inhibitor nanoparticle composition is nab-sirolimus. In some embodiments, the cancer vaccine is a vaccine prepared using autologous tumor cells. In some embodiments, the cancer vaccine is a vaccine prepared using allogeneic tumor cells. In some embodiments, the melanoma is recurrent melanoma. In some embodiments, the melanoma is refractory to one or more drugs used in a standard therapy for melanoma, such as, but not limited to, aldesleukin, dabrafenib, dacarbazine, interferon alfa-2b, ipilimumab, pembrolizumab, trametinib, nivolumab, and/or vemurafenib.

In some embodiments, there is provided a method of treating melanoma in an individual (such as a human) comprising administering to the individual a) an effective amount of a composition comprising nanoparticles comprising sirolimus or a derivative thereof and an albumin; and b) an effective amount of a cancer vaccine. In some embodiments, the method comprises administering to the individual a) an effective amount of a composition comprising nanoparticles comprising sirolimus or a derivative thereof and an albumin, wherein the sirolimus or derivative thereof in the nanoparticles is associated (e.g., coated) with the albumin; and b) an effective amount of a cancer vaccine. In some embodiments, the method comprises administering to the individual a) an effective amount of a composition comprising nanoparticles comprising sirolimus or a derivative thereof and an albumin, wherein the nanoparticles have an average particle size of no greater than about 150 nm (such as no greater than about 120 nm); and b) an effective amount of a cancer vaccine. In some embodiments, the method comprises administering to the individual a) an effective amount of a composition comprising nanoparticles comprising sirolimus or a derivative thereof and an albumin, wherein the nanoparticles comprise the sirolimus or derivative thereof associated (e.g., coated) with albumin, wherein the nanoparticles have an average particle size of no greater than about 150 nm (such as no greater than about 120 nm); and b) an effective amount of a cancer vaccine. In some embodiments, the method comprises administering to the individual a) an effective amount of a composition comprising nanoparticles comprising sirolimus or a derivative thereof and an albumin, wherein the nanoparticles comprise the sirolimus or derivative thereof associated (e.g., coated) with the albumin, wherein the nanoparticles have an average particle size of no greater than about 150 nm (such as no greater than about 120 nm, for example about 100 nm), wherein the weight ratio of albumin and the sirolimus or derivative thereof in the sirolimus nanoparticle composition is about 9:1 or less (such as about 9:1 or about 8:1); and b) an effective amount of a cancer vaccine. In some embodiments, the method further comprises administering to the individual at least one therapeutic agent used in a standard combination therapy with the cancer vaccine. In some embodiments, the sirolimus or derivative thereof is sirolimus. In some embodiments, the sirolimus nanoparticle composition comprises nab-sirolimus. In some embodiments, the sirolimus nanoparticle composition is nab-sirolimus. In some embodiments, the cancer vaccine is a vaccine prepared using autologous tumor cells. In some embodiments, the cancer vaccine is a vaccine prepared using allogeneic tumor cells. In some embodiments, the melanoma is recurrent melanoma. In some embodiments, the melanoma is refractory to one or more drugs used in a standard therapy for melanoma, such as, but not limited to, aldesleukin, dabrafenib, dacarbazine, interferon alfa-2b, ipilimumab, pembrolizumab, trametinib, nivolumab, and/or vemurafenib.

In some embodiments, there is provided a method of treating melanoma in an individual (such as a human) comprising administering to the individual a) an effective amount of a composition comprising nanoparticles comprising sirolimus or a derivative thereof and an albumin; and b) an effective amount of a second therapeutic agent selected from the group consisting of aldesleukin, dabrafenib, dacarbazine, interferon alfa-2b, ipilimumab, pembrolizumab, trametinib, nivolumab, and vemurafenib. In some embodiments, the method comprises administering to the individual a) an effective amount of a composition comprising nanoparticles comprising sirolimus or a derivative thereof and an albumin, wherein the sirolimus or derivative thereof in the nanoparticles is associated (e.g., coated) with the albumin; and b) an effective amount of a second therapeutic agent selected from the group consisting of aldesleukin, dabrafenib, dacarbazine, interferon alfa-2b, ipilimumab, pembrolizumab, trametinib, nivolumab, and vemurafenib. In some embodiments, the method comprises administering to the individual a) an effective amount of a composition comprising nanoparticles comprising sirolimus or a derivative thereof and an albumin, wherein the nanoparticles have an average particle size of no greater than about 150 nm (such as no greater than about 120 nm); and b) an effective amount of a second therapeutic agent selected from the group consisting of aldesleukin, dabrafenib, dacarbazine, interferon alfa-2b, ipilimumab, pembrolizumab, trametinib, nivolumab, and vemurafenib. In some embodiments, the method comprises administering to the individual a) an effective amount of a composition comprising nanoparticles comprising sirolimus or a derivative thereof and an albumin, wherein the nanoparticles comprise the sirolimus or derivative thereof associated (e.g., coated) with albumin, wherein the nanoparticles have an average particle size of no greater than about 150 nm (such as no greater than about 120 nm); and b) an effective amount of a second therapeutic agent selected from the group consisting of aldesleukin, dabrafenib, dacarbazine, interferon alfa-2b, ipilimumab, pembrolizumab, trametinib, nivolumab, and vemurafenib. In some embodiments, the method comprises administering to the individual a) an effective amount of a composition comprising nanoparticles comprising sirolimus or a derivative thereof and an albumin, wherein the nanoparticles comprise the sirolimus or derivative thereof associated (e.g., coated) with the albumin, wherein the nanoparticles have an average particle size of no greater than about 150 nm (such as no greater than about 120 nm, for example about 100 nm), wherein the weight ratio of albumin and the sirolimus or derivative thereof in the sirolimus nanoparticle composition is about 9:1 or less (such as about 9:1 or about 8:1); and b) an effective amount of a second therapeutic agent selected from the group consisting of aldesleukin, dabrafenib, dacarbazine, interferon alfa-2b, ipilimumab, pembrolizumab, trametinib, nivolumab, and vemurafenib. In some embodiments, the method further comprises administering to the individual at least one therapeutic agent used in a standard combination therapy with the second therapeutic agent. In some embodiments, the sirolimus or derivative thereof is sirolimus. In some embodiments, the sirolimus nanoparticle composition comprises nab-sirolimus. In some embodiments, the sirolimus nanoparticle composition is nab-sirolimus. In some embodiments, the melanoma is recurrent melanoma. In some embodiments, the melanoma is refractory to one or more drugs used in a standard therapy for melanoma, such as, but not limited to, aldesleukin, dabrafenib, dacarbazine, interferon alfa-2b, ipilimumab, pembrolizumab, trametinib, nivolumab, and/or vemurafenib.

In some embodiments, according to any of the methods of treating melanoma in an individual described herein, the individual is a human who exhibits one or more symptoms associated with melanoma. In some embodiments, the individual is at an early stage of melanoma. In some embodiments, the individual is at an advanced stage of melanoma. In some of embodiments, the individual is genetically or otherwise predisposed (e.g., having a risk factor) to developing melanoma. Individuals at risk for melanoma include, e.g., those having relatives who have experienced melanoma, and those whose risk is determined by analysis of genetic or biochemical markers. In some embodiments, the individual may be a human who has a gene, genetic mutation, or polymorphism associated with melanoma (e.g., CDKN2A, CDK4, BRCA2, BRAF, NRAS, KIT, MC1R, or MDM2) or has one or more extra copies of a gene associated with melanoma. In some embodiments, the individual has a ras or PTEN mutation. In some embodiments, the cancer cells are dependent on an mTOR pathway to translate one or more mRNAs. In some embodiments, the cancer cells are not capable of synthesizing mRNAs by an mTOR-independent pathway. In some embodiments, the cancer cells have decreased or no PTEN activity or have decreased or no expression of PTEN compared to non-cancerous cells. In some embodiments, the individual has at least one tumor biomarker selected from the group consisting of elevated PI3K activity, elevated mTOR activity, presence of FLT-3ITD, elevated AKT activity, elevated KRAS activity, and elevated NRAS activity. In some embodiments, the individual has a variation in at least one gene selected from the group consisting of drug metabolism genes, cancer genes, and drug target genes.

### Breast Cancer

In some embodiments, there is provided a method of treating breast cancer (such as hormone receptor positive (HR+) breast cancer) in an individual (such as a human) comprising administering to the individual a) an effective amount of a composition comprising nanoparticles comprising an mTOR inhibitor (such as a limus drug, e.g., sirolimus or a derivative thereof) and an albumin; and b) an effective amount of a second therapeutic agent. In some embodiments, the method comprises administering to the individual a) an effective amount of a composition comprising nanoparticles comprising an mTOR inhibitor (such as a limus drug, e.g., sirolimus or a derivative thereof) and an albumin, wherein the mTOR inhibitor in the nanoparticles is associated (e.g., coated) with the albumin; and b) an effective amount of a second therapeutic agent. In some embodiments, the method comprises administering to the individual a) an effective amount of a composition comprising nanoparticles comprising an mTOR inhibitor (such as a limus drug, e.g., sirolimus or a derivative thereof) and an albumin, wherein the nanoparticles have an average particle size of no greater than about 150 nm (such as no greater than about 120 nm); and b) an effective amount of a second therapeutic agent. In some embodiments, the method comprises administering to the individual a) an effective amount of a composition comprising nanoparticles comprising an mTOR inhibitor (such as a limus drug, e.g., sirolimus or a derivative thereof) and an albumin, wherein the nanoparticles comprise the mTOR inhibitor associated (e.g., coated) with albumin, wherein the nanoparticles have an average particle size of no greater than about 150 nm (such as no greater than about 120 nm); and b) an effective amount of a second therapeutic agent. In some embodiments, the method comprises administering to the individual a) an effective amount of a composition comprising nanoparticles comprising an mTOR inhibitor (such as a limus drug, e.g., sirolimus or a derivative thereof) and an albumin, wherein the nanoparticles comprise the mTOR inhibitor associated (e.g., coated) with the albumin, wherein the nanoparticles have an average particle size of no greater than about 150 nm (such as no greater than about 120 nm, for example about 100 nm), wherein the weight ratio of albumin and the mTOR inhibitor in the mTOR inhibitor nanoparticle composition is about 9:1 or less (such as about 9:1 or about 8:1); and b) an effective amount of a second therapeutic agent. In some embodiments, the method further comprises administering to the individual at least one therapeutic agent used in a standard combination therapy with the second therapeutic agent. In some embodiments, the mTOR inhibitor is a limus drug. In some embodiments, the mTOR inhibitor is sirolimus or a derivative thereof. In some embodiments, the mTOR inhibitor nanoparticle composition comprises nab-sirolimus. In some embodiments, the mTOR inhibitor nanoparticle composition is nab-sirolimus. In some embodiments, the second therapeutic agent is selected from the group consisting of an immunomodulator (such as an immunostimulator or an immune checkpoint inhibitor), a histone deacetylase inhibitor, a kinase inhibitor (such as a tyrosine kinase inhibitor), and a cancer vaccine (such as a vaccine prepared using tumor cells or at least one tumor-associated antigen). In some embodiments, the second therapeutic agent is an immunomodulator. In some embodiments, the immunomodulator is an immunostimulator that directly stimulates the immune system of an individual. In some embodiments, the immunomodulator is an agonistic antibody that targets an activating receptor on an immune cell (such as a T cell). In some embodiments, the immunomodulator is an immune checkpoint inhibitor. In some embodiments, the immune checkpoint inhibitor is an antagonistic antibody that targets an immune checkpoint protein. In some embodiments, the immunomodulator is an IMiDs® compound (small molecule immunomodulator, such as lenalidomide or pomalidomide). In some embodiments, the immunomodulator is lenalidomide. In some embodiments, the immunomodulator is pomalidomide. In some embodiments, the immunomodulator is small molecule or antibody-based IDO inhibitor. In some embodiments, the second therapeutic agent is a histone deacetylase inhibitor. In some embodiments, the histone deacetylase inhibitor is specific to only one HDAC. In some embodiments, the histone deacetylase inhibitor is specific to only one class of HDAC. In some embodiments, the histone deacetylase inhibitor is specific to two or more HDACs or two or more classes of HDACs. In some embodiments, the histone deacetylase inhibitor is specific to class I and II HDACs. In some embodiments, the histone deacetylase inhibitor is specific to class III HDACs. In some embodiments, the histone deacetylase inhibitor is selected from the group consisting of romidepsin, panobinostat, ricolinostat, and belinostat. In some embodiments, the histone deacetylase inhibitor is romidepsin. In some embodiments, the second therapeutic agent is a kinase inhibitor, such as a tyrosine kinase inhibitor. In some embodiments, the kinase inhibitor is a serine/threonine kinase inhibitor. In some embodiments, the kinase inhibitor is a Raf kinase inhibitor. In some embodiments, the kinase inhibitor inhibits more than one class of kinase (e.g., an inhibitor of more than one of a tyrosine kinase, a Raf kinase, and a serine/threonine kinase). In some embodiments, the kinase inhibitor is selected from the group consisting of erlotinib, imatinib, lapatinib, nilotinib, sorafenib, and sunitinib. In some embodiments, the kinase inhibitor is sorafenib. In some embodiments, the kinase inhibitor is nilotinib. In some embodiments, the second therapeutic agent is a cancer vaccine, such as a vaccine prepared using tumor cells or at least one tumor-associated antigen. In some embodiments, the second therapeutic agent and the nanoparticle composition are administered sequentially. In some embodiments, the second therapeutic agent and the nanoparticle composition are administered simultaneously. In some embodiments, the second therapeutic agent and the nanoparticle composition are administered concurrently.

In some embodiments, the breast cancer is early stage breast cancer, non-metastatic breast cancer, advanced breast cancer, stage IV breast cancer, locally advanced breast cancer, metastatic breast cancer, breast cancer in remission, breast cancer in an adjuvant setting, or breast cancer in a neoadjuvant setting. In some embodiments, the breast cancer is in a neoadjuvant setting. In some embodiments, the breast cancer is at an advanced stage. In some embodiments, the breast cancer (which may be HER2 positive or HER2 negative) includes, for example, advanced breast cancer, stage IV breast cancer, locally advanced breast cancer, and metastatic breast cancer.

In some embodiments, there is provided a method of treating breast cancer (such as HR+ breast cancer) in an individual (such as a human) comprising administering to the individual a) an effective amount of a composition comprising nanoparticles comprising an mTOR inhibitor (such as a limus drug, e.g., sirolimus or a derivative thereof) and an albumin; and b) an effective amount of an immunomodulator (such as lenalidomide, pomalidomide, or an immune checkpoint inhibitor). In some embodiments, the method comprises administering to the individual a) an effective amount of a composition comprising nanoparticles comprising an mTOR inhibitor (such as a limus drug, e.g., sirolimus or a derivative thereof) and an albumin, wherein the mTOR inhibitor in the nanoparticles is associated (e.g., coated) with the albumin; and b) an effective amount of an immunomodulator (such as lenalidomide, pomalidomide, or an immune checkpoint inhibitor). In some embodiments, the method comprises administering to the individual a) an effective amount of a composition comprising nanoparticles comprising an mTOR inhibitor (such as a limus drug, e.g., sirolimus or a derivative thereof) and an albumin, wherein the nanoparticles have an average particle size of no greater than about 150 nm (such as no greater than about 120 nm); and b) an effective amount of an immunomodulator (such as lenalidomide, pomalidomide, or an immune checkpoint inhibitor). In some embodiments, the method comprises administering to the individual a) an effective amount of a composition comprising nanoparticles comprising an mTOR inhibitor (such as a limus drug, e.g., sirolimus or a derivative thereof) and an albumin, wherein the nanoparticles comprise the mTOR inhibitor associated (e.g., coated) with albumin, wherein the nanoparticles have an average particle size of no greater than about 150 nm (such as no greater than about 120 nm); and b) an effective amount of an immunomodulator (such as lenalidomide, pomalidomide, or an immune checkpoint inhibitor). In some embodiments, the method comprises administering to the individual a) an effective amount of a composition comprising nanoparticles comprising an mTOR inhibitor (such as a limus drug, e.g., sirolimus or a derivative thereof) and an albumin, wherein the nanoparticles comprise the mTOR inhibitor associated (e.g., coated) with the albumin, wherein the nanoparticles have an average particle size of no greater than about 150 nm (such as no greater than about 120 nm, for example about 100 nm), wherein the weight ratio of albumin and the mTOR inhibitor in the mTOR inhibitor nanoparticle composition is about 9:1 or less (such as about 9:1 or about 8:1); and b) an effective amount of an immunomodulator (such as lenalidomide, pomalidomide, or an immune checkpoint inhibitor). In some embodiments, the method further comprises administering to the individual at least one therapeutic agent used in a standard combination therapy with the immunomodulator. In some embodiments, the mTOR inhibitor is a limus drug. In some embodiments, the mTOR inhibitor is sirolimus or a derivative thereof. In some embodiments, the mTOR inhibitor nanoparticle composition comprises nab-sirolimus. In some embodiments, the mTOR inhibitor nanoparticle composition is nab-sirolimus. In some embodiments, the immunomodulator is an immunostimulator that directly stimulates the immune system of an individual. In some embodiments, the immunomodulator is an agonistic antibody that targets an activating receptor on an immune cell (such as a T cell). In some embodiments, the immunomodulator is an immune checkpoint inhibitor. In some embodiments, the immune checkpoint inhibitor is an antagonistic antibody that targets an immune checkpoint protein. In some embodiments, the immunomodulator is an IMiDs® compound (small molecule immunomodulator, such as lenalidomide or pomalidomide). In some embodiments, the immunomodulator is lenalidomide. In some embodiments, the immunomodulator is pomalidomide. In some embodiments, the immunomodulator is small molecule or antibody-based IDO inhibitor. In some embodiments, the breast cancer (such as HR+ breast cancer) is recurrent breast cancer (such as HR+ breast cancer). In some embodiments, the breast cancer (such as HR+ breast cancer) is refractory to one or more drugs used in a standard therapy for breast cancer (such as HR+ breast cancer), such as, but not limited to, docetaxel, paclitaxel, cisplatin, carboplatin, vinorelbine, capecitabine, liposomal doxorubicin, gemcitabine, mitoxantrone, ixabepilone, nab-paclitaxel, and/or eribulin.

In some embodiments, there is provided a method of treating breast cancer (such as HR+ breast cancer) in an individual (such as a human) comprising administering to the individual a) an effective amount of a composition comprising nanoparticles comprising sirolimus or a derivative thereof and an albumin; and b) an effective amount of an immunomodulator (such as an immunostimulator, e.g., pomalidomide). In some embodiments, the method comprises administering to the individual a) an effective amount of a composition comprising nanoparticles comprising sirolimus or a derivative thereof and an albumin, wherein the sirolimus or derivative thereof in the nanoparticles is associated (e.g., coated) with the albumin; and b) an effective amount of an immunomodulator (such as an immunostimulator, e.g., pomalidomide). In some embodiments, the method comprises administering to the individual a) an effective amount of a composition comprising nanoparticles comprising sirolimus or a derivative thereof and an albumin, wherein the nanoparticles have an average particle size of no greater than about 150 nm (such as no greater than about 120 nm); and b) an effective amount of an immunomodulator (such as an immunostimulator, e.g., pomalidomide). In some embodiments, the method comprises administering to the individual a) an effective amount of a composition comprising nanoparticles comprising sirolimus or a derivative thereof and an albumin, wherein the nanoparticles comprise the sirolimus or derivative thereof associated (e.g., coated) with albumin, wherein the nanoparticles have an average particle size of no greater than about 150 nm (such as no greater than about 120 nm); and b) an effective amount of an immunomodulator (such as an immunostimulator, e.g., pomalidomide). In some embodiments, the method comprises administering to the individual a) an effective amount of a composition comprising nanoparticles comprising sirolimus or a derivative thereof and albumin, wherein the nanoparticles comprise the sirolimus or derivative thereof associated (e.g., coated) with the albumin, wherein the nanoparticles have an average particle size of no greater than about 150 nm (such as no greater than about 120 nm, for example about 100 nm), wherein the weight ratio of albumin and sirolimus or a derivative thereof in the sirolimus nanoparticle composition is about 9:1 or less (such as about 9:1 or about 8:1); and b) an effective amount of an immunomodulator (such as an immunostimulator, e.g., pomalidomide). In some embodiments, the method further comprises administering to the individual at least one therapeutic agent used in a standard combination therapy with the immunomodulator. In some embodiments, the sirolimus or derivative thereof is sirolimus. In some embodiments, the sirolimus nanoparticle composition comprises nab-sirolimus. In some embodiments, the sirolimus nanoparticle composition is nab-sirolimus. In some embodiments, the immunomodulator is an immunostimulator that directly stimulates the immune system of an individual. In some embodiments, the immunomodulator is an agonistic antibody that targets an activating receptor on an immune cell (such as a T cell). In some embodiments, the immunomodulator is an immune checkpoint inhibitor. In some embodiments, the immune checkpoint inhibitor is an antagonistic antibody that targets an immune checkpoint protein. In some embodiments, the immunomodulator is an IMiDs® compound (small molecule immunomodulator, such as lenalidomide or pomalidomide). In some embodiments, the immunomodulator is lenalidomide. In some embodiments, the immunomodulator is pomalidomide. In some embodiments, the immunomodulator is small molecule or antibody-based IDO inhibitor. In some embodiments, the breast cancer (such as HR+ breast cancer) is recurrent breast cancer (such as HR+ breast cancer). In some embodiments, the breast cancer (such as HR+ breast cancer) is refractory to one or more drugs used in a standard therapy for breast cancer (such as HR+ breast cancer), such as, but not limited to, docetaxel, paclitaxel, cisplatin, carboplatin, vinorelbine, capecitabine, liposomal doxorubicin, gemcitabine, mitoxantrone, ixabepilone, nab-paclitaxel, and/or eribulin.

In some embodiments, there is provided a method of treating breast cancer (such as HR+ breast cancer) in an individual (such as a human) comprising administering to the individual a) an effective amount of a composition comprising nanoparticles comprising an mTOR inhibitor (such as a limus drug, e.g., sirolimus or a derivative thereof) and an albumin; and b) an effective amount of a histone deacetylase inhibitor (such as romidepsin). In some embodiments, the method comprises administering to the individual a) an effective amount of a composition comprising nanoparticles comprising an mTOR inhibitor (such as a limus drug, e.g., sirolimus or a derivative thereof) and an albumin, wherein the mTOR inhibitor in the nanoparticles is associated (e.g., coated) with the albumin; and b) an effective amount of a histone deacetylase inhibitor (such as romidepsin). In some embodiments, the method comprises administering to the individual a) an effective amount of a composition comprising nanoparticles comprising an mTOR inhibitor (such as a limus drug, e.g., sirolimus or a derivative thereof) and an albumin, wherein the nanoparticles have an average particle size of no greater than about 150 nm (such as no greater than about 120 nm); and b) an effective amount of a histone deacetylase inhibitor (such as romidepsin). In some embodiments, the method comprises administering to the individual a) an effective amount of a composition comprising nanoparticles comprising an mTOR inhibitor (such as a limus drug, e.g., sirolimus or a derivative thereof) and an albumin, wherein the nanoparticles comprise the mTOR inhibitor associated (e.g., coated) with albumin, wherein the nanoparticles have an average particle size of no greater than about 150 nm (such as no greater than about 120 nm); and b) an effective amount of a histone deacetylase inhibitor (such as romidepsin). In some embodiments, the method comprises administering to the individual a) an effective amount of a composition comprising nanoparticles comprising an mTOR inhibitor (such as a limus drug, e.g., sirolimus or a derivative thereof) and an albumin, wherein the nanoparticles comprise the mTOR inhibitor associated (e.g., coated) with the albumin, wherein the nanoparticles have an average particle size of no greater than about 150 nm (such as no greater than about 120 nm, for example about 100 nm), wherein the weight ratio of albumin and the mTOR inhibitor in the mTOR inhibitor nanoparticle composition is about 9:1 or less (such as about 9:1 or about 8:1); and b) an effective amount of a histone deacetylase inhibitor (such as romidepsin). In some embodiments, the method further comprises administering to the individual at least one therapeutic agent used in a standard combination therapy with the histone deacetylase inhibitor. In some embodiments, the mTOR inhibitor is a limus drug. In some embodiments, the mTOR inhibitor is sirolimus or a derivative thereof. In some embodiments, the mTOR inhibitor nanoparticle composition comprises nab-sirolimus. In some embodiments, the mTOR inhibitor nanoparticle composition is nab-sirolimus. In some embodiments, the histone deacetylase inhibitor is selected from the group consisting of romidepsin, panobinostat, ricolinostat, and belinostat. In some embodiments, the histone deacetylase inhibitor is romidepsin. In some embodiments, the breast cancer (such as HR+ breast cancer) is recurrent breast cancer (such as HR+ breast cancer). In some embodiments, the breast cancer (such as HR+ breast cancer) is refractory to one or more drugs used in a standard therapy for breast cancer (such as HR+ breast cancer), such as, but not limited to, docetaxel, paclitaxel, cisplatin, carboplatin, vinorelbine, capecitabine, liposomal doxorubicin, gemcitabine, mitoxantrone, ixabepilone, nab-paclitaxel, and/or eribulin.

In some embodiments, there is provided a method of treating breast cancer (such as HR+ breast cancer) in an individual (such as a human) comprising administering to the individual a) an effective amount of a composition comprising nanoparticles comprising sirolimus or a derivative thereof and an albumin; and b) an effective amount of a histone deacetylase inhibitor (such as romidepsin). In some embodiments, the method comprises administering to the individual a) an effective amount of a composition comprising nanoparticles comprising sirolimus or a derivative thereof and an albumin, wherein the sirolimus or derivative thereof in the nanoparticles is associated (e.g., coated) with the albumin; and b) an effective amount of a histone deacetylase inhibitor (such as romidepsin). In some embodiments, the method comprises administering to the individual a) an effective amount of a composition comprising nanoparticles comprising sirolimus or a derivative thereof and an albumin, wherein the nanoparticles have an average particle size of no greater than about 150 nm (such as no greater than about 120 nm); and b) an effective amount of a histone deacetylase inhibitor (such as romidepsin). In some embodiments, the method comprises administering to the individual a) an effective amount of a composition comprising nanoparticles comprising sirolimus or a derivative thereof and an albumin, wherein the nanoparticles comprise the sirolimus or derivative thereof associated (e.g., coated) with albumin, wherein the nanoparticles have an average particle size of no greater than about 150 nm (such as no greater than about 120 nm); and b) an effective amount of a histone deacetylase inhibitor (such as romidepsin). In some embodiments, the method comprises administering to the individual a) an effective amount of a composition comprising nanoparticles comprising sirolimus or a derivative thereof and albumin, wherein the nanoparticles comprise the sirolimus or derivative thereof associated (e.g., coated) with the albumin, wherein the nanoparticles have an average particle size of no greater than about 150 nm (such as no greater than about 120 nm, for example about 100 nm), wherein the weight ratio of albumin and sirolimus or a derivative thereof in the sirolimus nanoparticle composition is about 9:1 or less (such as about 9:1 or about 8:1); and b) an effective amount of a histone deacetylase inhibitor (such as romidepsin). In some embodiments, the method further comprises administering to the individual at least one therapeutic agent used in a standard combination therapy with the histone deacetylase inhibitor. In some embodiments, the sirolimus or derivative thereof is sirolimus. In some embodiments, the sirolimus nanoparticle composition comprises nab-sirolimus. In some embodiments, the sirolimus nanoparticle composition is nab-sirolimus. In some embodiments, the histone deacetylase inhibitor is selected from the group consisting of romidepsin, panobinostat, ricolinostat, and belinostat. In some embodiments, the histone deacetylase inhibitor is romidepsin. In some embodiments, the breast cancer (such as HR+ breast cancer) is recurrent breast cancer (such as HR+ breast cancer). In some embodiments, the breast cancer (such as HR+ breast cancer) is refractory to one or more drugs used in a standard therapy for breast cancer (such as HR+ breast cancer), such as, but not limited to, docetaxel, paclitaxel, cisplatin, carboplatin, vinorelbine, capecitabine, liposomal doxorubicin, gemcitabine, mitoxantrone, ixabepilone, nab-paclitaxel, and/or eribulin.

In some embodiments, there is provided a method of treating breast cancer (such as HR+ breast cancer) in an individual (such as a human) comprising administering to the individual a) an effective amount of a composition comprising nanoparticles comprising an mTOR inhibitor (such as a limus drug, e.g., sirolimus or a derivative thereof) and an albumin; and b) an effective amount of a kinase inhibitor (such as a tyrosine kinase inhibitor, e.g., nilotinib or sorafenib). In some embodiments, the method comprises administering to the individual a) an effective amount of a composition comprising nanoparticles comprising an mTOR inhibitor (such as a limus drug, e.g., sirolimus or a derivative thereof) and an albumin, wherein the mTOR inhibitor in the nanoparticles is associated (e.g., coated) with the albumin; and b) an effective amount of a kinase inhibitor (such as a tyrosine kinase inhibitor, e.g., nilotinib or sorafenib). In some embodiments, the method comprises administering to the individual a) an effective amount of a composition comprising nanoparticles comprising an mTOR inhibitor (such as a limus drug, e.g., sirolimus or a derivative thereof) and an albumin, wherein the nanoparticles have an average particle size of no greater than about 150 nm (such as no greater than about 120 nm); and b) an effective amount of a kinase inhibitor (such as a tyrosine kinase inhibitor, e.g., nilotinib or sorafenib). In some embodiments, the method comprises administering to the individual a) an effective amount of a composition comprising nanoparticles comprising an mTOR inhibitor (such as a limus drug, e.g., sirolimus or a derivative thereof) and an albumin, wherein the nanoparticles comprise the mTOR inhibitor associated (e.g., coated) with albumin, wherein the nanoparticles have an average particle size of no greater than about 150 nm (such as no greater than about 120 nm); and b) an effective amount of a kinase inhibitor (such as a tyrosine kinase inhibitor, e.g., nilotinib or sorafenib). In some embodiments, the method comprises administering to the individual a) an effective amount of a composition comprising nanoparticles comprising an mTOR inhibitor (such as a limus drug, e.g., sirolimus or a derivative thereof) and an albumin, wherein the nanoparticles comprise the mTOR inhibitor associated (e.g., coated) with the albumin, wherein the nanoparticles have an average particle size of no greater than about 150 nm (such as no greater than about 120 nm, for example about 100 nm), wherein the weight ratio of albumin and the mTOR inhibitor in the mTOR inhibitor nanoparticle composition is about 9:1 or less (such as about 9:1 or about 8:1); and b) an effective amount of a kinase inhibitor (such as a tyrosine kinase inhibitor, e.g., nilotinib or sorafenib). In some embodiments, the method further comprises administering to the individual at least one therapeutic agent used in a standard combination therapy with the kinase inhibitor. In some embodiments, the mTOR inhibitor is a limus drug. In some embodiments, the mTOR inhibitor is sirolimus or a derivative thereof. In some embodiments, the mTOR inhibitor nanoparticle composition comprises nab-sirolimus. In some embodiments, the mTOR inhibitor nanoparticle composition is nab-sirolimus. In some embodiments, the kinase inhibitor is a tyrosine kinase inhibitor. In some embodiments, the kinase inhibitor is a serine/threonine kinase inhibitor. In some embodiments, the kinase inhibitor is a Raf kinase inhibitor. In some embodiments, the kinase inhibitor inhibits more than one class of kinase (e.g., an inhibitor of more than one of a tyrosine kinase, a Raf kinase, and a serine/threonine kinase). In some embodiments, the kinase inhibitor is selected from the group consisting of erlotinib, imatinib, lapatinib, nilotinib, sorafenib, and sunitinib. In some embodiments, the kinase inhibitor is nilotinib. In some embodiments, the breast cancer (such as HR+ breast cancer) is recurrent breast cancer (such as HR+ breast cancer). In some embodiments, the breast cancer (such as HR+ breast cancer) is refractory to one or more drugs used in a standard therapy for breast cancer (such as HR+ breast cancer), such as, but not limited to, docetaxel, paclitaxel, cisplatin, carboplatin, vinorelbine, capecitabine, liposomal doxorubicin, gemcitabine, mitoxantrone, ixabepilone, nab-paclitaxel, and/or eribulin.

In some embodiments, there is provided a method of treating breast cancer (such as HR+ breast cancer) in an individual (such as a human) comprising administering to the individual a) an effective amount of a composition comprising nanoparticles comprising sirolimus or a derivative thereof and an albumin; and b) an effective amount of a kinase inhibitor (such as a tyrosine kinase inhibitor, e.g., nilotinib or sorafenib). In some embodiments, the method comprises administering to the individual a) an effective amount of a composition comprising nanoparticles comprising sirolimus or a derivative thereof and an albumin, wherein the sirolimus or derivative thereof in the nanoparticles is associated (e.g., coated) with the albumin; and b) an effective amount of a kinase inhibitor (such as a tyrosine kinase inhibitor, e.g., nilotinib or sorafenib). In some embodiments, the method comprises administering to the individual a) an effective amount of a composition comprising nanoparticles comprising sirolimus or a derivative thereof and an albumin, wherein the nanoparticles have an average particle size of no greater than about 150 nm (such as no greater than about 120 nm); and b) an effective amount of a kinase inhibitor (such as a tyrosine kinase inhibitor, e.g., nilotinib or sorafenib). In some embodiments, the method comprises administering to the individual a) an effective amount of a composition comprising nanoparticles comprising sirolimus or a derivative thereof and an albumin, wherein the nanoparticles comprise the sirolimus or derivative thereof associated (e.g., coated) with albumin, wherein the nanoparticles have an average particle size of no greater than about 150 nm (such as no greater than about 120 nm); and b) an effective amount of a kinase inhibitor (such as a tyrosine kinase inhibitor, e.g., nilotinib or sorafenib). In some embodiments, the method comprises administering to the individual a) an effective amount of a composition comprising nanoparticles comprising sirolimus or a derivative thereof and an albumin, wherein the nanoparticles comprise the sirolimus or derivative thereof associated (e.g., coated) with the albumin, wherein the nanoparticles have an average particle size of no greater than about 150 nm (such as no greater than about 120 nm, for example about 100 nm), wherein the weight ratio of albumin and the sirolimus or derivative thereof in the sirolimus nanoparticle composition is about 9:1 or less (such as about 9:1 or about 8:1); and b) an effective amount of a kinase inhibitor (such as a tyrosine kinase inhibitor, e.g., nilotinib or sorafenib). In some embodiments, the method further comprises administering to the individual at least one therapeutic agent used in a standard combination therapy with the kinase inhibitor. In some embodiments, the sirolimus or derivative thereof is sirolimus. In some embodiments, the sirolimus nanoparticle composition comprises nab-sirolimus. In some embodiments, the sirolimus nanoparticle composition is nab-sirolimus. In some embodiments, the kinase inhibitor is a tyrosine kinase inhibitor. In some embodiments, the kinase inhibitor is a serine/threonine kinase inhibitor. In some embodiments, the kinase inhibitor is a Raf kinase inhibitor. In some embodiments, the kinase inhibitor inhibits more than one class of kinase (e.g., an inhibitor of more than one of a tyrosine kinase, a Raf kinase, and a serine/threonine kinase). In some embodiments, the kinase inhibitor is selected from the group consisting of erlotinib, imatinib, lapatinib, nilotinib, sorafenib, and sunitinib. In some embodiments, the kinase inhibitor is nilotinib. In some embodiments, the breast cancer (such as HR+ breast cancer) is recurrent breast cancer (such as HR+ breast cancer). In some embodiments, the breast cancer (such as HR+ breast cancer) is refractory to one or more drugs used in a standard therapy for breast cancer (such as HR+ breast cancer), such as, but not limited to, docetaxel, paclitaxel, cisplatin, carboplatin, vinorelbine, capecitabine, liposomal doxorubicin, gemcitabine, mitoxantrone, ixabepilone, nab-paclitaxel, and/or eribulin.

In some embodiments, there is provided a method of treating breast cancer (such as HR+ breast cancer) in an individual (such as a human) comprising administering to the individual a) an effective amount of a composition comprising nanoparticles comprising an mTOR inhibitor (such as a limus drug, e.g., sirolimus or a derivative thereof) and an albumin; and b) an effective amount of a cancer vaccine. In some embodiments, the method comprises administering to the individual a) an effective amount of a composition comprising nanoparticles comprising an mTOR inhibitor (such as a limus drug, e.g., sirolimus or a derivative thereof) and an albumin, wherein the mTOR inhibitor in the nanoparticles is associated (e.g., coated) with the albumin; and b) an effective amount of a cancer vaccine. In some embodiments, the method comprises administering to the individual a) an effective amount of a composition comprising nanoparticles comprising an mTOR inhibitor (such as a limus drug, e.g., sirolimus or a derivative thereof) and an albumin, wherein the nanoparticles have an average particle size of no greater than about 150 nm (such as no greater than about 120 nm); and b) an effective amount of a cancer vaccine. In some embodiments, the method comprises administering to the individual a) an effective amount of a composition comprising nanoparticles comprising an mTOR inhibitor (such as a limus drug, e.g., sirolimus or a derivative thereof) and an albumin, wherein the nanoparticles comprise the mTOR inhibitor associated (e.g., coated) with albumin, wherein the nanoparticles have an average particle size of no greater than about 150 nm (such as no greater than about 120 nm); and b) an effective amount of a cancer vaccine. In some embodiments, the method comprises administering to the individual a) an effective amount of a composition comprising nanoparticles comprising an mTOR inhibitor (such as a limus drug, e.g., sirolimus or a derivative thereof) and an albumin, wherein the nanoparticles comprise the mTOR inhibitor associated (e.g., coated) with the albumin, wherein the nanoparticles have an average particle size of no greater than about 150 nm (such as no greater than about 120 nm, for example about 100 nm), wherein the weight ratio of albumin and the mTOR inhibitor in the mTOR inhibitor nanoparticle composition is about 9:1 or less (such as about 9:1 or about 8:1); and b) an effective amount of a cancer vaccine. In some embodiments, the method further comprises administering to the individual at least one therapeutic agent used in a standard combination therapy with the cancer vaccine. In some embodiments, the mTOR inhibitor is a limus drug. In some embodiments, the mTOR inhibitor is sirolimus or a derivative thereof. In some embodiments, the mTOR inhibitor nanoparticle composition comprises nab-sirolimus. In some embodiments, the mTOR inhibitor nanoparticle composition is nab-sirolimus. In some embodiments, the cancer vaccine is a vaccine prepared using autologous tumor cells. In some embodiments, the cancer vaccine is a vaccine prepared using allogeneic tumor cells. In some embodiments, the breast cancer (such as HR+ breast cancer) is recurrent breast cancer (such as HR+ breast cancer). In some embodiments, the breast cancer (such as HR+ breast cancer) is refractory to one or more drugs used in a standard therapy for breast cancer (such as HR+ breast cancer), such as, but not limited to, docetaxel, paclitaxel, cisplatin, carboplatin, vinorelbine, capecitabine, liposomal doxorubicin, gemcitabine, mitoxantrone, ixabepilone, nab-paclitaxel, and/or eribulin.

In some embodiments, there is provided a method of treating breast cancer (such as HR+ breast cancer) in an individual (such as a human) comprising administering to the individual a) an effective amount of a composition comprising nanoparticles comprising sirolimus or a derivative thereof and an albumin; and b) an effective amount of a cancer vaccine. In some embodiments, the method comprises administering to the individual a) an effective amount of a composition comprising nanoparticles comprising sirolimus or a derivative thereof and an albumin, wherein the sirolimus or derivative thereof in the nanoparticles is associated (e.g., coated) with the albumin; and b) an effective amount of a cancer vaccine. In some embodiments, the method comprises administering to the individual a) an effective amount of a composition comprising nanoparticles comprising sirolimus or a derivative thereof and an albumin, wherein the nanoparticles have an average particle size of no greater than about 150 nm (such as no greater than about 120 nm); and b) an effective amount of a cancer vaccine. In some embodiments, the method comprises administering to the individual a) an effective amount of a composition comprising nanoparticles comprising sirolimus or a derivative thereof and an albumin, wherein the nanoparticles comprise the sirolimus or derivative thereof associated (e.g., coated) with albumin, wherein the nanoparticles have an average particle size of no greater than about 150 nm (such as no greater than about 120 nm); and b) an effective amount of a cancer vaccine. In some embodiments, the method comprises administering to the individual a) an effective amount of a composition comprising nanoparticles comprising sirolimus or a derivative thereof and an albumin, wherein the nanoparticles comprise the sirolimus or derivative thereof associated (e.g., coated) with the albumin, wherein the nanoparticles have an average particle size of no greater than about 150 nm (such as no greater than about 120 nm, for example about 100 nm), wherein the weight ratio of albumin and the sirolimus or derivative thereof in the sirolimus nanoparticle composition is about 9:1 or less (such as about 9:1 or about 8:1); and b) an effective amount of a cancer vaccine. In some embodiments, the method further comprises administering to the individual at least one therapeutic agent used in a standard combination therapy with the cancer vaccine. In some embodiments, the sirolimus or derivative thereof is sirolimus. In some embodiments, the sirolimus nanoparticle composition comprises nab-sirolimus. In some embodiments, the sirolimus nanoparticle composition is nab-sirolimus. In some embodiments, the cancer vaccine is a vaccine prepared using autologous tumor cells. In some embodiments, the cancer vaccine is a vaccine prepared using allogeneic tumor cells. In some embodiments, the breast cancer (such as HR+ breast cancer) is recurrent breast cancer (such as HR+ breast cancer). In some embodiments, the breast cancer (such as HR+ breast cancer) is refractory to one or more drugs used in a standard therapy for breast cancer (such as HR+ breast cancer), such as, but not limited to, docetaxel, paclitaxel, cisplatin, carboplatin, vinorelbine, capecitabine, liposomal doxorubicin, gemcitabine, mitoxantrone, ixabepilone, nab-paclitaxel, and/or eribulin.

In some embodiments, there is provided a method of treating breast cancer (such as HR+ breast cancer) in an individual (such as a human) comprising administering to the individual a) an effective amount of a composition comprising nanoparticles comprising sirolimus or a derivative thereof and an albumin; and b) an effective amount of a second therapeutic agent selected from the group consisting of docetaxel, paclitaxel, cisplatin, carboplatin, vinorelbine, capecitabine, liposomal doxorubicin, gemcitabine, mitoxantrone, ixabepilone, nab-paclitaxel, and eribulin. In some embodiments, the method comprises administering to the individual a) an effective amount of a composition comprising nanoparticles comprising sirolimus or a derivative thereof and an albumin, wherein the sirolimus or derivative thereof in the nanoparticles is associated (e.g., coated) with the albumin; and b) an effective amount of a second therapeutic agent selected from the group consisting of docetaxel, paclitaxel, cisplatin, carboplatin, vinorelbine, capecitabine, liposomal doxorubicin, gemcitabine, mitoxantrone, ixabepilone, nab-paclitaxel, and eribulin. In some embodiments, the method comprises administering to the individual a) an effective amount of a composition comprising nanoparticles comprising sirolimus or a derivative thereof and an albumin, wherein the nanoparticles have an average particle size of no greater than about 150 nm (such as no greater than about 120 nm); and b) an effective amount of a second therapeutic agent selected from the group consisting of docetaxel, paclitaxel, cisplatin, carboplatin, vinorelbine, capecitabine, liposomal doxorubicin, gemcitabine, mitoxantrone, ixabepilone, nab-paclitaxel, and eribulin. In some embodiments, the method comprises administering to the individual a) an effective amount of a composition comprising nanoparticles comprising sirolimus or a derivative thereof and an albumin, wherein the nanoparticles comprise the sirolimus or derivative thereof associated (e.g., coated) with albumin, wherein the nanoparticles have an average particle size of no greater than about 150 nm (such as no greater than about 120 nm); and b) an effective amount of a second therapeutic agent selected from the group consisting of docetaxel, paclitaxel, cisplatin, carboplatin, vinorelbine, capecitabine, liposomal doxorubicin, gemcitabine, mitoxantrone, ixabepilone, nab-paclitaxel, and eribulin. In some embodiments, the method comprises administering to the individual a) an effective amount of a composition comprising nanoparticles comprising sirolimus or a derivative thereof and an albumin, wherein the nanoparticles comprise the sirolimus or derivative thereof associated (e.g., coated) with the albumin, wherein the nanoparticles have an average particle size of no greater than about 150 nm (such as no greater than about 120 nm, for example about 100 nm), wherein the weight ratio of albumin and the sirolimus or derivative thereof in the sirolimus nanoparticle composition is about 9:1 or less (such as about 9:1 or about 8:1); and b) an effective amount of a second therapeutic agent selected from the group consisting of docetaxel, paclitaxel, cisplatin, carboplatin, vinorelbine, capecitabine, liposomal doxorubicin, gemcitabine, mitoxantrone, ixabepilone, nab-paclitaxel, and eribulin. In some embodiments, the method further comprises administering to the individual at least one therapeutic agent used in a standard combination therapy with the second therapeutic agent. In some embodiments, the sirolimus or derivative thereof is sirolimus. In some embodiments, the sirolimus nanoparticle composition comprises nab-sirolimus. In some embodiments, the sirolimus nanoparticle composition is nab-sirolimus. In some embodiments, the breast cancer (such as HR+ breast cancer) is recurrent breast cancer (such as HR+ breast cancer). In some embodiments, the breast cancer (such as HR+ breast cancer) is refractory to one or more drugs used in a standard therapy for breast cancer (such as HR+ breast cancer), such as, but not limited to, docetaxel, paclitaxel, cisplatin, carboplatin, vinorelbine, capecitabine, liposomal doxorubicin, gemcitabine, mitoxantrone, ixabepilone, nab-paclitaxel, and/or eribulin.

In some embodiments, according to any of the methods of treating breast cancer (such as HR+ breast cancer) in an individual described herein, the individual is a human who exhibits one or more symptoms associated with breast cancer (such as HR+ breast cancer). In some embodiments, the individual is at an early stage of breast cancer (such as HR+ breast cancer). In some embodiments, the individual is at an advanced stage of breast cancer (such as HR+ breast cancer). In some of embodiments, the individual is genetically or otherwise predisposed (e.g., having a risk factor) to developing breast cancer (such as HR+ breast cancer). Individuals at risk for breast cancer (such as HR+ breast cancer) include, e.g., those having relatives who have experienced breast cancer (such as HR+ breast cancer), and those whose risk is determined by analysis of genetic or biochemical markers. In some embodiments, the individual may be a human who has a gene, genetic mutation, or polymorphism associated with breast cancer (such as HR+ breast cancer) (e.g., BRCA1, BRCA2, ATM, CHEK2, RAD51, AR, DIRAS3, ERBB2, TP53, AKT, PTEN, and/or PDK) or has one or more extra copies of a gene associated with breast cancer (such as HR+ breast cancer). In some embodiments, the individual has a ras or PTEN mutation. In some embodiments, the method further comprises identifying a patient population (i.e. breast cancer (such as HR+ breast cancer) population) based on a hormone receptor status of patients having tumor tissue not expressing both ER and PgR. In some embodiments, the cancer cells are dependent on an mTOR pathway to translate one or more mRNAs. In some embodiments, the cancer cells are not capable of synthesizing mRNAs by an mTOR-independent pathway. In some embodiments, the cancer cells have decreased or no PTEN activity or have decreased or no expression of PTEN compared to non-cancerous cells. In some embodiments, the individual has at least one tumor biomarker selected from the group consisting of elevated PI3K activity, elevated mTOR activity, presence of FLT-3ITD, elevated AKT activity, elevated KRAS activity, and elevated NRAS activity. In some embodiments, the individual has a variation in at least one gene selected from the group consisting of drug metabolism genes, cancer genes, and drug target genes.

### Endometrial Cancer

In some embodiments, there is provided a method of treating endometrial cancer in an individual (such as a human) comprising administering to the individual a) an effective amount of a composition comprising nanoparticles comprising an mTOR inhibitor (such as a limus drug, e.g., sirolimus or a derivative thereof) and an albumin; and b) an effective amount of a second therapeutic agent. In some embodiments, the method comprises administering to the individual a) an effective amount of a composition comprising nanoparticles comprising an mTOR inhibitor (such as a limus drug, e.g., sirolimus or a derivative thereof) and an albumin, wherein the mTOR inhibitor in the nanoparticles is associated (e.g., coated) with the albumin; and b) an effective amount of a second therapeutic agent. In some embodiments, the method comprises administering to the individual a) an effective amount of a composition comprising nanoparticles comprising an mTOR inhibitor (such as a limus drug, e.g., sirolimus or a derivative thereof) and an albumin, wherein the nanoparticles have an average particle size of no greater than about 150 nm (such as no greater than about 120 nm); and b) an effective amount of a second therapeutic agent. In some embodiments, the method comprises administering to the individual a) an effective amount of a composition comprising nanoparticles comprising an mTOR inhibitor (such as a limus drug, e.g., sirolimus or a derivative thereof) and an albumin, wherein the nanoparticles comprise the mTOR inhibitor associated (e.g., coated) with albumin, wherein the nanoparticles have an average particle size of no greater than about 150 nm (such as no greater than about 120 nm); and b) an effective amount of a second therapeutic agent. In some embodiments, the method comprises administering to the individual a) an effective amount of a composition comprising nanoparticles comprising an mTOR inhibitor (such as a limus drug, e.g., sirolimus or a derivative thereof) and an albumin, wherein the nanoparticles comprise the mTOR inhibitor associated (e.g., coated) with the albumin, wherein the nanoparticles have an average particle size of no greater than about 150 nm (such as no greater than about 120 nm, for example about 100 nm), wherein the weight ratio of albumin and the mTOR inhibitor in the mTOR inhibitor nanoparticle composition is about 9:1 or less (such as about 9:1 or about 8:1); and b) an effective amount of a second therapeutic agent. In some embodiments, the method further comprises administering to the individual at least one therapeutic agent used in a standard combination therapy with the second therapeutic agent. In some embodiments, the mTOR inhibitor is a limus drug. In some embodiments, the mTOR inhibitor is sirolimus or a derivative thereof. In some embodiments, the mTOR inhibitor nanoparticle composition comprises nab-sirolimus. In some embodiments, the mTOR inhibitor nanoparticle composition is nab-sirolimus. In some embodiments, the second therapeutic agent is selected from the group consisting of an immunomodulator (such as an immunostimulator or an immune checkpoint inhibitor), a histone deacetylase inhibitor, a kinase inhibitor (such as a tyrosine kinase inhibitor), and a cancer vaccine (such as a vaccine prepared using tumor cells or at least one tumor-associated antigen). In some embodiments, the second therapeutic agent is an immunomodulator. In some embodiments, the immunomodulator is an immunostimulator that directly stimulates the immune system of an individual. In some embodiments, the immunomodulator is an agonistic antibody that targets an activating receptor on an immune cell (such as a T cell). In some embodiments, the immunomodulator is an immune checkpoint inhibitor. In some embodiments, the immune checkpoint inhibitor is an antagonistic antibody that targets an immune checkpoint protein. In some embodiments, the immunomodulator is an IMiDs® compound (small molecule immunomodulator, such as lenalidomide or pomalidomide). In some embodiments, the immunomodulator is lenalidomide. In some embodiments, the immunomodulator is pomalidomide. In some embodiments, the immunomodulator is small molecule or antibody-based IDO inhibitor. In some embodiments, the second therapeutic agent is a histone deacetylase inhibitor. In some embodiments, the histone deacetylase inhibitor is specific to only one HDAC. In some embodiments, the histone deacetylase inhibitor is specific to only one class of HDAC. In some embodiments, the histone deacetylase inhibitor is specific to two or more HDACs or two or more classes of HDACs. In some embodiments, the histone deacetylase inhibitor is specific to class I and II HDACs. In some embodiments, the histone deacetylase inhibitor is specific to class III HDACs. In some embodiments, the histone deacetylase inhibitor is selected from the group consisting of romidepsin, panobinostat, ricolinostat, and belinostat. In some embodiments, the histone deacetylase inhibitor is romidepsin. In some embodiments, the second therapeutic agent is a kinase inhibitor, such as a tyrosine kinase inhibitor. In some embodiments, the kinase inhibitor is a serine/threonine kinase inhibitor. In some embodiments, the kinase inhibitor is a Raf kinase inhibitor. In some embodiments, the kinase inhibitor inhibits more than one class of kinase (e.g., an inhibitor of more than one of a tyrosine kinase, a Raf kinase, and a serine/threonine kinase). In some embodiments, the kinase inhibitor is selected from the group consisting of erlotinib, imatinib, lapatinib, nilotinib, sorafenib, and sunitinib. In some embodiments, the kinase inhibitor is sorafenib. In some embodiments, the kinase inhibitor is nilotinib. In some embodiments, the second therapeutic agent is a cancer vaccine, such as a vaccine prepared using tumor cells or at least one tumor-associated antigen. In some embodiments, the second therapeutic agent and the nanoparticle composition are administered sequentially. In some embodiments, the second therapeutic agent and the nanoparticle composition are administered simultaneously. In some embodiments, the second therapeutic agent and the nanoparticle composition are administered concurrently.

In some embodiments, the endometrial cancer is adenocarcinoma, carcinosarcoma, squamous cell carcinoma, undifferentiated carcinoma, small cell carcinoma, or transitional carcinoma. In some embodiments, the endometrial cancer is endometroid cancer, adenocarcinoma with squamous differentiation, adenoacanthoma, adenosquamous carcinoma, secretory carcinoma, ciliated carcinoma, or villoglandular adenocarcinoma. In some embodiments, the endometrial cancer is clear-cell carcinoma, mucinous adenocarcinoma, or papillary serous adenocarcinoma. In some embodiments, the endometrial cancer is grade 1, grade 2, or grade 3. In some embodiments, the endometrial cancer is type 1 endometrial cancer. In some embodiments, the endometrial cancer is type 2 endometrial cancer. In some embodiments, the endometrial cancer is uterine carcinosarcoma.

In some embodiments, there is provided a method of treating endometrial cancer in an individual (such as a human) comprising administering to the individual a) an effective amount of a composition comprising nanoparticles comprising an mTOR inhibitor (such as a limus drug, e.g., sirolimus or a derivative thereof) and an albumin; and b) an effective amount of an immunomodulator (such as lenalidomide, pomalidomide, or an immune checkpoint inhibitor). In some embodiments, the method comprises administering to the individual a) an effective amount of a composition comprising nanoparticles comprising an mTOR inhibitor (such as a limus drug, e.g., sirolimus or a derivative thereof) and an albumin, wherein the mTOR inhibitor in the nanoparticles is associated (e.g., coated) with the albumin; and b) an effective amount of an immunomodulator (such as lenalidomide, pomalidomide, or an immune checkpoint inhibitor). In some embodiments, the method comprises administering to the individual a) an effective amount of a composition comprising nanoparticles comprising an mTOR inhibitor (such as a limus drug, e.g., sirolimus or a derivative thereof) and an albumin, wherein the nanoparticles have an average particle size of no greater than about 150 nm (such as no greater than about 120 nm); and b) an effective amount of an immunomodulator (such as lenalidomide, pomalidomide, or an immune checkpoint inhibitor). In some embodiments, the method comprises administering to the individual a) an effective amount of a composition comprising nanoparticles comprising an mTOR inhibitor (such as a limus drug, e.g., sirolimus or a derivative thereof) and an albumin, wherein the nanoparticles comprise the mTOR inhibitor associated (e.g., coated) with albumin, wherein the nanoparticles have an average particle size of no greater than about 150 nm (such as no greater than about 120 nm); and b) an effective amount of an immunomodulator (such as lenalidomide, pomalidomide, or an immune checkpoint inhibitor). In some embodiments, the method comprises administering to the individual a) an effective amount of a composition comprising nanoparticles comprising an mTOR inhibitor (such as a limus drug, e.g., sirolimus or a derivative thereof) and an albumin, wherein the nanoparticles comprise the mTOR inhibitor associated (e.g., coated) with the albumin, wherein the nanoparticles have an average particle size of no greater than about 150 nm (such as no greater than about 120 nm, for example about 100 nm), wherein the weight ratio of albumin and the mTOR inhibitor in the mTOR inhibitor nanoparticle composition is about 9:1 or less (such as about 9:1 or about 8:1); and b) an effective amount of an immunomodulator (such as lenalidomide, pomalidomide, or an immune checkpoint inhibitor). In some embodiments, the method further comprises administering to the individual at least one therapeutic agent used in a standard combination therapy with the immunomodulator. In some embodiments, the mTOR inhibitor is a limus drug. In some embodiments, the mTOR inhibitor is sirolimus or a derivative thereof. In some embodiments, the mTOR inhibitor nanoparticle composition comprises nab-sirolimus. In some embodiments, the mTOR inhibitor nanoparticle composition is nab-sirolimus. In some embodiments, the immunomodulator is an immunostimulator that directly stimulates the immune system of an individual. In some embodiments, the immunomodulator is an agonistic antibody that targets an activating receptor on an immune cell (such as a T cell). In some embodiments, the immunomodulator is an immune checkpoint inhibitor. In some embodiments, the immune checkpoint inhibitor is an antagonistic antibody that targets an immune checkpoint protein. In some embodiments, the immunomodulator is an IMiDs® compound (small molecule immunomodulator, such as lenalidomide or pomalidomide). In some embodiments, the immunomodulator is lenalidomide. In some embodiments, the immunomodulator is pomalidomide. In some embodiments, the immunomodulator is small molecule or antibody-based IDO inhibitor. In some embodiments, the endometrial cancer is recurrent endometrial cancer. In some embodiments, the endometrial cancer is refractory to one or more drugs used in a standard therapy for endometrial cancer, such as, but not limited to, paclitaxel, carboplatin, doxorubicin, and/or cisplatin.

In some embodiments, there is provided a method of treating endometrial cancer in an individual (such as a human) comprising administering to the individual a) an effective amount of a composition comprising nanoparticles comprising sirolimus or a derivative thereof and an albumin; and b) an effective amount of an immunomodulator (such as an immunostimulator, e.g., pomalidomide). In some embodiments, the method comprises administering to the individual a) an effective amount of a composition comprising nanoparticles comprising sirolimus or a derivative thereof and an albumin, wherein the sirolimus or derivative thereof in the nanoparticles is associated (e.g., coated) with the albumin; and b) an effective amount of an immunomodulator (such as an immunostimulator, e.g., pomalidomide). In some embodiments, the method comprises administering to the individual a) an effective amount of a composition comprising nanoparticles comprising sirolimus or a derivative thereof and an albumin, wherein the nanoparticles have an average particle size of no greater than about 150 nm (such as no greater than about 120 nm); and b) an effective amount of an immunomodulator (such as an immunostimulator, e.g., pomalidomide). In some embodiments, the method comprises administering to the individual a) an effective amount of a composition comprising nanoparticles comprising sirolimus or a derivative thereof and an albumin, wherein the nanoparticles comprise the sirolimus or derivative thereof associated (e.g., coated) with albumin, wherein the nanoparticles have an average particle size of no greater than about 150 nm (such as no greater than about 120 nm); and b) an effective amount of an immunomodulator (such as an immunostimulator, e.g., pomalidomide). In some embodiments, the method comprises administering to the individual a) an effective amount of a composition comprising nanoparticles comprising sirolimus or a derivative thereof and albumin, wherein the nanoparticles comprise the sirolimus or derivative thereof associated (e.g., coated) with the albumin, wherein the nanoparticles have an average particle size of no greater than about 150 nm (such as no greater than about 120 nm, for example about 100 nm), wherein the weight ratio of albumin and sirolimus or a derivative thereof in the sirolimus nanoparticle composition is about 9:1 or less (such as about 9:1 or about 8:1); and b) an effective amount of an immunomodulator (such as an immunostimulator, e.g., pomalidomide). In some embodiments, the method further comprises administering to the individual at least one therapeutic agent used in a standard combination therapy with the immunomodulator. In some embodiments, the sirolimus or derivative thereof is sirolimus. In some embodiments, the sirolimus nanoparticle composition comprises nab-sirolimus. In some embodiments, the sirolimus nanoparticle composition is nab-sirolimus. In some embodiments, the immunomodulator is an immunostimulator that directly stimulates the immune system of an individual. In some embodiments, the immunomodulator is an agonistic antibody that targets an activating receptor on an immune cell (such as a T cell). In some embodiments, the immunomodulator is an immune checkpoint inhibitor. In some embodiments, the immune checkpoint inhibitor is an antagonistic antibody that targets an immune checkpoint protein. In some embodiments, the immunomodulator is an IMiDs® compound (small molecule immunomodulator, such as lenalidomide or pomalidomide). In some embodiments, the immunomodulator is lenalidomide. In some embodiments, the immunomodulator is pomalidomide. In some embodiments, the immunomodulator is small molecule or antibody-based IDO inhibitor. In some embodiments, the endometrial cancer is recurrent endometrial cancer. In some embodiments, the endometrial cancer is refractory to one or more drugs used in a standard therapy for endometrial cancer, such as, but not limited to, paclitaxel, carboplatin, doxorubicin, and/or cisplatin.

In some embodiments, there is provided a method of treating endometrial cancer in an individual (such as a human) comprising administering to the individual a) an effective amount of a composition comprising nanoparticles comprising an mTOR inhibitor (such as a limus drug, e.g., sirolimus or a derivative thereof) and an albumin; and b) an effective amount of a histone deacetylase inhibitor (such as romidepsin). In some embodiments, the method comprises administering to the individual a) an effective amount of a composition comprising nanoparticles comprising an mTOR inhibitor (such as a limus drug, e.g., sirolimus or a derivative thereof) and an albumin, wherein the mTOR inhibitor in the nanoparticles is associated (e.g., coated) with the albumin; and b) an effective amount of a histone deacetylase inhibitor (such as romidepsin). In some embodiments, the method comprises administering to the individual a) an effective amount of a composition comprising nanoparticles comprising an mTOR inhibitor (such as a limus drug, e.g., sirolimus or a derivative thereof) and an albumin, wherein the nanoparticles have an average particle size of no greater than about 150 nm (such as no greater than about 120 nm); and b) an effective amount of a histone deacetylase inhibitor (such as romidepsin). In some embodiments, the method comprises administering to the individual a) an effective amount of a composition comprising nanoparticles comprising an mTOR inhibitor (such as a limus drug, e.g., sirolimus or a derivative thereof) and an albumin, wherein the nanoparticles comprise the mTOR inhibitor associated (e.g., coated) with albumin, wherein the nanoparticles have an average particle size of no greater than about 150 nm (such as no greater than about 120 nm); and b) an effective amount of a histone deacetylase inhibitor (such as romidepsin). In some embodiments, the method comprises administering to the individual a) an effective amount of a composition comprising nanoparticles comprising an mTOR inhibitor (such as a limus drug, e.g., sirolimus or a derivative thereof) and an albumin, wherein the nanoparticles comprise the mTOR inhibitor associated (e.g., coated) with the albumin, wherein the nanoparticles have an average particle size of no greater than about 150 nm (such as no greater than about 120 nm, for example about 100 nm), wherein the weight ratio of albumin and the mTOR inhibitor in the mTOR inhibitor nanoparticle composition is about 9:1 or less (such as about 9:1 or about 8:1); and b) an effective amount of a histone deacetylase inhibitor (such as romidepsin). In some embodiments, the method further comprises administering to the individual at least one therapeutic agent used in a standard combination therapy with the histone deacetylase inhibitor. In some embodiments, the mTOR inhibitor is a limus drug. In some embodiments, the mTOR inhibitor is sirolimus or a derivative thereof. In some embodiments, the mTOR inhibitor nanoparticle composition comprises nab-sirolimus. In some embodiments, the mTOR inhibitor nanoparticle composition is nab-sirolimus. In some embodiments, the histone deacetylase inhibitor is selected from the group consisting of romidepsin, panobinostat, ricolinostat, and belinostat. In some embodiments, the histone deacetylase inhibitor is romidepsin. In some embodiments, the endometrial cancer is recurrent endometrial cancer. In some embodiments, the endometrial cancer is refractory to one or more drugs used in a standard therapy for endometrial cancer, such as, but not limited to, paclitaxel, carboplatin, doxorubicin, and/or cisplatin.

In some embodiments, there is provided a method of treating endometrial cancer in an individual (such as a human) comprising administering to the individual a) an effective amount of a composition comprising nanoparticles comprising sirolimus or a derivative thereof and an albumin; and b) an effective amount of a histone deacetylase inhibitor (such as romidepsin). In some embodiments, the method comprises administering to the individual a) an effective amount of a composition comprising nanoparticles comprising sirolimus or a derivative thereof and an albumin, wherein the sirolimus or derivative thereof in the nanoparticles is associated (e.g., coated) with the albumin; and b) an effective amount of a histone deacetylase inhibitor (such as romidepsin). In some embodiments, the method comprises administering to the individual a) an effective amount of a composition comprising nanoparticles comprising sirolimus or a derivative thereof and an albumin, wherein the nanoparticles have an average particle size of no greater than about 150 nm (such as no greater than about 120 nm); and b) an effective amount of a histone deacetylase inhibitor (such as romidepsin). In some embodiments, the method comprises administering to the individual a) an effective amount of a composition comprising nanoparticles comprising sirolimus or a derivative thereof and an albumin, wherein the nanoparticles comprise the sirolimus or derivative thereof associated (e.g., coated) with albumin, wherein the nanoparticles have an average particle size of no greater than about 150 nm (such as no greater than about 120 nm); and b) an effective amount of a histone deacetylase inhibitor (such as romidepsin). In some embodiments, the method comprises administering to the individual a) an effective amount of a composition comprising nanoparticles comprising sirolimus or a derivative thereof and albumin, wherein the nanoparticles comprise the sirolimus or derivative thereof associated (e.g., coated) with the albumin, wherein the nanoparticles have an average particle size of no greater than about 150 nm (such as no greater than about 120 nm, for example about 100 nm), wherein the weight ratio of albumin and sirolimus or a derivative thereof in the sirolimus nanoparticle composition is about 9:1 or less (such as about 9:1 or about 8:1); and b) an effective amount of a histone deacetylase inhibitor (such as romidepsin). In some embodiments, the method further comprises administering to the individual at least one therapeutic agent used in a standard combination therapy with the histone deacetylase inhibitor. In some embodiments, the sirolimus or derivative thereof is sirolimus. In some embodiments, the sirolimus nanoparticle composition comprises nab-sirolimus. In some embodiments, the sirolimus nanoparticle composition is nab-sirolimus. In some embodiments, the histone deacetylase inhibitor is selected from the group consisting of romidepsin, panobinostat, ricolinostat, and belinostat. In some embodiments, the histone deacetylase inhibitor is romidepsin. In some embodiments, the endometrial cancer is recurrent endometrial cancer. In some embodiments, the endometrial cancer is refractory to one or more drugs used in a standard therapy for endometrial cancer, such as, but not limited to, paclitaxel, carboplatin, doxorubicin, and/or cisplatin.

In some embodiments, there is provided a method of treating endometrial cancer in an individual (such as a human) comprising administering to the individual a) an effective amount of a composition comprising nanoparticles comprising an mTOR inhibitor (such as a limus drug, e.g., sirolimus or a derivative thereof) and an albumin; and b) an effective amount of a kinase inhibitor (such as a tyrosine kinase inhibitor, e.g., nilotinib or sorafenib). In some embodiments, the method comprises administering to the individual a) an effective amount of a composition comprising nanoparticles comprising an mTOR inhibitor (such as a limus drug, e.g., sirolimus or a derivative thereof) and an albumin, wherein the mTOR inhibitor in the nanoparticles is associated (e.g., coated) with the albumin; and b) an effective amount of a kinase inhibitor (such as a tyrosine kinase inhibitor, e.g., nilotinib or sorafenib). In some embodiments, the method comprises administering to the individual a) an effective amount of a composition comprising nanoparticles comprising an mTOR inhibitor (such as a limus drug, e.g., sirolimus or a derivative thereof) and an albumin, wherein the nanoparticles have an average particle size of no greater than about 150 nm (such as no greater than about 120 nm); and b) an effective amount of a kinase inhibitor (such as a tyrosine kinase inhibitor, e.g., nilotinib or sorafenib). In some embodiments, the method comprises administering to the individual a) an effective amount of a composition comprising nanoparticles comprising an mTOR inhibitor (such as a limus drug, e.g., sirolimus or a derivative thereof) and an albumin, wherein the nanoparticles comprise the mTOR inhibitor associated (e.g., coated) with albumin, wherein the nanoparticles have an average particle size of no greater than about 150 nm (such as no greater than about 120 nm); and b) an effective amount of a kinase inhibitor (such as a tyrosine kinase inhibitor, e.g., nilotinib or sorafenib). In some embodiments, the method comprises administering to the individual a) an effective amount of a composition comprising nanoparticles comprising an mTOR inhibitor (such as a limus drug, e.g., sirolimus or a derivative thereof) and an albumin, wherein the nanoparticles comprise the mTOR inhibitor associated (e.g., coated) with the albumin, wherein the nanoparticles have an average particle size of no greater than about 150 nm (such as no greater than about 120 nm, for example about 100 nm), wherein the weight ratio of albumin and the mTOR inhibitor in the mTOR inhibitor nanoparticle composition is about 9:1 or less (such as about 9:1 or about 8:1); and b) an effective amount of a kinase inhibitor (such as a tyrosine kinase inhibitor, e.g., nilotinib or sorafenib). In some embodiments, the method further comprises administering to the individual at least one therapeutic agent used in a standard combination therapy with the kinase inhibitor. In some embodiments, the mTOR inhibitor is a limus drug. In some embodiments, the mTOR inhibitor is sirolimus or a derivative thereof. In some embodiments, the mTOR inhibitor nanoparticle composition comprises nab-sirolimus. In some embodiments, the mTOR inhibitor nanoparticle composition is nab-sirolimus. In some embodiments, the kinase inhibitor is a tyrosine kinase inhibitor. In some embodiments, the kinase inhibitor is a serine/threonine kinase inhibitor. In some embodiments, the kinase inhibitor is a Raf kinase inhibitor. In some embodiments, the kinase inhibitor inhibits more than one class of kinase (e.g., an inhibitor of more than one of a tyrosine kinase, a Raf kinase, and a serine/threonine kinase). In some embodiments, the kinase inhibitor is selected from the group consisting of erlotinib, imatinib, lapatinib, nilotinib, sorafenib, and sunitinib. In some embodiments, the kinase inhibitor is nilotinib. In some embodiments, the endometrial cancer is recurrent endometrial cancer. In some embodiments, the endometrial cancer is refractory to one or more drugs used in a standard therapy for endometrial cancer, such as, but not limited to, paclitaxel, carboplatin, doxorubicin, and/or cisplatin.

In some embodiments, there is provided a method of treating endometrial cancer in an individual (such as a human) comprising administering to the individual a) an effective amount of a composition comprising nanoparticles comprising sirolimus or a derivative thereof and an albumin; and b) an effective amount of a kinase inhibitor (such as a tyrosine kinase inhibitor, e.g., nilotinib or sorafenib). In some embodiments, the method comprises administering to the individual a) an effective amount of a composition comprising nanoparticles comprising sirolimus or a derivative thereof and an albumin, wherein the sirolimus or derivative thereof in the nanoparticles is associated (e.g., coated) with the albumin; and b) an effective amount of a kinase inhibitor (such as a tyrosine kinase inhibitor, e.g., nilotinib or sorafenib). In some embodiments, the method comprises administering to the individual a) an effective amount of a composition comprising nanoparticles comprising sirolimus or a derivative thereof and an albumin, wherein the nanoparticles have an average particle size of no greater than about 150 nm (such as no greater than about 120 nm); and b) an effective amount of a kinase inhibitor (such as a tyrosine kinase inhibitor, e.g., nilotinib or sorafenib). In some embodiments, the method comprises administering to the individual a) an effective amount of a composition comprising nanoparticles comprising sirolimus or a derivative thereof and an albumin, wherein the nanoparticles comprise the sirolimus or derivative thereof associated (e.g., coated) with albumin, wherein the nanoparticles have an average particle size of no greater than about 150 nm (such as no greater than about 120 nm); and b) an effective amount of a kinase inhibitor (such as a tyrosine kinase inhibitor, e.g., nilotinib or sorafenib). In some embodiments, the method comprises administering to the individual a) an effective amount of a composition comprising nanoparticles comprising sirolimus or a derivative thereof and an albumin, wherein the nanoparticles comprise the sirolimus or derivative thereof associated (e.g., coated) with the albumin, wherein the nanoparticles have an average particle size of no greater than about 150 nm (such as no greater than about 120 nm, for example about 100 nm), wherein the weight ratio of albumin and the sirolimus or derivative thereof in the sirolimus nanoparticle composition is about 9:1 or less (such as about 9:1 or about 8:1); and b) an effective amount of a kinase inhibitor (such as a tyrosine kinase inhibitor, e.g., nilotinib or sorafenib). In some embodiments, the method further comprises administering to the individual at least one therapeutic agent used in a standard combination therapy with the kinase inhibitor. In some embodiments, the sirolimus or derivative thereof is sirolimus. In some embodiments, the sirolimus nanoparticle composition comprises nab-sirolimus. In some embodiments, the sirolimus nanoparticle composition is nab-sirolimus. In some embodiments, the kinase inhibitor is a tyrosine kinase inhibitor. In some embodiments, the kinase inhibitor is a serine/threonine kinase inhibitor. In some embodiments, the kinase inhibitor is a Raf kinase inhibitor. In some embodiments, the kinase inhibitor inhibits more than one class of kinase (e.g., an inhibitor of more than one of a tyrosine kinase, a Raf kinase, and a serine/threonine kinase). In some embodiments, the kinase inhibitor is selected from the group consisting of erlotinib, imatinib, lapatinib, nilotinib, sorafenib, and sunitinib. In some embodiments, the kinase inhibitor is nilotinib. In some embodiments, the endometrial cancer is recurrent endometrial cancer. In some embodiments, the endometrial cancer is refractory to one or more drugs used in a standard therapy for endometrial cancer, such as, but not limited to, paclitaxel, carboplatin, doxorubicin, and/or cisplatin.

In some embodiments, there is provided a method of treating endometrial cancer in an individual (such as a human) comprising administering to the individual a) an effective amount of a composition comprising nanoparticles comprising an mTOR inhibitor (such as a limus drug, e.g., sirolimus or a derivative thereof) and an albumin; and b) an effective amount of a cancer vaccine. In some embodiments, the method comprises administering to the individual a) an effective amount of a composition comprising nanoparticles comprising an mTOR inhibitor (such as a limus drug, e.g., sirolimus or a derivative thereof) and an albumin, wherein the mTOR inhibitor in the nanoparticles is associated (e.g., coated) with the albumin; and b) an effective amount of a cancer vaccine. In some embodiments, the method comprises administering to the individual a) an effective amount of a composition comprising nanoparticles comprising an mTOR inhibitor (such as a limus drug, e.g., sirolimus or a derivative thereof) and an albumin, wherein the nanoparticles have an average particle size of no greater than about 150 nm (such as no greater than about 120 nm); and b) an effective amount of a cancer vaccine. In some embodiments, the method comprises administering to the individual a) an effective amount of a composition comprising nanoparticles comprising an mTOR inhibitor (such as a limus drug, e.g., sirolimus or a derivative thereof) and an albumin, wherein the nanoparticles comprise the mTOR inhibitor associated (e.g., coated) with albumin, wherein the nanoparticles have an average particle size of no greater than about 150 nm (such as no greater than about 120 nm); and b) an effective amount of a cancer vaccine. In some embodiments, the method comprises administering to the individual a) an effective amount of a composition comprising nanoparticles comprising an mTOR inhibitor (such as a limus drug, e.g., sirolimus or a derivative thereof) and an albumin, wherein the nanoparticles comprise the mTOR inhibitor associated (e.g., coated) with the albumin, wherein the nanoparticles have an average particle size of no greater than about 150 nm (such as no greater than about 120 nm, for example about 100 nm), wherein the weight ratio of albumin and the mTOR inhibitor in the mTOR inhibitor nanoparticle composition is about 9:1 or less (such as about 9:1 or about 8:1); and b) an effective amount of a cancer vaccine. In some embodiments, the method further comprises administering to the individual at least one therapeutic agent used in a standard combination therapy with the cancer vaccine. In some embodiments, the mTOR inhibitor is a limus drug. In some embodiments, the mTOR inhibitor is sirolimus or a derivative thereof. In some embodiments, the mTOR inhibitor nanoparticle composition comprises nab-sirolimus. In some embodiments, the mTOR inhibitor nanoparticle composition is nab-sirolimus. In some embodiments, the cancer vaccine is a vaccine prepared using autologous tumor cells. In some embodiments, the cancer vaccine is a vaccine prepared using allogeneic tumor cells. In some embodiments, the endometrial cancer is recurrent endometrial cancer. In some embodiments, the endometrial cancer is refractory to one or more drugs used in a standard therapy for endometrial cancer, such as, but not limited to, paclitaxel, carboplatin, doxorubicin, and/or cisplatin.

In some embodiments, there is provided a method of treating endometrial cancer in an individual (such as a human) comprising administering to the individual a) an effective amount of a composition comprising nanoparticles comprising sirolimus or a derivative thereof and an albumin; and b) an effective amount of a cancer vaccine. In some embodiments, the method comprises administering to the individual a) an effective amount of a composition comprising nanoparticles comprising sirolimus or a derivative thereof and an albumin, wherein the sirolimus or derivative thereof in the nanoparticles is associated (e.g., coated) with the albumin; and b) an effective amount of a cancer vaccine. In some embodiments, the method comprises administering to the individual a) an effective amount of a composition comprising nanoparticles comprising sirolimus or a derivative thereof and an albumin, wherein the nanoparticles have an average particle size of no greater than about 150 nm (such as no greater than about 120 nm); and b) an effective amount of a cancer vaccine. In some embodiments, the method comprises administering to the individual a) an effective amount of a composition comprising nanoparticles comprising sirolimus or a derivative thereof and an albumin, wherein the nanoparticles comprise the sirolimus or derivative thereof associated (e.g., coated) with albumin, wherein the nanoparticles have an average particle size of no greater than about 150 nm (such as no greater than about 120 nm); and b) an effective amount of a cancer vaccine. In some embodiments, the method comprises administering to the individual a) an effective amount of a composition comprising nanoparticles comprising sirolimus or a derivative thereof and an albumin, wherein the nanoparticles comprise the sirolimus or derivative thereof associated (e.g., coated) with the albumin, wherein the nanoparticles have an average particle size of no greater than about 150 nm (such as no greater than about 120 nm, for example about 100 nm), wherein the weight ratio of albumin and the sirolimus or derivative thereof in the sirolimus nanoparticle composition is about 9:1 or less (such as about 9:1 or about 8:1); and b) an effective amount of a cancer vaccine. In some embodiments, the method further comprises administering to the individual at least one therapeutic agent used in a standard combination therapy with the cancer vaccine. In some embodiments, the sirolimus or derivative thereof is sirolimus. In some embodiments, the sirolimus nanoparticle composition comprises nab-sirolimus. In some embodiments, the sirolimus nanoparticle composition is nab-sirolimus. In some embodiments, the cancer vaccine is a vaccine prepared using autologous tumor cells. In some embodiments, the cancer vaccine is a vaccine prepared using allogeneic tumor cells. In some embodiments, the endometrial cancer is recurrent endometrial cancer. In some embodiments, the endometrial cancer is refractory to one or more drugs used in a standard therapy for endometrial cancer, such as, but not limited to, paclitaxel, carboplatin, doxorubicin, and/or cisplatin.

In some embodiments, there is provided a method of treating endometrial cancer in an individual (such as a human) comprising administering to the individual a) an effective amount of a composition comprising nanoparticles comprising sirolimus or a derivative thereof and an albumin; and b) an effective amount of a second therapeutic agent selected from the group consisting of paclitaxel, carboplatin, doxorubicin, and cisplatin. In some embodiments, the method comprises administering to the individual a) an effective amount of a composition comprising nanoparticles comprising sirolimus or a derivative thereof and an albumin, wherein the sirolimus or derivative thereof in the nanoparticles is associated (e.g., coated) with the albumin; and b) an effective amount of a second therapeutic agent selected from the group consisting of paclitaxel, carboplatin, doxorubicin, and cisplatin. In some embodiments, the method comprises administering to the individual a) an effective amount of a composition comprising nanoparticles comprising sirolimus or a derivative thereof and an albumin, wherein the nanoparticles have an average particle size of no greater than about 150 nm (such as no greater than about 120 nm); and b) an effective amount of a second therapeutic agent selected from the group consisting of paclitaxel, carboplatin, doxorubicin, and cisplatin. In some embodiments, the method comprises administering to the individual a) an effective amount of a composition comprising nanoparticles comprising sirolimus or a derivative thereof and an albumin, wherein the nanoparticles comprise the sirolimus or derivative thereof associated (e.g., coated) with albumin, wherein the nanoparticles have an average particle size of no greater than about 150 nm (such as no greater than about 120 nm); and b) an effective amount of a second therapeutic agent selected from the group consisting of paclitaxel, carboplatin, doxorubicin, and cisplatin. In some embodiments, the method comprises administering to the individual a) an effective amount of a composition comprising nanoparticles comprising sirolimus or a derivative thereof and an albumin, wherein the nanoparticles comprise the sirolimus or derivative thereof associated (e.g., coated) with the albumin, wherein the nanoparticles have an average particle size of no greater than about 150 nm (such as no greater than about 120 nm, for example about 100 nm), wherein the weight ratio of albumin and the sirolimus or derivative thereof in the sirolimus nanoparticle composition is about 9:1 or less (such as about 9:1 or about 8:1); and b) an effective amount of a second therapeutic agent selected from the group consisting of paclitaxel, carboplatin, doxorubicin, and cisplatin. In some embodiments, the method further comprises administering to the individual at least one therapeutic agent used in a standard combination therapy with the second therapeutic agent. In some embodiments, the sirolimus or derivative thereof is sirolimus. In some embodiments, the sirolimus nanoparticle composition comprises nab-sirolimus. In some embodiments, the sirolimus nanoparticle composition is nab-sirolimus. In some embodiments, the endometrial cancer is recurrent endometrial cancer. In some embodiments, the endometrial cancer is refractory to one or more drugs used in a standard therapy for endometrial cancer, such as, but not limited to, paclitaxel, carboplatin, doxorubicin, and/or cisplatin.

In some embodiments, according to any of the methods of treating endometrial cancer in an individual described herein, the individual is a human who exhibits one or more symptoms associated with endometrial cancer. In some embodiments, the individual is at an early stage of endometrial cancer. In some embodiments, the individual is at an advanced stage of endometrial cancer. In some of embodiments, the individual is genetically or otherwise predisposed (e.g., having a risk factor) to developing endometrial cancer. Individuals at risk for endometrial cancer include, e.g., those having relatives who have experienced endometrial cancer, and those whose risk is determined by analysis of genetic or biochemical markers. In some embodiments, the individual may be a human who has a gene, genetic mutation, or polymorphism associated with endometrial cancer (e.g., MLH1, MLH2, MSH2, MLH3, MSH6, TGBR2, PMS1, and/or PMS2) or has one or more extra copies of a gene associated with endometrial cancer. In some embodiments, the individual has a ras or PTEN mutation. In some embodiments, the cancer cells are dependent on an mTOR pathway to translate one or more mRNAs. In some embodiments, the cancer cells are not capable of synthesizing mRNAs by an mTOR-independent pathway. In some embodiments, the cancer cells have decreased or no PTEN activity or have decreased or no expression of PTEN compared to non-cancerous cells. In some embodiments, the individual has at least one tumor biomarker selected from the group consisting of elevated PI3K activity, elevated mTOR activity, presence of FLT-3ITD, elevated AKT activity, elevated KRAS activity, and elevated NRAS activity. In some embodiments, the individual has a variation in at least one gene selected from the group consisting of drug metabolism genes, cancer genes, and drug target genes.

### Pancreatic Neuroendocrine Cancer

In some embodiments, there is provided a method of treating pancreatic neuroendocrine cancer in an individual (such as a human) comprising administering to the individual a) an effective amount of a composition comprising nanoparticles comprising an mTOR inhibitor (such as a limus drug, e.g., sirolimus or a derivative thereof) and an albumin; and b) an effective amount of a second therapeutic agent. In some embodiments, the method comprises administering to the individual a) an effective amount of a composition comprising nanoparticles comprising an mTOR inhibitor (such as a limus drug, e.g., sirolimus or a derivative thereof) and an albumin, wherein the mTOR inhibitor in the nanoparticles is associated (e.g., coated) with the albumin; and b) an effective amount of a second therapeutic agent. In some embodiments, the method comprises administering to the individual a) an effective amount of a composition comprising nanoparticles comprising an mTOR inhibitor (such as a limus drug, e.g., sirolimus or a derivative thereof) and an albumin, wherein the nanoparticles have an average particle size of no greater than about 150 nm (such as no greater than about 120 nm); and b) an effective amount of a second therapeutic agent. In some embodiments, the method comprises administering to the individual a) an effective amount of a composition comprising nanoparticles comprising an mTOR inhibitor (such as a limus drug, e.g., sirolimus or a derivative thereof) and an albumin, wherein the nanoparticles comprise the mTOR inhibitor associated (e.g., coated) with albumin, wherein the nanoparticles have an average particle size of no greater than about 150 nm (such as no greater than about 120 nm); and b) an effective amount of a second therapeutic agent. In some embodiments, the method comprises administering to the individual a) an effective amount of a composition comprising nanoparticles comprising an mTOR inhibitor (such as a limus drug, e.g., sirolimus or a derivative thereof) and an albumin, wherein the nanoparticles comprise the mTOR inhibitor associated (e.g., coated) with the albumin, wherein the nanoparticles have an average particle size of no greater than about 150 nm (such as no greater than about 120 nm, for example about 100 nm), wherein the weight ratio of albumin and the mTOR inhibitor in the mTOR inhibitor nanoparticle composition is about 9:1 or less (such as about 9:1 or about 8:1); and b) an effective amount of a second therapeutic agent. In some embodiments, the method further comprises administering to the individual at least one therapeutic agent used in a standard combination therapy with the second therapeutic agent. In some embodiments, the mTOR inhibitor is a limus drug. In some embodiments, the mTOR inhibitor is sirolimus or a derivative thereof. In some embodiments, the mTOR inhibitor nanoparticle composition comprises nab-sirolimus. In some embodiments, the mTOR inhibitor nanoparticle composition is nab-sirolimus. In some embodiments, the second therapeutic agent is selected from the group consisting of an immunomodulator (such as an immunostimulator or an immune checkpoint inhibitor), a histone deacetylase inhibitor, a kinase inhibitor (such as a tyrosine kinase inhibitor), and a cancer vaccine (such as a vaccine prepared using tumor cells or at least one tumor-associated antigen). In some embodiments, the second therapeutic agent is an immunomodulator. In some embodiments, the immunomodulator is an immunostimulator that directly stimulates the immune system of an individual. In some embodiments, the immunomodulator is an agonistic antibody that targets an activating receptor on an immune cell (such as a T cell). In some embodiments, the immunomodulator is an immune checkpoint inhibitor. In some embodiments, the immune checkpoint inhibitor is an antagonistic antibody that targets an immune checkpoint protein. In some embodiments, the immunomodulator is an IMiDs® compound (small molecule immunomodulator, such as lenalidomide or pomalidomide). In some embodiments, the immunomodulator is lenalidomide. In some embodiments, the immunomodulator is pomalidomide. In some embodiments, the immunomodulator is small molecule or antibody-based IDO inhibitor. In some embodiments, the second therapeutic agent is a histone deacetylase inhibitor. In some embodiments, the histone deacetylase inhibitor is specific to only one HDAC. In some embodiments, the histone deacetylase inhibitor is specific to only one class of HDAC. In some embodiments, the histone deacetylase inhibitor is specific to two or more HDACs or two or more classes of HDACs. In some embodiments, the histone deacetylase inhibitor is specific to class I and II HDACs. In some embodiments, the histone deacetylase inhibitor is specific to class III HDACs. In some embodiments, the histone deacetylase inhibitor is selected from the group consisting of romidepsin, panobinostat, ricolinostat, and belinostat. In some embodiments, the histone deacetylase inhibitor is romidepsin. In some embodiments, the second therapeutic agent is a kinase inhibitor, such as a tyrosine kinase inhibitor. In some embodiments, the kinase inhibitor is a serine/threonine kinase inhibitor. In some embodiments, the kinase inhibitor is a Raf kinase inhibitor. In some embodiments, the kinase inhibitor inhibits more than one class of kinase (e.g., an inhibitor of more than one of a tyrosine kinase, a Raf kinase, and a serine/threonine kinase). In some embodiments, the kinase inhibitor is selected from the group consisting of erlotinib, imatinib, lapatinib, nilotinib, sorafenib, and sunitinib. In some embodiments, the kinase inhibitor is sorafenib. In some embodiments, the kinase inhibitor is nilotinib. In some embodiments, the second therapeutic agent is a cancer vaccine, such as a vaccine prepared using tumor cells or at least one tumor-associated antigen. In some embodiments, the second therapeutic agent and the nanoparticle composition are administered sequentially. In some embodiments, the second therapeutic agent and the nanoparticle composition are administered simultaneously. In some embodiments, the second therapeutic agent and the nanoparticle composition are administered concurrently.

In some embodiments, the pancreatic neuroendocrine cancer is a well-differentiated neuroendocrine tumor, a well-differentiated (low grade) neuroendocrine carcinoma, or a poorly differentiated (high grade) neuroendocrine carcinoma. In some embodiments, the pancreatic neuroendocrine cancer is a functional or a nonfunctional pancreatic neuroendocrine tumor. In some embodiments, the pancreatic neuroendocrine cancer is insulinoma, glucagonoma, somatostatinoma, gastrinoma, VIPoma, GRFoma, or ACTHoma.

In some embodiments, there is provided a method of treating pancreatic neuroendocrine cancer in an individual (such as a human) comprising administering to the individual a) an effective amount of a composition comprising nanoparticles comprising an mTOR inhibitor (such as a limus drug, e.g., sirolimus or a derivative thereof) and an albumin; and b) an effective amount of an immunomodulator (such as lenalidomide, pomalidomide, or an immune checkpoint inhibitor). In some embodiments, the method comprises administering to the individual a) an effective amount of a composition comprising nanoparticles comprising an mTOR inhibitor (such as a limus drug, e.g., sirolimus or a derivative thereof) and an albumin, wherein the mTOR inhibitor in the nanoparticles is associated (e.g., coated) with the albumin; and b) an effective amount of an immunomodulator (such as lenalidomide, pomalidomide, or an immune checkpoint inhibitor). In some embodiments, the method comprises administering to the individual a) an effective amount of a composition comprising nanoparticles comprising an mTOR inhibitor (such as a limus drug, e.g., sirolimus or a derivative thereof) and an albumin, wherein the nanoparticles have an average particle size of no greater than about 150 nm (such as no greater than about 120 nm); and b) an effective amount of an immunomodulator (such as lenalidomide, pomalidomide, or an immune checkpoint inhibitor). In some embodiments, the method comprises administering to the individual a) an effective amount of a composition comprising nanoparticles comprising an mTOR inhibitor (such as a limus drug, e.g., sirolimus or a derivative thereof) and an albumin, wherein the nanoparticles comprise the mTOR inhibitor associated (e.g., coated) with albumin, wherein the nanoparticles have an average particle size of no greater than about 150 nm (such as no greater than about 120 nm); and b) an effective amount of an immunomodulator (such as lenalidomide, pomalidomide, or an immune checkpoint inhibitor). In some embodiments, the method comprises administering to the individual a) an effective amount of a composition comprising nanoparticles comprising an mTOR inhibitor (such as a limus drug, e.g., sirolimus or a derivative thereof) and an albumin, wherein the nanoparticles comprise the mTOR inhibitor associated (e.g., coated) with the albumin, wherein the nanoparticles have an average particle size of no greater than about 150 nm (such as no greater than about 120 nm, for example about 100 nm), wherein the weight ratio of albumin and the mTOR inhibitor in the mTOR inhibitor nanoparticle composition is about 9:1 or less (such as about 9:1 or about 8:1); and b) an effective amount of an immunomodulator (such as lenalidomide, pomalidomide, or an immune checkpoint inhibitor). In some embodiments, the method further comprises administering to the individual at least one therapeutic agent used in a standard combination therapy with the immunomodulator. In some embodiments, the mTOR inhibitor is a limus drug. In some embodiments, the mTOR inhibitor is sirolimus or a derivative thereof. In some embodiments, the mTOR inhibitor nanoparticle composition comprises nab-sirolimus. In some embodiments, the mTOR inhibitor nanoparticle composition is nab-sirolimus. In some embodiments, the immunomodulator is an immunostimulator that directly stimulates the immune system of an individual. In some embodiments, the immunomodulator is an agonistic antibody that targets an activating receptor on an immune cell (such as a T cell). In some embodiments, the immunomodulator is an immune checkpoint inhibitor. In some embodiments, the immune checkpoint inhibitor is an antagonistic antibody that targets an immune checkpoint protein. In some embodiments, the immunomodulator is an IMiDs® compound (small molecule immunomodulator, such as lenalidomide or pomalidomide). In some embodiments, the immunomodulator is lenalidomide. In some embodiments, the immunomodulator is pomalidomide. In some embodiments, the immunomodulator is small molecule or antibody-based IDO inhibitor. In some embodiments, the pancreatic neuroendocrine cancer is recurrent pancreatic neuroendocrine cancer. In some embodiments, the pancreatic neuroendocrine cancer is refractory to one or more drugs used in a standard therapy for pancreatic neuroendocrine cancer, such as, but not limited to, doxorubicin, streptozocin, fluorouracil (5-FU), dacarbazine, temozolomide, thalidomide, capecitabine, sunitinib, somatostatin analogs (e.g., octreotide, lanreotide, or pasireotide), and/or everolimus.

In some embodiments, there is provided a method of treating pancreatic neuroendocrine cancer in an individual (such as a human) comprising administering to the individual a) an effective amount of a composition comprising nanoparticles comprising sirolimus or a derivative thereof and an albumin; and b) an effective amount of an immunomodulator (such as an immunostimulator, e.g., pomalidomide). In some embodiments, the method comprises administering to the individual a) an effective amount of a composition comprising nanoparticles comprising sirolimus or a derivative thereof and an albumin, wherein the sirolimus or derivative thereof in the nanoparticles is associated (e.g., coated) with the albumin; and b) an effective amount of an immunomodulator (such as an immunostimulator, e.g., pomalidomide). In some embodiments, the method comprises administering to the individual a) an effective amount of a composition comprising nanoparticles comprising sirolimus or a derivative thereof and an albumin, wherein the nanoparticles have an average particle size of no greater than about 150 nm (such as no greater than about 120 nm); and b) an effective amount of an immunomodulator (such as an immunostimulator, e.g., pomalidomide). In some embodiments, the method comprises administering to the individual a) an effective amount of a composition comprising nanoparticles comprising sirolimus or a derivative thereof and an albumin, wherein the nanoparticles comprise the sirolimus or derivative thereof associated (e.g., coated) with albumin, wherein the nanoparticles have an average particle size of no greater than about 150 nm (such as no greater than about 120 nm); and b) an effective amount of an immunomodulator (such as an immunostimulator, e.g., pomalidomide). In some embodiments, the method comprises administering to the individual a) an effective amount of a composition comprising nanoparticles comprising sirolimus or a derivative thereof and albumin, wherein the nanoparticles comprise the sirolimus or derivative thereof associated (e.g., coated) with the albumin, wherein the nanoparticles have an average particle size of no greater than about 150 nm (such as no greater than about 120 nm, for example about 100 nm), wherein the weight ratio of albumin and sirolimus or a derivative thereof in the sirolimus nanoparticle composition is about 9:1 or less (such as about 9:1 or about 8:1); and b) an effective amount of an immunomodulator (such as an immunostimulator, e.g., pomalidomide). In some embodiments, the method further comprises administering to the individual at least one therapeutic agent used in a standard combination therapy with the immunomodulator. In some embodiments, the sirolimus or derivative thereof is sirolimus. In some embodiments, the sirolimus nanoparticle composition comprises nab-sirolimus. In some embodiments, the sirolimus nanoparticle composition is nab-sirolimus. In some embodiments, the immunomodulator is an immunostimulator that directly stimulates the immune system of an individual. In some embodiments, the immunomodulator is an agonistic antibody that targets an activating receptor on an immune cell (such as a T cell). In some embodiments, the immunomodulator is an immune checkpoint inhibitor. In some embodiments, the immune checkpoint inhibitor is an antagonistic antibody that targets an immune checkpoint protein. In some embodiments, the immunomodulator is an IMiDs® compound (small molecule immunomodulator, such as lenalidomide or pomalidomide). In some embodiments, the immunomodulator is lenalidomide. In some embodiments, the immunomodulator is pomalidomide. In some embodiments, the immunomodulator is small molecule or antibody-based IDO inhibitor. In some embodiments, the pancreatic neuroendocrine cancer is recurrent pancreatic neuroendocrine cancer. In some embodiments, the pancreatic neuroendocrine cancer is refractory to one or more drugs used in a standard therapy for pancreatic neuroendocrine cancer, such as, but not limited to, doxorubicin, streptozocin, fluorouracil (5-FU), dacarbazine, temozolomide, thalidomide, capecitabine, sunitinib, somatostatin analogs (e.g., octreotide, lanreotide, or pasireotide), and/or everolimus.

In some embodiments, there is provided a method of treating pancreatic neuroendocrine cancer in an individual (such as a human) comprising administering to the individual a) an effective amount of a composition comprising nanoparticles comprising an mTOR inhibitor (such as a limus drug, e.g., sirolimus or a derivative thereof) and an albumin; and b) an effective amount of a histone deacetylase inhibitor (such as romidepsin). In some embodiments, the method comprises administering to the individual a) an effective amount of a composition comprising nanoparticles comprising an mTOR inhibitor (such as a limus drug, e.g., sirolimus or a derivative thereof) and an albumin, wherein the mTOR inhibitor in the nanoparticles is associated (e.g., coated) with the albumin; and b) an effective amount of a histone deacetylase inhibitor (such as romidepsin). In some embodiments, the method comprises administering to the individual a) an effective amount of a composition comprising nanoparticles comprising an mTOR inhibitor (such as a limus drug, e.g., sirolimus or a derivative thereof) and an albumin, wherein the nanoparticles have an average particle size of no greater than about 150 nm (such as no greater than about 120 nm); and b) an effective amount of a histone deacetylase inhibitor (such as romidepsin). In some embodiments, the method comprises administering to the individual a) an effective amount of a composition comprising nanoparticles comprising an mTOR inhibitor (such as a limus drug, e.g., sirolimus or a derivative thereof) and an albumin, wherein the nanoparticles comprise the mTOR inhibitor associated (e.g., coated) with albumin, wherein the nanoparticles have an average particle size of no greater than about 150 nm (such as no greater than about 120 nm); and b) an effective amount of a histone deacetylase inhibitor (such as romidepsin). In some embodiments, the method comprises administering to the individual a) an effective amount of a composition comprising nanoparticles comprising an mTOR inhibitor (such as a limus drug, e.g., sirolimus or a derivative thereof) and an albumin, wherein the nanoparticles comprise the mTOR inhibitor associated (e.g., coated) with the albumin, wherein the nanoparticles have an average particle size of no greater than about 150 nm (such as no greater than about 120 nm, for example about 100 nm), wherein the weight ratio of albumin and the mTOR inhibitor in the mTOR inhibitor nanoparticle composition is about 9:1 or less (such as about 9:1 or about 8:1); and b) an effective amount of a histone deacetylase inhibitor (such as romidepsin). In some embodiments, the method further comprises administering to the individual at least one therapeutic agent used in a standard combination therapy with the histone deacetylase inhibitor. In some embodiments, the mTOR inhibitor is a limus drug. In some embodiments, the mTOR inhibitor is sirolimus or a derivative thereof. In some embodiments, the mTOR inhibitor nanoparticle composition comprises nab-sirolimus. In some embodiments, the mTOR inhibitor nanoparticle composition is nab-sirolimus. In some embodiments, the histone deacetylase inhibitor is selected from the group consisting of romidepsin, panobinostat, ricolinostat, and belinostat. In some embodiments, the histone deacetylase inhibitor is romidepsin. In some embodiments, the pancreatic neuroendocrine cancer is recurrent pancreatic neuroendocrine cancer. In some embodiments, the pancreatic neuroendocrine cancer is refractory to one or more drugs used in a standard therapy for pancreatic neuroendocrine cancer, such as, but not limited to, doxorubicin, streptozocin, fluorouracil (5-FU), dacarbazine, temozolomide, thalidomide, capecitabine, sunitinib, somatostatin analogs (e.g., octreotide, lanreotide, or pasireotide), and/or everolimus.

In some embodiments, there is provided a method of treating pancreatic neuroendocrine cancer in an individual (such as a human) comprising administering to the individual a) an effective amount of a composition comprising nanoparticles comprising sirolimus or a derivative thereof and an albumin; and b) an effective amount of a histone deacetylase inhibitor (such as romidepsin). In some embodiments, the method comprises administering to the individual a) an effective amount of a composition comprising nanoparticles comprising sirolimus or a derivative thereof and an albumin, wherein the sirolimus or derivative thereof in the nanoparticles is associated (e.g., coated) with the albumin; and b) an effective amount of a histone deacetylase inhibitor (such as romidepsin). In some embodiments, the method comprises administering to the individual a) an effective amount of a composition comprising nanoparticles comprising sirolimus or a derivative thereof and an albumin, wherein the nanoparticles have an average particle size of no greater than about 150 nm (such as no greater than about 120 nm); and b) an effective amount of a histone deacetylase inhibitor (such as romidepsin). In some embodiments, the method comprises administering to the individual a) an effective amount of a composition comprising nanoparticles comprising sirolimus or a derivative thereof and an albumin, wherein the nanoparticles comprise the sirolimus or derivative thereof associated (e.g., coated) with albumin, wherein the nanoparticles have an average particle size of no greater than about 150 nm (such as no greater than about 120 nm); and b) an effective amount of a histone deacetylase inhibitor (such as romidepsin). In some embodiments, the method comprises administering to the individual a) an effective amount of a composition comprising nanoparticles comprising sirolimus or a derivative thereof and albumin, wherein the nanoparticles comprise the sirolimus or derivative thereof associated (e.g., coated) with the albumin, wherein the nanoparticles have an average particle size of no greater than about 150 nm (such as no greater than about 120 nm, for example about 100 nm), wherein the weight ratio of albumin and sirolimus or a derivative thereof in the sirolimus nanoparticle composition is about 9:1 or less (such as about 9:1 or about 8:1); and b) an effective amount of a histone deacetylase inhibitor (such as romidepsin). In some embodiments, the method further comprises administering to the individual at least one therapeutic agent used in a standard combination therapy with the histone deacetylase inhibitor. In some embodiments, the sirolimus or derivative thereof is sirolimus. In some embodiments, the sirolimus nanoparticle composition comprises nab-sirolimus. In some embodiments, the sirolimus nanoparticle composition is nab-sirolimus. In some embodiments, the histone deacetylase inhibitor is selected from the group consisting of romidepsin, panobinostat, ricolinostat, and belinostat. In some embodiments, the histone deacetylase inhibitor is romidepsin. In some embodiments, the pancreatic neuroendocrine cancer is recurrent pancreatic neuroendocrine cancer. In some embodiments, the pancreatic neuroendocrine cancer is refractory to one or more drugs used in a standard therapy for pancreatic neuroendocrine cancer, such as, but not limited to, doxorubicin, streptozocin, fluorouracil (5-FU), dacarbazine, temozolomide, thalidomide, capecitabine, sunitinib, somatostatin analogs (e.g., octreotide, lanreotide, or pasireotide), and/or everolimus.

In some embodiments, there is provided a method of treating pancreatic neuroendocrine cancer in an individual (such as a human) comprising administering to the individual a) an effective amount of a composition comprising nanoparticles comprising an mTOR inhibitor (such as a limus drug, e.g., sirolimus or a derivative thereof) and an albumin; and b) an effective amount of a kinase inhibitor (such as a tyrosine kinase inhibitor, e.g., nilotinib or sorafenib). In some embodiments, the method comprises administering to the individual a) an effective amount of a composition comprising nanoparticles comprising an mTOR inhibitor (such as a limus drug, e.g., sirolimus or a derivative thereof) and an albumin, wherein the mTOR inhibitor in the nanoparticles is associated (e.g., coated) with the albumin; and b) an effective amount of a kinase inhibitor (such as a tyrosine kinase inhibitor, e.g., nilotinib or sorafenib). In some embodiments, the method comprises administering to the individual a) an effective amount of a composition comprising nanoparticles comprising an mTOR inhibitor (such as a limus drug, e.g., sirolimus or a derivative thereof) and an albumin, wherein the nanoparticles have an average particle size of no greater than about 150 nm (such as no greater than about 120 nm); and b) an effective amount of a kinase inhibitor (such as a tyrosine kinase inhibitor, e.g., nilotinib or sorafenib). In some embodiments, the method comprises administering to the individual a) an effective amount of a composition comprising nanoparticles comprising an mTOR inhibitor (such as a limus drug, e.g., sirolimus or a derivative thereof) and an albumin, wherein the nanoparticles comprise the mTOR inhibitor associated (e.g., coated) with albumin, wherein the nanoparticles have an average particle size of no greater than about 150 nm (such as no greater than about 120 nm); and b) an effective amount of a kinase inhibitor (such as a tyrosine kinase inhibitor, e.g., nilotinib or sorafenib). In some embodiments, the method comprises administering to the individual a) an effective amount of a composition comprising nanoparticles comprising an mTOR inhibitor (such as a limus drug, e.g., sirolimus or a derivative thereof) and an albumin, wherein the nanoparticles comprise the mTOR inhibitor associated (e.g., coated) with the albumin, wherein the nanoparticles have an average particle size of no greater than about 150 nm (such as no greater than about 120 nm, for example about 100 nm), wherein the weight ratio of albumin and the mTOR inhibitor in the mTOR inhibitor nanoparticle composition is about 9:1 or less (such as about 9:1 or about 8:1); and b) an effective amount of a kinase inhibitor (such as a tyrosine kinase inhibitor, e.g., nilotinib or sorafenib). In some embodiments, the method further comprises administering to the individual at least one therapeutic agent used in a standard combination therapy with the kinase inhibitor. In some embodiments, the mTOR inhibitor is a limus drug. In some embodiments, the mTOR inhibitor is sirolimus or a derivative thereof. In some embodiments, the mTOR inhibitor nanoparticle composition comprises nab-sirolimus. In some embodiments, the mTOR inhibitor nanoparticle composition is nab-sirolimus. In some embodiments, the kinase inhibitor is a tyrosine kinase inhibitor. In some embodiments, the kinase inhibitor is a serine/threonine kinase inhibitor. In some embodiments, the kinase inhibitor is a Raf kinase inhibitor. In some embodiments, the kinase inhibitor inhibits more than one class of kinase (e.g., an inhibitor of more than one of a tyrosine kinase, a Raf kinase, and a serine/threonine kinase). In some embodiments, the kinase inhibitor is selected from the group consisting of erlotinib, imatinib, lapatinib, nilotinib, sorafenib, and sunitinib. In some embodiments, the kinase inhibitor is nilotinib. In some embodiments, the pancreatic neuroendocrine cancer is recurrent pancreatic neuroendocrine cancer. In some embodiments, the pancreatic neuroendocrine cancer is refractory to one or more drugs used in a standard therapy for pancreatic neuroendocrine cancer, such as, but not limited to, doxorubicin, streptozocin, fluorouracil (5-FU), dacarbazine, temozolomide, thalidomide, capecitabine, sunitinib, somatostatin analogs (e.g., octreotide, lanreotide, or pasireotide), and/or everolimus.

In some embodiments, there is provided a method of treating pancreatic neuroendocrine cancer in an individual (such as a human) comprising administering to the individual a) an effective amount of a composition comprising nanoparticles comprising sirolimus or a derivative thereof and an albumin; and b) an effective amount of a kinase inhibitor (such as a tyrosine kinase inhibitor, e.g., nilotinib or sorafenib). In some embodiments, the method comprises administering to the individual a) an effective amount of a composition comprising nanoparticles comprising sirolimus or a derivative thereof and an albumin, wherein the sirolimus or derivative thereof in the nanoparticles is associated (e.g., coated) with the albumin; and b) an effective amount of a kinase inhibitor (such as a tyrosine kinase inhibitor, e.g., nilotinib or sorafenib). In some embodiments, the method comprises administering to the individual a) an effective amount of a composition comprising nanoparticles comprising sirolimus or a derivative thereof and an albumin, wherein the nanoparticles have an average particle size of no greater than about 150 nm (such as no greater than about 120 nm); and b) an effective amount of a kinase inhibitor (such as a tyrosine kinase inhibitor, e.g., nilotinib or sorafenib). In some embodiments, the method comprises administering to the individual a) an effective amount of a composition comprising nanoparticles comprising sirolimus or a derivative thereof and an albumin, wherein the nanoparticles comprise the sirolimus or derivative thereof associated (e.g., coated) with albumin, wherein the nanoparticles have an average particle size of no greater than about 150 nm (such as no greater than about 120 nm); and b) an effective amount of a kinase inhibitor (such as a tyrosine kinase inhibitor, e.g., nilotinib or sorafenib). In some embodiments, the method comprises administering to the individual a) an effective amount of a composition comprising nanoparticles comprising sirolimus or a derivative thereof and an albumin, wherein the nanoparticles comprise the sirolimus or derivative thereof associated (e.g., coated) with the albumin, wherein the nanoparticles have an average particle size of no greater than about 150 nm (such as no greater than about 120 nm, for example about 100 nm), wherein the weight ratio of albumin and the sirolimus or derivative thereof in the sirolimus nanoparticle composition is about 9:1 or less (such as about 9:1 or about 8:1); and b) an effective amount of a kinase inhibitor (such as a tyrosine kinase inhibitor, e.g., nilotinib or sorafenib). In some embodiments, the method further comprises administering to the individual at least one therapeutic agent used in a standard combination therapy with the kinase inhibitor. In some embodiments, the sirolimus or derivative thereof is sirolimus. In some embodiments, the sirolimus nanoparticle composition comprises nab-sirolimus. In some embodiments, the sirolimus nanoparticle composition is nab-sirolimus. In some embodiments, the kinase inhibitor is a tyrosine kinase inhibitor. In some embodiments, the kinase inhibitor is a serine/threonine kinase inhibitor. In some embodiments, the kinase inhibitor is a Raf kinase inhibitor. In some embodiments, the kinase inhibitor inhibits more than one class of kinase (e.g., an inhibitor of more than one of a tyrosine kinase, a Raf kinase, and a serine/threonine kinase). In some embodiments, the kinase inhibitor is selected from the group consisting of erlotinib, imatinib, lapatinib, nilotinib, sorafenib, and sunitinib. In some embodiments, the kinase inhibitor is nilotinib. In some embodiments, the pancreatic neuroendocrine cancer is recurrent pancreatic neuroendocrine cancer. In some embodiments, the pancreatic neuroendocrine cancer is refractory to one or more drugs used in a standard therapy for pancreatic neuroendocrine cancer, such as, but not limited to, doxorubicin, streptozocin, fluorouracil (5-FU), dacarbazine, temozolomide, thalidomide, capecitabine, sunitinib, somatostatin analogs (e.g., octreotide, lanreotide, or pasireotide), and/or everolimus.

In some embodiments, there is provided a method of treating pancreatic neuroendocrine cancer in an individual (such as a human) comprising administering to the individual a) an effective amount of a composition comprising nanoparticles comprising an mTOR inhibitor (such as a limus drug, e.g., sirolimus or a derivative thereof) and an albumin; and b) an effective amount of a cancer vaccine. In some embodiments, the method comprises administering to the individual a) an effective amount of a composition comprising nanoparticles comprising an mTOR inhibitor (such as a limus drug, e.g., sirolimus or a derivative thereof) and an albumin, wherein the mTOR inhibitor in the nanoparticles is associated (e.g., coated) with the albumin; and b) an effective amount of a cancer vaccine. In some embodiments, the method comprises administering to the individual a) an effective amount of a composition comprising nanoparticles comprising an mTOR inhibitor (such as a limus drug, e.g., sirolimus or a derivative thereof) and an albumin, wherein the nanoparticles have an average particle size of no greater than about 150 nm (such as no greater than about 120 nm); and b) an effective amount of a cancer vaccine. In some embodiments, the method comprises administering to the individual a) an effective amount of a composition comprising nanoparticles comprising an mTOR inhibitor (such as a limus drug, e.g., sirolimus or a derivative thereof) and an albumin, wherein the nanoparticles comprise the mTOR inhibitor associated (e.g., coated) with albumin, wherein the nanoparticles have an average particle size of no greater than about 150 nm (such as no greater than about 120 nm); and b) an effective amount of a cancer vaccine. In some embodiments, the method comprises administering to the individual a) an effective amount of a composition comprising nanoparticles comprising an mTOR inhibitor (such as a limus drug, e.g., sirolimus or a derivative thereof) and an albumin, wherein the nanoparticles comprise the mTOR inhibitor associated (e.g., coated) with the albumin, wherein the nanoparticles have an average particle size of no greater than about 150 nm (such as no greater than about 120 nm, for example about 100 nm), wherein the weight ratio of albumin and the mTOR inhibitor in the mTOR inhibitor nanoparticle composition is about 9:1 or less (such as about 9:1 or about 8:1); and b) an effective amount of a cancer vaccine. In some embodiments, the method further comprises administering to the individual at least one therapeutic agent used in a standard combination therapy with the cancer vaccine. In some embodiments, the mTOR inhibitor is a limus drug. In some embodiments, the mTOR inhibitor is sirolimus or a derivative thereof. In some embodiments, the mTOR inhibitor nanoparticle composition comprises nab-sirolimus. In some embodiments, the mTOR inhibitor nanoparticle composition is nab-sirolimus. In some embodiments, the cancer vaccine is a vaccine prepared using autologous tumor cells. In some embodiments, the cancer vaccine is a vaccine prepared using allogeneic tumor cells. In some embodiments, the pancreatic neuroendocrine cancer is recurrent pancreatic neuroendocrine cancer. In some embodiments, the pancreatic neuroendocrine cancer is refractory to one or more drugs used in a standard therapy for pancreatic neuroendocrine cancer, such as, but not limited to, doxorubicin, streptozocin, fluorouracil (5-FU), dacarbazine, temozolomide, thalidomide, capecitabine, sunitinib, somatostatin analogs (e.g., octreotide, lanreotide, or pasireotide), and/or everolimus.

In some embodiments, there is provided a method of treating pancreatic neuroendocrine cancer in an individual (such as a human) comprising administering to the individual a) an effective amount of a composition comprising nanoparticles comprising sirolimus or a derivative thereof and an albumin; and b) an effective amount of a cancer vaccine. In some embodiments, the method comprises administering to the individual a) an effective amount of a composition comprising nanoparticles comprising sirolimus or a derivative thereof and an albumin, wherein the sirolimus or derivative thereof in the nanoparticles is associated (e.g., coated) with the albumin; and b) an effective amount of a cancer vaccine. In some embodiments, the method comprises administering to the individual a) an effective amount of a composition comprising nanoparticles comprising sirolimus or a derivative thereof and an albumin, wherein the nanoparticles have an average particle size of no greater than about 150 nm (such as no greater than about 120 nm); and b) an effective amount of a cancer vaccine. In some embodiments, the method comprises administering to the individual a) an effective amount of a composition comprising nanoparticles comprising sirolimus or a derivative thereof and an albumin, wherein the nanoparticles comprise the sirolimus or derivative thereof associated (e.g., coated) with albumin, wherein the nanoparticles have an average particle size of no greater than about 150 nm (such as no greater than about 120 nm); and b) an effective amount of a cancer vaccine. In some embodiments, the method comprises administering to the individual a) an effective amount of a composition comprising nanoparticles comprising sirolimus or a derivative thereof and an albumin, wherein the nanoparticles comprise the sirolimus or derivative thereof associated (e.g., coated) with the albumin, wherein the nanoparticles have an average particle size of no greater than about 150 nm (such as no greater than about 120 nm, for example about 100 nm), wherein the weight ratio of albumin and the sirolimus or derivative thereof in the sirolimus nanoparticle composition is about 9:1 or less (such as about 9:1 or about 8:1); and b) an effective amount of a cancer vaccine. In some embodiments, the method further comprises administering to the individual at least one therapeutic agent used in a standard combination therapy with the cancer vaccine. In some embodiments, the sirolimus or derivative thereof is sirolimus. In some embodiments, the sirolimus nanoparticle composition comprises nab-sirolimus. In some embodiments, the sirolimus nanoparticle composition is nab-sirolimus. In some embodiments, the cancer vaccine is a vaccine prepared using autologous tumor cells. In some embodiments, the cancer vaccine is a vaccine prepared using allogeneic tumor cells. In some embodiments, the pancreatic neuroendocrine cancer is recurrent pancreatic neuroendocrine cancer. In some embodiments, the pancreatic neuroendocrine cancer is refractory to one or more drugs used in a standard therapy for pancreatic neuroendocrine cancer, such as, but not limited to, doxorubicin, streptozocin, fluorouracil (5-FU), dacarbazine, temozolomide, thalidomide, capecitabine, sunitinib, somatostatin analogs (e.g., octreotide, lanreotide, or pasireotide), and/or everolimus.

In some embodiments, there is provided a method of treating pancreatic neuroendocrine cancer in an individual (such as a human) comprising administering to the individual a) an effective amount of a composition comprising nanoparticles comprising sirolimus or a derivative thereof and an albumin; and b) an effective amount of a second therapeutic agent selected from the group consisting of doxorubicin, streptozocin, fluorouracil (5-FU), dacarbazine, temozolomide, thalidomide, capecitabine, sunitinib, somatostatin analogs (e.g., octreotide, lanreotide, or pasireotide), and everolimus. In some embodiments, the method comprises administering to the individual a) an effective amount of a composition comprising nanoparticles comprising sirolimus or a derivative thereof and an albumin, wherein the sirolimus or derivative thereof in the nanoparticles is associated (e.g., coated) with the albumin; and b) an effective amount of a second therapeutic agent selected from the group consisting of doxorubicin, streptozocin, fluorouracil (5-FU), dacarbazine, temozolomide, thalidomide, capecitabine, sunitinib, somatostatin analogs (e.g., octreotide, lanreotide, or pasireotide), and everolimus. In some embodiments, the method comprises administering to the individual a) an effective amount of a composition comprising nanoparticles comprising sirolimus or a derivative thereof and an albumin, wherein the nanoparticles have an average particle size of no greater than about 150 nm (such as no greater than about 120 nm); and b) an effective amount of a second therapeutic agent selected from the group consisting of doxorubicin, streptozocin, fluorouracil (5-FU), dacarbazine, temozolomide, thalidomide, capecitabine, sunitinib, somatostatin analogs (e.g., octreotide, lanreotide, or pasireotide), and everolimus. In some embodiments, the method comprises administering to the individual a) an effective amount of a composition comprising nanoparticles comprising sirolimus or a derivative thereof and an albumin, wherein the nanoparticles comprise the sirolimus or derivative thereof associated (e.g., coated) with albumin, wherein the nanoparticles have an average particle size of no greater than about 150 nm (such as no greater than about 120 nm); and b) an effective amount of a second therapeutic agent selected from the group consisting of doxorubicin, streptozocin, fluorouracil (5-FU), dacarbazine, temozolomide, thalidomide, capecitabine, sunitinib, somatostatin analogs (e.g., octreotide, lanreotide, or pasireotide), and everolimus. In some embodiments, the method comprises administering to the individual a) an effective amount of a composition comprising nanoparticles comprising sirolimus or a derivative thereof and an albumin, wherein the nanoparticles comprise the sirolimus or derivative thereof associated (e.g., coated) with the albumin, wherein the nanoparticles have an average particle size of no greater than about 150 nm (such as no greater than about 120 nm, for example about 100 nm), wherein the weight ratio of albumin and the sirolimus or derivative thereof in the sirolimus nanoparticle composition is about 9:1 or less (such as about 9:1 or about 8:1); and b) an effective amount of a second therapeutic agent selected from the group consisting of doxorubicin, streptozocin, fluorouracil (5-FU), dacarbazine, temozolomide, thalidomide, capecitabine, sunitinib, somatostatin analogs (e.g., octreotide, lanreotide, or pasireotide), and everolimus. In some embodiments, the method further comprises administering to the individual at least one therapeutic agent used in a standard combination therapy with the second therapeutic agent. In some embodiments, the sirolimus or derivative thereof is sirolimus. In some embodiments, the sirolimus nanoparticle composition comprises nab-sirolimus. In some embodiments, the sirolimus nanoparticle composition is nab-sirolimus. In some embodiments, the pancreatic neuroendocrine cancer is recurrent pancreatic neuroendocrine cancer. In some embodiments, the pancreatic neuroendocrine cancer is refractory to one or more drugs used in a standard therapy for pancreatic neuroendocrine cancer, such as, but not limited to, doxorubicin, streptozocin, fluorouracil (5-FU), dacarbazine, temozolomide, thalidomide, capecitabine, sunitinib, somatostatin analogs (e.g., octreotide, lanreotide, or pasireotide), and/or everolimus.

In some embodiments, according to any of the methods of treating pancreatic neuroendocrine cancer in an individual described herein, the individual is a human who exhibits one or more symptoms associated with pancreatic neuroendocrine cancer. In some embodiments, the individual is at an early stage of pancreatic neuroendocrine cancer. In some embodiments, the individual is at an advanced stage of pancreatic neuroendocrine cancer. In some of embodiments, the individual is genetically or otherwise predisposed (e.g., having a risk factor) to developing pancreatic neuroendocrine cancer. Individuals at risk for pancreatic neuroendocrine cancer include, e.g., those having relatives who have experienced pancreatic neuroendocrine cancer, and those whose risk is determined by analysis of genetic or biochemical markers. In some embodiments, the individual may be a human who has a gene, genetic mutation, or polymorphism associated with pancreatic neuroendocrine cancer (e.g., NF1 and/or MEN1) or has one or more extra copies of a gene associated with pancreatic neuroendocrine cancer. In some embodiments, the individual has a ras or PTEN mutation. In some embodiments, the cancer cells are dependent on an mTOR pathway to translate one or more mRNAs. In some embodiments, the cancer cells are not capable of synthesizing mRNAs by an mTOR-independent pathway. In some embodiments, the cancer cells have decreased or no PTEN activity or have decreased or no expression of PTEN compared to non-cancerous cells. In some embodiments, the individual has at least one tumor biomarker selected from the group consisting of elevated PI3K activity, elevated mTOR activity, presence of FLT-3ITD, elevated AKT activity, elevated KRAS activity, and elevated NRAS activity. In some embodiments, the individual has a variation in at least one gene selected from the group consisting of drug metabolism genes, cancer genes, and drug target genes.

### Ovarian Cancer

In some embodiments, there is provided a method of treating ovarian cancer in an individual (such as a human) comprising administering to the individual a) an effective amount of a composition comprising nanoparticles comprising an mTOR inhibitor (such as a limus drug, e.g., sirolimus or a derivative thereof) and an albumin; and b) an effective amount of a second therapeutic agent. In some embodiments, the method comprises administering to the individual a) an effective amount of a composition comprising nanoparticles comprising an mTOR inhibitor (such as a limus drug, e.g., sirolimus or a derivative thereof) and an albumin, wherein the mTOR inhibitor in the nanoparticles is associated (e.g., coated) with the albumin; and b) an effective amount of a second therapeutic agent. In some embodiments, the method comprises administering to the individual a) an effective amount of a composition comprising nanoparticles comprising an mTOR inhibitor (such as a limus drug, e.g., sirolimus or a derivative thereof) and an albumin, wherein the nanoparticles have an average particle size of no greater than about 150 nm (such as no greater than about 120 nm); and b) an effective amount of a second therapeutic agent. In some embodiments, the method comprises administering to the individual a) an effective amount of a composition comprising nanoparticles comprising an mTOR inhibitor (such as a limus drug, e.g., sirolimus or a derivative thereof) and an albumin, wherein the nanoparticles comprise the mTOR inhibitor associated (e.g., coated) with albumin, wherein the nanoparticles have an average particle size of no greater than about 150 nm (such as no greater than about 120 nm); and b) an effective amount of a second therapeutic agent. In some embodiments, the method comprises administering to the individual a) an effective amount of a composition comprising nanoparticles comprising an mTOR inhibitor (such as a limus drug, e.g., sirolimus or a derivative thereof) and an albumin, wherein the nanoparticles comprise the mTOR inhibitor associated (e.g., coated) with the albumin, wherein the nanoparticles have an average particle size of no greater than about 150 nm (such as no greater than about 120 nm, for example about 100 nm), wherein the weight ratio of albumin and the mTOR inhibitor in the mTOR inhibitor nanoparticle composition is about 9:1 or less (such as about 9:1 or about 8:1); and b) an effective amount of a second therapeutic agent. In some embodiments, the method further comprises administering to the individual at least one therapeutic agent used in a standard combination therapy with the second therapeutic agent. In some embodiments, the mTOR inhibitor is a limus drug. In some embodiments, the mTOR inhibitor is sirolimus or a derivative thereof. In some embodiments, the mTOR inhibitor nanoparticle composition comprises nab-sirolimus. In some embodiments, the mTOR inhibitor nanoparticle composition is nab-sirolimus. In some embodiments, the second therapeutic agent is selected from the group consisting of an immunomodulator (such as an immunostimulator or an immune checkpoint inhibitor), a histone deacetylase inhibitor, a kinase inhibitor (such as a tyrosine kinase inhibitor), and a cancer vaccine (such as a vaccine prepared using tumor cells or at least one tumor-associated antigen). In some embodiments, the second therapeutic agent is an immunomodulator. In some embodiments, the immunomodulator is an immunostimulator that directly stimulates the immune system of an individual. In some embodiments, the immunomodulator is an agonistic antibody that targets an activating receptor on an immune cell (such as a T cell). In some embodiments, the immunomodulator is an immune checkpoint inhibitor. In some embodiments, the immune checkpoint inhibitor is an antagonistic antibody that targets an immune checkpoint protein. In some embodiments, the immunomodulator is an IMiDs® compound (small molecule immunomodulator, such as lenalidomide or pomalidomide). In some embodiments, the immunomodulator is lenalidomide. In some embodiments, the immunomodulator is pomalidomide. In some embodiments, the immunomodulator is small molecule or antibody-based IDO inhibitor. In some embodiments, the second therapeutic agent is a histone deacetylase inhibitor. In some embodiments, the histone deacetylase inhibitor is specific to only one HDAC. In some embodiments, the histone deacetylase inhibitor is specific to only one class of HDAC. In some embodiments, the histone deacetylase inhibitor is specific to two or more HDACs or two or more classes of HDACs. In some embodiments, the histone deacetylase inhibitor is specific to class I and II HDACs. In some embodiments, the histone deacetylase inhibitor is specific to class III HDACs. In some embodiments, the histone deacetylase inhibitor is selected from the group consisting of romidepsin, panobinostat, ricolinostat, and belinostat. In some embodiments, the histone deacetylase inhibitor is romidepsin. In some embodiments, the second therapeutic agent is a kinase inhibitor, such as a tyrosine kinase inhibitor. In some embodiments, the kinase inhibitor is a serine/threonine kinase inhibitor. In some embodiments, the kinase inhibitor is a Raf kinase inhibitor. In some embodiments, the kinase inhibitor inhibits more than one class of kinase (e.g., an inhibitor of more than one of a tyrosine kinase, a Raf kinase, and a serine/threonine kinase). In some embodiments, the kinase inhibitor is selected from the group consisting of erlotinib, imatinib, lapatinib, nilotinib, sorafenib, and sunitinib. In some embodiments, the kinase inhibitor is sorafenib. In some embodiments, the kinase inhibitor is nilotinib. In some embodiments, the second therapeutic agent is a cancer vaccine, such as a vaccine prepared using tumor cells or at least one tumor-associated antigen. In some embodiments, the second therapeutic agent and the nanoparticle composition are administered sequentially. In some embodiments, the second therapeutic agent and the nanoparticle composition are administered simultaneously. In some embodiments, the second therapeutic agent and the nanoparticle composition are administered concurrently.

In some embodiments, the ovarian cancer is ovarian epithelial cancer. Exemplary ovarian epithelial cancer histological classifications include: serous cystomas (e.g., serous benign cystadenomas, serous cystadenomas with proliferating activity of the epithelial cells and nuclear abnormalities but with no infiltrative destructive growth, or serous cystadenocarcinomas), mucinous cystomas (e.g., mucinous benign cystadenomas, mucinous cystadenomas with proliferating activity of the epithelial cells and nuclear abnormalities but with no infiltrative destructive growth, or mucinous cystadenocarcinomas), endometrioid tumors (e.g., endometrioid benign cysts, endometrioid tumors with proliferating activity of the epithelial cells and nuclear abnormalities but with no infiltrative destructive growth, or endometrioid adenocarcinomas), clear cell (mesonephroid) tumors (e.g., begin clear cell tumors, clear cell tumors with proliferating activity of the epithelial cells and nuclear abnormalities but with no infiltrative destructive growth, or clear cell cystadenocarcinomas), unclassified tumors that cannot be allotted to one of the above groups, or other malignant tumors. In some embodiments, the ovarian epithelial cancer is stage I (e.g., stage IA, IB, or IC), stage II (e.g., stage HA, HB, or IIC), stage III (e.g., stage IIIA, HIB, or HIC), or stage IV.

In some embodiments, the ovarian cancer is an ovarian germ cell tumor. Exemplary histologic subtypes include dysgerminomas or other germ cell tumors (e.g., endodermal sinus tumors such as hepatoid or intestinal tumors, embryonal carcinomas, olyembryomas, choriocarcinomas, teratomas, or mixed form tumors). Exemplary teratomas are immature teratomas, mature teratomas, solid teratomas, and cystic teratomas (e.g., dermoid cysts such as mature cystic teratomas, and dermoid cysts with malignant transformation). Some teratomas are monodermal and highly specialized, such as struma ovarii, carcinoid, struma ovarii and carcinoid, or others (e.g., malignant neuroectodermal and ependymomas). In some embodiments, the ovarian germ cell tumor is stage I (e.g., stage IA, IB, or IC), stage II (e.g., stage HA, HB, or IIC), stage III (e.g., stage IIIA, HIB, or IIIC), or stage IV.

In some embodiments, there is provided a method of treating ovarian cancer in an individual (such as a human) comprising administering to the individual a) an effective amount of a composition comprising nanoparticles comprising an mTOR inhibitor (such as a limus drug, e.g., sirolimus or a derivative thereof) and an albumin; and b) an effective amount of an immunomodulator (such as lenalidomide, pomalidomide, or an immune checkpoint inhibitor). In some embodiments, the method comprises administering to the individual a) an effective amount of a composition comprising nanoparticles comprising an mTOR inhibitor (such as a limus drug, e.g., sirolimus or a derivative thereof) and an albumin, wherein the mTOR inhibitor in the nanoparticles is associated (e.g., coated) with the albumin; and b) an effective amount of an immunomodulator (such as lenalidomide, pomalidomide, or an immune checkpoint inhibitor). In some embodiments, the method comprises administering to the individual a) an effective amount of a composition comprising nanoparticles comprising an mTOR inhibitor (such as a limus drug, e.g., sirolimus or a derivative thereof) and an albumin, wherein the nanoparticles have an average particle size of no greater than about 150 nm (such as no greater than about 120 nm); and b) an effective amount of an immunomodulator (such as lenalidomide, pomalidomide, or an immune checkpoint inhibitor). In some embodiments, the method comprises administering to the individual a) an effective amount of a composition comprising nanoparticles comprising an mTOR inhibitor (such as a limus drug, e.g., sirolimus or a derivative thereof) and an albumin, wherein the nanoparticles comprise the mTOR inhibitor associated (e.g., coated) with albumin, wherein the nanoparticles have an average particle size of no greater than about 150 nm (such as no greater than about 120 nm); and b) an effective amount of an immunomodulator (such as lenalidomide, pomalidomide, or an immune checkpoint inhibitor). In some embodiments, the method comprises administering to the individual a) an effective amount of a composition comprising nanoparticles comprising an mTOR inhibitor (such as a limus drug, e.g., sirolimus or a derivative thereof) and an albumin, wherein the nanoparticles comprise the mTOR inhibitor associated (e.g., coated) with the albumin, wherein the nanoparticles have an average particle size of no greater than about 150 nm (such as no greater than about 120 nm, for example about 100 nm), wherein the weight ratio of albumin and the mTOR inhibitor in the mTOR inhibitor nanoparticle composition is about 9:1 or less (such as about 9:1 or about 8:1); and b) an effective amount of an immunomodulator (such as lenalidomide, pomalidomide, or an immune checkpoint inhibitor). In some embodiments, the method further comprises administering to the individual at least one therapeutic agent used in a standard combination therapy with the immunomodulator. In some embodiments, the mTOR inhibitor is a limus drug. In some embodiments, the mTOR inhibitor is sirolimus or a derivative thereof. In some embodiments, the mTOR inhibitor nanoparticle composition comprises nab-sirolimus. In some embodiments, the mTOR inhibitor nanoparticle composition is nab-sirolimus. In some embodiments, the immunomodulator is an immunostimulator that directly stimulates the immune system of an individual. In some embodiments, the immunomodulator is an agonistic antibody that targets an activating receptor on an immune cell (such as a T cell). In some embodiments, the immunomodulator is an immune checkpoint inhibitor. In some embodiments, the immune checkpoint inhibitor is an antagonistic antibody that targets an immune checkpoint protein. In some embodiments, the immunomodulator is an IMiDs® compound (small molecule immunomodulator, such as lenalidomide or pomalidomide). In some embodiments, the immunomodulator is lenalidomide. In some embodiments, the immunomodulator is pomalidomide. In some embodiments, the immunomodulator is small molecule or antibody-based IDO inhibitor. In some embodiments, the ovarian cancer is recurrent ovarian cancer. In some embodiments, the ovarian cancer is refractory to one or more drugs used in a standard therapy for ovarian cancer, such as, but not limited to, nab-paclitaxel, paclitaxel, cisplatin, vinblastine, altretamine, capecitabine, cyclophosphamide, etoposide, gemcitabine, ifosfamide, irinotecan, liposomal doxorubicin, melphalan, pemetrexed, topotecan, and/or vinorelbine.

In some embodiments, there is provided a method of treating ovarian cancer in an individual (such as a human) comprising administering to the individual a) an effective amount of a composition comprising nanoparticles comprising sirolimus or a derivative thereof and an albumin; and b) an effective amount of an immunomodulator (such as an immunostimulator, e.g., pomalidomide). In some embodiments, the method comprises administering to the individual a) an effective amount of a composition comprising nanoparticles comprising sirolimus or a derivative thereof and an albumin, wherein the sirolimus or derivative thereof in the nanoparticles is associated (e.g., coated) with the albumin; and b) an effective amount of an immunomodulator (such as an immunostimulator, e.g., pomalidomide). In some embodiments, the method comprises administering to the individual a) an effective amount of a composition comprising nanoparticles comprising sirolimus or a derivative thereof and an albumin, wherein the nanoparticles have an average particle size of no greater than about 150 nm (such as no greater than about 120 nm); and b) an effective amount of an immunomodulator (such as an immunostimulator, e.g., pomalidomide). In some embodiments, the method comprises administering to the individual a) an effective amount of a composition comprising nanoparticles comprising sirolimus or a derivative thereof and an albumin, wherein the nanoparticles comprise the sirolimus or derivative thereof associated (e.g., coated) with albumin, wherein the nanoparticles have an average particle size of no greater than about 150 nm (such as no greater than about 120 nm); and b) an effective amount of an immunomodulator (such as an immunostimulator, e.g., pomalidomide). In some embodiments, the method comprises administering to the individual a) an effective amount of a composition comprising nanoparticles comprising sirolimus or a derivative thereof and albumin, wherein the nanoparticles comprise the sirolimus or derivative thereof associated (e.g., coated) with the albumin, wherein the nanoparticles have an average particle size of no greater than about 150 nm (such as no greater than about 120 nm, for example about 100 nm), wherein the weight ratio of albumin and sirolimus or a derivative thereof in the sirolimus nanoparticle composition is about 9:1 or less (such as about 9:1 or about 8:1); and b) an effective amount of an immunomodulator (such as an immunostimulator, e.g., pomalidomide). In some embodiments, the method further comprises administering to the individual at least one therapeutic agent used in a standard combination therapy with the immunomodulator. In some embodiments, the sirolimus or derivative thereof is sirolimus. In some embodiments, the sirolimus nanoparticle composition comprises nab-sirolimus. In some embodiments, the sirolimus nanoparticle composition is nab-sirolimus. In some embodiments, the immunomodulator is an immunostimulator that directly stimulates the immune system of an individual. In some embodiments, the immunomodulator is an agonistic antibody that targets an activating receptor on an immune cell (such as a T cell). In some embodiments, the immunomodulator is an immune checkpoint inhibitor. In some embodiments, the immune checkpoint inhibitor is an antagonistic antibody that targets an immune checkpoint protein. In some embodiments, the immunomodulator is an IMiDs® compound (small molecule immunomodulator, such as lenalidomide or pomalidomide). In some embodiments, the immunomodulator is lenalidomide. In some embodiments, the immunomodulator is pomalidomide. In some embodiments, the immunomodulator is small molecule or antibody-based IDO inhibitor. In some embodiments, the ovarian cancer is recurrent ovarian cancer. In some embodiments, the ovarian cancer is refractory to one or more drugs used in a standard therapy for ovarian cancer, such as, but not limited to, nab-paclitaxel, paclitaxel, cisplatin, vinblastine, altretamine, capecitabine, cyclophosphamide, etoposide, gemcitabine, ifosfamide, irinotecan, liposomal doxorubicin, melphalan, pemetrexed, topotecan, and/or vinorelbine.

In some embodiments, there is provided a method of treating ovarian cancer in an individual (such as a human) comprising administering to the individual a) an effective amount of a composition comprising nanoparticles comprising an mTOR inhibitor (such as a limus drug, e.g., sirolimus or a derivative thereof) and an albumin; and b) an effective amount of a histone deacetylase inhibitor (such as romidepsin). In some embodiments, the method comprises administering to the individual a) an effective amount of a composition comprising nanoparticles comprising an mTOR inhibitor (such as a limus drug, e.g., sirolimus or a derivative thereof) and an albumin, wherein the mTOR inhibitor in the nanoparticles is associated (e.g., coated) with the albumin; and b) an effective amount of a histone deacetylase inhibitor (such as romidepsin). In some embodiments, the method comprises administering to the individual a) an effective amount of a composition comprising nanoparticles comprising an mTOR inhibitor (such as a limus drug, e.g., sirolimus or a derivative thereof) and an albumin, wherein the nanoparticles have an average particle size of no greater than about 150 nm (such as no greater than about 120 nm); and b) an effective amount of a histone deacetylase inhibitor (such as romidepsin). In some embodiments, the method comprises administering to the individual a) an effective amount of a composition comprising nanoparticles comprising an mTOR inhibitor (such as a limus drug, e.g., sirolimus or a derivative thereof) and an albumin, wherein the nanoparticles comprise the mTOR inhibitor associated (e.g., coated) with albumin, wherein the nanoparticles have an average particle size of no greater than about 150 nm (such as no greater than about 120 nm); and b) an effective amount of a histone deacetylase inhibitor (such as romidepsin). In some embodiments, the method comprises administering to the individual a) an effective amount of a composition comprising nanoparticles comprising an mTOR inhibitor (such as a limus drug, e.g., sirolimus or a derivative thereof) and an albumin, wherein the nanoparticles comprise the mTOR inhibitor associated (e.g., coated) with the albumin, wherein the nanoparticles have an average particle size of no greater than about 150 nm (such as no greater than about 120 nm, for example about 100 nm), wherein the weight ratio of albumin and the mTOR inhibitor in the mTOR inhibitor nanoparticle composition is about 9:1 or less (such as about 9:1 or about 8:1); and b) an effective amount of a histone deacetylase inhibitor (such as romidepsin). In some embodiments, the method further comprises administering to the individual at least one therapeutic agent used in a standard combination therapy with the histone deacetylase inhibitor. In some embodiments, the mTOR inhibitor is a limus drug. In some embodiments, the mTOR inhibitor is sirolimus or a derivative thereof. In some embodiments, the mTOR inhibitor nanoparticle composition comprises nab-sirolimus. In some embodiments, the mTOR inhibitor nanoparticle composition is nab-sirolimus. In some embodiments, the histone deacetylase inhibitor is selected from the group consisting of romidepsin, panobinostat, ricolinostat, and belinostat. In some embodiments, the histone deacetylase inhibitor is romidepsin. In some embodiments, the ovarian cancer is recurrent ovarian cancer. In some embodiments, the ovarian cancer is refractory to one or more drugs used in a standard therapy for ovarian cancer, such as, but not limited to, nab-paclitaxel, paclitaxel, cisplatin, vinblastine, altretamine, capecitabine, cyclophosphamide, etoposide, gemcitabine, ifosfamide, irinotecan, liposomal doxorubicin, melphalan, pemetrexed, topotecan, and/or vinorelbine.

In some embodiments, there is provided a method of treating ovarian cancer in an individual (such as a human) comprising administering to the individual a) an effective amount of a composition comprising nanoparticles comprising sirolimus or a derivative thereof and an albumin; and b) an effective amount of a histone deacetylase inhibitor (such as romidepsin). In some embodiments, the method comprises administering to the individual a) an effective amount of a composition comprising nanoparticles comprising sirolimus or a derivative thereof and an albumin, wherein the sirolimus or derivative thereof in the nanoparticles is associated (e.g., coated) with the albumin; and b) an effective amount of a histone deacetylase inhibitor (such as romidepsin). In some embodiments, the method comprises administering to the individual a) an effective amount of a composition comprising nanoparticles comprising sirolimus or a derivative thereof and an albumin, wherein the nanoparticles have an average particle size of no greater than about 150 nm (such as no greater than about 120 nm); and b) an effective amount of a histone deacetylase inhibitor (such as romidepsin). In some embodiments, the method comprises administering to the individual a) an effective amount of a composition comprising nanoparticles comprising sirolimus or a derivative thereof and an albumin, wherein the nanoparticles comprise the sirolimus or derivative thereof associated (e.g., coated) with albumin, wherein the nanoparticles have an average particle size of no greater than about 150 nm (such as no greater than about 120 nm); and b) an effective amount of a histone deacetylase inhibitor (such as romidepsin). In some embodiments, the method comprises administering to the individual a) an effective amount of a composition comprising nanoparticles comprising sirolimus or a derivative thereof and albumin, wherein the nanoparticles comprise the sirolimus or derivative thereof associated (e.g., coated) with the albumin, wherein the nanoparticles have an average particle size of no greater than about 150 nm (such as no greater than about 120 nm, for example about 100 nm), wherein the weight ratio of albumin and sirolimus or a derivative thereof in the sirolimus nanoparticle composition is about 9:1 or less (such as about 9:1 or about 8:1); and b) an effective amount of a histone deacetylase inhibitor (such as romidepsin). In some embodiments, the method further comprises administering to the individual at least one therapeutic agent used in a standard combination therapy with the histone deacetylase inhibitor. In some embodiments, the sirolimus or derivative thereof is sirolimus. In some embodiments, the sirolimus nanoparticle composition comprises nab-sirolimus. In some embodiments, the sirolimus nanoparticle composition is nab-sirolimus. In some embodiments, the histone deacetylase inhibitor is selected from the group consisting of romidepsin, panobinostat, ricolinostat, and belinostat. In some embodiments, the histone deacetylase inhibitor is romidepsin. In some embodiments, the ovarian cancer is recurrent ovarian cancer. In some embodiments, the ovarian cancer is refractory to one or more drugs used in a standard therapy for ovarian cancer, such as, but not limited to, nab-paclitaxel, paclitaxel, cisplatin, vinblastine, altretamine, capecitabine, cyclophosphamide, etoposide, gemcitabine, ifosfamide, irinotecan, liposomal doxorubicin, melphalan, pemetrexed, topotecan, and/or vinorelbine.

In some embodiments, there is provided a method of treating ovarian cancer in an individual (such as a human) comprising administering to the individual a) an effective amount of a composition comprising nanoparticles comprising an mTOR inhibitor (such as a limus drug, e.g., sirolimus or a derivative thereof) and an albumin; and b) an effective amount of a kinase inhibitor (such as a tyrosine kinase inhibitor, e.g., nilotinib or sorafenib). In some embodiments, the method comprises administering to the individual a) an effective amount of a composition comprising nanoparticles comprising an mTOR inhibitor (such as a limus drug, e.g., sirolimus or a derivative thereof) and an albumin, wherein the mTOR inhibitor in the nanoparticles is associated (e.g., coated) with the albumin; and b) an effective amount of a kinase inhibitor (such as a tyrosine kinase inhibitor, e.g., nilotinib or sorafenib). In some embodiments, the method comprises administering to the individual a) an effective amount of a composition comprising nanoparticles comprising an mTOR inhibitor (such as a limus drug, e.g., sirolimus or a derivative thereof) and an albumin, wherein the nanoparticles have an average particle size of no greater than about 150 nm (such as no greater than about 120 nm); and b) an effective amount of a kinase inhibitor (such as a tyrosine kinase inhibitor, e.g., nilotinib or sorafenib). In some embodiments, the method comprises administering to the individual a) an effective amount of a composition comprising nanoparticles comprising an mTOR inhibitor (such as a limus drug, e.g., sirolimus or a derivative thereof) and an albumin, wherein the nanoparticles comprise the mTOR inhibitor associated (e.g., coated) with albumin, wherein the nanoparticles have an average particle size of no greater than about 150 nm (such as no greater than about 120 nm); and b) an effective amount of a kinase inhibitor (such as a tyrosine kinase inhibitor, e.g., nilotinib or sorafenib). In some embodiments, the method comprises administering to the individual a) an effective amount of a composition comprising nanoparticles comprising an mTOR inhibitor (such as a limus drug, e.g., sirolimus or a derivative thereof) and an albumin, wherein the nanoparticles comprise the mTOR inhibitor associated (e.g., coated) with the albumin, wherein the nanoparticles have an average particle size of no greater than about 150 nm (such as no greater than about 120 nm, for example about 100 nm), wherein the weight ratio of albumin and the mTOR inhibitor in the mTOR inhibitor nanoparticle composition is about 9:1 or less (such as about 9:1 or about 8:1); and b) an effective amount of a kinase inhibitor (such as a tyrosine kinase inhibitor, e.g., nilotinib or sorafenib). In some embodiments, the method further comprises administering to the individual at least one therapeutic agent used in a standard combination therapy with the kinase inhibitor. In some embodiments, the mTOR inhibitor is a limus drug. In some embodiments, the mTOR inhibitor is sirolimus or a derivative thereof. In some embodiments, the mTOR inhibitor nanoparticle composition comprises nab-sirolimus. In some embodiments, the mTOR inhibitor nanoparticle composition is nab-sirolimus. In some embodiments, the kinase inhibitor is a tyrosine kinase inhibitor. In some embodiments, the kinase inhibitor is a serine/threonine kinase inhibitor. In some embodiments, the kinase inhibitor is a Raf kinase inhibitor. In some embodiments, the kinase inhibitor inhibits more than one class of kinase (e.g., an inhibitor of more than one of a tyrosine kinase, a Raf kinase, and a serine/threonine kinase). In some embodiments, the kinase inhibitor is selected from the group consisting of erlotinib, imatinib, lapatinib, nilotinib, sorafenib, and sunitinib. In some embodiments, the kinase inhibitor is nilotinib. In some embodiments, the ovarian cancer is recurrent ovarian cancer. In some embodiments, the ovarian cancer is refractory to one or more drugs used in a standard therapy for ovarian cancer, such as, but not limited to, nab-paclitaxel, paclitaxel, cisplatin, vinblastine, altretamine, capecitabine, cyclophosphamide, etoposide, gemcitabine, ifosfamide, irinotecan, liposomal doxorubicin, melphalan, pemetrexed, topotecan, and/or vinorelbine.

In some embodiments, there is provided a method of treating ovarian cancer in an individual (such as a human) comprising administering to the individual a) an effective amount of a composition comprising nanoparticles comprising sirolimus or a derivative thereof and an albumin; and b) an effective amount of a kinase inhibitor (such as a tyrosine kinase inhibitor, e.g., nilotinib or sorafenib). In some embodiments, the method comprises administering to the individual a) an effective amount of a composition comprising nanoparticles comprising sirolimus or a derivative thereof and an albumin, wherein the sirolimus or derivative thereof in the nanoparticles is associated (e.g., coated) with the albumin; and b) an effective amount of a kinase inhibitor (such as a tyrosine kinase inhibitor, e.g., nilotinib or sorafenib). In some embodiments, the method comprises administering to the individual a) an effective amount of a composition comprising nanoparticles comprising sirolimus or a derivative thereof and an albumin, wherein the nanoparticles have an average particle size of no greater than about 150 nm (such as no greater than about 120 nm); and b) an effective amount of a kinase inhibitor (such as a tyrosine kinase inhibitor, e.g., nilotinib or sorafenib). In some embodiments, the method comprises administering to the individual a) an effective amount of a composition comprising nanoparticles comprising sirolimus or a derivative thereof and an albumin, wherein the nanoparticles comprise the sirolimus or derivative thereof associated (e.g., coated) with albumin, wherein the nanoparticles have an average particle size of no greater than about 150 nm (such as no greater than about 120 nm); and b) an effective amount of a kinase inhibitor (such as a tyrosine kinase inhibitor, e.g., nilotinib or sorafenib). In some embodiments, the method comprises administering to the individual a) an effective amount of a composition comprising nanoparticles comprising sirolimus or a derivative thereof and an albumin, wherein the nanoparticles comprise the sirolimus or derivative thereof associated (e.g., coated) with the albumin, wherein the nanoparticles have an average particle size of no greater than about 150 nm (such as no greater than about 120 nm, for example about 100 nm), wherein the weight ratio of albumin and the sirolimus or derivative thereof in the sirolimus nanoparticle composition is about 9:1 or less (such as about 9:1 or about 8:1); and b) an effective amount of a kinase inhibitor (such as a tyrosine kinase inhibitor, e.g., nilotinib or sorafenib). In some embodiments, the method further comprises administering to the individual at least one therapeutic agent used in a standard combination therapy with the kinase inhibitor. In some embodiments, the sirolimus or derivative thereof is sirolimus. In some embodiments, the sirolimus nanoparticle composition comprises nab-sirolimus. In some embodiments, the sirolimus nanoparticle composition is nab-sirolimus. In some embodiments, the kinase inhibitor is a tyrosine kinase inhibitor. In some embodiments, the kinase inhibitor is a serine/threonine kinase inhibitor. In some embodiments, the kinase inhibitor is a Raf kinase inhibitor. In some embodiments, the kinase inhibitor inhibits more than one class of kinase (e.g., an inhibitor of more than one of a tyrosine kinase, a Raf kinase, and a serine/threonine kinase). In some embodiments, the kinase inhibitor is selected from the group consisting of erlotinib, imatinib, lapatinib, nilotinib, sorafenib, and sunitinib. In some embodiments, the kinase inhibitor is nilotinib. In some embodiments, the ovarian cancer is recurrent ovarian cancer. In some embodiments, the ovarian cancer is refractory to one or more drugs used in a standard therapy for ovarian cancer, such as, but not limited to, nab-paclitaxel, paclitaxel, cisplatin, vinblastine, altretamine, capecitabine, cyclophosphamide, etoposide, gemcitabine, ifosfamide, irinotecan, liposomal doxorubicin, melphalan, pemetrexed, topotecan, and/or vinorelbine.

In some embodiments, there is provided a method of treating ovarian cancer in an individual (such as a human) comprising administering to the individual a) an effective amount of a composition comprising nanoparticles comprising an mTOR inhibitor (such as a limus drug, e.g., sirolimus or a derivative thereof) and an albumin; and b) an effective amount of a cancer vaccine. In some embodiments, the method comprises administering to the individual a) an effective amount of a composition comprising nanoparticles comprising an mTOR inhibitor (such as a limus drug, e.g., sirolimus or a derivative thereof) and an albumin, wherein the mTOR inhibitor in the nanoparticles is associated (e.g., coated) with the albumin; and b) an effective amount of a cancer vaccine. In some embodiments, the method comprises administering to the individual a) an effective amount of a composition comprising nanoparticles comprising an mTOR inhibitor (such as a limus drug, e.g., sirolimus or a derivative thereof) and an albumin, wherein the nanoparticles have an average particle size of no greater than about 150 nm (such as no greater than about 120 nm); and b) an effective amount of a cancer vaccine. In some embodiments, the method comprises administering to the individual a) an effective amount of a composition comprising nanoparticles comprising an mTOR inhibitor (such as a limus drug, e.g., sirolimus or a derivative thereof) and an albumin, wherein the nanoparticles comprise the mTOR inhibitor associated (e.g., coated) with albumin, wherein the nanoparticles have an average particle size of no greater than about 150 nm (such as no greater than about 120 nm); and b) an effective amount of a cancer vaccine. In some embodiments, the method comprises administering to the individual a) an effective amount of a composition comprising nanoparticles comprising an mTOR inhibitor (such as a limus drug, e.g., sirolimus or a derivative thereof) and an albumin, wherein the nanoparticles comprise the mTOR inhibitor associated (e.g., coated) with the albumin, wherein the nanoparticles have an average particle size of no greater than about 150 nm (such as no greater than about 120 nm, for example about 100 nm), wherein the weight ratio of albumin and the mTOR inhibitor in the mTOR inhibitor nanoparticle composition is about 9:1 or less (such as about 9:1 or about 8:1); and b) an effective amount of a cancer vaccine. In some embodiments, the method further comprises administering to the individual at least one therapeutic agent used in a standard combination therapy with the cancer vaccine. In some embodiments, the mTOR inhibitor is a limus drug. In some embodiments, the mTOR inhibitor is sirolimus or a derivative thereof. In some embodiments, the mTOR inhibitor nanoparticle composition comprises nab-sirolimus. In some embodiments, the mTOR inhibitor nanoparticle composition is nab-sirolimus. In some embodiments, the cancer vaccine is a vaccine prepared using autologous tumor cells. In some embodiments, the cancer vaccine is a vaccine prepared using allogeneic tumor cells. In some embodiments, the ovarian cancer is recurrent ovarian cancer. In some embodiments, the ovarian cancer is refractory to one or more drugs used in a standard therapy for ovarian cancer, such as, but not limited to, nab-paclitaxel, paclitaxel, cisplatin, vinblastine, altretamine, capecitabine, cyclophosphamide, etoposide, gemcitabine, ifosfamide, irinotecan, liposomal doxorubicin, melphalan, pemetrexed, topotecan, and/or vinorelbine.

In some embodiments, there is provided a method of treating ovarian cancer in an individual (such as a human) comprising administering to the individual a) an effective amount of a composition comprising nanoparticles comprising sirolimus or a derivative thereof and an albumin; and b) an effective amount of a cancer vaccine. In some embodiments, the method comprises administering to the individual a) an effective amount of a composition comprising nanoparticles comprising sirolimus or a derivative thereof and an albumin, wherein the sirolimus or derivative thereof in the nanoparticles is associated (e.g., coated) with the albumin; and b) an effective amount of a cancer vaccine. In some embodiments, the method comprises administering to the individual a) an effective amount of a composition comprising nanoparticles comprising sirolimus or a derivative thereof and an albumin, wherein the nanoparticles have an average particle size of no greater than about 150 nm (such as no greater than about 120 nm); and b) an effective amount of a cancer vaccine. In some embodiments, the method comprises administering to the individual a) an effective amount of a composition comprising nanoparticles comprising sirolimus or a derivative thereof and an albumin, wherein the nanoparticles comprise the sirolimus or derivative thereof associated (e.g., coated) with albumin, wherein the nanoparticles have an average particle size of no greater than about 150 nm (such as no greater than about 120 nm); and b) an effective amount of a cancer vaccine. In some embodiments, the method comprises administering to the individual a) an effective amount of a composition comprising nanoparticles comprising sirolimus or a derivative thereof and an albumin, wherein the nanoparticles comprise the sirolimus or derivative thereof associated (e.g., coated) with the albumin, wherein the nanoparticles have an average particle size of no greater than about 150 nm (such as no greater than about 120 nm, for example about 100 nm), wherein the weight ratio of albumin and the sirolimus or derivative thereof in the sirolimus nanoparticle composition is about 9:1 or less (such as about 9:1 or about 8:1); and b) an effective amount of a cancer vaccine. In some embodiments, the method further comprises administering to the individual at least one therapeutic agent used in a standard combination therapy with the cancer vaccine. In some embodiments, the sirolimus or derivative thereof is sirolimus. In some embodiments, the sirolimus nanoparticle composition comprises nab-sirolimus. In some embodiments, the sirolimus nanoparticle composition is nab-sirolimus. In some embodiments, the cancer vaccine is a vaccine prepared using autologous tumor cells. In some embodiments, the cancer vaccine is a vaccine prepared using allogeneic tumor cells. In some embodiments, the ovarian cancer is recurrent ovarian cancer. In some embodiments, the ovarian cancer is refractory to one or more drugs used in a standard therapy for ovarian cancer, such as, but not limited to, nab-paclitaxel, paclitaxel, cisplatin, vinblastine, altretamine, capecitabine, cyclophosphamide, etoposide, gemcitabine, ifosfamide, irinotecan, liposomal doxorubicin, melphalan, pemetrexed, topotecan, and/or vinorelbine.

In some embodiments, there is provided a method of treating ovarian cancer in an individual (such as a human) comprising administering to the individual a) an effective amount of a composition comprising nanoparticles comprising sirolimus or a derivative thereof and an albumin; and b) an effective amount of a second therapeutic agent selected from the group consisting of nab-paclitaxel, paclitaxel, cisplatin, vinblastine, altretamine, capecitabine, cyclophosphamide, etoposide, gemcitabine, ifosfamide, irinotecan, liposomal doxorubicin, melphalan, pemetrexed, topotecan, and vinorelbine. In some embodiments, the method comprises administering to the individual a) an effective amount of a composition comprising nanoparticles comprising sirolimus or a derivative thereof and an albumin, wherein the sirolimus or derivative thereof in the nanoparticles is associated (e.g., coated) with the albumin; and b) an effective amount of a second therapeutic agent selected from the group consisting of nab-paclitaxel, paclitaxel, cisplatin, vinblastine, altretamine, capecitabine, cyclophosphamide, etoposide, gemcitabine, ifosfamide, irinotecan, liposomal doxorubicin, melphalan, pemetrexed, topotecan, and vinorelbine. In some embodiments, the method comprises administering to the individual a) an effective amount of a composition comprising nanoparticles comprising sirolimus or a derivative thereof and an albumin, wherein the nanoparticles have an average particle size of no greater than about 150 nm (such as no greater than about 120 nm); and b) an effective amount of a second therapeutic agent selected from the group consisting of nab-paclitaxel, paclitaxel, cisplatin, vinblastine, altretamine, capecitabine, cyclophosphamide, etoposide, gemcitabine, ifosfamide, irinotecan, liposomal doxorubicin, melphalan, pemetrexed, topotecan, and vinorelbine. In some embodiments, the method comprises administering to the individual a) an effective amount of a composition comprising nanoparticles comprising sirolimus or a derivative thereof and an albumin, wherein the nanoparticles comprise the sirolimus or derivative thereof associated (e.g., coated) with albumin, wherein the nanoparticles have an average particle size of no greater than about 150 nm (such as no greater than about 120 nm); and b) an effective amount of a second therapeutic agent selected from the group consisting of nab-paclitaxel, paclitaxel, cisplatin, vinblastine, altretamine, capecitabine, cyclophosphamide, etoposide, gemcitabine, ifosfamide, irinotecan, liposomal doxorubicin, melphalan, pemetrexed, topotecan, and vinorelbine. In some embodiments, the method comprises administering to the individual a) an effective amount of a composition comprising nanoparticles comprising sirolimus or a derivative thereof and an albumin, wherein the nanoparticles comprise the sirolimus or derivative thereof associated (e.g., coated) with the albumin, wherein the nanoparticles have an average particle size of no greater than about 150 nm (such as no greater than about 120 nm, for example about 100 nm), wherein the weight ratio of albumin and the sirolimus or derivative thereof in the sirolimus nanoparticle composition is about 9:1 or less (such as about 9:1 or about 8:1); and b) an effective amount of a second therapeutic agent selected from the group consisting of nab-paclitaxel, paclitaxel, cisplatin, vinblastine, altretamine, capecitabine, cyclophosphamide, etoposide, gemcitabine, ifosfamide, irinotecan, liposomal doxorubicin, melphalan, pemetrexed, topotecan, and vinorelbine. In some embodiments, the method further comprises administering to the individual at least one therapeutic agent used in a standard combination therapy with the second therapeutic agent. In some embodiments, the sirolimus or derivative thereof is sirolimus. In some embodiments, the sirolimus nanoparticle composition comprises nab-sirolimus. In some embodiments, the sirolimus nanoparticle composition is nab-sirolimus. In some embodiments, the ovarian cancer is recurrent ovarian cancer. In some embodiments, the ovarian cancer is refractory to one or more drugs used in a standard therapy for ovarian cancer, such as, but not limited to, nab-paclitaxel, paclitaxel, cisplatin, vinblastine, altretamine, capecitabine, cyclophosphamide, etoposide, gemcitabine, ifosfamide, irinotecan, liposomal doxorubicin, melphalan, pemetrexed, topotecan, and/or vinorelbine.

In some embodiments, according to any of the methods of treating ovarian cancer in an individual described herein, the individual is a human who exhibits one or more symptoms associated with ovarian cancer. In some embodiments, the individual is at an early stage of ovarian cancer. In some embodiments, the individual is at an advanced stage of ovarian cancer. In some of embodiments, the individual is genetically or otherwise predisposed (e.g., having a risk factor) to developing ovarian cancer. Individuals at risk for ovarian cancer include, e.g., those having relatives who have experienced ovarian cancer, and those whose risk is determined by analysis of genetic or biochemical markers. In some embodiments, the individual may be a human who has a gene, genetic mutation, or polymorphism associated with ovarian cancer (e.g., MLH1, MLH3, MSH2, MSH6, TGFBR2, PMS1, PMS2, BRCA1 and/or BRCA2) or has one or more extra copies of a gene associated with ovarian cancer. In some embodiments, the individual has a ras or PTEN mutation. In some embodiments, the cancer cells are dependent on an mTOR pathway to translate one or more mRNAs. In some embodiments, the cancer cells are not capable of synthesizing mRNAs by an mTOR-independent pathway. In some embodiments, the cancer cells have decreased or no PTEN activity or have decreased or no expression of PTEN compared to non-cancerous cells. In some embodiments, the individual has at least one tumor biomarker selected from the group consisting of elevated PI3K activity, elevated mTOR activity, presence of FLT-3ITD, elevated AKT activity, elevated KRAS activity, and elevated NRAS activity. In some embodiments, the individual has a variation in at least one gene selected from the group consisting of drug metabolism genes, cancer genes, and drug target genes.

### Lymphangioleiomyomatosis (LAM)

In some embodiments, there is provided a method of treating lymphangioleiomyomatosis in an individual (such as a human) comprising administering to the individual a) an effective amount of a composition comprising nanoparticles comprising an mTOR inhibitor (such as a limus drug, e.g., sirolimus or a derivative thereof) and an albumin; and b) an effective amount of a second therapeutic agent. In some embodiments, the method comprises administering to the individual a) an effective amount of a composition comprising nanoparticles comprising an mTOR inhibitor (such as a limus drug, e.g., sirolimus or a derivative thereof) and an albumin, wherein the mTOR inhibitor in the nanoparticles is associated (e.g., coated) with the albumin; and b) an effective amount of a second therapeutic agent. In some embodiments, the method comprises administering to the individual a) an effective amount of a composition comprising nanoparticles comprising an mTOR inhibitor (such as a limus drug, e.g., sirolimus or a derivative thereof) and an albumin, wherein the nanoparticles have an average particle size of no greater than about 150 nm (such as no greater than about 120 nm); and b) an effective amount of a second therapeutic agent. In some embodiments, the method comprises administering to the individual a) an effective amount of a composition comprising nanoparticles comprising an mTOR inhibitor (such as a limus drug, e.g., sirolimus or a derivative thereof) and an albumin, wherein the nanoparticles comprise the mTOR inhibitor associated (e.g., coated) with albumin, wherein the nanoparticles have an average particle size of no greater than about 150 nm (such as no greater than about 120 nm); and b) an effective amount of a second therapeutic agent. In some embodiments, the method comprises administering to the individual a) an effective amount of a composition comprising nanoparticles comprising an mTOR inhibitor (such as a limus drug, e.g., sirolimus or a derivative thereof) and an albumin, wherein the nanoparticles comprise the mTOR inhibitor associated (e.g., coated) with the albumin, wherein the nanoparticles have an average particle size of no greater than about 150 nm (such as no greater than about 120 nm, for example about 100 nm), wherein the weight ratio of albumin and the mTOR inhibitor in the mTOR inhibitor nanoparticle composition is about 9:1 or less (such as about 9:1 or about 8:1); and b) an effective amount of a second therapeutic agent. In some embodiments, the method further comprises administering to the individual at least one therapeutic agent used in a standard combination therapy with the second therapeutic agent. In some embodiments, the mTOR inhibitor is a limus drug. In some embodiments, the mTOR inhibitor is sirolimus or a derivative thereof. In some embodiments, the mTOR inhibitor nanoparticle composition comprises nab-sirolimus. In some embodiments, the mTOR inhibitor nanoparticle composition is nab-sirolimus. In some embodiments, the second therapeutic agent is selected from the group consisting of an immunomodulator (such as an immunostimulator or an immune checkpoint inhibitor), a histone deacetylase inhibitor, a kinase inhibitor (such as a tyrosine kinase inhibitor), and a cancer vaccine (such as a vaccine prepared using tumor cells or at least one tumor-associated antigen). In some embodiments, the second therapeutic agent is an immunomodulator. In some embodiments, the immunomodulator is an immunostimulator that directly stimulates the immune system of an individual. In some embodiments, the immunomodulator is an agonistic antibody that targets an activating receptor on an immune cell (such as a T cell). In some embodiments, the immunomodulator is an immune checkpoint inhibitor. In some embodiments, the immune checkpoint inhibitor is an antagonistic antibody that targets an immune checkpoint protein. In some embodiments, the immunomodulator is an IMiDs® compound (small molecule immunomodulator, such as lenalidomide or pomalidomide). In some embodiments, the immunomodulator is lenalidomide. In some embodiments, the immunomodulator is pomalidomide. In some embodiments, the immunomodulator is small molecule or antibody-based IDO inhibitor. In some embodiments, the second therapeutic agent is a histone deacetylase inhibitor. In some embodiments, the histone deacetylase inhibitor is specific to only one HDAC. In some embodiments, the histone deacetylase inhibitor is specific to only one class of HDAC. In some embodiments, the histone deacetylase inhibitor is specific to two or more HDACs or two or more classes of HDACs. In some embodiments, the histone deacetylase inhibitor is specific to class I and II HDACs. In some embodiments, the histone deacetylase inhibitor is specific to class III HDACs. In some embodiments, the histone deacetylase inhibitor is selected from the group consisting of romidepsin, panobinostat, ricolinostat, and belinostat. In some embodiments, the histone deacetylase inhibitor is romidepsin. In some embodiments, the second therapeutic agent is a kinase inhibitor, such as a tyrosine kinase inhibitor. In some embodiments, the kinase inhibitor is a serine/threonine kinase inhibitor. In some embodiments, the kinase inhibitor is a Raf kinase inhibitor. In some embodiments, the kinase inhibitor inhibits more than one class of kinase (e.g., an inhibitor of more than one of a tyrosine kinase, a Raf kinase, and a serine/threonine kinase). In some embodiments, the kinase inhibitor is selected from the group consisting of erlotinib, imatinib, lapatinib, nilotinib, sorafenib, and sunitinib. In some embodiments, the kinase inhibitor is sorafenib. In some embodiments, the kinase inhibitor is nilotinib. In some embodiments, the second therapeutic agent is a cancer vaccine, such as a vaccine prepared using tumor cells or at least one tumor-associated antigen. In some embodiments, the second therapeutic agent and the nanoparticle composition are administered sequentially. In some embodiments, the second therapeutic agent and the nanoparticle composition are administered simultaneously. In some embodiments, the second therapeutic agent and the nanoparticle composition are administered concurrently.

In some embodiments, there is provided a method of treating lymphangioleiomyomatosis in an individual (such as a human) comprising administering to the individual a) an effective amount of a composition comprising nanoparticles comprising an mTOR inhibitor (such as a limus drug, e.g., sirolimus or a derivative thereof) and an albumin; and b) an effective amount of an immunomodulator (such as lenalidomide, pomalidomide, or an immune checkpoint inhibitor). In some embodiments, the method comprises administering to the individual a) an effective amount of a composition comprising nanoparticles comprising an mTOR inhibitor (such as a limus drug, e.g., sirolimus or a derivative thereof) and an albumin, wherein the mTOR inhibitor in the nanoparticles is associated (e.g., coated) with the albumin; and b) an effective amount of an immunomodulator (such as lenalidomide, pomalidomide, or an immune checkpoint inhibitor). In some embodiments, the method comprises administering to the individual a) an effective amount of a composition comprising nanoparticles comprising an mTOR inhibitor (such as a limus drug, e.g., sirolimus or a derivative thereof) and an albumin, wherein the nanoparticles have an average particle size of no greater than about 150 nm (such as no greater than about 120 nm); and b) an effective amount of an immunomodulator (such as lenalidomide, pomalidomide, or an immune checkpoint inhibitor). In some embodiments, the method comprises administering to the individual a) an effective amount of a composition comprising nanoparticles comprising an mTOR inhibitor (such as a limus drug, e.g., sirolimus or a derivative thereof) and an albumin, wherein the nanoparticles comprise the mTOR inhibitor associated (e.g., coated) with albumin, wherein the nanoparticles have an average particle size of no greater than about 150 nm (such as no greater than about 120 nm); and b) an effective amount of an immunomodulator (such as lenalidomide, pomalidomide, or an immune checkpoint inhibitor). In some embodiments, the method comprises administering to the individual a) an effective amount of a composition comprising nanoparticles comprising an mTOR inhibitor (such as a limus drug, e.g., sirolimus or a derivative thereof) and an albumin, wherein the nanoparticles comprise the mTOR inhibitor associated (e.g., coated) with the albumin, wherein the nanoparticles have an average particle size of no greater than about 150 nm (such as no greater than about 120 nm, for example about 100 nm), wherein the weight ratio of albumin and the mTOR inhibitor in the mTOR inhibitor nanoparticle composition is about 9:1 or less (such as about 9:1 or about 8:1); and b) an effective amount of an immunomodulator (such as lenalidomide, pomalidomide, or an immune checkpoint inhibitor). In some embodiments, the method further comprises administering to the individual at least one therapeutic agent used in a standard combination therapy with the immunomodulator. In some embodiments, the mTOR inhibitor is a limus drug. In some embodiments, the mTOR inhibitor is sirolimus or a derivative thereof. In some embodiments, the mTOR inhibitor nanoparticle composition comprises nab-sirolimus. In some embodiments, the mTOR inhibitor nanoparticle composition is nab-sirolimus. In some embodiments, the immunomodulator is an immunostimulator that directly stimulates the immune system of an individual. In some embodiments, the immunomodulator is an agonistic antibody that targets an activating receptor on an immune cell (such as a T cell). In some embodiments, the immunomodulator is an immune checkpoint inhibitor. In some embodiments, the immune checkpoint inhibitor is an antagonistic antibody that targets an immune checkpoint protein. In some embodiments, the immunomodulator is an IMiDs® compound (small molecule immunomodulator, such as lenalidomide or pomalidomide). In some embodiments, the immunomodulator is lenalidomide. In some embodiments, the immunomodulator is pomalidomide. In some embodiments, the immunomodulator is small molecule or antibody-based IDO inhibitor. In some embodiments, the lymphangioleiomyomatosis is recurrent lymphangioleiomyomatosis. In some embodiments, the lymphangioleiomyomatosis is refractory to one or more drugs used in a standard therapy for lymphangioleiomyomatosis, such as, but not limited to, sirolimus and/or doxycycline.

In some embodiments, there is provided a method of treating lymphangioleiomyomatosis in an individual (such as a human) comprising administering to the individual a) an effective amount of a composition comprising nanoparticles comprising sirolimus or a derivative thereof and an albumin; and b) an effective amount of an immunomodulator (such as an immunostimulator, e.g., pomalidomide). In some embodiments, the method comprises administering to the individual a) an effective amount of a composition comprising nanoparticles comprising sirolimus or a derivative thereof and an albumin, wherein the sirolimus or derivative thereof in the nanoparticles is associated (e.g., coated) with the albumin; and b) an effective amount of an immunomodulator (such as an immunostimulator, e.g., pomalidomide). In some embodiments, the method comprises administering to the individual a) an effective amount of a composition comprising nanoparticles comprising sirolimus or a derivative thereof and an albumin, wherein the nanoparticles have an average particle size of no greater than about 150 nm (such as no greater than about 120 nm); and b) an effective amount of an immunomodulator (such as an immunostimulator, e.g., pomalidomide). In some embodiments, the method comprises administering to the individual a) an effective amount of a composition comprising nanoparticles comprising sirolimus or a derivative thereof and an albumin, wherein the nanoparticles comprise the sirolimus or derivative thereof associated (e.g., coated) with albumin, wherein the nanoparticles have an average particle size of no greater than about 150 nm (such as no greater than about 120 nm); and b) an effective amount of an immunomodulator (such as an immunostimulator, e.g., pomalidomide). In some embodiments, the method comprises administering to the individual a) an effective amount of a composition comprising nanoparticles comprising sirolimus or a derivative thereof and albumin, wherein the nanoparticles comprise the sirolimus or derivative thereof associated (e.g., coated) with the albumin, wherein the nanoparticles have an average particle size of no greater than about 150 nm (such as no greater than about 120 nm, for example about 100 nm), wherein the weight ratio of albumin and sirolimus or a derivative thereof in the sirolimus nanoparticle composition is about 9:1 or less (such as about 9:1 or about 8:1); and b) an effective amount of an immunomodulator (such as an immunostimulator, e.g., pomalidomide). In some embodiments, the method further comprises administering to the individual at least one therapeutic agent used in a standard combination therapy with the immunomodulator. In some embodiments, the sirolimus or derivative thereof is sirolimus. In some embodiments, the sirolimus nanoparticle composition comprises nab-sirolimus. In some embodiments, the sirolimus nanoparticle composition is nab-sirolimus. In some embodiments, the immunomodulator is an immunostimulator that directly stimulates the immune system of an individual. In some embodiments, the immunomodulator is an agonistic antibody that targets an activating receptor on an immune cell (such as a T cell). In some embodiments, the immunomodulator is an immune checkpoint inhibitor. In some embodiments, the immune checkpoint inhibitor is an antagonistic antibody that targets an immune checkpoint protein. In some embodiments, the immunomodulator is an IMiDs® compound (small molecule immunomodulator, such as lenalidomide or pomalidomide). In some embodiments, the immunomodulator is lenalidomide. In some embodiments, the immunomodulator is pomalidomide. In some embodiments, the immunomodulator is small molecule or antibody-based IDO inhibitor. In some embodiments, the lymphangioleiomyomatosis is recurrent lymphangioleiomyomatosis. In some embodiments, the lymphangioleiomyomatosis is refractory to one or more drugs used in a standard therapy for lymphangioleiomyomatosis, such as, but not limited to, sirolimus and/or doxycycline.

In some embodiments, there is provided a method of treating lymphangioleiomyomatosis in an individual (such as a human) comprising administering to the individual a) an effective amount of a composition comprising nanoparticles comprising an mTOR inhibitor (such as a limus drug, e.g., sirolimus or a derivative thereof) and an albumin; and b) an effective amount of a histone deacetylase inhibitor (such as romidepsin). In some embodiments, the method comprises administering to the individual a) an effective amount of a composition comprising nanoparticles comprising an mTOR inhibitor (such as a limus drug, e.g., sirolimus or a derivative thereof) and an albumin, wherein the mTOR inhibitor in the nanoparticles is associated (e.g., coated) with the albumin; and b) an effective amount of a histone deacetylase inhibitor (such as romidepsin). In some embodiments, the method comprises administering to the individual a) an effective amount of a composition comprising nanoparticles comprising an mTOR inhibitor (such as a limus drug, e.g., sirolimus or a derivative thereof) and an albumin, wherein the nanoparticles have an average particle size of no greater than about 150 nm (such as no greater than about 120 nm); and b) an effective amount of a histone deacetylase inhibitor (such as romidepsin). In some embodiments, the method comprises administering to the individual a) an effective amount of a composition comprising nanoparticles comprising an mTOR inhibitor (such as a limus drug, e.g., sirolimus or a derivative thereof) and an albumin, wherein the nanoparticles comprise the mTOR inhibitor associated (e.g., coated) with albumin, wherein the nanoparticles have an average particle size of no greater than about 150 nm (such as no greater than about 120 nm); and b) an effective amount of a histone deacetylase inhibitor (such as romidepsin). In some embodiments, the method comprises administering to the individual a) an effective amount of a composition comprising nanoparticles comprising an mTOR inhibitor (such as a limus drug, e.g., sirolimus or a derivative thereof) and an albumin, wherein the nanoparticles comprise the mTOR inhibitor associated (e.g., coated) with the albumin, wherein the nanoparticles have an average particle size of no greater than about 150 nm (such as no greater than about 120 nm, for example about 100 nm), wherein the weight ratio of albumin and the mTOR inhibitor in the mTOR inhibitor nanoparticle composition is about 9:1 or less (such as about 9:1 or about 8:1); and b) an effective amount of a histone deacetylase inhibitor (such as romidepsin). In some embodiments, the method further comprises administering to the individual at least one therapeutic agent used in a standard combination therapy with the histone deacetylase inhibitor. In some embodiments, the mTOR inhibitor is a limus drug. In some embodiments, the mTOR inhibitor is sirolimus or a derivative thereof. In some embodiments, the mTOR inhibitor nanoparticle composition comprises nab-sirolimus. In some embodiments, the mTOR inhibitor nanoparticle composition is nab-sirolimus. In some embodiments, the histone deacetylase inhibitor is selected from the group consisting of romidepsin, panobinostat, ricolinostat, and belinostat. In some embodiments, the histone deacetylase inhibitor is romidepsin. In some embodiments, the lymphangioleiomyomatosis is recurrent lymphangioleiomyomatosis. In some embodiments, the lymphangioleiomyomatosis is refractory to one or more drugs used in a standard therapy for lymphangioleiomyomatosis, such as, but not limited to, sirolimus and/or doxycycline.

In some embodiments, there is provided a method of treating lymphangioleiomyomatosis in an individual (such as a human) comprising administering to the individual a) an effective amount of a composition comprising nanoparticles comprising sirolimus or a derivative thereof and an albumin; and b) an effective amount of a histone deacetylase inhibitor (such as romidepsin). In some embodiments, the method comprises administering to the individual a) an effective amount of a composition comprising nanoparticles comprising sirolimus or a derivative thereof and an albumin, wherein the sirolimus or derivative thereof in the nanoparticles is associated (e.g., coated) with the albumin; and b) an effective amount of a histone deacetylase inhibitor (such as romidepsin). In some embodiments, the method comprises administering to the individual a) an effective amount of a composition comprising nanoparticles comprising sirolimus or a derivative thereof and an albumin, wherein the nanoparticles have an average particle size of no greater than about 150 nm (such as no greater than about 120 nm); and b) an effective amount of a histone deacetylase inhibitor (such as romidepsin). In some embodiments, the method comprises administering to the individual a) an effective amount of a composition comprising nanoparticles comprising sirolimus or a derivative thereof and an albumin, wherein the nanoparticles comprise the sirolimus or derivative thereof associated (e.g., coated) with albumin, wherein the nanoparticles have an average particle size of no greater than about 150 nm (such as no greater than about 120 nm); and b) an effective amount of a histone deacetylase inhibitor (such as romidepsin). In some embodiments, the method comprises administering to the individual a) an effective amount of a composition comprising nanoparticles comprising sirolimus or a derivative thereof and albumin, wherein the nanoparticles comprise the sirolimus or derivative thereof associated (e.g., coated) with the albumin, wherein the nanoparticles have an average particle size of no greater than about 150 nm (such as no greater than about 120 nm, for example about 100 nm), wherein the weight ratio of albumin and sirolimus or a derivative thereof in the sirolimus nanoparticle composition is about 9:1 or less (such as about 9:1 or about 8:1); and b) an effective amount of a histone deacetylase inhibitor (such as romidepsin). In some embodiments, the method further comprises administering to the individual at least one therapeutic agent used in a standard combination therapy with the histone deacetylase inhibitor. In some embodiments, the sirolimus or derivative thereof is sirolimus. In some embodiments, the sirolimus nanoparticle composition comprises nab-sirolimus. In some embodiments, the sirolimus nanoparticle composition is nab-sirolimus. In some embodiments, the histone deacetylase inhibitor is selected from the group consisting of romidepsin, panobinostat, ricolinostat, and belinostat. In some embodiments, the histone deacetylase inhibitor is romidepsin. In some embodiments, the lymphangioleiomyomatosis is recurrent lymphangioleiomyomatosis. In some embodiments, the lymphangioleiomyomatosis is refractory to one or more drugs used in a standard therapy for lymphangioleiomyomatosis, such as, but not limited to, sirolimus and/or doxycycline.

In some embodiments, there is provided a method of treating lymphangioleiomyomatosis in an individual (such as a human) comprising administering to the individual a) an effective amount of a composition comprising nanoparticles comprising an mTOR inhibitor (such as a limus drug, e.g., sirolimus or a derivative thereof) and an albumin; and b) an effective amount of a kinase inhibitor (such as a tyrosine kinase inhibitor, e.g., nilotinib or sorafenib). In some embodiments, the method comprises administering to the individual a) an effective amount of a composition comprising nanoparticles comprising an mTOR inhibitor (such as a limus drug, e.g., sirolimus or a derivative thereof) and an albumin, wherein the mTOR inhibitor in the nanoparticles is associated (e.g., coated) with the albumin; and b) an effective amount of a kinase inhibitor (such as a tyrosine kinase inhibitor, e.g., nilotinib or sorafenib). In some embodiments, the method comprises administering to the individual a) an effective amount of a composition comprising nanoparticles comprising an mTOR inhibitor (such as a limus drug, e.g., sirolimus or a derivative thereof) and an albumin, wherein the nanoparticles have an average particle size of no greater than about 150 nm (such as no greater than about 120 nm); and b) an effective amount of a kinase inhibitor (such as a tyrosine kinase inhibitor, e.g., nilotinib or sorafenib). In some embodiments, the method comprises administering to the individual a) an effective amount of a composition comprising nanoparticles comprising an mTOR inhibitor (such as a limus drug, e.g., sirolimus or a derivative thereof) and an albumin, wherein the nanoparticles comprise the mTOR inhibitor associated (e.g., coated) with albumin, wherein the nanoparticles have an average particle size of no greater than about 150 nm (such as no greater than about 120 nm); and b) an effective amount of a kinase inhibitor (such as a tyrosine kinase inhibitor, e.g., nilotinib or sorafenib). In some embodiments, the method comprises administering to the individual a) an effective amount of a composition comprising nanoparticles comprising an mTOR inhibitor (such as a limus drug, e.g., sirolimus or a derivative thereof) and an albumin, wherein the nanoparticles comprise the mTOR inhibitor associated (e.g., coated) with the albumin, wherein the nanoparticles have an average particle size of no greater than about 150 nm (such as no greater than about 120 nm, for example about 100 nm), wherein the weight ratio of albumin and the mTOR inhibitor in the mTOR inhibitor nanoparticle composition is about 9:1 or less (such as about 9:1 or about 8:1); and b) an effective amount of a kinase inhibitor (such as a tyrosine kinase inhibitor, e.g., nilotinib or sorafenib). In some embodiments, the method further comprises administering to the individual at least one therapeutic agent used in a standard combination therapy with the kinase inhibitor. In some embodiments, the mTOR inhibitor is a limus drug. In some embodiments, the mTOR inhibitor is sirolimus or a derivative thereof. In some embodiments, the mTOR inhibitor nanoparticle composition comprises nab-sirolimus. In some embodiments, the mTOR inhibitor nanoparticle composition is nab-sirolimus. In some embodiments, the kinase inhibitor is a tyrosine kinase inhibitor. In some embodiments, the kinase inhibitor is a serine/threonine kinase inhibitor. In some embodiments, the kinase inhibitor is a Raf kinase inhibitor. In some embodiments, the kinase inhibitor inhibits more than one class of kinase (e.g., an inhibitor of more than one of a tyrosine kinase, a Raf kinase, and a serine/threonine kinase). In some embodiments, the kinase inhibitor is selected from the group consisting of erlotinib, imatinib, lapatinib, nilotinib, sorafenib, and sunitinib. In some embodiments, the kinase inhibitor is nilotinib. In some embodiments, the lymphangioleiomyomatosis is recurrent lymphangioleiomyomatosis. In some embodiments, the lymphangioleiomyomatosis is refractory to one or more drugs used in a standard therapy for lymphangioleiomyomatosis, such as, but not limited to, sirolimus and/or doxycycline.

In some embodiments, there is provided a method of treating lymphangioleiomyomatosis in an individual (such as a human) comprising administering to the individual a) an effective amount of a composition comprising nanoparticles comprising sirolimus or a derivative thereof and an albumin; and b) an effective amount of a kinase inhibitor (such as a tyrosine kinase inhibitor, e.g., nilotinib or sorafenib). In some embodiments, the method comprises administering to the individual a) an effective amount of a composition comprising nanoparticles comprising sirolimus or a derivative thereof and an albumin, wherein the sirolimus or derivative thereof in the nanoparticles is associated (e.g., coated) with the albumin; and b) an effective amount of a kinase inhibitor (such as a tyrosine kinase inhibitor, e.g., nilotinib or sorafenib). In some embodiments, the method comprises administering to the individual a) an effective amount of a composition comprising nanoparticles comprising sirolimus or a derivative thereof and an albumin, wherein the nanoparticles have an average particle size of no greater than about 150 nm (such as no greater than about 120 nm); and b) an effective amount of a kinase inhibitor (such as a tyrosine kinase inhibitor, e.g., nilotinib or sorafenib). In some embodiments, the method comprises administering to the individual a) an effective amount of a composition comprising nanoparticles comprising sirolimus or a derivative thereof and an albumin, wherein the nanoparticles comprise the sirolimus or derivative thereof associated (e.g., coated) with albumin, wherein the nanoparticles have an average particle size of no greater than about 150 nm (such as no greater than about 120 nm); and b) an effective amount of a kinase inhibitor (such as a tyrosine kinase inhibitor, e.g., nilotinib or sorafenib). In some embodiments, the method comprises administering to the individual a) an effective amount of a composition comprising nanoparticles comprising sirolimus or a derivative thereof and an albumin, wherein the nanoparticles comprise the sirolimus or derivative thereof associated (e.g., coated) with the albumin, wherein the nanoparticles have an average particle size of no greater than about 150 nm (such as no greater than about 120 nm, for example about 100 nm), wherein the weight ratio of albumin and the sirolimus or derivative thereof in the sirolimus nanoparticle composition is about 9:1 or less (such as about 9:1 or about 8:1); and b) an effective amount of a kinase inhibitor (such as a tyrosine kinase inhibitor, e.g., nilotinib or sorafenib). In some embodiments, the method further comprises administering to the individual at least one therapeutic agent used in a standard combination therapy with the kinase inhibitor. In some embodiments, the sirolimus or derivative thereof is sirolimus. In some embodiments, the sirolimus nanoparticle composition comprises nab-sirolimus. In some embodiments, the sirolimus nanoparticle composition is nab-sirolimus. In some embodiments, the kinase inhibitor is a tyrosine kinase inhibitor. In some embodiments, the kinase inhibitor is a serine/threonine kinase inhibitor. In some embodiments, the kinase inhibitor is a Raf kinase inhibitor. In some embodiments, the kinase inhibitor inhibits more than one class of kinase (e.g., an inhibitor of more than one of a tyrosine kinase, a Raf kinase, and a serine/threonine kinase). In some embodiments, the kinase inhibitor is selected from the group consisting of erlotinib, imatinib, lapatinib, nilotinib, sorafenib, and sunitinib. In some embodiments, the kinase inhibitor is nilotinib. In some embodiments, the lymphangioleiomyomatosis is recurrent lymphangioleiomyomatosis. In some embodiments, the lymphangioleiomyomatosis is refractory to one or more drugs used in a standard therapy for lymphangioleiomyomatosis, such as, but not limited to, sirolimus and/or doxycycline.

In some embodiments, there is provided a method of treating lymphangioleiomyomatosis in an individual (such as a human) comprising administering to the individual a) an effective amount of a composition comprising nanoparticles comprising an mTOR inhibitor (such as a limus drug, e.g., sirolimus or a derivative thereof) and an albumin; and b) an effective amount of a cancer vaccine. In some embodiments, the method comprises administering to the individual a) an effective amount of a composition comprising nanoparticles comprising an mTOR inhibitor (such as a limus drug, e.g., sirolimus or a derivative thereof) and an albumin, wherein the mTOR inhibitor in the nanoparticles is associated (e.g., coated) with the albumin; and b) an effective amount of a cancer vaccine. In some embodiments, the method comprises administering to the individual a) an effective amount of a composition comprising nanoparticles comprising an mTOR inhibitor (such as a limus drug, e.g., sirolimus or a derivative thereof) and an albumin, wherein the nanoparticles have an average particle size of no greater than about 150 nm (such as no greater than about 120 nm); and b) an effective amount of a cancer vaccine. In some embodiments, the method comprises administering to the individual a) an effective amount of a composition comprising nanoparticles comprising an mTOR inhibitor (such as a limus drug, e.g., sirolimus or a derivative thereof) and an albumin, wherein the nanoparticles comprise the mTOR inhibitor associated (e.g., coated) with albumin, wherein the nanoparticles have an average particle size of no greater than about 150 nm (such as no greater than about 120 nm); and b) an effective amount of a cancer vaccine. In some embodiments, the method comprises administering to the individual a) an effective amount of a composition comprising nanoparticles comprising an mTOR inhibitor (such as a limus drug, e.g., sirolimus or a derivative thereof) and an albumin, wherein the nanoparticles comprise the mTOR inhibitor associated (e.g., coated) with the albumin, wherein the nanoparticles have an average particle size of no greater than about 150 nm (such as no greater than about 120 nm, for example about 100 nm), wherein the weight ratio of albumin and the mTOR inhibitor in the mTOR inhibitor nanoparticle composition is about 9:1 or less (such as about 9:1 or about 8:1); and b) an effective amount of a cancer vaccine. In some embodiments, the method further comprises administering to the individual at least one therapeutic agent used in a standard combination therapy with the cancer vaccine. In some embodiments, the mTOR inhibitor is a limus drug. In some embodiments, the mTOR inhibitor is sirolimus or a derivative thereof. In some embodiments, the mTOR inhibitor nanoparticle composition comprises nab-sirolimus. In some embodiments, the mTOR inhibitor nanoparticle composition is nab-sirolimus. In some embodiments, the cancer vaccine is a vaccine prepared using autologous tumor cells. In some embodiments, the cancer vaccine is a vaccine prepared using allogeneic tumor cells. In some embodiments, the lymphangioleiomyomatosis is recurrent lymphangioleiomyomatosis. In some embodiments, the lymphangioleiomyomatosis is refractory to one or more drugs used in a standard therapy for lymphangioleiomyomatosis, such as, but not limited to, sirolimus and/or doxycycline.

In some embodiments, there is provided a method of treating lymphangioleiomyomatosis in an individual (such as a human) comprising administering to the individual a) an effective amount of a composition comprising nanoparticles comprising sirolimus or a derivative thereof and an albumin; and b) an effective amount of a cancer vaccine. In some embodiments, the method comprises administering to the individual a) an effective amount of a composition comprising nanoparticles comprising sirolimus or a derivative thereof and an albumin, wherein the sirolimus or derivative thereof in the nanoparticles is associated (e.g., coated) with the albumin; and b) an effective amount of a cancer vaccine. In some embodiments, the method comprises administering to the individual a) an effective amount of a composition comprising nanoparticles comprising sirolimus or a derivative thereof and an albumin, wherein the nanoparticles have an average particle size of no greater than about 150 nm (such as no greater than about 120 nm); and b) an effective amount of a cancer vaccine. In some embodiments, the method comprises administering to the individual a) an effective amount of a composition comprising nanoparticles comprising sirolimus or a derivative thereof and an albumin, wherein the nanoparticles comprise the sirolimus or derivative thereof associated (e.g., coated) with albumin, wherein the nanoparticles have an average particle size of no greater than about 150 nm (such as no greater than about 120 nm); and b) an effective amount of a cancer vaccine. In some embodiments, the method comprises administering to the individual a) an effective amount of a composition comprising nanoparticles comprising sirolimus or a derivative thereof and an albumin, wherein the nanoparticles comprise the sirolimus or derivative thereof associated (e.g., coated) with the albumin, wherein the nanoparticles have an average particle size of no greater than about 150 nm (such as no greater than about 120 nm, for example about 100 nm), wherein the weight ratio of albumin and the sirolimus or derivative thereof in the sirolimus nanoparticle composition is about 9:1 or less (such as about 9:1 or about 8:1); and b) an effective amount of a cancer vaccine. In some embodiments, the method further comprises administering to the individual at least one therapeutic agent used in a standard combination therapy with the cancer vaccine. In some embodiments, the sirolimus or derivative thereof is sirolimus. In some embodiments, the sirolimus nanoparticle composition comprises nab-sirolimus. In some embodiments, the sirolimus nanoparticle composition is nab-sirolimus. In some embodiments, the cancer vaccine is a vaccine prepared using autologous tumor cells. In some embodiments, the cancer vaccine is a vaccine prepared using allogeneic tumor cells. In some embodiments, the lymphangioleiomyomatosis is recurrent lymphangioleiomyomatosis. In some embodiments, the lymphangioleiomyomatosis is refractory to one or more drugs used in a standard therapy for lymphangioleiomyomatosis, such as, but not limited to, sirolimus and/or doxycycline.

In some embodiments, there is provided a method of treating lymphangioleiomyomatosis in an individual (such as a human) comprising administering to the individual a) an effective amount of a composition comprising nanoparticles comprising sirolimus or a derivative thereof and an albumin; and b) an effective amount of doxycycline. In some embodiments, the method comprises administering to the individual a) an effective amount of a composition comprising nanoparticles comprising sirolimus or a derivative thereof and an albumin, wherein the sirolimus or derivative thereof in the nanoparticles is associated (e.g., coated) with the albumin; and b) an effective amount of doxycycline. In some embodiments, the method comprises administering to the individual a) an effective amount of a composition comprising nanoparticles comprising sirolimus or a derivative thereof and an albumin, wherein the nanoparticles have an average particle size of no greater than about 150 nm (such as no greater than about 120 nm); and b) an effective amount of doxycycline. In some embodiments, the method comprises administering to the individual a) an effective amount of a composition comprising nanoparticles comprising sirolimus or a derivative thereof and an albumin, wherein the nanoparticles comprise the sirolimus or derivative thereof associated (e.g., coated) with albumin, wherein the nanoparticles have an average particle size of no greater than about 150 nm (such as no greater than about 120 nm); and b) an effective amount of doxycycline. In some embodiments, the method comprises administering to the individual a) an effective amount of a composition comprising nanoparticles comprising sirolimus or a derivative thereof and an albumin, wherein the nanoparticles comprise the sirolimus or derivative thereof associated (e.g., coated) with the albumin, wherein the nanoparticles have an average particle size of no greater than about 150 nm (such as no greater than about 120 nm, for example about 100 nm), wherein the weight ratio of albumin and the sirolimus or derivative thereof in the sirolimus nanoparticle composition is about 9:1 or less (such as about 9:1 or about 8:1); and b) an effective amount of doxycycline. In some embodiments, the method further comprises administering to the individual at least one therapeutic agent used in a standard combination therapy with doxycycline. In some embodiments, the sirolimus or derivative thereof is sirolimus. In some embodiments, the sirolimus nanoparticle composition comprises nab-sirolimus. In some embodiments, the sirolimus nanoparticle composition is nab-sirolimus. In some embodiments, the lymphangioleiomyomatosis is recurrent lymphangioleiomyomatosis. In some embodiments, the lymphangioleiomyomatosis is refractory to one or more drugs used in a standard therapy for lymphangioleiomyomatosis, such as, but not limited to, sirolimus and/or doxycycline.

In some embodiments, according to any of the methods of treating lymphangioleiomyomatosis in an individual described herein, the individual is a human who exhibits one or more symptoms associated with lymphangioleiomyomatosis. In some embodiments, the individual is at an early stage of lymphangioleiomyomatosis. In some embodiments, the individual is at an advanced stage of lymphangioleiomyomatosis. In some of embodiments, the individual is genetically or otherwise predisposed (e.g., having a risk factor) to developing lymphangioleiomyomatosis. Individuals at risk for lymphangioleiomyomatosis include, e.g., those having relatives who have experienced lymphangioleiomyomatosis, and those whose risk is determined by analysis of genetic or biochemical markers. In some embodiments, the individual may be a human who has a gene, genetic mutation, or polymorphism associated with lymphangioleiomyomatosis (e.g., TSC1 and/or TSC2) or has one or more extra copies of a gene associated with lymphangioleiomyomatosis. In some embodiments, the individual has a ras or PTEN mutation. In some embodiments, the cancer cells are dependent on an mTOR pathway to translate one or more mRNAs. In some embodiments, the cancer cells are not capable of synthesizing mRNAs by an mTOR-independent pathway. In some embodiments, the cancer cells have decreased or no PTEN activity or have decreased or no expression of PTEN compared to non-cancerous cells. In some embodiments, the individual has at least one tumor biomarker selected from the group consisting of elevated PI3K activity, elevated mTOR activity, presence of FLT-3ITD, elevated AKT activity, elevated KRAS activity, and elevated NRAS activity. In some embodiments, the individual has a variation in at least one gene selected from the group consisting of drug metabolism genes, cancer genes, and drug target genes.

### Prostate Cancer

In some embodiments, there is provided a method of treating prostate cancer in an individual (such as a human) comprising administering to the individual a) an effective amount of a composition comprising nanoparticles comprising an mTOR inhibitor (such as a limus drug, e.g., sirolimus or a derivative thereof) and an albumin; and b) an effective amount of a second therapeutic agent. In some embodiments, the method comprises administering to the individual a) an effective amount of a composition comprising nanoparticles comprising an mTOR inhibitor (such as a limus drug, e.g., sirolimus or a derivative thereof) and an albumin, wherein the mTOR inhibitor in the nanoparticles is associated (e.g., coated) with the albumin; and b) an effective amount of a second therapeutic agent. In some embodiments, the method comprises administering to the individual a) an effective amount of a composition comprising nanoparticles comprising an mTOR inhibitor (such as a limus drug, e.g., sirolimus or a derivative thereof) and an albumin, wherein the nanoparticles have an average particle size of no greater than about 150 nm (such as no greater than about 120 nm); and b) an effective amount of a second therapeutic agent. In some embodiments, the method comprises administering to the individual a) an effective amount of a composition comprising nanoparticles comprising an mTOR inhibitor (such as a limus drug, e.g., sirolimus or a derivative thereof) and an albumin, wherein the nanoparticles comprise the mTOR inhibitor associated (e.g., coated) with albumin, wherein the nanoparticles have an average particle size of no greater than about 150 nm (such as no greater than about 120 nm); and b) an effective amount of a second therapeutic agent. In some embodiments, the method comprises administering to the individual a) an effective amount of a composition comprising nanoparticles comprising an mTOR inhibitor (such as a limus drug, e.g., sirolimus or a derivative thereof) and an albumin, wherein the nanoparticles comprise the mTOR inhibitor associated (e.g., coated) with the albumin, wherein the nanoparticles have an average particle size of no greater than about 150 nm (such as no greater than about 120 nm, for example about 100 nm), wherein the weight ratio of albumin and the mTOR inhibitor in the mTOR inhibitor nanoparticle composition is about 9:1 or less (such as about 9:1 or about 8:1); and b) an effective amount of a second therapeutic agent. In some embodiments, the method further comprises administering to the individual at least one therapeutic agent used in a standard combination therapy with the second therapeutic agent. In some embodiments, the mTOR inhibitor is a limus drug. In some embodiments, the mTOR inhibitor is sirolimus or a derivative thereof. In some embodiments, the mTOR inhibitor nanoparticle composition comprises nab-sirolimus. In some embodiments, the mTOR inhibitor nanoparticle composition is nab-sirolimus. In some embodiments, the second therapeutic agent is selected from the group consisting of an immunomodulator (such as an immunostimulator or an immune checkpoint inhibitor), a histone deacetylase inhibitor, a kinase inhibitor (such as a tyrosine kinase inhibitor), and a cancer vaccine (such as a vaccine prepared using tumor cells or at least one tumor-associated antigen). In some embodiments, the second therapeutic agent is an immunomodulator. In some embodiments, the immunomodulator is an immunostimulator that directly stimulates the immune system of an individual. In some embodiments, the immunomodulator is an agonistic antibody that targets an activating receptor on an immune cell (such as a T cell). In some embodiments, the immunomodulator is an immune checkpoint inhibitor. In some embodiments, the immune checkpoint inhibitor is an antagonistic antibody that targets an immune checkpoint protein. In some embodiments, the immunomodulator is an IMiDs® compound (small molecule immunomodulator, such as lenalidomide or pomalidomide). In some embodiments, the immunomodulator is lenalidomide. In some embodiments, the immunomodulator is pomalidomide. In some embodiments, the immunomodulator is small molecule or antibody-based IDO inhibitor. In some embodiments, the second therapeutic agent is a histone deacetylase inhibitor. In some embodiments, the histone deacetylase inhibitor is specific to only one HDAC. In some embodiments, the histone deacetylase inhibitor is specific to only one class of HDAC. In some embodiments, the histone deacetylase inhibitor is specific to two or more HDACs or two or more classes of HDACs. In some embodiments, the histone deacetylase inhibitor is specific to class I and II HDACs. In some embodiments, the histone deacetylase inhibitor is specific to class III HDACs. In some embodiments, the histone deacetylase inhibitor is selected from the group consisting of romidepsin, panobinostat, ricolinostat, and belinostat. In some embodiments, the histone deacetylase inhibitor is romidepsin. In some embodiments, the second therapeutic agent is a kinase inhibitor, such as a tyrosine kinase inhibitor. In some embodiments, the kinase inhibitor is a serine/threonine kinase inhibitor. In some embodiments, the kinase inhibitor is a Raf kinase inhibitor. In some embodiments, the kinase inhibitor inhibits more than one class of kinase (e.g., an inhibitor of more than one of a tyrosine kinase, a Raf kinase, and a serine/threonine kinase). In some embodiments, the kinase inhibitor is selected from the group consisting of erlotinib, imatinib, lapatinib, nilotinib, sorafenib, and sunitinib. In some embodiments, the kinase inhibitor is sorafenib. In some embodiments, the kinase inhibitor is nilotinib. In some embodiments, the second therapeutic agent is a cancer vaccine, such as a vaccine prepared using tumor cells or at least one tumor-associated antigen. In some embodiments, the second therapeutic agent and the nanoparticle composition are administered sequentially. In some embodiments, the second therapeutic agent and the nanoparticle composition are administered simultaneously. In some embodiments, the second therapeutic agent and the nanoparticle composition are administered concurrently.

In some embodiments, the prostate cancer is an adenocarcinoma. In some embodiments, the prostate cancer is a sarcoma, neuroendocrine tumor, small cell cancer, ductal cancer, or a lymphoma. In some embodiments, the prostate cancer is at any of the four stages, A, B, C, or D, according to the Jewett staging system. In some embodiments, the prostate cancer is stage A prostate cancer (e.g., the cancer cannot be felt during a rectal exam). In some embodiments, the prostate cancer is stage B prostate cancer (e.g., the tumor involves more tissue within the prostate, and can be felt during a rectal exam, or is found with a biopsy that is done because of a high PSA level). In some embodiments, the prostate cancer is stage C prostate cancer (e.g., the cancer has spread outside the prostate to nearby tissues). In some embodiments, the prostate cancer is stage D prostate cancer. In some embodiments, the prostate cancer is androgen independent prostate cancer (AIPC). In some embodiments, the prostate cancer is androgen dependent prostate cancer. In some embodiments, the prostate cancer is refractory to hormone therapy.

In some embodiments, there is provided a method of treating prostate cancer in an individual (such as a human) comprising administering to the individual a) an effective amount of a composition comprising nanoparticles comprising an mTOR inhibitor (such as a limus drug, e.g., sirolimus or a derivative thereof) and an albumin; and b) an effective amount of an immunomodulator (such as lenalidomide, pomalidomide, or an immune checkpoint inhibitor). In some embodiments, the method comprises administering to the individual a) an effective amount of a composition comprising nanoparticles comprising an mTOR inhibitor (such as a limus drug, e.g., sirolimus or a derivative thereof) and an albumin, wherein the mTOR inhibitor in the nanoparticles is associated (e.g., coated) with the albumin; and b) an effective amount of an immunomodulator (such as lenalidomide, pomalidomide, or an immune checkpoint inhibitor). In some embodiments, the method comprises administering to the individual a) an effective amount of a composition comprising nanoparticles comprising an mTOR inhibitor (such as a limus drug, e.g., sirolimus or a derivative thereof) and an albumin, wherein the nanoparticles have an average particle size of no greater than about 150 nm (such as no greater than about 120 nm); and b) an effective amount of an immunomodulator (such as lenalidomide, pomalidomide, or an immune checkpoint inhibitor). In some embodiments, the method comprises administering to the individual a) an effective amount of a composition comprising nanoparticles comprising an mTOR inhibitor (such as a limus drug, e.g., sirolimus or a derivative thereof) and an albumin, wherein the nanoparticles comprise the mTOR inhibitor associated (e.g., coated) with albumin, wherein the nanoparticles have an average particle size of no greater than about 150 nm (such as no greater than about 120 nm); and b) an effective amount of an immunomodulator (such as lenalidomide, pomalidomide, or an immune checkpoint inhibitor). In some embodiments, the method comprises administering to the individual a) an effective amount of a composition comprising nanoparticles comprising an mTOR inhibitor (such as a limus drug, e.g., sirolimus or a derivative thereof) and an albumin, wherein the nanoparticles comprise the mTOR inhibitor associated (e.g., coated) with the albumin, wherein the nanoparticles have an average particle size of no greater than about 150 nm (such as no greater than about 120 nm, for example about 100 nm), wherein the weight ratio of albumin and the mTOR inhibitor in the mTOR inhibitor nanoparticle composition is about 9:1 or less (such as about 9:1 or about 8:1); and b) an effective amount of an immunomodulator (such as lenalidomide, pomalidomide, or an immune checkpoint inhibitor). In some embodiments, the method further comprises administering to the individual at least one therapeutic agent used in a standard combination therapy with the immunomodulator. In some embodiments, the mTOR inhibitor is a limus drug. In some embodiments, the mTOR inhibitor is sirolimus or a derivative thereof. In some embodiments, the mTOR inhibitor nanoparticle composition comprises nab-sirolimus. In some embodiments, the mTOR inhibitor nanoparticle composition is nab-sirolimus. In some embodiments, the immunomodulator is an immunostimulator that directly stimulates the immune system of an individual. In some embodiments, the immunomodulator is an agonistic antibody that targets an activating receptor on an immune cell (such as a T cell). In some embodiments, the immunomodulator is an immune checkpoint inhibitor. In some embodiments, the immune checkpoint inhibitor is an antagonistic antibody that targets an immune checkpoint protein. In some embodiments, the immunomodulator is an IMiDs® compound (small molecule immunomodulator, such as lenalidomide or pomalidomide). In some embodiments, the immunomodulator is lenalidomide. In some embodiments, the immunomodulator is pomalidomide. In some embodiments, the immunomodulator is small molecule or antibody-based IDO inhibitor. In some embodiments, the prostate cancer is recurrent prostate cancer. In some embodiments, the prostate cancer is refractory to one or more drugs used in a standard therapy for prostate cancer, such as, but not limited to, docetaxel, cabazitaxel, mitoxantrone, estramustine, doxorubicin, etoposide, vinblastine, paclitaxel, carboplatin, and/or vinorelbine.

In some embodiments, there is provided a method of treating prostate cancer in an individual (such as a human) comprising administering to the individual a) an effective amount of a composition comprising nanoparticles comprising sirolimus or a derivative thereof and an albumin; and b) an effective amount of an immunomodulator (such as an immunostimulator, e.g., pomalidomide). In some embodiments, the method comprises administering to the individual a) an effective amount of a composition comprising nanoparticles comprising sirolimus or a derivative thereof and an albumin, wherein the sirolimus or derivative thereof in the nanoparticles is associated (e.g., coated) with the albumin; and b) an effective amount of an immunomodulator (such as an immunostimulator, e.g., pomalidomide). In some embodiments, the method comprises administering to the individual a) an effective amount of a composition comprising nanoparticles comprising sirolimus or a derivative thereof and an albumin, wherein the nanoparticles have an average particle size of no greater than about 150 nm (such as no greater than about 120 nm); and b) an effective amount of an immunomodulator (such as an immunostimulator, e.g., pomalidomide). In some embodiments, the method comprises administering to the individual a) an effective amount of a composition comprising nanoparticles comprising sirolimus or a derivative thereof and an albumin, wherein the nanoparticles comprise the sirolimus or derivative thereof associated (e.g., coated) with albumin, wherein the nanoparticles have an average particle size of no greater than about 150 nm (such as no greater than about 120 nm); and b) an effective amount of an immunomodulator (such as an immunostimulator, e.g., pomalidomide). In some embodiments, the method comprises administering to the individual a) an effective amount of a composition comprising nanoparticles comprising sirolimus or a derivative thereof and albumin, wherein the nanoparticles comprise the sirolimus or derivative thereof associated (e.g., coated) with the albumin, wherein the nanoparticles have an average particle size of no greater than about 150 nm (such as no greater than about 120 nm, for example about 100 nm), wherein the weight ratio of albumin and sirolimus or a derivative thereof in the sirolimus nanoparticle composition is about 9:1 or less (such as about 9:1 or about 8:1); and b) an effective amount of an immunomodulator (such as an immunostimulator, e.g., pomalidomide). In some embodiments, the method further comprises administering to the individual at least one therapeutic agent used in a standard combination therapy with the immunomodulator. In some embodiments, the sirolimus or derivative thereof is sirolimus. In some embodiments, the sirolimus nanoparticle composition comprises nab-sirolimus. In some embodiments, the sirolimus nanoparticle composition is nab-sirolimus. In some embodiments, the immunomodulator is an immunostimulator that directly stimulates the immune system of an individual. In some embodiments, the immunomodulator is an agonistic antibody that targets an activating receptor on an immune cell (such as a T cell). In some embodiments, the immunomodulator is an immune checkpoint inhibitor. In some embodiments, the immune checkpoint inhibitor is an antagonistic antibody that targets an immune checkpoint protein. In some embodiments, the immunomodulator is an IMiDs® compound (small molecule immunomodulator, such as lenalidomide or pomalidomide). In some embodiments, the immunomodulator is lenalidomide. In some embodiments, the immunomodulator is pomalidomide. In some embodiments, the immunomodulator is small molecule or antibody-based IDO inhibitor. In some embodiments, the prostate cancer is recurrent prostate cancer. In some embodiments, the prostate cancer is refractory to one or more drugs used in a standard therapy for prostate cancer, such as, but not limited to, docetaxel, cabazitaxel, mitoxantrone, estramustine, doxorubicin, etoposide, vinblastine, paclitaxel, carboplatin, and/or vinorelbine.

In some embodiments, there is provided a method of treating prostate cancer in an individual (such as a human) comprising administering to the individual a) an effective amount of a composition comprising nanoparticles comprising an mTOR inhibitor (such as a limus drug, e.g., sirolimus or a derivative thereof) and an albumin; and b) an effective amount of a histone deacetylase inhibitor (such as romidepsin). In some embodiments, the method comprises administering to the individual a) an effective amount of a composition comprising nanoparticles comprising an mTOR inhibitor (such as a limus drug, e.g., sirolimus or a derivative thereof) and an albumin, wherein the mTOR inhibitor in the nanoparticles is associated (e.g., coated) with the albumin; and b) an effective amount of a histone deacetylase inhibitor (such as romidepsin). In some embodiments, the method comprises administering to the individual a) an effective amount of a composition comprising nanoparticles comprising an mTOR inhibitor (such as a limus drug, e.g., sirolimus or a derivative thereof) and an albumin, wherein the nanoparticles have an average particle size of no greater than about 150 nm (such as no greater than about 120 nm); and b) an effective amount of a histone deacetylase inhibitor (such as romidepsin). In some embodiments, the method comprises administering to the individual a) an effective amount of a composition comprising nanoparticles comprising an mTOR inhibitor (such as a limus drug, e.g., sirolimus or a derivative thereof) and an albumin, wherein the nanoparticles comprise the mTOR inhibitor associated (e.g., coated) with albumin, wherein the nanoparticles have an average particle size of no greater than about 150 nm (such as no greater than about 120 nm); and b) an effective amount of a histone deacetylase inhibitor (such as romidepsin). In some embodiments, the method comprises administering to the individual a) an effective amount of a composition comprising nanoparticles comprising an mTOR inhibitor (such as a limus drug, e.g., sirolimus or a derivative thereof) and an albumin, wherein the nanoparticles comprise the mTOR inhibitor associated (e.g., coated) with the albumin, wherein the nanoparticles have an average particle size of no greater than about 150 nm (such as no greater than about 120 nm, for example about 100 nm), wherein the weight ratio of albumin and the mTOR inhibitor in the mTOR inhibitor nanoparticle composition is about 9:1 or less (such as about 9:1 or about 8:1); and b) an effective amount of a histone deacetylase inhibitor (such as romidepsin). In some embodiments, the method further comprises administering to the individual at least one therapeutic agent used in a standard combination therapy with the histone deacetylase inhibitor. In some embodiments, the mTOR inhibitor is a limus drug. In some embodiments, the mTOR inhibitor is sirolimus or a derivative thereof. In some embodiments, the mTOR inhibitor nanoparticle composition comprises nab-sirolimus. In some embodiments, the mTOR inhibitor nanoparticle composition is nab-sirolimus. In some embodiments, the histone deacetylase inhibitor is selected from the group consisting of romidepsin, panobinostat, ricolinostat, and belinostat. In some embodiments, the histone deacetylase inhibitor is romidepsin. In some embodiments, the prostate cancer is recurrent prostate cancer. In some embodiments, the prostate cancer is refractory to one or more drugs used in a standard therapy for prostate cancer, such as, but not limited to, docetaxel, cabazitaxel, mitoxantrone, estramustine, doxorubicin, etoposide, vinblastine, paclitaxel, carboplatin, and/or vinorelbine.

In some embodiments, there is provided a method of treating prostate cancer in an individual (such as a human) comprising administering to the individual a) an effective amount of a composition comprising nanoparticles comprising sirolimus or a derivative thereof and an albumin; and b) an effective amount of a histone deacetylase inhibitor (such as romidepsin). In some embodiments, the method comprises administering to the individual a) an effective amount of a composition comprising nanoparticles comprising sirolimus or a derivative thereof and an albumin, wherein the sirolimus or derivative thereof in the nanoparticles is associated (e.g., coated) with the albumin; and b) an effective amount of a histone deacetylase inhibitor (such as romidepsin). In some embodiments, the method comprises administering to the individual a) an effective amount of a composition comprising nanoparticles comprising sirolimus or a derivative thereof and an albumin, wherein the nanoparticles have an average particle size of no greater than about 150 nm (such as no greater than about 120 nm); and b) an effective amount of a histone deacetylase inhibitor (such as romidepsin). In some embodiments, the method comprises administering to the individual a) an effective amount of a composition comprising nanoparticles comprising sirolimus or a derivative thereof and an albumin, wherein the nanoparticles comprise the sirolimus or derivative thereof associated (e.g., coated) with albumin, wherein the nanoparticles have an average particle size of no greater than about 150 nm (such as no greater than about 120 nm); and b) an effective amount of a histone deacetylase inhibitor (such as romidepsin). In some embodiments, the method comprises administering to the individual a) an effective amount of a composition comprising nanoparticles comprising sirolimus or a derivative thereof and albumin, wherein the nanoparticles comprise the sirolimus or derivative thereof associated (e.g., coated) with the albumin, wherein the nanoparticles have an average particle size of no greater than about 150 nm (such as no greater than about 120 nm, for example about 100 nm), wherein the weight ratio of albumin and sirolimus or a derivative thereof in the sirolimus nanoparticle composition is about 9:1 or less (such as about 9:1 or about 8:1); and b) an effective amount of a histone deacetylase inhibitor (such as romidepsin). In some embodiments, the method further comprises administering to the individual at least one therapeutic agent used in a standard combination therapy with the histone deacetylase inhibitor. In some embodiments, the sirolimus or derivative thereof is sirolimus. In some embodiments, the sirolimus nanoparticle composition comprises nab-sirolimus. In some embodiments, the sirolimus nanoparticle composition is nab-sirolimus. In some embodiments, the histone deacetylase inhibitor is selected from the group consisting of romidepsin, panobinostat, ricolinostat, and belinostat. In some embodiments, the histone deacetylase inhibitor is romidepsin. In some embodiments, the prostate cancer is recurrent prostate cancer. In some embodiments, the prostate cancer is refractory to one or more drugs used in a standard therapy for prostate cancer, such as, but not limited to, docetaxel, cabazitaxel, mitoxantrone, estramustine, doxorubicin, etoposide, vinblastine, paclitaxel, carboplatin, and/or vinorelbine.

In some embodiments, there is provided a method of treating prostate cancer in an individual (such as a human) comprising administering to the individual a) an effective amount of a composition comprising nanoparticles comprising an mTOR inhibitor (such as a limus drug, e.g., sirolimus or a derivative thereof) and an albumin; and b) an effective amount of a kinase inhibitor (such as a tyrosine kinase inhibitor, e.g., nilotinib or sorafenib). In some embodiments, the method comprises administering to the individual a) an effective amount of a composition comprising nanoparticles comprising an mTOR inhibitor (such as a limus drug, e.g., sirolimus or a derivative thereof) and an albumin, wherein the mTOR inhibitor in the nanoparticles is associated (e.g., coated) with the albumin; and b) an effective amount of a kinase inhibitor (such as a tyrosine kinase inhibitor, e.g., nilotinib or sorafenib). In some embodiments, the method comprises administering to the individual a) an effective amount of a composition comprising nanoparticles comprising an mTOR inhibitor (such as a limus drug, e.g., sirolimus or a derivative thereof) and an albumin, wherein the nanoparticles have an average particle size of no greater than about 150 nm (such as no greater than about 120 nm); and b) an effective amount of a kinase inhibitor (such as a tyrosine kinase inhibitor, e.g., nilotinib or sorafenib). In some embodiments, the method comprises administering to the individual a) an effective amount of a composition comprising nanoparticles comprising an mTOR inhibitor (such as a limus drug, e.g., sirolimus or a derivative thereof) and an albumin, wherein the nanoparticles comprise the mTOR inhibitor associated (e.g., coated) with albumin, wherein the nanoparticles have an average particle size of no greater than about 150 nm (such as no greater than about 120 nm); and b) an effective amount of a kinase inhibitor (such as a tyrosine kinase inhibitor, e.g., nilotinib or sorafenib). In some embodiments, the method comprises administering to the individual a) an effective amount of a composition comprising nanoparticles comprising an mTOR inhibitor (such as a limus drug, e.g., sirolimus or a derivative thereof) and an albumin, wherein the nanoparticles comprise the mTOR inhibitor associated (e.g., coated) with the albumin, wherein the nanoparticles have an average particle size of no greater than about 150 nm (such as no greater than about 120 nm, for example about 100 nm), wherein the weight ratio of albumin and the mTOR inhibitor in the mTOR inhibitor nanoparticle composition is about 9:1 or less (such as about 9:1 or about 8:1); and b) an effective amount of a kinase inhibitor (such as a tyrosine kinase inhibitor, e.g., nilotinib or sorafenib). In some embodiments, the method further comprises administering to the individual at least one therapeutic agent used in a standard combination therapy with the kinase inhibitor. In some embodiments, the mTOR inhibitor is a limus drug. In some embodiments, the mTOR inhibitor is sirolimus or a derivative thereof. In some embodiments, the mTOR inhibitor nanoparticle composition comprises nab-sirolimus. In some embodiments, the mTOR inhibitor nanoparticle composition is nab-sirolimus. In some embodiments, the kinase inhibitor is a tyrosine kinase inhibitor. In some embodiments, the kinase inhibitor is a serine/threonine kinase inhibitor. In some embodiments, the kinase inhibitor is a Raf kinase inhibitor. In some embodiments, the kinase inhibitor inhibits more than one class of kinase (e.g., an inhibitor of more than one of a tyrosine kinase, a Raf kinase, and a serine/threonine kinase). In some embodiments, the kinase inhibitor is selected from the group consisting of erlotinib, imatinib, lapatinib, nilotinib, sorafenib, and sunitinib. In some embodiments, the kinase inhibitor is nilotinib. In some embodiments, the prostate cancer is recurrent prostate cancer. In some embodiments, the prostate cancer is refractory to one or more drugs used in a standard therapy for prostate cancer, such as, but not limited to, docetaxel, cabazitaxel, mitoxantrone, estramustine, doxorubicin, etoposide, vinblastine, paclitaxel, carboplatin, and/or vinorelbine.

In some embodiments, there is provided a method of treating prostate cancer in an individual (such as a human) comprising administering to the individual a) an effective amount of a composition comprising nanoparticles comprising sirolimus or a derivative thereof and an albumin; and b) an effective amount of a kinase inhibitor (such as a tyrosine kinase inhibitor, e.g., nilotinib or sorafenib). In some embodiments, the method comprises administering to the individual a) an effective amount of a composition comprising nanoparticles comprising sirolimus or a derivative thereof and an albumin, wherein the sirolimus or derivative thereof in the nanoparticles is associated (e.g., coated) with the albumin; and b) an effective amount of a kinase inhibitor (such as a tyrosine kinase inhibitor, e.g., nilotinib or sorafenib). In some embodiments, the method comprises administering to the individual a) an effective amount of a composition comprising nanoparticles comprising sirolimus or a derivative thereof and an albumin, wherein the nanoparticles have an average particle size of no greater than about 150 nm (such as no greater than about 120 nm); and b) an effective amount of a kinase inhibitor (such as a tyrosine kinase inhibitor, e.g., nilotinib or sorafenib). In some embodiments, the method comprises administering to the individual a) an effective amount of a composition comprising nanoparticles comprising sirolimus or a derivative thereof and an albumin, wherein the nanoparticles comprise the sirolimus or derivative thereof associated (e.g., coated) with albumin, wherein the nanoparticles have an average particle size of no greater than about 150 nm (such as no greater than about 120 nm); and b) an effective amount of a kinase inhibitor (such as a tyrosine kinase inhibitor, e.g., nilotinib or sorafenib). In some embodiments, the method comprises administering to the individual a) an effective amount of a composition comprising nanoparticles comprising sirolimus or a derivative thereof and an albumin, wherein the nanoparticles comprise the sirolimus or derivative thereof associated (e.g., coated) with the albumin, wherein the nanoparticles have an average particle size of no greater than about 150 nm (such as no greater than about 120 nm, for example about 100 nm), wherein the weight ratio of albumin and the sirolimus or derivative thereof in the sirolimus nanoparticle composition is about 9:1 or less (such as about 9:1 or about 8:1); and b) an effective amount of a kinase inhibitor (such as a tyrosine kinase inhibitor, e.g., nilotinib or sorafenib). In some embodiments, the method further comprises administering to the individual at least one therapeutic agent used in a standard combination therapy with the kinase inhibitor. In some embodiments, the sirolimus or derivative thereof is sirolimus. In some embodiments, the sirolimus nanoparticle composition comprises nab-sirolimus. In some embodiments, the sirolimus nanoparticle composition is nab-sirolimus. In some embodiments, the kinase inhibitor is a tyrosine kinase inhibitor. In some embodiments, the kinase inhibitor is a serine/threonine kinase inhibitor. In some embodiments, the kinase inhibitor is a Raf kinase inhibitor. In some embodiments, the kinase inhibitor inhibits more than one class of kinase (e.g., an inhibitor of more than one of a tyrosine kinase, a Raf kinase, and a serine/threonine kinase). In some embodiments, the kinase inhibitor is selected from the group consisting of erlotinib, imatinib, lapatinib, nilotinib, sorafenib, and sunitinib. In some embodiments, the kinase inhibitor is nilotinib. In some embodiments, the prostate cancer is recurrent prostate cancer. In some embodiments, the prostate cancer is refractory to one or more drugs used in a standard therapy for prostate cancer, such as, but not limited to, docetaxel, cabazitaxel, mitoxantrone, estramustine, doxorubicin, etoposide, vinblastine, paclitaxel, carboplatin, and/or vinorelbine.

In some embodiments, there is provided a method of treating prostate cancer in an individual (such as a human) comprising administering to the individual a) an effective amount of a composition comprising nanoparticles comprising an mTOR inhibitor (such as a limus drug, e.g., sirolimus or a derivative thereof) and an albumin; and b) an effective amount of a cancer vaccine. In some embodiments, the method comprises administering to the individual a) an effective amount of a composition comprising nanoparticles comprising an mTOR inhibitor (such as a limus drug, e.g., sirolimus or a derivative thereof) and an albumin, wherein the mTOR inhibitor in the nanoparticles is associated (e.g., coated) with the albumin; and b) an effective amount of a cancer vaccine. In some embodiments, the method comprises administering to the individual a) an effective amount of a composition comprising nanoparticles comprising an mTOR inhibitor (such as a limus drug, e.g., sirolimus or a derivative thereof) and an albumin, wherein the nanoparticles have an average particle size of no greater than about 150 nm (such as no greater than about 120 nm); and b) an effective amount of a cancer vaccine. In some embodiments, the method comprises administering to the individual a) an effective amount of a composition comprising nanoparticles comprising an mTOR inhibitor (such as a limus drug, e.g., sirolimus or a derivative thereof) and an albumin, wherein the nanoparticles comprise the mTOR inhibitor associated (e.g., coated) with albumin, wherein the nanoparticles have an average particle size of no greater than about 150 nm (such as no greater than about 120 nm); and b) an effective amount of a cancer vaccine. In some embodiments, the method comprises administering to the individual a) an effective amount of a composition comprising nanoparticles comprising an mTOR inhibitor (such as a limus drug, e.g., sirolimus or a derivative thereof) and an albumin, wherein the nanoparticles comprise the mTOR inhibitor associated (e.g., coated) with the albumin, wherein the nanoparticles have an average particle size of no greater than about 150 nm (such as no greater than about 120 nm, for example about 100 nm), wherein the weight ratio of albumin and the mTOR inhibitor in the mTOR inhibitor nanoparticle composition is about 9:1 or less (such as about 9:1 or about 8:1); and b) an effective amount of a cancer vaccine. In some embodiments, the method further comprises administering to the individual at least one therapeutic agent used in a standard combination therapy with the cancer vaccine. In some embodiments, the mTOR inhibitor is a limus drug. In some embodiments, the mTOR inhibitor is sirolimus or a derivative thereof. In some embodiments, the mTOR inhibitor nanoparticle composition comprises nab-sirolimus. In some embodiments, the mTOR inhibitor nanoparticle composition is nab-sirolimus. In some embodiments, the cancer vaccine is a vaccine prepared using autologous tumor cells. In some embodiments, the cancer vaccine is a vaccine prepared using allogeneic tumor cells. In some embodiments, the prostate cancer is recurrent prostate cancer. In some embodiments, the prostate cancer is refractory to one or more drugs used in a standard therapy for prostate cancer, such as, but not limited to, docetaxel, cabazitaxel, mitoxantrone, estramustine, doxorubicin, etoposide, vinblastine, paclitaxel, carboplatin, and/or vinorelbine.

In some embodiments, there is provided a method of treating prostate cancer in an individual (such as a human) comprising administering to the individual a) an effective amount of a composition comprising nanoparticles comprising sirolimus or a derivative thereof and an albumin; and b) an effective amount of a cancer vaccine. In some embodiments, the method comprises administering to the individual a) an effective amount of a composition comprising nanoparticles comprising sirolimus or a derivative thereof and an albumin, wherein the sirolimus or derivative thereof in the nanoparticles is associated (e.g., coated) with the albumin; and b) an effective amount of a cancer vaccine. In some embodiments, the method comprises administering to the individual a) an effective amount of a composition comprising nanoparticles comprising sirolimus or a derivative thereof and an albumin, wherein the nanoparticles have an average particle size of no greater than about 150 nm (such as no greater than about 120 nm); and b) an effective amount of a cancer vaccine. In some embodiments, the method comprises administering to the individual a) an effective amount of a composition comprising nanoparticles comprising sirolimus or a derivative thereof and an albumin, wherein the nanoparticles comprise the sirolimus or derivative thereof associated (e.g., coated) with albumin, wherein the nanoparticles have an average particle size of no greater than about 150 nm (such as no greater than about 120 nm); and b) an effective amount of a cancer vaccine. In some embodiments, the method comprises administering to the individual a) an effective amount of a composition comprising nanoparticles comprising sirolimus or a derivative thereof and an albumin, wherein the nanoparticles comprise the sirolimus or derivative thereof associated (e.g., coated) with the albumin, wherein the nanoparticles have an average particle size of no greater than about 150 nm (such as no greater than about 120 nm, for example about 100 nm), wherein the weight ratio of albumin and the sirolimus or derivative thereof in the sirolimus nanoparticle composition is about 9:1 or less (such as about 9:1 or about 8:1); and b) an effective amount of a cancer vaccine. In some embodiments, the method further comprises administering to the individual at least one therapeutic agent used in a standard combination therapy with the cancer vaccine. In some embodiments, the sirolimus or derivative thereof is sirolimus. In some embodiments, the sirolimus nanoparticle composition comprises nab-sirolimus. In some embodiments, the sirolimus nanoparticle composition is nab-sirolimus. In some embodiments, the cancer vaccine is a vaccine prepared using autologous tumor cells. In some embodiments, the cancer vaccine is a vaccine prepared using allogeneic tumor cells. In some embodiments, the prostate cancer is recurrent prostate cancer. In some embodiments, the prostate cancer is refractory to one or more drugs used in a standard therapy for prostate cancer, such as, but not limited to, docetaxel, cabazitaxel, mitoxantrone, estramustine, doxorubicin, etoposide, vinblastine, paclitaxel, carboplatin, and/or vinorelbine.

In some embodiments, there is provided a method of treating prostate cancer in an individual (such as a human) comprising administering to the individual a) an effective amount of a composition comprising nanoparticles comprising sirolimus or a derivative thereof and an albumin; and b) an effective amount of a second therapeutic agent selected from the group consisting of docetaxel, cabazitaxel, mitoxantrone, estramustine, doxorubicin, etoposide, vinblastine, paclitaxel, carboplatin, and vinorelbine. In some embodiments, the method comprises administering to the individual a) an effective amount of a composition comprising nanoparticles comprising sirolimus or a derivative thereof and an albumin, wherein the sirolimus or derivative thereof in the nanoparticles is associated (e.g., coated) with the albumin; and b) an effective amount of a second therapeutic agent selected from the group consisting of docetaxel, cabazitaxel, mitoxantrone, estramustine, doxorubicin, etoposide, vinblastine, paclitaxel, carboplatin, and vinorelbine. In some embodiments, the method comprises administering to the individual a) an effective amount of a composition comprising nanoparticles comprising sirolimus or a derivative thereof and an albumin, wherein the nanoparticles have an average particle size of no greater than about 150 nm (such as no greater than about 120 nm); and b) an effective amount of a second therapeutic agent selected from the group consisting of docetaxel, cabazitaxel, mitoxantrone, estramustine, doxorubicin, etoposide, vinblastine, paclitaxel, carboplatin, and vinorelbine. In some embodiments, the method comprises administering to the individual a) an effective amount of a composition comprising nanoparticles comprising sirolimus or a derivative thereof and an albumin, wherein the nanoparticles comprise the sirolimus or derivative thereof associated (e.g., coated) with albumin, wherein the nanoparticles have an average particle size of no greater than about 150 nm (such as no greater than about 120 nm); and b) an effective amount of a second therapeutic agent selected from the group consisting of docetaxel, cabazitaxel, mitoxantrone, estramustine, doxorubicin, etoposide, vinblastine, paclitaxel, carboplatin, and vinorelbine. In some embodiments, the method comprises administering to the individual a) an effective amount of a composition comprising nanoparticles comprising sirolimus or a derivative thereof and an albumin, wherein the nanoparticles comprise the sirolimus or derivative thereof associated (e.g., coated) with the albumin, wherein the nanoparticles have an average particle size of no greater than about 150 nm (such as no greater than about 120 nm, for example about 100 nm), wherein the weight ratio of albumin and the sirolimus or derivative thereof in the sirolimus nanoparticle composition is about 9:1 or less (such as about 9:1 or about 8:1); and b) an effective amount of a second therapeutic agent selected from the group consisting of docetaxel, cabazitaxel, mitoxantrone, estramustine, doxorubicin, etoposide, vinblastine, paclitaxel, carboplatin, and vinorelbine. In some embodiments, the method further comprises administering to the individual at least one therapeutic agent used in a standard combination therapy with the second therapeutic agent. In some embodiments, the sirolimus or derivative thereof is sirolimus. In some embodiments, the sirolimus nanoparticle composition comprises nab-sirolimus. In some embodiments, the sirolimus nanoparticle composition is nab-sirolimus. In some embodiments, the prostate cancer is recurrent prostate cancer. In some embodiments, the prostate cancer is refractory to one or more drugs used in a standard therapy for prostate cancer, such as, but not limited to, docetaxel, cabazitaxel, mitoxantrone, estramustine, doxorubicin, etoposide, vinblastine, paclitaxel, carboplatin, and/or vinorelbine.

In some embodiments, according to any of the methods of treating prostate cancer in an individual described herein, the individual is a human who exhibits one or more symptoms associated with prostate cancer. In some embodiments, the individual is at an early stage of prostate cancer. In some embodiments, the individual is at an advanced stage of prostate cancer. In some of embodiments, the individual is genetically or otherwise predisposed (e.g., having a risk factor) to developing prostate cancer. Individuals at risk for prostate cancer include, e.g., those having relatives who have experienced prostate cancer, and those whose risk is determined by analysis of genetic or biochemical markers. In some embodiments, the individual may be a human who has a gene, genetic mutation, or polymorphism associated with prostate cancer (e.g., RNASEL/HPC1, ELAC2/HPC2, SR-A/MSR1, CHEK2, BRCA2, PON1, OGG1, MIC-I, TLR4, and/or PTEN) or has one or more extra copies of a gene associated with prostate cancer. In some embodiments, the individual has a ras or PTEN mutation. In some embodiments, the cancer cells are dependent on an mTOR pathway to translate one or more mRNAs. In some embodiments, the cancer cells are not capable of synthesizing mRNAs by an mTOR-independent pathway. In some embodiments, the cancer cells have decreased or no PTEN activity or have decreased or no expression of PTEN compared to non-cancerous cells. In some embodiments, the individual has at least one tumor biomarker selected from the group consisting of elevated PI3K activity, elevated mTOR activity, presence of FLT-3ITD, elevated AKT activity, elevated KRAS activity, and elevated NRAS activity. In some embodiments, the individual has a variation in at least one gene selected from the group consisting of drug metabolism genes, cancer genes, and drug target genes.

### Vascular Tumors

In some embodiments, there is provided a method of treating a vascular tumor in an individual (such as a human) comprising administering to the individual a) an effective amount of a composition comprising nanoparticles comprising an mTOR inhibitor (such as a limus drug, e.g., sirolimus or a derivative thereof) and an albumin; and b) an effective amount of a second therapeutic agent. In some embodiments, the method comprises administering to the individual a) an effective amount of a composition comprising nanoparticles comprising an mTOR inhibitor (such as a limus drug, e.g., sirolimus or a derivative thereof) and an albumin, wherein the mTOR inhibitor in the nanoparticles is associated (e.g., coated) with the albumin; and b) an effective amount of a second therapeutic agent. In some embodiments, the method comprises administering to the individual a) an effective amount of a composition comprising nanoparticles comprising an mTOR inhibitor (such as a limus drug, e.g., sirolimus or a derivative thereof) and an albumin, wherein the nanoparticles have an average particle size of no greater than about 150 nm (such as no greater than about 120 nm); and b) an effective amount of a second therapeutic agent. In some embodiments, the method comprises administering to the individual a) an effective amount of a composition comprising nanoparticles comprising an mTOR inhibitor (such as a limus drug, e.g., sirolimus or a derivative thereof) and an albumin, wherein the nanoparticles comprise the mTOR inhibitor associated (e.g., coated) with albumin, wherein the nanoparticles have an average particle size of no greater than about 150 nm (such as no greater than about 120 nm); and b) an effective amount of a second therapeutic agent. In some embodiments, the method comprises administering to the individual a) an effective amount of a composition comprising nanoparticles comprising an mTOR inhibitor (such as a limus drug, e.g., sirolimus or a derivative thereof) and an albumin, wherein the nanoparticles comprise the mTOR inhibitor associated (e.g., coated) with the albumin, wherein the nanoparticles have an average particle size of no greater than about 150 nm (such as no greater than about 120 nm, for example about 100 nm), wherein the weight ratio of albumin and the mTOR inhibitor in the mTOR inhibitor nanoparticle composition is about 9:1 or less (such as about 9:1 or about 8:1); and b) an effective amount of a second therapeutic agent. In some embodiments, the method further comprises administering to the individual at least one therapeutic agent used in a standard combination therapy with the second therapeutic agent. In some embodiments, the mTOR inhibitor is a limus drug. In some embodiments, the mTOR inhibitor is sirolimus or a derivative thereof. In some embodiments, the mTOR inhibitor nanoparticle composition comprises nab-sirolimus. In some embodiments, the mTOR inhibitor nanoparticle composition is nab-sirolimus. In some embodiments, the second therapeutic agent is selected from the group consisting of an immunomodulator (such as an immunostimulator or an immune checkpoint inhibitor), a histone deacetylase inhibitor, a kinase inhibitor (such as a tyrosine kinase inhibitor), and a cancer vaccine (such as a vaccine prepared using tumor cells or at least one tumor-associated antigen). In some embodiments, the second therapeutic agent is an immunomodulator. In some embodiments, the immunomodulator is an immunostimulator that directly stimulates the immune system of an individual. In some embodiments, the immunomodulator is an agonistic antibody that targets an activating receptor on an immune cell (such as a T cell). In some embodiments, the immunomodulator is an immune checkpoint inhibitor. In some embodiments, the immune checkpoint inhibitor is an antagonistic antibody that targets an immune checkpoint protein. In some embodiments, the immunomodulator is an IMiDs® compound (small molecule immunomodulator, such as lenalidomide or pomalidomide). In some embodiments, the immunomodulator is lenalidomide. In some embodiments, the immunomodulator is pomalidomide. In some embodiments, the immunomodulator is small molecule or antibody-based IDO inhibitor. In some embodiments, the second therapeutic agent is a histone deacetylase inhibitor. In some embodiments, the histone deacetylase inhibitor is specific to only one HDAC. In some embodiments, the histone deacetylase inhibitor is specific to only one class of HDAC. In some embodiments, the histone deacetylase inhibitor is specific to two or more HDACs or two or more classes of HDACs. In some embodiments, the histone deacetylase inhibitor is specific to class I and II HDACs. In some embodiments, the histone deacetylase inhibitor is specific to class III HDACs. In some embodiments, the histone deacetylase inhibitor is selected from the group consisting of romidepsin, panobinostat, ricolinostat, and belinostat. In some embodiments, the histone deacetylase inhibitor is romidepsin. In some embodiments, the second therapeutic agent is a kinase inhibitor, such as a tyrosine kinase inhibitor. In some embodiments, the kinase inhibitor is a serine/threonine kinase inhibitor. In some embodiments, the kinase inhibitor is a Raf kinase inhibitor. In some embodiments, the kinase inhibitor inhibits more than one class of kinase (e.g., an inhibitor of more than one of a tyrosine kinase, a Raf kinase, and a serine/threonine kinase). In some embodiments, the kinase inhibitor is selected from the group consisting of erlotinib, imatinib, lapatinib, nilotinib, sorafenib, and sunitinib. In some embodiments, the kinase inhibitor is sorafenib. In some embodiments, the kinase inhibitor is nilotinib. In some embodiments, the second therapeutic agent is a cancer vaccine, such as a vaccine prepared using tumor cells or at least one tumor-associated antigen. In some embodiments, the second therapeutic agent is vincristine. In some embodiments, the second therapeutic agent and the nanoparticle composition are administered sequentially. In some embodiments, the second therapeutic agent and the nanoparticle composition are administered simultaneously. In some embodiments, the second therapeutic agent and the nanoparticle composition are administered concurrently. In some embodiments, the vascular tumor is Kaposi sarcoma, angiosarcoma, tufted angioma, or kaposiform hemangioendothelioma (KHE). In some embodiments, the vascular tumor is refractory to a prior therapy.

In some embodiments, there is provided a method of treating a vascular tumor (such as Kaposi sarcoma, angiosarcoma, tufted angioma, or kaposiform hemangioendothelioma) in an individual (such as a human) comprising administering to the individual a) an effective amount of a composition comprising nanoparticles comprising an mTOR inhibitor (such as a limus drug, e.g., sirolimus or a derivative thereof) and an albumin; and b) an effective amount of vincristine. In some embodiments, the method comprises administering to the individual a) an effective amount of a composition comprising nanoparticles comprising an mTOR inhibitor (such as a limus drug, e.g., sirolimus or a derivative thereof) and an albumin, wherein the mTOR inhibitor in the nanoparticles is associated (e.g., coated) with the albumin; and b) an effective amount of vincristine. In some embodiments, the method comprises administering to the individual a) an effective amount of a composition comprising nanoparticles comprising an mTOR inhibitor (such as a limus drug, e.g., sirolimus or a derivative thereof) and an albumin, wherein the nanoparticles have an average particle size of no greater than about 150 nm (such as no greater than about 120 nm); and b) an effective amount of vincristine. In some embodiments, the method comprises administering to the individual a) an effective amount of a composition comprising nanoparticles comprising an mTOR inhibitor (such as a limus drug, e.g., sirolimus or a derivative thereof) and an albumin, wherein the nanoparticles comprise the mTOR inhibitor associated (e.g., coated) with albumin, wherein the nanoparticles have an average particle size of no greater than about 150 nm (such as no greater than about 120 nm); and b) an effective amount of vincristine. In some embodiments, the method comprises administering to the individual a) an effective amount of a composition comprising nanoparticles comprising an mTOR inhibitor (such as a limus drug, e.g., sirolimus or a derivative thereof) and an albumin, wherein the nanoparticles comprise the mTOR inhibitor associated (e.g., coated) with the albumin, wherein the nanoparticles have an average particle size of no greater than about 150 nm (such as no greater than about 120 nm, for example about 100 nm), wherein the weight ratio of albumin and the mTOR inhibitor in the mTOR inhibitor nanoparticle composition is about 9:1 or less (such as about 9:1 or about 8:1); and b) an effective amount of vincristine. In some embodiments, the method further comprises administering to the individual at least one therapeutic agent used in a standard combination therapy with vincristine. In some embodiments, the mTOR inhibitor is a limus drug. In some embodiments, the mTOR inhibitor is sirolimus or a derivative thereof. In some embodiments, the mTOR inhibitor nanoparticle composition comprises nab-sirolimus. In some embodiments, the mTOR inhibitor nanoparticle composition is nab-sirolimus. In some embodiments, the mTOR inhibitor nanoparticle is in the dosage range of about 10 mg/m² to about 200 mg/m² (including for example about any of 10 mg/m² to about 40 mg/m², about 40 mg/m² to about 75 mg/m², about 75 mg/m² to about 100 mg/m², about 100 mg/m² to about 200 mg/m², about 20 mg/m² to about 55 mg/m², and any ranges between these values). In some embodiments, the mTOR inhibitor nanoparticle is in the dosage range of about 20 mg/m² to about 55 mg/m² (such as about any of 20 mg/m², 35 mg/m², 45 mg/m², or 55 mg/m²). In some embodiments, the vincristine is in the dosage range of about 0.5 mg/m² to about 5 mg/m² (including for example about any of 0.5 mg/m² to about 1 mg/m², about 1 mg/m² to about 1.5 mg/m², about 1.5 mg/m² to about 2 mg/m², about 2 mg/m² to about 2.5 mg/m², about 2.5 mg/m² to about 3 mg/m², about 3 mg/m² to about 4 mg/m², about 4 mg/m² to about 5 mg/m², about 1.5 mg/m², and any ranges between these values). In some embodiments, the vincristine is in a dosage of about 1.5 mg/m². In some embodiments, the vascular tumor is a recurrent vascular tumor. In some embodiments, the vascular tumor is refractory to one or more drugs used in a standard therapy for the vascular tumor. In some embodiments, the vascular tumor is Kaposi sarcoma. In some embodiments, the vascular tumor is angiosarcoma. In some embodiments, the vascular tumor is tufted angioma. or In some embodiments, the vascular tumor is kaposiform hemangioendothelioma.

### Pediatric Tumors

The present application in one aspect provides a method of treating solid tumor in a human individual comprising administering to the individual an effective amount of a composition comprising nanoparticles comprising an mTOR inhibitor (such as sirolimus) and albumin and an effective amount of a second therapeutic agent (such as vincristine), wherein the individual is no more than about 21 years old (such as no more than about 18 years old). The solid tumor includes, for example, neuroblastoma, osteosarcoma, Ewing sarcoma, rhabdomyosarcoma, medulloblastoma, glioma, hepatic tumor, renal tumor, tufted angioma, and kaposiform hemangioendothelioma (KHE). In some embodiments, the individual is resistant or refractory to a prior treatment. In some embodiments, the individual has progressed on the prior treatment. In some embodiments, the individual has a recurrent solid tumor.

In some embodiments, there is provided a method of treating a solid tumor in an individual (such as a human) comprising administering to the individual a) an effective amount of a composition comprising nanoparticles comprising an mTOR inhibitor (such as a limus drug, e.g., sirolimus or a derivative thereof) and an albumin; and b) an effective amount of a second therapeutic agent, wherein the individual is no more than about 21 years old (such as no more than about 18 years old). In some embodiments, the method comprises administering to the individual a) an effective amount of a composition comprising nanoparticles comprising an mTOR inhibitor (such as a limus drug, e.g., sirolimus or a derivative thereof) and an albumin, wherein the mTOR inhibitor in the nanoparticles is associated (e.g., coated) with the albumin; and b) an effective amount of a second therapeutic agent. In some embodiments, the method comprises administering to the individual a) an effective amount of a composition comprising nanoparticles comprising an mTOR inhibitor (such as a limus drug, e.g., sirolimus or a derivative thereof) and an albumin, wherein the nanoparticles have an average particle size of no greater than about 150 nm (such as no greater than about 120 nm); and b) an effective amount of a second therapeutic agent. In some embodiments, the method comprises administering to the individual a) an effective amount of a composition comprising nanoparticles comprising an mTOR inhibitor (such as a limus drug, e.g., sirolimus or a derivative thereof) and an albumin, wherein the nanoparticles comprise the mTOR inhibitor associated (e.g., coated) with albumin, wherein the nanoparticles have an average particle size of no greater than about 150 nm (such as no greater than about 120 nm); and b) an effective amount of a second therapeutic agent. In some embodiments, the method comprises administering to the individual a) an effective amount of a composition comprising nanoparticles comprising an mTOR inhibitor (such as a limus drug, e.g., sirolimus or a derivative thereof) and an albumin, wherein the nanoparticles comprise the mTOR inhibitor associated (e.g., coated) with the albumin, wherein the nanoparticles have an average particle size of no greater than about 150 nm (such as no greater than about 120 nm, for example about 100 nm), wherein the weight ratio of albumin and the mTOR inhibitor in the mTOR inhibitor nanoparticle composition is about 9:1 or less (such as about 9:1 or about 8:1); and b) an effective amount of a second therapeutic agent. In some embodiments, the method further comprises administering to the individual at least one therapeutic agent used in a standard combination therapy with the second therapeutic agent. In some embodiments, the mTOR inhibitor is a limus drug. In some embodiments, the mTOR inhibitor is sirolimus or a derivative thereof. In some embodiments, the mTOR inhibitor nanoparticle composition comprises nab-sirolimus. In some embodiments, the mTOR inhibitor nanoparticle composition is nab-sirolimus. In some embodiments, the second therapeutic agent is selected from the group consisting of an immunomodulator (such as an immunostimulator or an immune checkpoint inhibitor), a histone deacetylase inhibitor, a kinase inhibitor (such as a tyrosine kinase inhibitor), and a cancer vaccine (such as a vaccine prepared using tumor cells or at least one tumor-associated antigen). In some embodiments, the second therapeutic agent is an immunomodulator. In some embodiments, the immunomodulator is an immunostimulator that directly stimulates the immune system of an individual. In some embodiments, the immunomodulator is an agonistic antibody that targets an activating receptor on an immune cell (such as a T cell). In some embodiments, the immunomodulator is an immune checkpoint inhibitor. In some embodiments, the immune checkpoint inhibitor is an antagonistic antibody that targets an immune checkpoint protein. In some embodiments, the immunomodulator is an IMiDs® compound (small molecule immunomodulator, such as lenalidomide or pomalidomide). In some embodiments, the immunomodulator is lenalidomide. In some embodiments, the immunomodulator is pomalidomide. In some embodiments, the immunomodulator is small molecule or antibody-based IDO inhibitor. In some embodiments, the second therapeutic agent is a histone deacetylase inhibitor. In some embodiments, the histone deacetylase inhibitor is specific to only one HDAC. In some embodiments, the histone deacetylase inhibitor is specific to only one class of HDAC. In some embodiments, the histone deacetylase inhibitor is specific to two or more HDACs or two or more classes of HDACs. In some embodiments, the histone deacetylase inhibitor is specific to class I and II HDACs. In some embodiments, the histone deacetylase inhibitor is specific to class III HDACs. In some embodiments, the histone deacetylase inhibitor is selected from the group consisting of romidepsin, panobinostat, ricolinostat, and belinostat. In some embodiments, the histone deacetylase inhibitor is romidepsin. In some embodiments, the second therapeutic agent is a kinase inhibitor, such as a tyrosine kinase inhibitor. In some embodiments, the kinase inhibitor is a serine/threonine kinase inhibitor. In some embodiments, the kinase inhibitor is a Raf kinase inhibitor. In some embodiments, the kinase inhibitor inhibits more than one class of kinase (e.g., an inhibitor of more than one of a tyrosine kinase, a Raf kinase, and a serine/threonine kinase). In some embodiments, the kinase inhibitor is selected from the group consisting of erlotinib, imatinib, lapatinib, nilotinib, sorafenib, and sunitinib. In some embodiments, the kinase inhibitor is sorafenib. In some embodiments, the kinase inhibitor is nilotinib. In some embodiments, the second therapeutic agent is a cancer vaccine, such as a vaccine prepared using tumor cells or at least one tumor-associated antigen. In some embodiments, the second therapeutic agent is temozolomide, irinotecan, vincristine, or a combination thereof. For example, in some embodiments, the method comprises administering to the individual a) an effective amount of a composition comprising nanoparticles comprising an mTOR inhibitor (such as a limus drug, e.g., sirolimus or a derivative thereof) and an albumin; and b) an effective amount of i) temozolomide and irinotecan; or ii) vincristine. In some embodiments, the second therapeutic agent and the nanoparticle composition are administered sequentially. In some embodiments, the second therapeutic agent and the nanoparticle composition are administered simultaneously. In some embodiments, the second therapeutic agent and the nanoparticle composition are administered concurrently. In some embodiments, the solid tumor is neuroblastoma, osteosarcoma, Ewing sarcoma, rhabdomyosarcoma, medulloblastoma, glioma, hepatic tumor, renal tumor, tufted angioma, or kaposiform hemangioendothelioma. In some embodiments, the solid tumor is refractory to a prior therapy. In some embodiments, the individual is no more than about any of 17, 16, 15, 14, 13, 12, 11, 10, 9, 8, 7, 6, 5, 4, 3, 2, or 1 year old. In some embodiments, the individual is about 9 to about 15 years old. In some embodiments, the individual is about 5 to about 9 years old. In some embodiments, the individual is about 1 to about 5 years old. In some embodiments, the individual is no more than about 1 year old, such as about 6 months old to about 1 year old, less than about 6 months old, or less than about 3 months old.

In some embodiments, there is provided a method of treating a solid tumor (such as neuroblastoma, osteosarcoma, Ewing sarcoma, rhabdomyosarcoma, medulloblastoma, glioma, hepatic tumor, or renal tumor) in an individual (such as a human) comprising administering to the individual a) an effective amount of a composition comprising nanoparticles comprising an mTOR inhibitor (such as a limus drug, e.g., sirolimus or a derivative thereof) and an albumin; and b) an effective amount of temozolomide and irinotecan, wherein the individual is no more than about 21 years old (such as no more than about 18 years old). In some embodiments, the method comprises administering to the individual a) an effective amount of a composition comprising nanoparticles comprising an mTOR inhibitor (such as a limus drug, e.g., sirolimus or a derivative thereof) and an albumin, wherein the mTOR inhibitor in the nanoparticles is associated (e.g., coated) with the albumin; and b) an effective amount of temozolomide and irinotecan. In some embodiments, the method comprises administering to the individual a) an effective amount of a composition comprising nanoparticles comprising an mTOR inhibitor (such as a limus drug, e.g., sirolimus or a derivative thereof) and an albumin, wherein the nanoparticles have an average particle size of no greater than about 150 nm (such as no greater than about 120 nm); and b) an effective amount of temozolomide and irinotecan. In some embodiments, the method comprises administering to the individual a) an effective amount of a composition comprising nanoparticles comprising an mTOR inhibitor (such as a limus drug, e.g., sirolimus or a derivative thereof) and an albumin, wherein the nanoparticles comprise the mTOR inhibitor associated (e.g., coated) with albumin, wherein the nanoparticles have an average particle size of no greater than about 150 nm (such as no greater than about 120 nm); and b) an effective amount of temozolomide and irinotecan. In some embodiments, the method comprises administering to the individual a) an effective amount of a composition comprising nanoparticles comprising an mTOR inhibitor (such as a limus drug, e.g., sirolimus or a derivative thereof) and an albumin, wherein the nanoparticles comprise the mTOR inhibitor associated (e.g., coated) with the albumin, wherein the nanoparticles have an average particle size of no greater than about 150 nm (such as no greater than about 120 nm, for example about 100 nm), wherein the weight ratio of albumin and the mTOR inhibitor in the mTOR inhibitor nanoparticle composition is about 9:1 or less (such as about 9:1 or about 8:1); and b) an effective amount of temozolomide and irinotecan. In some embodiments, the method further comprises administering to the individual at least one therapeutic agent used in a standard combination therapy with temozolomide and irinotecan. In some embodiments, the mTOR inhibitor is a limus drug. In some embodiments, the mTOR inhibitor is sirolimus or a derivative thereof. In some embodiments, the mTOR inhibitor nanoparticle composition comprises nab-sirolimus. In some embodiments, the mTOR inhibitor nanoparticle composition is nab-sirolimus. In some embodiments, the mTOR inhibitor nanoparticle is in the dosage range of about 10 mg/m² to about 200 mg/m² (including for example about any of 10 mg/m² to about 40 mg/m², about 40 mg/m² to about 75 mg/m², about 75 mg/m² to about 100 mg/m², about 100 mg/m² to about 200 mg/m², about 20 mg/m² to about 55 mg/m², and any ranges between these values). In some embodiments, the mTOR inhibitor nanoparticle is in the dosage range of about 20 mg/m² to about 55 mg/m² (such as about any of 20 mg/m², 35 mg/m², 45 mg/m², or 55 mg/m²). In some embodiments, the temozolomide is in the dosage range of about 10 mg/m² to about 200 mg/m² (including for example about any of 10 mg/m² to about 40 mg/m², about 40 mg/m² to about 75 mg/m², about 75 mg/m² to about 100 mg/m², about 100 mg/m² to about 200 mg/m², about 125 mg/m², and any ranges between these values). In some embodiments, the temozolomide is in a dosage of about 125 mg/m². In some embodiments, the irinotecan is in the dosage range of about 10 mg/m² to about 200 mg/m² (including for example about any of 10 mg/m² to about 40 mg/m², about 40 mg/m² to about 75 mg/m², about 75 mg/m² to about 100 mg/m², about 100 mg/m² to about 200 mg/m², about 90 mg/m², and any ranges between these values). In some embodiments, the irinotecan is in a dosage of about 90 mg/m². In some embodiments, the solid tumor is a recurrent solid tumor. In some embodiments, the solid tumor is refractory to one or more drugs used in a standard therapy for the solid tumor. In some embodiments, the solid tumor is neuroblastoma. In some embodiments, the solid tumor is osteosarcoma. In some embodiments, the solid tumor is Ewing sarcoma. In some embodiments, the solid tumor is rhabdomyosarcoma. In some embodiments, the solid tumor is medulloblastoma. In some embodiments, the solid tumor is glioma. In some embodiments, the solid tumor is hepatic tumor. In some embodiments, the solid tumor is renal tumor. In some embodiments, the individual is no more than about any of 17, 16, 15, 14, 13, 12, 11, 10, 9, 8, 7, 6, 5, 4, 3, 2, or 1 year old. In some embodiments, the individual is about 9 to about 15 years old. In some embodiments, the individual is about 5 to about 9 years old. In some embodiments, the individual is about 1 to about 5 years old. In some embodiments, the individual is no more than about 1 year old, such as about 6 months old to about 1 year old, less than about 6 months old, or less than about 3 months old.

In some embodiments, there is provided a method of treating a solid tumor (such as tufted angioma or kaposiform hemangioendothelioma) in an individual (such as a human) comprising administering to the individual a) an effective amount of a composition comprising nanoparticles comprising an mTOR inhibitor (such as a limus drug, e.g., sirolimus or a derivative thereof) and an albumin; and b) an effective amount of vincristine, wherein the individual is no more than about 21 years old (such as no more than about 18 years old). In some embodiments, the method comprises administering to the individual a) an effective amount of a composition comprising nanoparticles comprising an mTOR inhibitor (such as a limus drug, e.g., sirolimus or a derivative thereof) and an albumin, wherein the mTOR inhibitor in the nanoparticles is associated (e.g., coated) with the albumin; and b) an effective amount of vincristine. In some embodiments, the method comprises administering to the individual a) an effective amount of a composition comprising nanoparticles comprising an mTOR inhibitor (such as a limus drug, e.g., sirolimus or a derivative thereof) and an albumin, wherein the nanoparticles have an average particle size of no greater than about 150 nm (such as no greater than about 120 nm); and b) an effective amount of vincristine. In some embodiments, the method comprises administering to the individual a) an effective amount of a composition comprising nanoparticles comprising an mTOR inhibitor (such as a limus drug, e.g., sirolimus or a derivative thereof) and an albumin, wherein the nanoparticles comprise the mTOR inhibitor associated (e.g., coated) with albumin, wherein the nanoparticles have an average particle size of no greater than about 150 nm (such as no greater than about 120 nm); and b) an effective amount of vincristine. In some embodiments, the method comprises administering to the individual a) an effective amount of a composition comprising nanoparticles comprising an mTOR inhibitor (such as a limus drug, e.g., sirolimus or a derivative thereof) and an albumin, wherein the nanoparticles comprise the mTOR inhibitor associated (e.g., coated) with the albumin, wherein the nanoparticles have an average particle size of no greater than about 150 nm (such as no greater than about 120 nm, for example about 100 nm), wherein the weight ratio of albumin and the mTOR inhibitor in the mTOR inhibitor nanoparticle composition is about 9:1 or less (such as about 9:1 or about 8:1); and b) an effective amount of vincristine. In some embodiments, the method further comprises administering to the individual at least one therapeutic agent used in a standard combination therapy with vincristine. In some embodiments, the mTOR inhibitor is a limus drug. In some embodiments, the mTOR inhibitor is sirolimus or a derivative thereof. In some embodiments, the mTOR inhibitor nanoparticle composition comprises nab-sirolimus. In some embodiments, the mTOR inhibitor nanoparticle composition is nab-sirolimus. In some embodiments, the mTOR inhibitor nanoparticle is in the dosage range of about 10 mg/m² to about 200 mg/m² (including for example about any of 10 mg/m² to about 40 mg/m², about 40 mg/m² to about 75 mg/m², about 75 mg/m² to about 100 mg/m², about 100 mg/m² to about 200 mg/m², about 20 mg/m² to about 55 mg/m², and any ranges between these values). In some embodiments, the mTOR inhibitor nanoparticle is in the dosage range of about 20 mg/m² to about 55 mg/m² (such as about any of 20 mg/m², 35 mg/m², 45 mg/m², or 55 mg/m²). In some embodiments, the vincristine is in the dosage range of about 0.5 mg/m² to about 5 mg/m² (including for example about any of 0.5 mg/m² to about 1 mg/m², about 1 mg/m² to about 1.5 mg/m², about 1.5 mg/m² to about 2 mg/m², about 2 mg/m² to about 2.5 mg/m², about 2.5 mg/m² to about 3 mg/m², about 3 mg/m² to about 4 mg/m², about 4 mg/m² to about 5 mg/m², about 1.5 mg/m², and any ranges between these values). In some embodiments, the vincristine is in a dosage of about 1.5 mg/m². In some embodiments, the solid tumor is a recurrent solid tumor. In some embodiments, the solid tumor is refractory to one or more drugs used in a standard therapy for the solid tumor. In some embodiments, the solid tumor is tufted angioma. In some embodiments, the solid tumor is kaposiform hemangioendothelioma. In some embodiments, the individual is no more than about any of 17, 16, 15, 14, 13, 12, 11, 10, 9, 8, 7, 6, 5, 4, 3, 2, or 1 year old. In some embodiments, the individual is about 9 to about 15 years old. In some embodiments, the individual is about 5 to about 9 years old. In some embodiments, the individual is about 1 to about 5 years old. In some embodiments, the individual is no more than about 1 year old, such as about 6 months old to about 1 year old, less than about 6 months old, or less than about 3 months old.

### Pharmaceutical Compositions

The nanoparticle compositions (such as mTOR inhibitor nanoparticle compositions) and/or second therapeutic agents described herein can be used in the preparation of a formulation, such as a pharmaceutical composition, by combining the nanoparticle composition(s) or second therapeutic agent(s) described above with a pharmaceutically acceptable carrier, an excipient, a stabilizing agent, and/or another agent known in the art for use in the methods of treatment, methods of administration, and dosage regimes described herein.

To increase stability by increasing the negative zeta potential of nanoparticles in a pharmaceutical composition, certain negatively charged components can be added. Such negatively charged components include, but are not limited to, bile salts, bile acids, glycocholic acid, cholic acid, chenodeoxycholic acid, taurocholic acid, glycochenodeoxycholic acid, taurochenodeoxycholic acid, litocholic acid, ursodeoxycholic acid, dehydrocholic acid, and others; and phospholipids including lecithin (egg yolk) based phospholipids, which includes the following phosphatidylcholines: palmitoyloleoylphosphatidylcholine, palmitoyllinoleoylphosphatidylcholine, stearoyllinoleoylphosphatidylcholine, stearoyloleoylphosphatidylcholine, stearoylarachidoylphosphatidylcholine, and dipalmitoylphosphatidylcholine. Other phospholipids include L-α-dimyristoylphosphatidylcholine (DMPC), dioleoylphosphatidylcholine (DOPC), distearoylphosphatidylcholine (DSPC), hydrogenated soy phosphatidylcholine (HSPC), and other related compounds. Negatively charged surfactants or emulsifiers are also suitable as additives, e.g., sodium cholesteryl sulfate and the like.

In some embodiments, the pharmaceutical composition is suitable for administration to a human. In some embodiments, the pharmaceutical composition is suitable for administration to a mammal, such as, in the veterinary context, domestic pets and agricultural animals. There are a wide variety of suitable formulations of the inventive composition (see, e.g., U.S. Pat. Nos. 5,916,596 and 6,096,331, which are hereby incorporated by reference in their entireties). The following formulations and methods are merely exemplary and are in no way limiting. Formulations suitable for oral administration can comprise (a) liquid solutions, such as an effective amount of the active ingredient (e.g., nanoparticle composition or second therapeutic agent) dissolved in diluents, such as water, saline, or orange juice, (b) capsules, sachets or tablets, each containing a predetermined amount of the active ingredient, as solids or granules, (c) suspensions in an appropriate liquid, (d) suitable emulsions, and (e) powders. Tablet forms can include one or more of lactose, mannitol, corn starch, potato starch, microcrystalline cellulose, acacia, gelatin, colloidal silicon dioxide, croscarmellose sodium, talc, magnesium stearate, stearic acid, and other excipients, colorants, diluents, buffering agents, moistening agents, preservatives, flavoring agents, and pharmacologically compatible excipients. Lozenge forms can comprise the active ingredient in a flavor, usually sucrose and acacia or tragacanth, as well as pastilles comprising the active ingredient in an inert base, such as gelatin and glycerin, or sucrose and acacia, emulsions, gels, and the like containing, in addition to the active ingredient, such excipients as are known in the art.

Formulations suitable for parenteral administration include aqueous and non-aqueous, isotonic sterile injection solutions, which can contain anti-oxidants, buffers, bacteriostats, and solutes that render the formulation compatible with the blood of the intended recipient, and aqueous and non-aqueous sterile suspensions that can include suspending agents, solubilizers, thickening agents, stabilizing agents, and preservatives. The formulations can be presented in unit-dose or multi-dose sealed containers, such as ampules and vials, and can be stored in a freeze-dried (lyophilized) condition requiring only the addition of a sterile liquid excipient (e.g., water) for injection, immediately prior to use. Extemporaneous injection solutions and suspensions can be prepared from sterile powders, granules, and tablets of the kind previously described.

Formulations suitable for aerosol administration are provided that comprise the inventive compositions described above. In some embodiments, the formulation suitable for aerosol administration is an aqueous or non-aqueous isotonic sterile solutions, and can contain anti-oxidants, buffers, bacteriostats, and/or solutes. In some embodiments, the formulation suitable for aerosol administration is an aqueous or non-aqueous sterile suspensions that can include suspending agents, solubilizers, thickening agents, stabilizing agents, and/or preservatives, alone or in combination with other suitable components. These aerosol formulations can be placed into pressurized acceptable propellants, such as dichlorodifluoromethane, propane, nitrogen, and the like. They can also be formulated as pharmaceuticals for non-pressured preparations, such as for use in a nebulizer or an atomizer.

In some embodiments, the pharmaceutical composition is formulated to have a pH in the range of about 4.5 to about 9.0, including for example pH ranges of any of about 5.0 to about 8.0, about 6.5 to about 7.5, and about 6.5 to about 7.0. In some embodiments, the pH of the pharmaceutical composition is formulated to no less than about 6, including for example no less than about any of 6.5, 7, or 8 (e.g., about 8). The pharmaceutical composition can also be made to be isotonic with blood by the addition of a suitable tonicity modifier, such as glycerol.

The nanoparticles of this invention can be enclosed in a hard or soft capsule, can be compressed into tablets, or can be incorporated with beverages or food or otherwise incorporated into the diet. Capsules can be formulated by mixing the nanoparticles with an inert pharmaceutical diluent and inserting the mixture into a hard gelatin capsule of the appropriate size. If soft capsules are desired, a slurry of the nanoparticles with an acceptable vegetable oil, light petroleum or other inert oil can be encapsulated by machine into a gelatin capsule.

Also provided are unit dosage forms comprising the compositions and formulations described herein. These unit dosage forms can be stored in a suitable packaging in single or multiple unit dosages and may also be further sterilized and sealed. For example, the pharmaceutical composition (e.g., a dosage or unit dosage form of a pharmaceutical composition) may include (i) nanoparticles that comprise sirolimus or a derivative thereof and an albumin and (ii) a pharmaceutically acceptable carrier. In other examples, the pharmaceutical composition (e.g., a dosage or unit dosage form of a pharmaceutical composition includes a) nanoparticles comprising sirolimus or a derivative thereof and an albumin and b) at least one other therapeutic agent. In some embodiments, the other therapeutic agent comprises any of the second therapeutic agents described herein). In some embodiments, the pharmaceutical composition also includes one or more other compounds (or pharmaceutically acceptable salts thereof) that are useful for treating cancer. In some embodiments, the amount of mTOR inhibitor (such as a limus drug, e.g., sirolimus or a derivative thereof) in the composition is included in any of the following ranges: about 20 to about 50 mg, about 50 to about 100 mg, about 100 to about 125 mg, about 125 to about 150 mg, about 150 to about 175 mg, about 175 to about 200 mg, about 200 to about 225 mg, about 225 to about 250 mg, about 250 to about 300 mg, or about 300 to about 350 mg. In some embodiments, the amount of mTOR inhibitor (such as a limus drug, e.g., sirolimus or a derivative thereof) in the composition (e.g., a dosage or unit dosage form) is in the range of about 54 mg to about 540 mg, such as about 180 mg to about 270 mg or about 216 mg, of the mTOR inhibitor. In some embodiments, the carrier is suitable for parental administration (e.g., intravenous administration). In some embodiments, a taxane is not contained in the composition. In some embodiments, the mTOR inhibitor (such as a limus drug, e.g., sirolimus or a derivative thereof) is the only pharmaceutically active agent for the treatment of solid tumors that is contained in the composition.

Thus, in some embodiments, there is provided a pharmaceutical composition according to any of the pharmaceutical compositions described above comprising nanoparticles comprising an mTOR inhibitor (such as a limus drug, e.g., sirolimus or a derivative thereof) and/or a second therapeutic agent for use in any of the methods of treating a solid tumor described herein. In some embodiments, the pharmaceutical composition comprises nanoparticles comprising an mTOR inhibitor (such as a limus drug, e.g., sirolimus or a derivative thereof) and albumin (such as human albumin). In some embodiments, the pharmaceutical composition comprises a second therapeutic agent. In some embodiments, the pharmaceutical composition comprises a) nanoparticles comprising an mTOR inhibitor (such as a limus drug, e.g., sirolimus or a derivative thereof) and albumin (such as human albumin); and b) a second therapeutic agent. In some embodiments, the second therapeutic agent is an immunomodulator. In some embodiments, the second therapeutic agent is an immunostimulator. In some embodiments, the second therapeutic agent is an immunostimulator that directly stimulates the immune system of an individual. In some embodiments, the immunomodulator is an agonistic antibody that targets an activating receptor on an immune cell (such as a T cell). In some embodiments, the immunomodulator is an immune checkpoint inhibitor. In some embodiments, the immune checkpoint inhibitor is an antagonistic antibody that targets an immune checkpoint protein. In some embodiments, the immunomodulator is an IMiDs® compound (small molecule immunomodulator, such as lenalidomide or pomalidomide). In some embodiments, the second therapeutic agent is an immunomodulator selected from the group consisting of pomalidomide and lenalidomide. In some embodiments, the immunomodulator is small molecule or antibody-based IDO inhibitor. In some embodiments, the second therapeutic agent is a histone deacetylase inhibitor. In some embodiments, the histone deacetylase inhibitor is specific to only one HDAC. In some embodiments, the histone deacetylase inhibitor is specific to only one class of HDAC. In some embodiments, the histone deacetylase inhibitor is specific to two or more HDACs or two or more classes of HDACs. In some embodiments, the histone deacetylase inhibitor is specific to class I and II HDACs. In some embodiments, the histone deacetylase inhibitor is specific to class III HDACs. In some embodiments, the histone deacetylase inhibitor is selected from the group consisting of romidepsin, panobinostat, ricolinostat, and belinostat. In some embodiments, the second therapeutic agent is a kinase inhibitor, such as a tyrosine kinase inhibitor. In some embodiments, the kinase inhibitor is a serine/threonine kinase inhibitor. In some embodiments, the kinase inhibitor is a Raf kinase inhibitor. In some embodiments, the kinase inhibitor inhibits more than one class of kinase (e.g., an inhibitor of more than one of a tyrosine kinase, a Raf kinase, and a serine/threonine kinase). In some embodiments, the kinase inhibitor is selected from the group consisting of erlotinib, imatinib, lapatinib, nilotinib, sorafenib, and sunitinib. In some embodiments, the second therapeutic agent is a cancer vaccine, such as a vaccine prepared using tumor cells or at least one tumor-associated antigen.

### Diseases to be Treated

In some embodiments, according to any of the methods described above, the solid tumor is selected from the group consisting of pancreatic neuroendocrine cancer, endometrial cancer, ovarian cancer, breast cancer, renal cell carcinoma, lymphangioleiomyomatosis (LAM), prostate cancer, and bladder cancer. The methods are applicable to solid tumors of all stages, including stages, I, II, III, and IV, according to the American Joint Committee on Cancer (AJCC) staging groups. In some embodiments, the solid tumor is an/a: early stage cancer, non-metastatic cancer, primary cancer, advanced cancer, locally advanced cancer, metastatic cancer, cancer in remission, cancer in an adjuvant setting, or cancer in a neoadjuvant setting. In some embodiments, the solid tumor is localized resectable, localized unresectable, or unresectable. In some embodiments, the solid tumor is localized resectable or borderline resectable. In some embodiments, the cancer has been refractory to prior therapy. In some embodiments, the cancer is resistant to the treatment with a non-nanoparticle formulation of a chemotherapeutic agent (such as non-nanoparticle formulation of a limus drug).

In some embodiments, according to any of the methods described above, the solid tumor is breast cancer. In some embodiments, the breast cancer is early stage breast cancer, non-metastatic breast cancer, advanced breast cancer, stage IV breast cancer, locally advanced breast cancer, metastatic breast cancer, breast cancer in remission, breast cancer in an adjuvant setting, or breast cancer in a neoadjuvant setting. In some embodiments, the breast cancer is in a neoadjuvant setting. In some embodiments, the breast cancer is at an advanced stage. In some embodiments, the breast cancer (which may be HER2 positive or HER2 negative) includes, for example, advanced breast cancer, stage IV breast cancer, locally advanced breast cancer, and metastatic breast cancer. In some embodiments, the individual may be a human who has a gene, genetic mutation, or polymorphism associated with breast cancer (e.g., BRCA1, BRCA2, ATM, CHEK2, RAD51, AR, DIRAS3, ERBB2, TP53, AKT, PTEN, and/or PDK) or has one or more extra copies of a gene (e.g., one or more extra copies of the HER2 gene) associated with breast cancer. In some embodiments, the method further comprises identifying a cancer patient population (i.e. breast cancer population) based on a hormone receptor status of patients having tumor tissue not expressing both ER and PgR.

In some embodiments, according to any of the methods described above, the cancer is renal cell carcinoma. In some embodiments, the renal cell carcinoma is an adenocarcinoma. In some embodiments, the renal cell carcinoma is a clear cell renal cell carcinoma, papillary renal cell carcinoma (also called chromophilic renal cell carcinoma), chromophobe renal cell carcinoma, collecting duct renal cell carcinoma, granular renal cell carcinoma, mixed granular renal cell carcinoma, renal angiomyolipomas, or spindle renal cell carcinoma. In some embodiments, the individual may be a human who has a gene, genetic mutation, or polymorphism associated with renal cell carcinoma (e.g., VHL, TSC1, TSC2, CUL2, MSH2, MLH1, INK4a/ARF, MET, TGF-α, TGF-β1, IGF-I, IGF-IR, AKT, and/or PTEN) or has one or more extra copies of a gene associated with renal cell carcinoma. In some embodiments, the renal cell carcinoma is associated with (1) von Hippel-Lindau (VHL) syndrome, (2) hereditary papillary renal carcinoma (HPRC), (3) familial renal oncocytoma (FRO) associated with Birt-Hogg-Dube syndrome (BHDS), or (4) hereditary renal carcinoma (HRC). In some embodiments, the renal cell carcinoma is at any of stage I, II, III, or IV, according to the American Joint Committee on Cancer (AJCC) staging groups. In some embodiments, the renal cell carcinoma is stage IV renal cell carcinoma.

In some embodiments, according to any of the methods described above, the solid tumor is prostate cancer. In some embodiments, the prostate cancer is an adenocarcinoma. In some embodiments, the prostate cancer is a sarcoma, neuroendocrine tumor, small cell cancer, ductal cancer, or a lymphoma. In some embodiments, the prostate cancer is at any of the four stages, A, B, C, or D, according to the Jewett staging system. In some embodiments, the prostate cancer is stage A prostate cancer (e.g., the cancer cannot be felt during a rectal exam). In some embodiments, the prostate cancer is stage B prostate cancer (e.g., the tumor involves more tissue within the prostate, and can be felt during a rectal exam, or is found with a biopsy that is done because of a high PSA level). In some embodiments, the prostate cancer is stage C prostate cancer (e.g., the cancer has spread outside the prostate to nearby tissues). In some embodiments, the prostate cancer is stage D prostate cancer. In some embodiments, the prostate cancer is androgen independent prostate cancer (AIPC). In some embodiments, the prostate cancer is androgen dependent prostate cancer. In some embodiments, the prostate cancer is refractory to hormone therapy. In some embodiments, the prostate cancer is substantially refractory to hormone therapy. In some embodiments, the individual is a human who has a gene, genetic mutation, or polymorphism associated with prostate cancer (e.g., RNASEL/HPC1, ELAC2/HPC2, SR-A/MSR1, CHEK2, BRCA2, PON1, OGG1, MIC-I, TLR4, and/or PTEN) or has one or more extra copies of a gene associated with prostate cancer.

In some embodiments, according to any of the methods described above, the solid tumor is lung cancer. In some embodiments, the lung cancer is a non-small cell lung cancer (NSCLC). Examples of NSCLC include, but are not limited to, large-cell carcinoma (e.g., large-cell neuroendocrine carcinoma, combined large-cell neuroendocrine carcinoma, basaloid carcinoma, lymphoepithelioma-like carcinoma, clear cell carcinoma, and large-cell carcinoma with rhabdoid phenotype), adenocarcinoma (e.g., acinar, papillary (e.g., bronchioloalveolar carcinoma, nonmucinous, mucinous, mixed mucinous and nonmucinous and indeterminate cell type), solid adenocarcinoma with mucin, adenocarcinoma with mixed subtypes, well-differentiated fetal adenocarcinoma, mucinous (colloid) adenocarcinoma, mucinous cystadenocarcinoma, signet ring adenocarcinoma, and clear cell adenocarcinoma), neuroendocrine lung tumors, and squamous cell carcinoma (e.g., papillary, clear cell, small cell, and basaloid). In some embodiments, the NSCLC is, according to TNM classifications, a stage T tumor (primary tumor), a stage N tumor (regional lymph nodes), or a stage M tumor (distant metastasis). In some embodiments, the lung cancer is a carcinoid (typical or atypical), adenosquamous carcinoma, cylindroma, or carcinoma of the salivary gland (e.g., adenoid cystic carcinoma or mucoepidermoid carcinoma). In some embodiments, the lung cancer is a carcinoma with pleomorphic, sarcomatoid, or sarcomatous elements (e.g., carcinomas with spindle and/or giant cells, spindle cell carcinoma, giant cell carcinoma, carcinosarcoma, or pulmonary blastoma). In some embodiments, the cancer is small cell lung cancer (SCLC; also called oat cell carcinoma). The small cell lung cancer may be limited-stage, extensive stage or recurrent small cell lung cancer. In some embodiments, the individual may be a human who has a gene, genetic mutation, or polymorphism suspected or shown to be associated with lung cancer (e.g., SASH1, LATS1, IGF2R, PARK2, KRAS, PTEN, Kras2, Krag, Pas1, ERCC1, XPD, IL8RA, EGFR, Oti-AD, EPHX, MMP1, MMP2, MMP3, MMP12, IL1 β, RAS, and/or AKT) or has one or more extra copies of a gene associated with lung cancer.

Thus, in some embodiments, there is provided a method of treating lung cancer in an individual (such as a human) comprising administering to the individual a) an effective amount of a composition comprising nanoparticles comprising an mTOR inhibitor (such as a limus drug, e.g., sirolimus or a derivative thereof) and an albumin; and b) an effective amount of a second therapeutic agent. In some embodiments, the mTOR inhibitor is a limus drug. In some embodiments, the mTOR inhibitor is sirolimus or a derivative thereof. In some embodiments, the mTOR inhibitor nanoparticle composition comprises nab-sirolimus. In some embodiments, the mTOR inhibitor nanoparticle composition is nab-sirolimus. In some embodiments, the second therapeutic agent is selected from the group consisting of an immunomodulator (such as an immunostimulator or an immune checkpoint inhibitor), a histone deacetylase inhibitor, a kinase inhibitor (such as a tyrosine kinase inhibitor), and a cancer vaccine (such as a vaccine prepared using tumor cells or at least one tumor-associated antigen). In some embodiments, the second therapeutic agent is an immunomodulator. In some embodiments, the immunomodulator is an immunostimulator that directly stimulates the immune system of an individual. In some embodiments, the immunomodulator is an agonistic antibody that targets an activating receptor on an immune cell (such as a T cell). In some embodiments, the immunomodulator is an immune checkpoint inhibitor. In some embodiments, the immune checkpoint inhibitor is an antagonistic antibody that targets an immune checkpoint protein. In some embodiments, the immunomodulator is an IMiDs® (small molecule immunomodulator, such as lenalidomide or pomalidomide). In some embodiments, the immunomodulator is lenalidomide. In some embodiments, the immunomodulator is pomalidomide. In some embodiments, the immunomodulator is small molecule or antibody-based IDO inhibitor. In some embodiments, the second therapeutic agent is a histone deacetylase inhibitor. In some embodiments, the histone deacetylase inhibitor is selected from the group consisting of romidepsin, panobinostat, ricolinostat, and belinostat. In some embodiments, the histone deacetylase inhibitor is romidepsin. In some embodiments, the second therapeutic agent is a kinase inhibitor, such as a tyrosine kinase inhibitor. In some embodiments, the kinase inhibitor is a serine/threonine kinase inhibitor. In some embodiments, the kinase inhibitor is selected from the group consisting of erlotinib, imatinib, lapatinib, nilotinib, sorafenib, and sunitinib. In some embodiments, the second therapeutic agent is a cancer vaccine, such as a vaccine prepared using tumor cells or at least one tumor-associated antigen. In some embodiments, the lung cancer is recurrent lung cancer. In some embodiments, the lung cancer is refractory to at least one drug used in a standard therapy for lung cancer.

In some embodiments, according to any of the methods described above, the solid tumor is brain cancer. In some embodiments, the brain cancer is glioma, brain stem glioma, cerebellar or cerebral astrocytoma (e.g., pilocytic astrocytoma, diffuse astrocytoma, or anaplastic (malignant) astrocytoma), malignant glioma, ependymoma, oligodenglioma, meningioma, craniopharyngioma, haemangioblastomas, medulloblastoma, supratentorial primitive neuroectodermal tumors, visual pathway and hypothalamic glioma, or glioblastoma. In some embodiments, the brain cancer is glioblastoma (also called glioblastoma multiforme or grade 4 astrocytoma). In some embodiments, the glioblastoma is radiation-resistant. In some embodiments, the glioblastoma is radiation-sensitive. In some embodiments, the glioblastoma may be infratentorial. In some embodiments, the glioblastoma is supratentorial. In some embodiments, the individual may be a human who has a gene, genetic mutation, or polymorphism associated with brain cancer (e.g., glioblastoma) (e.g., NRP/B, MAGE-E1, MMACI-E1, PTEN, LOH, p53, MDM2, DCC, TP-73, RbI, EGFR, PDGFR-α, PMS2, MLH1, and/or DMBT1) or has one or more extra copies of a gene associated with brain cancer (e.g., glioblastoma) (e.g., MDM2, EGFR, and PDGR-α).

Thus, in some embodiments, there is provided a method of treating brain cancer in an individual (such as a human) comprising administering to the individual a) an effective amount of a composition comprising nanoparticles comprising an mTOR inhibitor (such as a limus drug, e.g., sirolimus or a derivative thereof) and an albumin; and b) an effective amount of a second therapeutic agent. In some embodiments, the mTOR inhibitor is a limus drug. In some embodiments, the mTOR inhibitor is sirolimus or a derivative thereof. In some embodiments, the mTOR inhibitor nanoparticle composition comprises nab-sirolimus. In some embodiments, the mTOR inhibitor nanoparticle composition is nab-sirolimus. In some embodiments, the second therapeutic agent is selected from the group consisting of an immunomodulator (such as an immunostimulator or an immune checkpoint inhibitor), a histone deacetylase inhibitor, a kinase inhibitor (such as a tyrosine kinase inhibitor), and a cancer vaccine (such as a vaccine prepared using tumor cells or at least one tumor-associated antigen). In some embodiments, the second therapeutic agent is an immunomodulator. In some embodiments, the immunomodulator is an immunostimulator that directly stimulates the immune system of an individual. In some embodiments, the immunomodulator is an agonistic antibody that targets an activating receptor on an immune cell (such as a T cell). In some embodiments, the immunomodulator is an immune checkpoint inhibitor. In some embodiments, the immune checkpoint inhibitor is an antagonistic antibody that targets an immune checkpoint protein. In some embodiments, the immunomodulator is an IMiDs® (small molecule immunomodulator, such as lenalidomide or pomalidomide). In some embodiments, the immunomodulator is lenalidomide. In some embodiments, the immunomodulator is pomalidomide. In some embodiments, the immunomodulator is small molecule or antibody-based IDO inhibitor. In some embodiments, the second therapeutic agent is a histone deacetylase inhibitor. In some embodiments, the histone deacetylase inhibitor is selected from the group consisting of romidepsin, panobinostat, ricolinostat, and belinostat. In some embodiments, the histone deacetylase inhibitor is romidepsin. In some embodiments, the second therapeutic agent is a kinase inhibitor, such as a tyrosine kinase inhibitor. In some embodiments, the kinase inhibitor is a serine/threonine kinase inhibitor. In some embodiments, the kinase inhibitor is selected from the group consisting of erlotinib, imatinib, lapatinib, nilotinib, sorafenib, and sunitinib. In some embodiments, the second therapeutic agent is a cancer vaccine, such as a vaccine prepared using tumor cells or at least one tumor-associated antigen. In some embodiments, the brain cancer is recurrent brain cancer. In some embodiments, the brain cancer is refractory to at least one drug used in a standard therapy for brain cancer.

In some embodiments, according to any of the methods described above, the solid tumor is melanoma. In some embodiments, the melanoma is superficial spreading melanoma, lentigo maligna melanoma, nodular melanoma, mucosal melanoma, polypoid melanoma, desmoplastic melanoma, amelanotic melanoma, soft-tissue melanoma, or acral lentiginous melanoma. In some embodiments, the melanoma is at any of stage I, II, III, or IV, according to the American Joint Committee on Cancer (AJCC) staging groups. In some embodiments, the melanoma is recurrent.

In some embodiments, according to any of the methods described above, the solid tumor is ovarian cancer. In some embodiments, the ovarian cancer is ovarian epithelial cancer. Exemplary ovarian epithelial cancer histological classifications include: serous cystomas (e.g., serous benign cystadenomas, serous cystadenomas with proliferating activity of the epithelial cells and nuclear abnormalities but with no infiltrative destructive growth, or serous cystadenocarcinomas), mucinous cystomas (e.g., mucinous benign cystadenomas, mucinous cystadenomas with proliferating activity of the epithelial cells and nuclear abnormalities but with no infiltrative destructive growth, or mucinous cystadenocarcinomas), endometrioid tumors (e.g., endometrioid benign cysts, endometrioid tumors with proliferating activity of the epithelial cells and nuclear abnormalities but with no infiltrative destructive growth, or endometrioid adenocarcinomas), clear cell (mesonephroid) tumors (e.g., begin clear cell tumors, clear cell tumors with proliferating activity of the epithelial cells and nuclear abnormalities but with no infiltrative destructive growth, or clear cell cystadenocarcinomas), unclassified tumors that cannot be allotted to one of the above groups, or other malignant tumors. In some embodiments, the ovarian epithelial cancer is stage I (e.g., stage IA, IB, or IC), stage II (e.g., stage HA, HB, or IIC), stage III (e.g., stage IIIA, HIB, or HIC), or stage IV. In some embodiments, the individual is a human who has a gene, genetic mutation, or polymorphism associated with ovarian cancer (e.g., MLH1, MLH3, MSH2, MSH6, TGFBR2, PMS1, PMS2, BRCA1 and/or BRCA2) or has one or more extra copies of a gene associated with ovarian cancer (e.g., one or more extra copies of the HER2 gene).

In some embodiments, the ovarian cancer is an ovarian germ cell tumor. Exemplary histologic subtypes include dysgerminomas or other germ cell tumors (e.g., endodermal sinus tumors such as hepatoid or intestinal tumors, embryonal carcinomas, olyembryomas, choriocarcinomas, teratomas, or mixed form tumors). Exemplary teratomas are immature teratomas, mature teratomas, solid teratomas, and cystic teratomas (e.g., dermoid cysts such as mature cystic teratomas, and dermoid cysts with malignant transformation). Some teratomas are monodermal and highly specialized, such as struma ovarii, carcinoid, struma ovarii and carcinoid, or others (e.g., malignant neuroectodermal and ependymomas). In some embodiments, the ovarian germ cell tumor is stage I (e.g., stage IA, IB, or IC), stage II (e.g., stage HA, HB, or IIC), stage III (e.g., stage IIIA, HIB, or IIIC), or stage IV.

In some embodiments, according to any of the methods described above, the solid tumor is pancreatic neuroendocrine cancer. In some embodiments, the pancreatic neuroendocrine cancer is a well-differentiated neuroendocrine tumor, a well-differentiated (low grade) neuroendocrine carcinoma, or a poorly differentiated (high grade) neuroendocrine carcinoma. In some embodiments, the pancreatic neuroendocrine cancer is a functional pancreatic neuroendocrine tumor. In some embodiments, the pancreatic neuroendocrine tumor is a nonfunctional pancreatic neuroendocrine tumor. In some embodiments, the pancreatic neuroendocrine cancer is insulinoma, glucagonoma, somatostatinoma, gastrinoma, VIPoma, GRFoma, or ACTHoma. In some embodiments, the individual may be a human who has a gene, genetic mutation, or polymorphism associated with pancreatic neuroendocrine cancer (e.g., NF1 and/or MEN1) or has one or more extra copies of a gene associated with pancreatic neuroendocrine cancer.

In some embodiments, according to any of the methods described above, the solid tumor is endometrial cancer. In some embodiments, the endometrial cancer is adenocarcinoma, carcinosarcoma, squamous cell carcinoma, undifferentiated carcinoma, small cell carcinoma, or transitional carcinoma. In some embodiments, the endometrial cancer is endometroid cancer, adenocarcinoma with squamous differentiation, adenoacanthoma, adenosquamous carcinoma, secretory carcinoma, ciliated carcinoma, or villoglandular adenocarcinoma. In some embodiments, the endometrial cancer is clear-cell carcinoma, mucinous adenocarcinoma, or papillary serous adenocarcinoma. In some embodiments, the endometrial cancer is grade 1, grade 2, or grade 3. In some embodiments, the endometrial cancer is type 1 endometrial cancer. In some embodiments, the endometrial cancer is type 2 endometrial cancer. In some embodiments, the endometrial cancer is uterine carcinosarcoma. In some embodiments, the individual may be a human who has a gene, genetic mutation, or polymorphism associated with endometrial cancer (e.g., MLH1, MLH2, MSH2, MLH3, MSH6, TGBR2, PMS1, and/or PMS2) or has one or more extra copies of a gene associated with endometrial cancer.

In some embodiments, according to any of the methods described above, the solid tumor is lymphangioleiomyomatosis. In some embodiments, the individual may be a human who has a gene, genetic mutation, or polymorphism associated with lymphangioleiomyomatosis (e.g., TSC1 and/or TSC2) or has one or more extra copies of a gene associated with lymphangioleiomyomatosis.

In some embodiments, according to any of the methods described above, the solid tumor is colon cancer. In some embodiments, the individual may be a human who has a gene, genetic mutation, or polymorphism associated with colon cancer (e.g., RAS, AKT, PTEN, POK, and/or EGFR) or has one or more extra copies of a gene associated with colon cancer.

In some embodiments, according to any of the methods described above, the solid tumor is subependymal giant cell astrocytoma (SEGA) with tuberous sclerosis (TS). In some embodiments, the individual may be a human who has a gene, genetic mutation, or polymorphism associated with SEGA (e.g., TSC1 and/or TSC2) or has one or more extra copies of a gene associated with SEGA.

Thus, in some embodiments, there is provided a method of treating SEGA (such as SEGA with TS) in an individual (such as a human) comprising administering to the individual a) an effective amount of a composition comprising nanoparticles comprising an mTOR inhibitor (such as a limus drug, e.g., sirolimus or a derivative thereof) and an albumin; and b) an effective amount of a second therapeutic agent. In some embodiments, the mTOR inhibitor is a limus drug. In some embodiments, the mTOR inhibitor is sirolimus or a derivative thereof. In some embodiments, the mTOR inhibitor nanoparticle composition comprises nab-sirolimus. In some embodiments, the mTOR inhibitor nanoparticle composition is nab-sirolimus. In some embodiments, the second therapeutic agent is selected from the group consisting of an immunomodulator (such as an immunostimulator or an immune checkpoint inhibitor), a histone deacetylase inhibitor, a kinase inhibitor (such as a tyrosine kinase inhibitor), and a cancer vaccine (such as a vaccine prepared using tumor cells or at least one tumor-associated antigen). In some embodiments, the second therapeutic agent is an immunomodulator. In some embodiments, the immunomodulator is an immunostimulator that directly stimulates the immune system of an individual. In some embodiments, the immunomodulator is an agonistic antibody that targets an activating receptor on an immune cell (such as a T cell). In some embodiments, the immunomodulator is an immune checkpoint inhibitor. In some embodiments, the immune checkpoint inhibitor is an antagonistic antibody that targets an immune checkpoint protein. In some embodiments, the immunomodulator is an IMiDs® (small molecule immunomodulator, such as lenalidomide or pomalidomide). In some embodiments, the immunomodulator is lenalidomide. In some embodiments, the immunomodulator is pomalidomide. In some embodiments, the immunomodulator is small molecule or antibody-based IDO inhibitor. In some embodiments, the second therapeutic agent is a histone deacetylase inhibitor. In some embodiments, the histone deacetylase inhibitor is selected from the group consisting of romidepsin, panobinostat, ricolinostat, and belinostat. In some embodiments, the histone deacetylase inhibitor is romidepsin. In some embodiments, the second therapeutic agent is a kinase inhibitor, such as a tyrosine kinase inhibitor. In some embodiments, the kinase inhibitor is a serine/threonine kinase inhibitor. In some embodiments, the kinase inhibitor is selected from the group consisting of erlotinib, imatinib, lapatinib, nilotinib, sorafenib, and sunitinib. In some embodiments, the second therapeutic agent is a cancer vaccine, such as a vaccine prepared using tumor cells or at least one tumor-associated antigen. In some embodiments, the SEGA is recurrent SEGA. In some embodiments, the SEGA is refractory to at least one drug used in a standard therapy for SEGA (e.g., everolimus and/or sirolimus).

In some embodiments, according to any of the methods described above, the solid tumor is angiomyolipoma with tuberous sclerosis (TS). In some embodiments, the angiomyolipoma is PEComa. In some embodiments, the individual may be a human who has a gene, genetic mutation, or polymorphism associated with angiomyolipoma (e.g., TSC1 and/or TSC2) or has one or more extra copies of a gene associated with angiomyolipoma.

Thus, in some embodiments, there is provided a method of treating angiomyolipoma (such as angiomyolipoma with TS) in an individual (such as a human) comprising administering to the individual a) an effective amount of a composition comprising nanoparticles comprising an mTOR inhibitor (such as a limus drug, e.g., sirolimus or a derivative thereof) and an albumin; and b) an effective amount of a second therapeutic agent. In some embodiments, the mTOR inhibitor is a limus drug. In some embodiments, the mTOR inhibitor is sirolimus or a derivative thereof. In some embodiments, the mTOR inhibitor nanoparticle composition comprises nab-sirolimus. In some embodiments, the mTOR inhibitor nanoparticle composition is nab-sirolimus. In some embodiments, the second therapeutic agent is selected from the group consisting of an immunomodulator (such as an immunostimulator or an immune checkpoint inhibitor), a histone deacetylase inhibitor, a kinase inhibitor (such as a tyrosine kinase inhibitor), and a cancer vaccine (such as a vaccine prepared using tumor cells or at least one tumor-associated antigen). In some embodiments, the second therapeutic agent is an immunomodulator. In some embodiments, the immunomodulator is an immunostimulator that directly stimulates the immune system of an individual. In some embodiments, the immunomodulator is an agonistic antibody that targets an activating receptor on an immune cell (such as a T cell). In some embodiments, the immunomodulator is an immune checkpoint inhibitor. In some embodiments, the immune checkpoint inhibitor is an antagonistic antibody that targets an immune checkpoint protein. In some embodiments, the immunomodulator is an IMiDs® (small molecule immunomodulator, such as lenalidomide or pomalidomide). In some embodiments, the immunomodulator is lenalidomide. In some embodiments, the immunomodulator is pomalidomide. In some embodiments, the immunomodulator is small molecule or antibody-based IDO inhibitor. In some embodiments, the second therapeutic agent is a histone deacetylase inhibitor. In some embodiments, the histone deacetylase inhibitor is selected from the group consisting of romidepsin, panobinostat, ricolinostat, and belinostat. In some embodiments, the histone deacetylase inhibitor is romidepsin. In some embodiments, the second therapeutic agent is a kinase inhibitor, such as a tyrosine kinase inhibitor. In some embodiments, the kinase inhibitor is a serine/threonine kinase inhibitor. In some embodiments, the kinase inhibitor is selected from the group consisting of erlotinib, imatinib, lapatinib, nilotinib, sorafenib, and sunitinib. In some embodiments, the second therapeutic agent is a cancer vaccine, such as a vaccine prepared using tumor cells or at least one tumor-associated antigen. In some embodiments, the angiomyolipoma is recurrent angiomyolipoma. In some embodiments, the angiomyolipoma is refractory to at least one drug used in a standard therapy for angiomyolipoma (e.g., everolimus and/or sirolimus).

In some embodiments, according to any of the methods described above, the solid tumor is carcinoid. In some embodiments, the carcinoid is a gastrointestinal carcinoid, a lung carcinoid, or a rectal carcinoid. In some embodiments, the carcinoid is a functional carcinoid. In some embodiments, the carcinoid is a nonfunctional carcinoid. In some embodiments, the individual may be a human who has a gene, genetic mutation, or polymorphism associated with carcinoid (e.g., MEN1) or has one or more extra copies of a gene associated with carcinoid.

Thus, in some embodiments, there is provided a method of treating carcinoid in an individual (such as a human) comprising administering to the individual a) an effective amount of a composition comprising nanoparticles comprising an mTOR inhibitor (such as a limus drug, e.g., sirolimus or a derivative thereof) and an albumin; and b) an effective amount of a second therapeutic agent. In some embodiments, the mTOR inhibitor is a limus drug. In some embodiments, the mTOR inhibitor is sirolimus or a derivative thereof. In some embodiments, the mTOR inhibitor nanoparticle composition comprises nab-sirolimus. In some embodiments, the mTOR inhibitor nanoparticle composition is nab-sirolimus. In some embodiments, the second therapeutic agent is selected from the group consisting of an immunomodulator (such as an immunostimulator or an immune checkpoint inhibitor), a histone deacetylase inhibitor, a kinase inhibitor (such as a tyrosine kinase inhibitor), and a cancer vaccine (such as a vaccine prepared using tumor cells or at least one tumor-associated antigen). In some embodiments, the second therapeutic agent is an immunomodulator. In some embodiments, the immunomodulator is an immunostimulator that directly stimulates the immune system of an individual. In some embodiments, the immunomodulator is an agonistic antibody that targets an activating receptor on an immune cell (such as a T cell). In some embodiments, the immunomodulator is an immune checkpoint inhibitor. In some embodiments, the immune checkpoint inhibitor is an antagonistic antibody that targets an immune checkpoint protein. In some embodiments, the immunomodulator is an IMiDs® (small molecule immunomodulator, such as lenalidomide or pomalidomide). In some embodiments, the immunomodulator is lenalidomide. In some embodiments, the immunomodulator is pomalidomide. In some embodiments, the immunomodulator is small molecule or antibody-based IDO inhibitor. In some embodiments, the second therapeutic agent is a histone deacetylase inhibitor. In some embodiments, the histone deacetylase inhibitor is selected from the group consisting of romidepsin, panobinostat, ricolinostat, and belinostat. In some embodiments, the histone deacetylase inhibitor is romidepsin. In some embodiments, the second therapeutic agent is a kinase inhibitor, such as a tyrosine kinase inhibitor. In some embodiments, the kinase inhibitor is a serine/threonine kinase inhibitor. In some embodiments, the kinase inhibitor is selected from the group consisting of erlotinib, imatinib, lapatinib, nilotinib, sorafenib, and sunitinib. In some embodiments, the second therapeutic agent is a cancer vaccine, such as a vaccine prepared using tumor cells or at least one tumor-associated antigen. In some embodiments, the carcinoid is recurrent carcinoid. In some embodiments, the carcinoid is refractory to at least one drug used in a standard therapy for carcinoid (e.g., somatostatin analogs, interferon, and/or everolimus).

In some embodiments, according to any of the methods described above, the solid tumor is hepatocellular carcinoma (HCC). In some embodiments, the HCC is early stage HCC, non-metastatic HCC, primary HCC, advanced HCC, locally advanced HCC, metastatic HCC, HCC in remission, or recurrent HCC. In some embodiments, the HCC is localized resectable (i.e., tumors that are confined to a portion of the liver that allows for complete surgical removal), localized unresectable (i.e., the localized tumors may be unresectable because crucial blood vessel structures are involved or because the liver is impaired), or unresectable (i.e., the tumors involve all lobes of the liver and/or has spread to involve other organs (e.g., lung, lymph nodes, bone). In some embodiments, the HCC is, according to TNM classifications, a stage I tumor (single tumor without vascular invasion), a stage II tumor (single tumor with vascular invasion, or multiple tumors, none greater than 5 cm), a stage III tumor (multiple tumors, any greater than 5 cm, or tumors involving major branch of portal or hepatic veins), a stage IV tumor (tumors with direct invasion of adjacent organs other than the gallbladder, or perforation of visceral peritoneum), N1 tumor (regional lymph node metastasis), or M1 tumor (distant metastasis). In some embodiments, the HCC is, according to AJCC (American Joint Commission on Cancer) staging criteria, stage T1, T2, T3, or T4 HCC. In some embodiments, the HCC is any one of liver cell carcinomas, fibrolamellar variants of HCC, and mixed hepatocellular cholangiocarcinomas. In some embodiments, the individual may be a human who has a gene, genetic mutation, or polymorphism associated with hepatocellular carcinoma (e.g., CCND2, RAD23B, GRP78, CEP164, MDM2, and/or ALDH2) or has one or more extra copies of a gene associated with hepatocellular carcinoma.

Thus, in some embodiments, there is provided a method of treating hepatocellular carcinoma in an individual (such as a human) comprising administering to the individual a) an effective amount of a composition comprising nanoparticles comprising an mTOR inhibitor (such as a limus drug, e.g., sirolimus or a derivative thereof) and an albumin; and b) an effective amount of a second therapeutic agent. In some embodiments, the mTOR inhibitor is a limus drug. In some embodiments, the mTOR inhibitor is sirolimus or a derivative thereof. In some embodiments, the mTOR inhibitor nanoparticle composition comprises nab-sirolimus. In some embodiments, the mTOR inhibitor nanoparticle composition is nab-sirolimus. In some embodiments, the second therapeutic agent is selected from the group consisting of an immunomodulator (such as an immunostimulator or an immune checkpoint inhibitor), a histone deacetylase inhibitor, a kinase inhibitor (such as a tyrosine kinase inhibitor), and a cancer vaccine (such as a vaccine prepared using tumor cells or at least one tumor-associated antigen). In some embodiments, the second therapeutic agent is an immunomodulator. In some embodiments, the immunomodulator is an immunostimulator that directly stimulates the immune system of an individual. In some embodiments, the immunomodulator is an agonistic antibody that targets an activating receptor on an immune cell (such as a T cell). In some embodiments, the immunomodulator is an immune checkpoint inhibitor. In some embodiments, the immune checkpoint inhibitor is an antagonistic antibody that targets an immune checkpoint protein. In some embodiments, the immunomodulator is an IMiDs® (small molecule immunomodulator, such as lenalidomide or pomalidomide). In some embodiments, the immunomodulator is lenalidomide. In some embodiments, the immunomodulator is pomalidomide. In some embodiments, the immunomodulator is small molecule or antibody-based IDO inhibitor. In some embodiments, the second therapeutic agent is a histone deacetylase inhibitor. In some embodiments, the histone deacetylase inhibitor is selected from the group consisting of romidepsin, panobinostat, ricolinostat, and belinostat. In some embodiments, the histone deacetylase inhibitor is romidepsin. In some embodiments, the second therapeutic agent is a kinase inhibitor, such as a tyrosine kinase inhibitor. In some embodiments, the kinase inhibitor is a serine/threonine kinase inhibitor. In some embodiments, the kinase inhibitor is selected from the group consisting of erlotinib, imatinib, lapatinib, nilotinib, sorafenib, and sunitinib. In some embodiments, the second therapeutic agent is a cancer vaccine, such as a vaccine prepared using tumor cells or at least one tumor-associated antigen. In some embodiments, the hepatocellular carcinoma is recurrent hepatocellular carcinoma. In some embodiments, the hepatocellular carcinoma is refractory to at least one drug used in a standard therapy for hepatocellular carcinoma (e.g., sorafenib, floxuridine, cisplatin, mitomycin C, doxorubicin, and/or everolimus).

In some embodiments, according to any of the methods described above, the solid tumor is rhabdomyosarcoma (RMS). In some embodiments, the rhabdomyosarcoma is botryoid rhabdomyosarcoma, spindle cell rhabdomyosarcoma, embryonal rhabdomyosarcoma, alveolar rhabdomyosarcoma, or undifferentiated sarcoma. In some embodiments, the rhabdomyosarcoma is pleomorphic rhabdomyosarcoma or sclerosing rhabdomyosarcoma. In some embodiments, the individual may be a human who has a gene, genetic mutation, or polymorphism associated with rhabdomyosarcoma (e.g., PAX3, PAX7, FOXO1, and/or IGF2) or has one or more extra copies of a gene associated with rhabdomyosarcoma.

Thus, in some embodiments, there is provided a method of treating rhabdomyosarcoma in an individual (such as a human) comprising administering to the individual a) an effective amount of a composition comprising nanoparticles comprising an mTOR inhibitor (such as a limus drug, e.g., sirolimus or a derivative thereof) and an albumin; and b) an effective amount of a second therapeutic agent. In some embodiments, the mTOR inhibitor is a limus drug. In some embodiments, the mTOR inhibitor is sirolimus or a derivative thereof. In some embodiments, the mTOR inhibitor nanoparticle composition comprises nab-sirolimus. In some embodiments, the mTOR inhibitor nanoparticle composition is nab-sirolimus. In some embodiments, the second therapeutic agent is selected from the group consisting of an immunomodulator (such as an immunostimulator or an immune checkpoint inhibitor), a histone deacetylase inhibitor, a kinase inhibitor (such as a tyrosine kinase inhibitor), and a cancer vaccine (such as a vaccine prepared using tumor cells or at least one tumor-associated antigen). In some embodiments, the second therapeutic agent is an immunomodulator. In some embodiments, the immunomodulator is an immunostimulator that directly stimulates the immune system of an individual. In some embodiments, the immunomodulator is an agonistic antibody that targets an activating receptor on an immune cell (such as a T cell). In some embodiments, the immunomodulator is an immune checkpoint inhibitor. In some embodiments, the immune checkpoint inhibitor is an antagonistic antibody that targets an immune checkpoint protein. In some embodiments, the immunomodulator is an IMiDs® (small molecule immunomodulator, such as lenalidomide or pomalidomide). In some embodiments, the immunomodulator is lenalidomide. In some embodiments, the immunomodulator is pomalidomide. In some embodiments, the immunomodulator is small molecule or antibody-based IDO inhibitor. In some embodiments, the second therapeutic agent is a histone deacetylase inhibitor. In some embodiments, the histone deacetylase inhibitor is selected from the group consisting of romidepsin, panobinostat, ricolinostat, and belinostat. In some embodiments, the histone deacetylase inhibitor is romidepsin. In some embodiments, the second therapeutic agent is a kinase inhibitor, such as a tyrosine kinase inhibitor. In some embodiments, the kinase inhibitor is a serine/threonine kinase inhibitor. In some embodiments, the kinase inhibitor is selected from the group consisting of erlotinib, imatinib, lapatinib, nilotinib, sorafenib, and sunitinib. In some embodiments, the second therapeutic agent is a cancer vaccine, such as a vaccine prepared using tumor cells or at least one tumor-associated antigen. In some embodiments, the second therapeutic agent is a vinca alkaloid (such as vinblastine, vincristine, vindesine, or vinorelbine) or an alkylating agent (such as cyclophosphamide, melphalan, chlorambucil, ifosfamide, streptozocin, or busulfan). In some embodiments, the rhabdomyosarcoma is recurrent rhabdomyosarcoma. In some embodiments, the rhabdomyosarcoma is refractory to at least one drug used in a standard therapy for rhabdomyosarcoma (e.g., vincristine, dactinomycin, cyclophosphamide, irinotecan, topotecan, ifosfamide, etoposide, and/or doxorubicin).

In some embodiments, there is provided a method of treating rhabdomyosarcoma in an individual (such as a human) comprising administering to the individual a) an effective amount of a composition comprising nanoparticles comprising an mTOR inhibitor (such as a limus drug, e.g., sirolimus or a derivative thereof) and an albumin; and b) an effective amount of vinorelbine and cyclophosphamide. In some embodiments, the mTOR inhibitor is a limus drug. In some embodiments, the mTOR inhibitor is sirolimus or a derivative thereof. In some embodiments, the mTOR inhibitor nanoparticle composition comprises nab-sirolimus. In some embodiments, the mTOR inhibitor nanoparticle composition is nab-sirolimus. In some embodiments, the mTOR inhibitor nanoparticle is in the dosage range of about 10 mg/m² to about 200 mg/m² (including for example about any of 10 mg/m² to about 40 mg/m², about 40 mg/m² to about 75 mg/m², about 75 mg/m² to about 100 mg/m², about 100 mg/m² to about 200 mg/m², about 15 mg/m² to about 45 mg/m², and any ranges between these values). In some embodiments, the mTOR inhibitor nanoparticle is in the dosage range of about 15 mg/m² to about 45 mg/m² (such as about any of 15 mg/m², 25 mg/m², 35 mg/m², or 45 mg/m²). In some embodiments, the vinorelbine is in the dosage range of about 10 mg/m² to about 80 mg/m² (including for example about any of 10 mg/m² to about 20 mg/m², about 20 mg/m² to about 40 mg/m², about 40 mg/m² to about 60 mg/m², about 60 mg/m² to about 80 mg/m², about 25 mg/m², and any ranges between these values). In some embodiments, the vinorelbine is in a dosage of about 25 mg/m². In some embodiments, the cyclophosphamide is in the dosage range of about 0.5 g/m² to about 5 g/m² (including for example about any of 0.5 g/m² to about 1 g/m², about 1 g/m² to about 1.2 g/m², about 1.2 g/m² to about 1.4 g/m², about 1.4 g/m² to about 1.6 g/m², about 1.6 g/m² to about 1.8 g/m², about 1.8 g/m² to about 2.0 g/m², about 2.0 g/m² to about 2.2 g/m², about 2.2 g/m² to about 3 g/m², about 3 g/m² to about 4 g/m², about 4 g/m² to about 5 g/m², about 1.2 g/m², and any ranges between these values). In some embodiments, the cyclophosphamide is in a dosage of about 1.2 g/m². In some embodiments, the rhabdomyosarcoma is recurrent rhabdomyosarcoma. In some embodiments, the rhabdomyosarcoma is refractory to at least one drug used in a standard therapy for rhabdomyosarcoma (e.g., vincristine, dactinomycin, cyclophosphamide, irinotecan, topotecan, ifosfamide, etoposide, and/or doxorubicin).

In some embodiments, according to any of the methods described above, the solid tumor is neuroblastoma. In some embodiments, the neuroblastoma is neuroblastoma of the adrenal glands, neck, chest abdomen, or pelvis. In some embodiments, the neuroblastoma is a stage 1, stage 2A, stage 2B, stage 3, stage 4, or stage 4S neuroblastoma. In some embodiments, the neuroblastoma is a stage L1, stage L2, stage M, or stage M2 neuroblastoma. In some embodiments, the individual may be a human who has a gene, genetic mutation, or polymorphism associated with neuroblastoma (e.g., ALK, PHOX2B, MYCN, NTK1, KIF1B, LMO1, NBPF10, and/or ATRX) or has one or more extra copies of a gene associated with neuroblastoma.

Thus, in some embodiments, there is provided a method of treating neuroblastoma in an individual (such as a human) comprising administering to the individual a) an effective amount of a composition comprising nanoparticles comprising an mTOR inhibitor (such as a limus drug, e.g., sirolimus or a derivative thereof) and an albumin; and b) an effective amount of a second therapeutic agent. In some embodiments, the mTOR inhibitor is a limus drug. In some embodiments, the mTOR inhibitor is sirolimus or a derivative thereof. In some embodiments, the mTOR inhibitor nanoparticle composition comprises nab-sirolimus. In some embodiments, the mTOR inhibitor nanoparticle composition is nab-sirolimus. In some embodiments, the second therapeutic agent is selected from the group consisting of an immunomodulator (such as an immunostimulator or an immune checkpoint inhibitor), a histone deacetylase inhibitor, a kinase inhibitor (such as a tyrosine kinase inhibitor), and a cancer vaccine (such as a vaccine prepared using tumor cells or at least one tumor-associated antigen). In some embodiments, the second therapeutic agent is an immunomodulator. In some embodiments, the immunomodulator is an immunostimulator that directly stimulates the immune system of an individual. In some embodiments, the immunomodulator is an agonistic antibody that targets an activating receptor on an immune cell (such as a T cell). In some embodiments, the immunomodulator is an immune checkpoint inhibitor. In some embodiments, the immune checkpoint inhibitor is an antagonistic antibody that targets an immune checkpoint protein. In some embodiments, the immunomodulator is an IMiDs® (small molecule immunomodulator, such as lenalidomide or pomalidomide). In some embodiments, the immunomodulator is lenalidomide. In some embodiments, the immunomodulator is pomalidomide. In some embodiments, the immunomodulator is small molecule or antibody-based IDO inhibitor. In some embodiments, the second therapeutic agent is a histone deacetylase inhibitor. In some embodiments, the histone deacetylase inhibitor is selected from the group consisting of romidepsin, panobinostat, ricolinostat, and belinostat. In some embodiments, the histone deacetylase inhibitor is romidepsin. In some embodiments, the second therapeutic agent is a kinase inhibitor, such as a tyrosine kinase inhibitor. In some embodiments, the kinase inhibitor is a serine/threonine kinase inhibitor. In some embodiments, the kinase inhibitor is selected from the group consisting of erlotinib, imatinib, lapatinib, nilotinib, sorafenib, and sunitinib. In some embodiments, the second therapeutic agent is a cancer vaccine, such as a vaccine prepared using tumor cells or at least one tumor-associated antigen. In some embodiments, the second therapeutic agent is a vinca alkaloid (such as vinblastine, vincristine, vindesine, or vinorelbine) or an alkylating agent (such as cyclophosphamide, melphalan, chlorambucil, ifosfamide, streptozocin, or busulfan). In some embodiments, the neuroblastoma is recurrent neuroblastoma. In some embodiments, the neuroblastoma is refractory to at least one drug used in a standard therapy for neuroblastoma (e.g., cyclophosphamide, ifosfamide, cisplatin, carboplatin, vincristine, doxorubicin, etoposide, topotecan, busulfan, melphalan, and/or dinutuximab).

In some embodiments, according to any of the methods described above, the solid tumor is Ewing's sarcoma. In some embodiments, the Ewing's sarcoma is Ewing's sarcoma of the pelvis, femur, humerus, ribs or clavicle. In some embodiments, the individual may be a human who has a gene, genetic mutation, or polymorphism associated with Ewing's sarcoma (e.g., EWS and/or FLI1) or has one or more extra copies of a gene associated with Ewing's sarcoma.

Thus, in some embodiments, there is provided a method of treating Ewing's sarcoma in an individual (such as a human) comprising administering to the individual a) an effective amount of a composition comprising nanoparticles comprising an mTOR inhibitor (such as a limus drug, e.g., sirolimus or a derivative thereof) and an albumin; and b) an effective amount of a second therapeutic agent. In some embodiments, the mTOR inhibitor is a limus drug. In some embodiments, the mTOR inhibitor is sirolimus or a derivative thereof. In some embodiments, the mTOR inhibitor nanoparticle composition comprises nab-sirolimus. In some embodiments, the mTOR inhibitor nanoparticle composition is nab-sirolimus. In some embodiments, the second therapeutic agent is selected from the group consisting of an immunomodulator (such as an immunostimulator or an immune checkpoint inhibitor), a histone deacetylase inhibitor, a kinase inhibitor (such as a tyrosine kinase inhibitor), and a cancer vaccine (such as a vaccine prepared using tumor cells or at least one tumor-associated antigen). In some embodiments, the second therapeutic agent is an immunomodulator. In some embodiments, the immunomodulator is an immunostimulator that directly stimulates the immune system of an individual. In some embodiments, the immunomodulator is an agonistic antibody that targets an activating receptor on an immune cell (such as a T cell). In some embodiments, the immunomodulator is an immune checkpoint inhibitor. In some embodiments, the immune checkpoint inhibitor is an antagonistic antibody that targets an immune checkpoint protein. In some embodiments, the immunomodulator is an IMiDs® (small molecule immunomodulator, such as lenalidomide or pomalidomide). In some embodiments, the immunomodulator is lenalidomide. In some embodiments, the immunomodulator is pomalidomide. In some embodiments, the immunomodulator is small molecule or antibody-based IDO inhibitor. In some embodiments, the second therapeutic agent is a histone deacetylase inhibitor. In some embodiments, the histone deacetylase inhibitor is selected from the group consisting of romidepsin, panobinostat, ricolinostat, and belinostat. In some embodiments, the histone deacetylase inhibitor is romidepsin. In some embodiments, the second therapeutic agent is a kinase inhibitor, such as a tyrosine kinase inhibitor. In some embodiments, the kinase inhibitor is a serine/threonine kinase inhibitor. In some embodiments, the kinase inhibitor is selected from the group consisting of erlotinib, imatinib, lapatinib, nilotinib, sorafenib, and sunitinib. In some embodiments, the second therapeutic agent is a cancer vaccine, such as a vaccine prepared using tumor cells or at least one tumor-associated antigen. In some embodiments, the second therapeutic agent is a vinca alkaloid (such as vinblastine, vincristine, vindesine, or vinorelbine) or an alkylating agent (such as cyclophosphamide, melphalan, chlorambucil, ifosfamide, streptozocin, or busulfan). In some embodiments, the Ewing's sarcoma is recurrent Ewing's sarcoma. In some embodiments, the Ewing's sarcoma is refractory to at least one drug used in a standard therapy for Ewing's sarcoma (e.g., vincristine, doxorubicin, cyclophosphamide, ifosfamide, and/or etoposide).

In some embodiments, according to any of the methods described above, the solid tumor is characterized by PDK and/or AKT activation. In some embodiments, the solid tumor characterized by PDK and/or AKT activation is HER2⁺ breast cancer, ovarian cancer, endometrial cancer, sarcoma, squamous cell carcinoma of the head and neck, or thyroid cancer. In some variations, the solid tumor is further characterized by AKT gene amplification.

In some embodiments, according to any of the methods described above, the solid tumor is characterized by cyclin D1 overexpression. In some embodiments, the solid tumor characterized by cyclin D1 overexpression is breast cancer.

In some embodiments, according to any of the methods described above, the solid tumor is characterized by cMYC overexpression.

In some embodiments, according to any of the methods described above, the solid tumor is characterized by HIF overexpression. In some embodiments, the solid tumor characterized by HIF overexpression is renal cell carcinoma or Von Hippel-Lindau. In some embodiments, the solid tumor further comprises a VHL mutation.

In some embodiments, according to any of the methods described above, the solid tumor is characterized by TSC1 and/or TSC2 loss. In some embodiments, the solid tumor characterized by TSC1 and/or TSC2 is tuberous sclerosis or pulmonary lymphangiomyomatosis.

In some embodiments, according to any of the methods described above, the solid tumor is characterized by a TSC2 mutation. In some embodiments, the solid tumor characterized by TSC2 mutation is renal angiomyolipomas.

In some embodiments, according to any of the methods described above, the solid tumor is characterized by a PTEN mutation. In some embodiments, the PTEN mutation is a loss of PTEN function. In some embodiments, the solid tumor characterized by a PTEN mutation is glioblastoma, endometrial cancer, prostate cancer, sarcoma, or breast cancer.

### Methods of Treatment Based on Presence of a Biomarker

The present invention in one aspect provides methods of treating a solid tumor (such as bladder cancer, renal cell carcinoma, or melanoma) in an individual based on the status of one or more mTOR-activating aberrations in one or more mTOR-associated genes. In some embodiments, the one or more biomarkers are selected from the group consisting of biomarkers indicative of favorable response to treatment with an mTOR inhibitor, biomarkers indicative of favorable response to treatment with an immunomodulator (such as an immunostimulator or an immune checkpoint inhibitor), biomarkers indicative of favorable response to treatment with a histone deacetylase inhibitor, biomarkers indicative of favorable response to treatment with a kinase inhibitor (such as a tyrosine kinase inhibitor), and biomarkers indicative of favorable response to treatment with a cancer vaccine.

Thus, in some embodiments, there is provided a method of treating a solid tumor (such as bladder cancer, renal cell carcinoma, or melanoma) in an individual comprising administering to the individual a) an effective amount of a composition comprising nanoparticles comprising an mTOR inhibitor (such as a limus drug, e.g., sirolimus or a derivative thereof) and an albumin; and b) an effective amount of a second therapeutic agent, wherein the individual is selected for treatment based on the individual having an mTOR-activating aberration. In some embodiments, the mTOR-activating aberration comprises a mutation of an mTOR-associated gene. In some embodiments, the mTOR-activating aberration comprises a copy number variation of an mTOR-associated gene. In some embodiments, the mTOR-activating aberration comprises an aberrant expression level of an mTOR-associated gene. In some embodiments, the mTOR-activating aberration comprises an aberrant activity level of an mTOR-associated gene. In some embodiments, the at least one mTOR-associated biomarker comprises an aberrant phosphorylation level of the protein encoded by the mTOR-associated gene. In some embodiments, the mTOR-activating aberration leads to activation of mTORC1 (including for example activation of mTORC1 but not mTORC2). In some embodiments, the mTOR-activating aberration leads to activation of mTORC2 (including for example activation of mTORC2 but not mTORC1). In some embodiments, the mTOR-activating aberration leads to activation of both mTORC1 and mTORC2. In some embodiments, the mTOR-activating aberration is in at least one mTOR-associated gene selected from the group consisting of AKT1, FLT-3, MTOR, PIK3CA, PIK3CG, TSC1, TSC2, RHEB, STK11, NF1, NF2, TP53, FGFR4, BAP1, KRAS, NRAS and PTEN. In some embodiments, the mTOR-activating aberration is assessed by gene sequencing. In some embodiments, the gene sequencing is based on sequencing of DNA in a tumor sample. In some embodiments, the gene sequencing is based on sequencing of a circulating or a cell-free DNA in a blood sample. In some embodiments, the mutational status of TFE3 is further used as a basis for selecting the individual. In some embodiments, the mutational status of TFE3 comprises translocation of TFE3. In some embodiments, the mTOR-activating aberration comprises an aberrant phosphorylation level of the protein encoded by the mTOR-associated gene. In some embodiments, the mTOR-associated gene is selected from the group consisting of AKT, S6K, S6, and 4EBP1. In some embodiments, the aberrant phosphorylation level is determined by immunohistochemistry. In some embodiments, the second therapeutic agent and the nanoparticle composition are administered sequentially. In some embodiments, the second therapeutic agent and the nanoparticle composition are administered simultaneously. In some embodiments, the second therapeutic agent and the nanoparticle composition are administered concurrently.

In some embodiments, there is provided a method of treating a solid tumor (such as bladder cancer, renal cell carcinoma, or melanoma) in an individual comprising: (a) assessing an mTOR-activating aberration in the individual; and (b) administering to the individual i) an effective amount of a composition comprising nanoparticles comprising an mTOR inhibitor (such as a limus drug, e.g., sirolimus or a derivative thereof) and an albumin; and ii) an effective amount of a second therapeutic agent, wherein the individual is selected for treatment based on having the mTOR-activating aberration. In some embodiments, the mTOR-activating aberration comprises a mutation of an mTOR-associated gene. In some embodiments, the mTOR-activating aberration comprises a copy number variation of an mTOR-associated gene. In some embodiments, the mTOR-activating aberration comprises an aberrant expression level of an mTOR-associated gene. In some embodiments, the mTOR-activating aberration comprises an aberrant activity level of an mTOR-associated gene. In some embodiments, the at least one mTOR-associated biomarker comprises an aberrant phosphorylation level of the protein encoded by the mTOR-associated gene. In some embodiments, the mTOR-activating aberration leads to activation of mTORC1 (including for example activation of mTORC1 but not mTORC2). In some embodiments, the mTOR-activating aberration leads to activation of mTORC2 (including for example activation of mTORC2 but not mTORC1). In some embodiments, the mTOR-activating aberration leads to activation of both mTORC1 and mTORC2. In some embodiments, the mTOR-activating aberration is in at least one mTOR-associated gene selected from the group consisting of AKT1, FLT-3, MTOR, PIK3CA, PIK3CG, TSC1, TSC2, RHEB, STK11, NF1, NF2, TP53, FGFR4, BAP1, KRAS, NRAS and PTEN. In some embodiments, the mTOR-activating aberration is assessed by gene sequencing. In some embodiments, the gene sequencing is based on sequencing of DNA in a tumor sample. In some embodiments, the gene sequencing is based on sequencing of a circulating or a cell-free DNA in a blood sample. In some embodiments, the mutational status of TFE3 is further used as a basis for selecting the individual. In some embodiments, the mutational status of TFE3 comprises translocation of TFE3. In some embodiments, the mTOR-activating aberration comprises an aberrant phosphorylation level of the protein encoded by the mTOR-associated gene. In some embodiments, the mTOR-associated gene is selected from the group consisting of AKT, S6K, S6, and 4EBP1. In some embodiments, the aberrant phosphorylation level is determined by immunohistochemistry.

In some embodiments, there is provided a method of treating a solid tumor (such as bladder cancer, renal cell carcinoma, or melanoma) in an individual comprising: (a) assessing an mTOR-activating aberration in the individual; (b) selecting (e.g., identifying or recommending) the individual for treatment based on the individual having the mTOR-activating aberration; and (c) administering to the individual i) an effective amount of a composition comprising nanoparticles comprising an mTOR inhibitor (such as a limus drug, e.g., sirolimus or a derivative thereof) and an albumin; and ii) an effective amount of a second therapeutic agent. In some embodiments, the mTOR-activating aberration comprises a mutation of an mTOR-associated gene. In some embodiments, the mTOR-activating aberration comprises a copy number variation of an mTOR-associated gene. In some embodiments, the mTOR-activating aberration comprises an aberrant expression level of an mTOR-associated gene. In some embodiments, the mTOR-activating aberration comprises an aberrant activity level of an mTOR-associated gene. In some embodiments, the at least one mTOR-associated biomarker comprises an aberrant phosphorylation level of the protein encoded by the mTOR-associated gene. In some embodiments, the mTOR-activating aberration leads to activation of mTORC1 (including for example activation of mTORC1 but not mTORC2). In some embodiments, the mTOR-activating aberration leads to activation of mTORC2 (including for example activation of mTORC2 but not mTORC1). In some embodiments, the mTOR-activating aberration leads to activation of both mTORC1 and mTORC2. In some embodiments, the mTOR-activating aberration is in at least one mTOR-associated gene selected from the group consisting of AKT1, FLT-3, MTOR, PIK3CA, PIK3CG, TSC1, TSC2, RHEB, STK11, NF1, NF2, TP53, FGFR4, BAP1, KRAS, NRAS and PTEN. In some embodiments, the mTOR-activating aberration is assessed by gene sequencing. In some embodiments, the gene sequencing is based on sequencing of DNA in a tumor sample. In some embodiments, the gene sequencing is based on sequencing of a circulating or a cell-free DNA in a blood sample. In some embodiments, the mutational status of TFE3 is further used as a basis for selecting the individual. In some embodiments, the mutational status of TFE3 comprises translocation of TFE3. In some embodiments, the mTOR-activating aberration comprises an aberrant phosphorylation level of the protein encoded by the mTOR-associated gene. In some embodiments, the mTOR-associated gene is selected from the group consisting of AKT, S6K, S6, and 4EBP1. In some embodiments, the aberrant phosphorylation level is determined by immunohistochemistry.

In some embodiments, there is provided a method of selecting (including identifying or recommending) an individual having a solid tumor (such as bladder cancer, renal cell carcinoma, or melanoma) for treatment with i) an effective amount of a composition comprising nanoparticles comprising an mTOR inhibitor (such as a limus drug, e.g., sirolimus or a derivative thereof) and an albumin; and ii) an effective amount of a second therapeutic agent, wherein the method comprises (a) assessing an mTOR-activating aberration in the individual; and (b) selecting or recommending the individual for treatment based on the individual having the mTOR-activating aberration. In some embodiments, the mTOR-activating aberration comprises a mutation of an mTOR-associated gene. In some embodiments, the mTOR-activating aberration comprises a copy number variation of an mTOR-associated gene. In some embodiments, the mTOR-activating aberration comprises an aberrant expression level of an mTOR-associated gene. In some embodiments, the mTOR-activating aberration comprises an aberrant activity level of an mTOR-associated gene. In some embodiments, the at least one mTOR-associated biomarker comprises an aberrant phosphorylation level of the protein encoded by the mTOR-associated gene. In some embodiments, the mTOR-activating aberration leads to activation of mTORC1 (including for example activation of mTORC1 but not mTORC2). In some embodiments, the mTOR-activating aberration leads to activation of mTORC2 (including for example activation of mTORC2 but not mTORC1). In some embodiments, the mTOR-activating aberration leads to activation of both mTORC1 and mTORC2. In some embodiments, the mTOR-activating aberration is in at least one mTOR-associated gene selected from the group consisting of AKT1, FLT-3, MTOR, PIK3CA, PIK3CG, TSC1, TSC2, RHEB, STK11, NF1, NF2, TP53, FGFR4, BAP1, KRAS, NRAS and PTEN. In some embodiments, the mTOR-activating aberration is assessed by gene sequencing. In some embodiments, the gene sequencing is based on sequencing of DNA in a tumor sample. In some embodiments, the gene sequencing is based on sequencing of a circulating or a cell-free DNA in a blood sample. In some embodiments, the mutational status of TFE3 is further used as a basis for selecting the individual. In some embodiments, the mutational status of TFE3 comprises translocation of TFE3. In some embodiments, the mTOR-activating aberration comprises an aberrant phosphorylation level of the protein encoded by the mTOR-associated gene. In some embodiments, the mTOR-associated gene is selected from the group consisting of AKT, S6K, S6, and 4EBP1. In some embodiments, the aberrant phosphorylation level is determined by immunohistochemistry.

In some embodiments, there is provided a method of selecting (including identifying or recommending) and treating an individual having a solid tumor (such as bladder cancer, renal cell carcinoma, or melanoma), wherein the method comprises (a) assessing an mTOR-activating aberration in the individual; (b) selecting or recommending the individual for treatment based on the individual having the mTOR-activating aberration; and (c) administering to the individual i) an effective amount of a composition comprising an mTOR inhibitor (such as a limus drug, e.g., sirolimus or a derivative thereof) and an albumin; and ii) an effective amount of a second therapeutic agent. In some embodiments, the mTOR-activating aberration comprises a mutation of an mTOR-associated gene. In some embodiments, the mTOR-activating aberration comprises a copy number variation of an mTOR-associated gene. In some embodiments, the mTOR-activating aberration comprises an aberrant expression level of an mTOR-associated gene. In some embodiments, the mTOR-activating aberration comprises an aberrant activity level of an mTOR-associated gene. In some embodiments, the at least one mTOR-associated biomarker comprises an aberrant phosphorylation level of the protein encoded by the mTOR-associated gene. In some embodiments, the mTOR-activating aberration leads to activation of mTORC1 (including for example activation of mTORC1 but not mTORC2). In some embodiments, the mTOR-activating aberration leads to activation of mTORC2 (including for example activation of mTORC2 but not mTORC1). In some embodiments, the mTOR-activating aberration leads to activation of both mTORC1 and mTORC2. In some embodiments, the mTOR-activating aberration is in at least one mTOR-associated gene selected from the group consisting of AKT1, FLT-3, MTOR, PIK3CA, PIK3CG, TSC1, TSC2, RHEB, STK11, NF1, NF2, TP53, FGFR4, BAP1, KRAS, NRAS and PTEN. In some embodiments, the mTOR-activating aberration is assessed by gene sequencing. In some embodiments, the gene sequencing is based on sequencing of DNA in a tumor sample. In some embodiments, the gene sequencing is based on sequencing of a circulating or a cell-free DNA in a blood sample. In some embodiments, the mutational status of TFE3 is further used as a basis for selecting the individual. In some embodiments, the mutational status of TFE3 comprises translocation of TFE3. In some embodiments, the mTOR-activating aberration comprises an aberrant phosphorylation level of the protein encoded by the mTOR-associated gene. In some embodiments, the mTOR-associated gene is selected from the group consisting of AKT, S6K, S6, and 4EBP1. In some embodiments, the aberrant phosphorylation level is determined by immunohistochemistry.

Also provided herein are methods of assessing whether an individual with a solid tumor (such as bladder cancer, renal cell carcinoma, or melanoma) is more likely to respond or less likely to respond to treatment based on the individual having an mTOR-activating aberration, wherein the treatment comprises i) an effective amount of a composition comprising an mTOR inhibitor (such as a limus drug, e.g., sirolimus or a derivative thereof) and an albumin; and ii) an effective amount of a second therapeutic agent; the method comprising assessing the mTOR-activating aberration in the individual. In some embodiments, the method further comprises administering to the individual who is determined to be likely to respond to the treatment i) an effective amount of a composition comprising an mTOR inhibitor (such as a limus drug, e.g., sirolimus or a derivative thereof) and an albumin; and ii) an effective amount of a second therapeutic agent. In some embodiments, the presence of the mTOR-activating aberration indicates that the individual is more likely to respond to the treatment, and the absence of the mTOR-activating aberration indicates that the individual is less likely to respond to the treatment. In some embodiments, the amount of the mTOR inhibitor (such as a limus drug) is determined based on the status of the mTOR-activating aberration.

In some embodiments, there are also provided methods of aiding assessment of whether an individual with a solid tumor (such as bladder cancer, renal cell carcinoma, or melanoma) will likely respond to or is suitable for treatment based on the individual having an mTOR-activating aberration, wherein the treatment comprises i) an effective amount of a composition comprising an mTOR inhibitor (such as a limus drug, e.g., sirolimus or a derivative thereof) and an albumin; and ii) an effective amount of a second therapeutic agent; the method comprising assessing the mTOR-activating aberration in the individual. In some embodiments, the presence of the mTOR-activating aberration indicates that the individual will likely be responsive to the treatment, and the absence of the mTOR-activating aberration indicates that the individual is less likely to respond to the treatment. In some embodiments, the method further comprises administering to the individual i) an effective amount of a composition comprising an mTOR inhibitor (such as a limus drug, e.g., sirolimus or a derivative thereof) and an albumin; and ii) an effective amount of a second therapeutic agent.

In some embodiments, there is provided a method of identifying an individual with a solid tumor (such as bladder cancer, renal cell carcinoma, or melanoma) likely to respond to treatment comprising i) an effective amount of a composition comprising an mTOR inhibitor (such as a limus drug, e.g., sirolimus or a derivative thereof) and an albumin; and ii) an effective amount of a second therapeutic agent; the method comprising: a) assessing an mTOR-activating aberration in the individual; and b) identifying the individual based on the individual having the mTOR-activating aberration. In some embodiments, the method further comprises administering i) an effective amount of a composition comprising an mTOR inhibitor (such as a limus drug, e.g., sirolimus or a derivative thereof) and an albumin; and ii) an effective amount of a second therapeutic agent. In some embodiments, the amount of the mTOR inhibitor (such as a limus drug, e.g., sirolimus or a derivative thereof) is determined based on the status of the mTOR-activating aberration.

Also provided herein are methods of adjusting therapy treatment of an individual with a solid tumor (such as bladder cancer, renal cell carcinoma, or melanoma) receiving i) an effective amount of a composition comprising an mTOR inhibitor (such as a limus drug, e.g., sirolimus or a derivative thereof) and an albumin; and ii) an effective amount of a second therapeutic agent; the method comprising assessing an mTOR-activating aberration in a sample isolated from the individual, and adjusting the therapy treatment based on the status of the mTOR-activating aberration. In some embodiments, the amount of the mTOR inhibitor (such as a limus drug, e.g., sirolimus or a derivative thereof) is adjusted.

Also provided herein are methods of marketing a therapy comprising i) an effective amount of a composition comprising an mTOR inhibitor (such as a limus drug, e.g., sirolimus or a derivative thereof) and an albumin; and ii) an effective amount of a second therapeutic agent for use in a solid tumor (such as bladder cancer, renal cell carcinoma, or melanoma) in an individual subpopulation, the methods comprising informing a target audience about the use of the therapy for treating the individual subpopulation characterized by the individuals of such subpopulation having a sample which has an mTOR-activating aberration.

“MTOR-activating aberration” refers to a genetic aberration, an aberrant expression level and/or an aberrant activity level of one or more mTOR-associated gene that may lead to hyperactivation of the mTOR signaling pathway. “Hyperactivate” refers to increase of an activity level of a molecule (such as a protein or protein complex) or a signaling pathway (such as the mTOR a signaling pathway) to a level that is above a reference activity level or range, such as at least about any of 10%, 20%, 30%, 40%, 60%, 70%, 80%, 90%, 100%, 200%, 500% or more above the reference activity level or the median of the reference activity range. In some embodiments, the reference activity level is a clinically accepted normal activity level in a standardized test, or an activity level in a healthy individual (or tissue or cell isolated from the individual) free of the mTOR-activating aberration.

The mTOR-activating aberration contemplated herein may include one type of aberration in one mTOR-associated gene, more than one type (such as at least about any of 2, 3, 4, 5, 6, or more) of aberrations in one mTOR-associated gene, one type of aberration in more than one (such as at least about any of 2, 3, 4, 5, 6, or more) mTOR-associated genes, or more than one type (such as at least about any of 2, 3, 4, 5, 6, or more) of aberration in more than one (such as at least about any of 2, 3, 4, 5, 6, or more) mTOR-associated genes. Different types of mTOR-activating aberration may include, but are not limited to, genetic aberrations, aberrant expression levels (e.g. overexpression or under-expression), aberrant activity levels (e.g. high or low activity levels), and aberrant phosphorylation levels. In some embodiments, a genetic aberration comprises a change to the nucleic acid (such as DNA or RNA) or protein sequence (i.e. mutation) or an aberrant epigenetic feature associated with an mTOR-associated gene, including, but not limited to, coding, non-coding, regulatory, enhancer, silencer, promoter, intron, exon, and untranslated regions of the mTOR-associated gene. In some embodiments, the at least one molecule (such as a protein or protein complex) or a signaling pathway (such as the mTOR a signaling pathway) to a level that is above a reference activity level or range, such as at least about any of 10%, 20%, 30%, 40%, 60%, 70%, 80%, 90%, 100%, 200%, 500% or more above the reference activity level or the median of the reference activity range. In some embodiments, the reference activity level is a clinically accepted normal activity level in a standardized test, or an activity level in a healthy individual (or tissue or cell isolated from the individual) free of the mTOR-activating aberration.

The mTOR-activating aberration contemplated herein may include one type of aberration in one mTOR-associated gene, more than one type (such as at least about any of 2, 3, 4, 5, 6, or more) of aberrations in one mTOR-associated gene, one type of aberration in more than one (such as at least about any of 2, 3, 4, 5, 6, or more) mTOR-associated genes, or more than one type (such as at least about any of 2, 3, 4, 5, 6, or more) of aberration in more than one (such as at least about any of 2, 3, 4, 5, 6, or more) mTOR-associated genes. Different types of mTOR-activating aberration may include, but are not limited to, genetic aberrations, aberrant expression levels (e.g. overexpression or under-expression), aberrant activity levels (e.g. high or low activity levels), and aberrant phosphorylation levels. In some embodiments, a genetic aberration comprises a change to the nucleic acid (such as DNA or RNA) or protein sequence (i.e. mutation) or an aberrant epigenetic feature associated with an mTOR-associated gene, including, but not limited to, coding, non-coding, regulatory, enhancer, silencer, promoter, intron, exon, and untranslated regions of the mTOR-associated biomarker comprises an aberrant phosphorylation level of the protein encoded by the molecule (such as a protein or protein complex) or a signaling pathway (such as the mTOR a signaling pathway) to a level that is above a reference activity level or range, such as at least about any of 10%, 20%, 30%, 40%, 60%, 70%, 80%, 90%, 100%, 200%, 500% or more above the reference activity level or the median of the reference activity range. In some embodiments, the reference activity level is a clinically accepted normal activity level in a standardized test, or an activity level in a healthy individual (or tissue or cell isolated from the individual) free of the mTOR-activating aberration.

The mTOR-activating aberration contemplated herein may include one type of aberration in one mTOR-associated gene, more than one type (such as at least about any of 2, 3, 4, 5, 6, or more) of aberrations in one mTOR-associated gene, one type of aberration in more than one (such as at least about any of 2, 3, 4, 5, 6, or more) mTOR-associated genes, or more than one type (such as at least about any of 2, 3, 4, 5, 6, or more) of aberration in more than one (such as at least about any of 2, 3, 4, 5, 6, or more) mTOR-associated genes. Different types of mTOR-activating aberration may include, but are not limited to, genetic aberrations, aberrant expression levels (e.g. overexpression or under-expression), aberrant activity levels (e.g. high or low activity levels), and aberrant phosphorylation levels. In some embodiments, a genetic aberration comprises a change to the nucleic acid (such as DNA or RNA) or protein sequence (i.e. mutation) or an aberrant epigenetic feature associated with an mTOR-associated gene, including, but not limited to, coding, non-coding, regulatory, enhancer, silencer, promoter, intron, exon, and untranslated regions of the mTOR-associated gene. In some embodiments, the mTOR-activating aberration comprises a mutation of an mTOR-associated gene, including, but not limited to, deletion, frameshift, insertion, missense mutation, nonsense mutation, point mutation, silent mutation, splice site mutation, and translocation. In some embodiments, the mutation may be a loss of function mutation for a negative regulator of the mTOR signaling pathway or a gain of function mutation of a positive regulator of the mTOR signaling pathway. In some embodiments, the genetic aberration comprises a copy number variation of an mTOR-associated gene. In some embodiments, the copy number variation of the mTOR-associated gene is caused by structural rearrangement of the genome, including deletions, duplications, inversion, and translocations. In some embodiments, the genetic aberration comprises an aberrant epigenetic feature of an mTOR-associated gene, including, but not limited to, DNA methylation, hydroxymethylation, increased or decreased histone binding, chromatin remodeling, and the like.

The mTOR-activating aberration is determined in comparison to a control or reference, such as a reference sequence (such as a nucleic acid sequence or a protein sequence), a control expression (such as RNA or protein expression) level, a control activity (such as activation or inhibition of downstream targets) level, or a control protein phosphorylation level. The aberrant expression level or the aberrant activity level in an mTOR-associated gene may be above the control level (such as about any of 10%, 20%, 30%, 40%, 60%, 70%, 80%, 90%, 100%, 200%, 500% or more above the control level) if the mTOR-associated gene is a positive regulator (i.e. activator) of the mTOR signaling pathway, or below the control level (such as about any of 10%, 20%, 30%, 40%, 60%, 70%, 80%, 90% or more below the control level) if the mTOR-associated gene is a negative regulator (i.e. inhibitor) of the mTOR signaling pathway. In some embodiments, the control level (e.g., expression level or activity level) is the median level (e.g., expression level or activity level) of a control population. In some embodiments, the control population is a population having the same solid tumor (such as bladder cancer, renal cell carcinoma, or melanoma) as the individual being treated. In some embodiments, the control population is a healthy population that does not have the solid tumor (such as bladder cancer, renal cell carcinoma, or melanoma), and optionally with comparable demographic characteristics (e.g., gender, age, ethnicity, etc.) as the individual being treated. In some embodiments, the control level (e.g., expression level or activity level) is a level (e.g., expression level or activity level) of a healthy tissue from the same individual. A genetic aberration may be determined by comparing to a reference sequence, including epigenetic patterns of the reference sequence in a control sample. In some embodiments, the reference sequence is the sequence (DNA, RNA or protein sequence) corresponding to a fully functional allele of an mTOR-associated gene, such as an allele (e.g., the prevalent allele) of the mTOR-associated gene present in a healthy population of individuals that do not have the solid tumor (such as bladder cancer, renal cell carcinoma, or melanoma), but may optionally have similar demographic characteristics (such as gender, age, ethnicity etc.) as the individual being treated.

The “status” of an mTOR-activating aberration may refer to the presence or absence of the mTOR-activating aberration in one or more mTOR-associated genes, or the aberrant level (expression or activity level, including phosphorylation level of a protein). In some embodiments, the presence of a genetic aberration (such as a mutation or a copy number variation) in one or more mTOR-associated genes as compared to a control indicates that (a) the individual is more likely to respond to treatment or (b) the individual is selected for treatment. In some embodiments, the absence of a genetic aberration in an mTOR-associated gene, or a wildtype mTOR-associated gene compared to a control, indicates that (a) the individual is less likely to respond to treatment or (b) the individual is not selected for treatment. In some embodiments, an aberrant level (such as expression level or activity level, including phosphorylation level of a protein) of one or more mTOR-associated genes is correlated with the likelihood of the individual to respond to treatment. For example, a larger deviation of the level (such as expression level or activity level, including phosphorylation level of a protein) of one or more mTOR-associated genes in the direction of hyperactivating the mTOR signaling pathway indicates that the individual is more likely to respond to treatment. In some embodiments, a prediction model based on the level(s) (such as expression level or activity level, including phosphorylation level of a protein) of one or more mTOR-associated genes is used to predict (a) the likelihood of the individual to respond to treatment and (b) whether to select the individual for treatment. The prediction model, including, for example, coefficient for each level, may be obtained by statistical analysis, such as regression analysis, using clinical trial data.

The expression level, and/or activity level of the one or more mTOR-associated genes, and/or phosphorylation level of one or more proteins encoded by the one or more mTOR-associated genes, and/or the presence or absence of one or more genetic aberrations of the one or more mTOR-associated genes can be useful for determining any of the following: (a) probable or likely suitability of an individual to initially receive treatment(s); (b) probable or likely unsuitability of an individual to initially receive treatment(s); (c) responsiveness to treatment; (d) probable or likely suitability of an individual to continue to receive treatment(s); (e) probable or likely unsuitability of an individual to continue to receive treatment(s); (f) adjusting dosage; (g) predicting likelihood of clinical benefits.

As used herein, “based upon” includes assessing, determining, or measuring the individual's characteristics as described herein (and preferably selecting an individual suitable for receiving treatment). When the status of an mTOR-activating aberration is “used as a basis” for selection, assessing, measuring, or determining method of treatment as described herein, the mTOR-activating aberration in one or more mTOR-associated genes is determined before and/or during treatment, and the status (including presence, absence, expression level, and/or activity level of the mTOR-activating aberration) obtained is used by a clinician in assessing any of the following: (a) probable or likely suitability of an individual to initially receive treatment(s); (b) probable or likely unsuitability of an individual to initially receive treatment(s); (c) responsiveness to treatment; (d) probable or likely suitability of an individual to continue to receive treatment(s); (e) probable or likely unsuitability of an individual to continue to receive treatment(s); (f) adjusting dosage; or (g) predicting likelihood of clinical benefits.

The mTOR-activating aberration in an individual can be assessed or determined by analyzing a sample from the individual. The assessment may be based on fresh tissue samples or archived tissue samples. Suitable samples include, but are not limited to, solid tumor (such as bladder cancer, renal cell carcinoma, or melanoma) tissue, normal tissue adjacent to the solid tumor (such as bladder cancer, renal cell carcinoma, or melanoma) tissue, normal tissue distal to the solid tumor (such as bladder cancer, renal cell carcinoma, or melanoma) tissue, or peripheral blood lymphocytes. In some embodiments, the sample is a solid tumor (such as bladder cancer, renal cell carcinoma, or melanoma) tissue. In some embodiments, the sample is a biopsy containing solid tumor (such as bladder cancer, renal cell carcinoma, or melanoma) cells, such as fine needle aspiration of solid tumor (such as bladder cancer, renal cell carcinoma, or melanoma) cells or laparoscopy obtained solid tumor (such as bladder cancer, renal cell carcinoma, or melanoma) cells. In some embodiments, the biopsied cells are centrifuged into a pellet, fixed, and embedded in paraffin prior to the analysis. In some embodiments, the biopsied cells are flash frozen prior to the analysis. In some embodiments, the sample is a plasma sample.

In some embodiments, the sample comprises a circulating metastatic cancer cell. In some embodiments, the sample is obtained by sorting circulating tumor cells (CTCs) from blood. In some further embodiments, the CTCs have detached from a primary tumor and circulate in a bodily fluid. In some further embodiments, the CTCs have detached from a primary tumor and circulate in the bloodstream. In some embodiments, the CTCs are an indication of metastasis.

In some embodiments, the sample is mixed with an antibody that recognizes a molecule encoded by an mTOR-associated gene (such as a protein) or fragment thereof. In some embodiments, the sample is mixed with a nucleic acid that recognizes nucleic acids associated with the mTOR-associated gene (such as DNA or RNA) or fragment thereof. In some embodiments, the sample is used for sequencing analysis, such as next-generation DNA, RNA and/or exome sequencing analysis.

The mTOR-activating aberration may be assessed before the start of the treatment, at any time during the treatment, and/or at the end of the treatment. In some embodiments, the mTOR-activating aberration is assessed from about 3 days prior to the administration of an mTOR inhibitor nanoparticle composition (such as sirolimus/albumin nanoparticle composition) to about 3 days after the administration of the mTOR inhibitor nanoparticle composition in each cycle of the administration. In some embodiments, the mTOR-activating aberration is assessed on day 1 of each cycle of administration. In some embodiments, the mTOR-activating aberration is assessed in cycle 1, cycle 2 and cycle 3. In some embodiments, the mTOR-activating aberration is further assessed every 2 cycles after cycle 3.

In some embodiments, there is provided a method of treating a solid tumor (such as bladder cancer, renal cell carcinoma, or melanoma) in an individual comprising administering to the individual a) an effective amount of a composition comprising nanoparticles comprising an mTOR inhibitor (such as a limus drug, e.g., sirolimus or a derivative thereof) and an albumin; and b) an effective amount of an immunomodulator, wherein the individual is selected for treatment based on the individual having at least one biomarker indicative of favorable response to treatment with an immunomodulator (hereinafter also referred to as an “immunomodulator-associated biomarker”). In some embodiments, the immunomodulator-associated biomarker comprises an aberration in a gene that affects the response to treatment of a solid tumor (such as bladder cancer, renal cell carcinoma, or melanoma) in an individual with an immunomodulator (hereinafter also referred to as an “immunomodulator-associated gene”). In some embodiments, the at least one immunomodulator-associated biomarker comprises a mutation of an immunomodulator-associated gene. In some embodiments, the at least one immunomodulator-associated biomarker comprises a copy number variation of an immunomodulator-associated gene. In some embodiments, the at least one immunomodulator-associated biomarker comprises an aberrant expression level of an immunomodulator-associated gene. In some embodiments, the at least one immunomodulator-associated biomarker comprises an aberrant activity level of an immunomodulator-associated gene. In some embodiments, the at least one immunomodulator-associated biomarker comprises an aberrant phosphorylation level of the protein encoded by the immunomodulator-associated gene. In some embodiments, the immunomodulator-associated gene is selected from the group consisting of HbF, RANKL, PU.1, ERK, cathepsin K, OPG, MIP-1α, BAFF, APRIL, CRBN, Ikaros, Aiolos, TNF-α, IL-1, IL-12, IL-2, IL-10, IFN-γ, GM-CSF, erk1/2, Akt2, αVβ3-integrin, IRF4, C/EBPβ (NF-IL6), p21, and VEGF. In some embodiments, the immunomodulator is an immunostimulator. In some embodiments, the immunomodulator is an immunostimulator that directly stimulates the immune system of an individual. In some embodiments, the immunomodulator is an agonistic antibody that targets an activating receptor on an immune cell (such as a T cell). In some embodiments, the immunomodulator is an immune checkpoint inhibitor. In some embodiments, the immune checkpoint inhibitor is an antagonistic antibody that targets an immune checkpoint protein. In some embodiments, the immunomodulator is an IMiDs® compound (small molecule immunomodulator, such as lenalidomide or pomalidomide). In some embodiments, the immunomodulator is pomalidomide. In some embodiments, the immunomodulator is lenalidomide. In some embodiments, the immunomodulator is small molecule or antibody-based IDO inhibitor.

In some embodiments, there is provided a method of treating a solid tumor (such as bladder cancer, renal cell carcinoma, or melanoma) in an individual comprising: (a) assessing at least one immunomodulator-associated biomarker in the individual; and (b) administering to the individual i) an effective amount of a composition comprising nanoparticles comprising an mTOR inhibitor (such as a limus drug, e.g., sirolimus or a derivative thereof) and an albumin; and ii) an effective amount of an immunomodulator, wherein the individual is selected for treatment based on having the at least one immunomodulator-associated biomarker. In some embodiments, the at least one immunomodulator-associated biomarker comprises a mutation of an immunomodulator-associated gene. In some embodiments, the at least one immunomodulator-associated biomarker comprises a copy number variation of an immunomodulator-associated gene. In some embodiments, the at least one immunomodulator-associated biomarker comprises an aberrant expression level of an immunomodulator-associated gene. In some embodiments, the at least one immunomodulator-associated biomarker comprises an aberrant activity level of an immunomodulator-associated gene. In some embodiments, the at least one immunomodulator-associated biomarker comprises an aberrant phosphorylation level of the protein encoded by the immunomodulator-associated gene. In some embodiments, the immunomodulator-associated gene is selected from the group consisting of HbF, RANKL, PU.1, ERK, cathepsin K, OPG, MIP-1α, BAFF, APRIL, CRBN, Ikaros, Aiolos, TNF-α, IL-1, IL-12, IL-2, IL-10, IFN-γ, GM-CSF, erk1/2, Akt2, αVβ3-integrin, IRF4, C/EBPβ (NF-IL6), p21, and VEGF. In some embodiments, the immunomodulator is an immunostimulator. In some embodiments, the immunomodulator is an immunostimulator that directly stimulates the immune system of an individual. In some embodiments, the immunomodulator is an agonistic antibody that targets an activating receptor on an immune cell (such as a T cell). In some embodiments, the immunomodulator is an immune checkpoint inhibitor. In some embodiments, immune checkpoint inhibitor is an antagonistic antibody that targets an immune checkpoint protein. In some embodiments, the immunomodulator is an IMiDs® compound (small molecule immunomodulator, such as lenalidomide or pomalidomide). In some embodiments, the immunomodulator is pomalidomide. In some embodiments, the immunomodulator is lenalidomide. In some embodiments, the immunomodulator is small molecule or antibody-based IDO inhibitor.

In some embodiments, there is provided a method of treating a solid tumor (such as bladder cancer, renal cell carcinoma, or melanoma) in an individual comprising: (a) assessing at least one immunomodulator-associated biomarker in the individual; (b) selecting (e.g., identifying or recommending) the individual for treatment based on the individual having the at least one immunomodulator-associated biomarker; and (c) administering to the individual i) an effective amount of a composition comprising nanoparticles comprising an mTOR inhibitor (such as a limus drug, e.g., sirolimus or a derivative thereof) and an albumin; and ii) an effective amount of an immunomodulator. In some embodiments, the at least one immunomodulator-associated biomarker comprises a mutation of an immunomodulator-associated gene. In some embodiments, the at least one immunomodulator-associated biomarker comprises a copy number variation of an immunomodulator-associated gene. In some embodiments, the at least one immunomodulator-associated biomarker comprises an aberrant expression level of an immunomodulator-associated gene. In some embodiments, the at least one immunomodulator-associated biomarker comprises an aberrant activity level of an immunomodulator-associated gene. In some embodiments, the at least one immunomodulator-associated biomarker comprises an aberrant phosphorylation level of the protein encoded by the immunomodulator-associated gene. In some embodiments, the immunomodulator-associated gene is selected from the group consisting of HbF, RANKL, PU.1, ERK, cathepsin K, OPG, MIP-1α, BAFF, APRIL, CRBN, Ikaros, Aiolos, TNF-α, IL-1, IL-12, IL-2, IL-10, IFN-γ, GM-CSF, erk1/2, Akt2, αVβ3-integrin, IRF4, C/EBPβ (NF-IL6), p21, and VEGF. In some embodiments, the immunomodulator is an immunostimulator. In some embodiments, the immunomodulator is an immunostimulator that directly stimulates the immune system of an individual. In some embodiments, the immunomodulator is an agonistic antibody that targets an activating receptor on an immune cell (such as a T cell). In some embodiments, the immunomodulator is an immune checkpoint inhibitor. In some embodiments, the immune checkpoint inhibitor is an antagonistic antibody that targets an immune checkpoint protein. In some embodiments, the immunomodulator is an IMiDs® compound (small molecule immunomodulator, such as lenalidomide or pomalidomide). In some embodiments, the immunomodulator is pomalidomide. In some embodiments, the immunomodulator is lenalidomide. In some embodiments, the immunomodulator is small molecule or antibody-based IDO inhibitor.

In some embodiments, there is provided a method of selecting (including identifying or recommending) an individual having a solid tumor (such as bladder cancer, renal cell carcinoma, or melanoma) for treatment with i) an effective amount of a composition comprising nanoparticles comprising an mTOR inhibitor (such as a limus drug, e.g., sirolimus or a derivative thereof) and an albumin; and ii) an effective amount of an immunomodulator, wherein the method comprises (a) assessing at least one immunomodulator-associated biomarker in the individual; and (b) selecting or recommending the individual for treatment based on the individual having the at least one immunomodulator-associated biomarker. In some embodiments, the at least one immunomodulator-associated biomarker comprises a mutation of an immunomodulator-associated gene. In some embodiments, the at least one immunomodulator-associated biomarker comprises a copy number variation of an immunomodulator-associated gene. In some embodiments, the at least one immunomodulator-associated biomarker comprises an aberrant expression level of an immunomodulator-associated gene. In some embodiments, the at least one immunomodulator-associated biomarker comprises an aberrant activity level of an immunomodulator-associated gene. In some embodiments, the at least one immunomodulator-associated biomarker comprises an aberrant phosphorylation level of the protein encoded by the immunomodulator-associated gene. In some embodiments, the immunomodulator-associated gene is selected from the group consisting of HbF, RANKL, PU.1, ERK, cathepsin K, OPG, MIP-1α, BAFF, APRIL, CRBN, Ikaros, Aiolos, TNF-α, IL-1, IL-12, IL-2, IL-10, IFN-γ, GM-CSF, erk1/2, Akt2, αVβ3-integrin, IRF4, C/EBPβ (NF-IL6), p21, and VEGF. In some embodiments, the immunomodulator is an immunostimulator. In some embodiments, the immunomodulator is an immunostimulator that directly stimulates the immune system of an individual. In some embodiments, the immunomodulator is an agonistic antibody that targets an activating receptor on an immune cell (such as a T cell). In some embodiments, the immunomodulator is an immune checkpoint inhibitor. In some embodiments, the immune checkpoint inhibitor is an antagonistic antibody that targets an immune checkpoint protein. In some embodiments, the immunomodulator is an IMiDs® compound (small molecule immunomodulator, such as lenalidomide or pomalidomide). In some embodiments, the immunomodulator is pomalidomide. In some embodiments, the immunomodulator is lenalidomide. In some embodiments, the immunomodulator is small molecule or antibody-based IDO inhibitor.

In some embodiments, there is provided a method of selecting (including identifying or recommending) and treating an individual having a solid tumor (such as bladder cancer, renal cell carcinoma, or melanoma), wherein the method comprises (a) assessing at least one immunomodulator-associated biomarker in the individual; (b) selecting or recommending the individual for treatment based on the individual having the at least one immunomodulator-associated biomarker; and (c) administering to the individual i) an effective amount of a composition comprising an mTOR inhibitor (such as a limus drug, e.g., sirolimus or a derivative thereof) and an albumin; and ii) an effective amount of an immunomodulator. In some embodiments, the at least one immunomodulator-associated biomarker comprises a mutation of an immunomodulator-associated gene. In some embodiments, the at least one immunomodulator-associated biomarker comprises a copy number variation of an immunomodulator-associated gene. In some embodiments, the at least one immunomodulator-associated biomarker comprises an aberrant expression level of an immunomodulator-associated gene. In some embodiments, the at least one immunomodulator-associated biomarker comprises an aberrant activity level of an immunomodulator-associated gene. In some embodiments, the at least one immunomodulator-associated biomarker comprises an aberrant phosphorylation level of the protein encoded by the immunomodulator-associated gene. In some embodiments, the immunomodulator-associated gene is selected from the group consisting of HbF, RANKL, PU.1, ERK, cathepsin K, OPG, MIP-1α, BAFF, APRIL, CRBN, Ikaros, Aiolos, TNF-α, IL-1, IL-12, IL-2, IL-10, IFN-γ, GM-CSF, erk1/2, Akt2, αVβ3-integrin, IRF4, C/EBPβ (NF-IL6), p21, and VEGF. In some embodiments, the immunomodulator is an immunostimulator. In some embodiments, the immunomodulator is an immunostimulator that directly stimulates the immune system of an individual. In some embodiments, the immunomodulator is an agonistic antibody that targets an activating receptor on an immune cell (such as a T cell). In some embodiments, the immunomodulator is an immune checkpoint inhibitor. In some embodiments, the immune checkpoint inhibitor is an antagonistic antibody that targets an immune checkpoint protein. In some embodiments, the immunomodulator is an IMiDs® compound (small molecule immunomodulator, such as lenalidomide or pomalidomide). In some embodiments, the immunomodulator is pomalidomide. In some embodiments, the immunomodulator is lenalidomide. In some embodiments, the immunomodulator is small molecule or antibody-based IDO inhibitor.

Also provided herein are methods of assessing whether an individual with a solid tumor (such as bladder cancer, renal cell carcinoma, or melanoma) is more likely to respond or less likely to respond to treatment based on the individual having at least one immunomodulator-associated biomarker, wherein the treatment comprises i) an effective amount of a composition comprising an mTOR inhibitor (such as a limus drug, e.g., sirolimus or a derivative thereof) and an albumin; and ii) an effective amount of an immunomodulator; the method comprising assessing at least one immunomodulator-associated biomarker in the individual. In some embodiments, the method further comprises administering to the individual who is determined to be likely to respond to the treatment i) an effective amount of a composition comprising an mTOR inhibitor (such as a limus drug, e.g., sirolimus or a derivative thereof) and an albumin; and ii) an effective amount of an immunomodulator. In some embodiments, the presence of the at least one immunomodulator-associated biomarker indicates that the individual is more likely to respond to the treatment, and the absence of the at least one immunomodulator-associated biomarker indicates that the individual is less likely to respond to the treatment. In some embodiments, the amount of the immunomodulator is determined based on the presence of the at least one immunomodulator-associated biomarker in the individual. In some embodiments, the immunomodulator is an immunostimulator. In some embodiments, the immunomodulator is an immunostimulator that directly stimulates the immune system of an individual. In some embodiments, the immunomodulator is an agonistic antibody that targets an activating receptor on an immune cell (such as a T cell). In some embodiments, the immunomodulator is an immune checkpoint inhibitor. In some embodiments, the immune checkpoint inhibitor is an antagonistic antibody that targets an immune checkpoint protein. In some embodiments, the immunomodulator is an IMiDs® compound (small molecule immunomodulator, such as lenalidomide or pomalidomide). In some embodiments, the immunomodulator is pomalidomide. In some embodiments, the immunomodulator is lenalidomide. In some embodiments, the immunomodulator is small molecule or antibody-based IDO inhibitor.

Also provided herein are methods of adjusting therapy treatment of an individual with a solid tumor (such as bladder cancer, renal cell carcinoma, or melanoma) receiving i) an effective amount of a composition comprising an mTOR inhibitor (such as a limus drug, e.g., sirolimus or a derivative thereof) and an albumin; and ii) an effective amount of an immunomodulator, the method comprising assessing at least one immunomodulator-associated biomarker in a sample isolated from the individual, and adjusting the therapy treatment based on the individual having the at least one immunomodulator-associated biomarker. In some embodiments, the amount of the immunomodulator is adjusted.

In some embodiments, there is provided a method of treating a solid tumor (such as bladder cancer, renal cell carcinoma, or melanoma) in an individual comprising administering to the individual a) an effective amount of a composition comprising nanoparticles comprising an mTOR inhibitor (such as a limus drug, e.g., sirolimus or a derivative thereof) and an albumin; and b) an effective amount of a histone deacetylase inhibitor (HDACi), wherein the individual is selected for treatment based on the individual having at least one biomarker indicative of favorable response to treatment with a histone deacetylase inhibitor (hereinafter also referred to as an “HDACi-associated biomarker”). In some embodiments, the histone deacetylase inhibitor-associated biomarker comprises an aberration in a gene that affects the response to treatment of a solid tumor (such as bladder cancer, renal cell carcinoma, or melanoma) in an individual with a histone deacetylase inhibitor (hereinafter also referred to as an “HDACi-associated gene”). In some embodiments, the at least one HDACi-associated biomarker comprises a mutation of an HDACi-associated gene. In some embodiments, the at least one HDACi-associated biomarker comprises a copy number variation of an HDACi-associated gene. In some embodiments, the at least one HDACi-associated biomarker comprises an aberrant expression level of an HDACi-associated gene. In some embodiments, the at least one HDACi-associated biomarker comprises an aberrant activity level of an HDACi-associated gene. In some embodiments, the at least one HDACi-associated biomarker comprises an aberrant phosphorylation level of the protein encoded by the HDACi-associated gene. In some embodiments, the HDACi-associated gene is selected from the group consisting of HDAC1, HDAC2, HDAC3, HDAC4, HDAC5, HDAC6, HDAC7, HDAC8, HDAC9, HDAC10, SIRT1, SIRT2, SIRT3, SIRT 4, SIRT5, SIRT6, SIRT7, CBP, MOZ, MOF, MORF, P300, and PCAF. In some embodiments, the histone deacetylase inhibitor is selected from the group consisting of romidepsin, panobinostat, ricolinostat, and belinostat.

In some embodiments, there is provided a method of treating a solid tumor (such as bladder cancer, renal cell carcinoma, or melanoma) in an individual comprising: (a) assessing at least one HDACi-associated biomarker in the individual; and (b) administering to the individual i) an effective amount of a composition comprising nanoparticles comprising an mTOR inhibitor (such as a limus drug, e.g., sirolimus or a derivative thereof) and an albumin; and ii) an effective amount of a histone deacetylase inhibitor, wherein the individual is selected for treatment based on having the at least one HDACi-associated biomarker. In some embodiments, the at least one HDACi-associated biomarker comprises a mutation of an HDACi-associated gene. In some embodiments, the at least one HDACi-associated biomarker comprises a copy number variation of an HDACi-associated gene. In some embodiments, the at least one HDACi-associated biomarker comprises an aberrant expression level of an HDACi-associated gene. In some embodiments, the at least one HDACi-associated biomarker comprises an aberrant activity level of an HDACi-associated gene. In some embodiments, the at least one HDACi-associated biomarker comprises an aberrant phosphorylation level of the protein encoded by the HDACi-associated gene. In some embodiments, the HDACi-associated gene is selected from the group consisting of HDAC1, HDAC2, HDAC3, HDAC4, HDAC5, HDAC6, HDAC7, HDAC8, HDAC9, HDAC10, SIRT1, SIRT2, SIRT3, SIRT 4, SIRT5, SIRT6, SIRT7, CBP, MOZ, MOF, MORF, P300, and PCAF. In some embodiments, the histone deacetylase inhibitor is selected from the group consisting of romidepsin, panobinostat, ricolinostat, and belinostat.

In some embodiments, there is provided a method of treating a solid tumor (such as bladder cancer, renal cell carcinoma, or melanoma) in an individual comprising: (a) assessing at least one HDACi-associated biomarker in the individual; (b) selecting (e.g., identifying or recommending) the individual for treatment based on the individual having the at least one HDACi-associated biomarker; and (c) administering to the individual i) an effective amount of a composition comprising nanoparticles comprising an mTOR inhibitor (such as a limus drug, e.g., sirolimus or a derivative thereof) and an albumin; and ii) an effective amount of a histone deacetylase inhibitor. In some embodiments, the at least one HDACi-associated biomarker comprises a mutation of an HDACi-associated gene. In some embodiments, the at least one HDACi-associated biomarker comprises a copy number variation of an HDACi-associated gene. In some embodiments, the at least one HDACi-associated biomarker comprises an aberrant expression level of an HDACi-associated gene. In some embodiments, the at least one HDACi-associated biomarker comprises an aberrant activity level of an HDACi-associated gene. In some embodiments, the at least one HDACi-associated biomarker comprises an aberrant phosphorylation level of the protein encoded by the HDACi-associated gene. In some embodiments, the HDACi-associated gene is selected from the group consisting of HDAC1, HDAC2, HDAC3, HDAC4, HDAC5, HDAC6, HDAC7, HDAC8, HDAC9, HDAC10, SIRT1, SIRT2, SIRT3, SIRT 4, SIRT5, SIRT6, SIRT7, CBP, MOZ, MOF, MORF, P300, and PCAF. In some embodiments, the histone deacetylase inhibitor is selected from the group consisting of romidepsin, panobinostat, ricolinostat, and belinostat.

In some embodiments, there is provided a method of selecting (including identifying or recommending) an individual having a solid tumor (such as bladder cancer, renal cell carcinoma, or melanoma) for treatment with i) an effective amount of a composition comprising nanoparticles comprising an mTOR inhibitor (such as a limus drug, e.g., sirolimus or a derivative thereof) and an albumin; and ii) an effective amount of a histone deacetylase inhibitor, wherein the method comprises (a) assessing at least one HDACi-associated biomarker in the individual; and (b) selecting or recommending the individual for treatment based on the individual having the at least one HDACi-associated biomarker. In some embodiments, the at least one HDACi-associated biomarker comprises a mutation of an HDACi-associated gene. In some embodiments, the at least one HDACi-associated biomarker comprises a copy number variation of an HDACi-associated gene. In some embodiments, the at least one HDACi-associated biomarker comprises an aberrant expression level of an HDACi-associated gene. In some embodiments, the at least one HDACi-associated biomarker comprises an aberrant activity level of an HDACi-associated gene. In some embodiments, the at least one HDACi-associated biomarker comprises an aberrant phosphorylation level of the protein encoded by the HDACi-associated gene. In some embodiments, the HDACi-associated gene is selected from the group consisting of HDAC1, HDAC2, HDAC3, HDAC4, HDAC5, HDAC6, HDAC7, HDAC8, HDAC9, HDAC10, SIRT1, SIRT2, SIRT3, SIRT 4, SIRT5, SIRT6, SIRT7, CBP, MOZ, MOF, MORF, P300, and PCAF. In some embodiments, the histone deacetylase inhibitor is selected from the group consisting of romidepsin, panobinostat, ricolinostat, and belinostat.

In some embodiments, there is provided a method of selecting (including identifying or recommending) and treating an individual having a solid tumor (such as bladder cancer, renal cell carcinoma, or melanoma), wherein the method comprises (a) assessing at least one HDACi-associated biomarker in the individual; (b) selecting or recommending the individual for treatment based on the individual having the at least one HDACi-associated biomarker; and (c) administering to the individual i) an effective amount of a composition comprising an mTOR inhibitor (such as a limus drug, e.g., sirolimus or a derivative thereof) and an albumin; and ii) an effective amount of a histone deacetylase inhibitor. In some embodiments, the at least one HDACi-associated biomarker comprises a mutation of an HDACi-associated gene. In some embodiments, the at least one HDACi-associated biomarker comprises a copy number variation of an HDACi-associated gene. In some embodiments, the at least one HDACi-associated biomarker comprises an aberrant expression level of an HDACi-associated gene. In some embodiments, the at least one HDACi-associated biomarker comprises an aberrant activity level of an HDACi-associated gene. In some embodiments, the at least one HDACi-associated biomarker comprises an aberrant phosphorylation level of the protein encoded by the HDACi-associated gene. In some embodiments, the HDACi-associated gene is selected from the group consisting of HDAC1, HDAC2, HDAC3, HDAC4, HDAC5, HDAC6, HDAC7, HDAC8, HDAC9, HDAC10, SIRT1, SIRT2, SIRT3, SIRT 4, SIRT5, SIRT6, SIRT7, CBP, MOZ, MOF, MORF, P300, and PCAF. In some embodiments, the histone deacetylase inhibitor is selected from the group consisting of romidepsin, panobinostat, ricolinostat, and belinostat.

Also provided herein are methods of assessing whether an individual with a solid tumor (such as bladder cancer, renal cell carcinoma, or melanoma) is more likely to respond or less likely to respond to treatment with i) an effective amount of a composition comprising an mTOR inhibitor (such as a limus drug, e.g., sirolimus or a derivative thereof) and an albumin; and ii) an effective amount of a histone deacetylase inhibitor, the method comprising assessing the at least one HDACi-associated biomarker in the individual. In some embodiments, the method further comprises administering to the individual who is determined to be likely to respond to the treatment i) an effective amount of a composition comprising an mTOR inhibitor (such as a limus drug, e.g., sirolimus or a derivative thereof) and an albumin; and ii) an effective amount of an HDACi. In some embodiments, the presence of the at least one HDACi-associated biomarker indicates that the individual is more likely to respond to the treatment, and the absence of the at least one HDACi-associated biomarker indicates that the individual is less likely to respond to the treatment. In some embodiments, the amount of the HDACi is determined based on the presence of the at least one HDACi-associated biomarker in the individual. In some embodiments, the histone deacetylase inhibitor is selected from the group consisting of romidepsin, panobinostat, ricolinostat, and belinostat.

Also provided herein are methods of adjusting therapy treatment of an individual with a solid tumor (such as bladder cancer, renal cell carcinoma, or melanoma) receiving i) an effective amount of a composition comprising an mTOR inhibitor (such as a limus drug, e.g., sirolimus or a derivative thereof) and an albumin; and ii) an effective amount of an HDACi, the method comprising assessing at least one HDACi-associated biomarker in a sample isolated from the individual, and adjusting the therapy treatment based on the individual having the at least one HDACi-associated biomarker. In some embodiments, the amount of the HDACi is adjusted.

In some embodiments, there is provided a method of treating a solid tumor (such as bladder cancer, renal cell carcinoma, or melanoma) in an individual comprising administering to the individual a) an effective amount of a composition comprising nanoparticles comprising an mTOR inhibitor (such as a limus drug, e.g., sirolimus or a derivative thereof) and an albumin; and b) an effective amount of a kinase inhibitor (such as a tyrosine kinase inhibitor), wherein the individual is selected for treatment based on the individual having at least one biomarker indicative of favorable response to treatment with a kinase inhibitor (hereinafter also referred to as a “kinase inhibitor-associated biomarker”). In some embodiments, the kinase inhibitor-associated biomarker comprises an aberration in a gene that affects the response to treatment of a solid tumor (such as bladder cancer, renal cell carcinoma, or melanoma) in an individual with a kinase inhibitor (hereinafter also referred to as a “kinase inhibitor-associated gene”). In some embodiments, the at least one kinase inhibitor-associated biomarker comprises a mutation of a kinase inhibitor-associated gene. In some embodiments, the at least one kinase inhibitor-associated biomarker comprises a copy number variation of a kinase inhibitor-associated gene. In some embodiments, the at least one kinase inhibitor-associated biomarker comprises an aberrant expression level of a kinase inhibitor-associated gene. In some embodiments, the at least one kinase inhibitor-associated biomarker comprises an aberrant activity level of a kinase inhibitor-associated gene. In some embodiments, the at least one kinase inhibitor-associated biomarker comprises an aberrant phosphorylation level of the protein encoded by the kinase inhibitor-associated gene. In some embodiments, the kinase inhibitor-associated gene is selected from the group consisting of ERK, MCL-1, and PIN1. In some embodiments, the kinase inhibitor is selected from the group consisting of nilotinib and sorafenib.

In some embodiments, there is provided a method of treating a solid tumor (such as bladder cancer, renal cell carcinoma, or melanoma) in an individual comprising: (a) assessing at least one kinase inhibitor-associated biomarker in the individual; and (b) administering to the individual i) an effective amount of a composition comprising nanoparticles comprising an mTOR inhibitor (such as a limus drug, e.g., sirolimus or a derivative thereof) and an albumin; and ii) an effective amount of a kinase inhibitor (such as a tyrosine kinase inhibitor), wherein the individual is selected for treatment based on having the at least one kinase inhibitor-associated biomarker. In some embodiments, the at least one kinase inhibitor-associated biomarker comprises a mutation of a kinase inhibitor-associated gene. In some embodiments, the at least one kinase inhibitor-associated biomarker comprises a copy number variation of a kinase inhibitor-associated gene. In some embodiments, the at least one kinase inhibitor-associated biomarker comprises an aberrant expression level of a kinase inhibitor-associated gene. In some embodiments, the at least one kinase inhibitor-associated biomarker comprises an aberrant activity level of a kinase inhibitor-associated gene. In some embodiments, the at least one kinase inhibitor-associated biomarker comprises an aberrant phosphorylation level of the protein encoded by the kinase inhibitor-associated gene. In some embodiments, the kinase inhibitor-associated gene is selected from the group consisting of ERK, MCL-1, and PIN1. In some embodiments, the kinase inhibitor is selected from the group consisting of nilotinib and sorafenib.

In some embodiments, there is provided a method of treating a solid tumor (such as bladder cancer, renal cell carcinoma, or melanoma) in an individual comprising: (a) assessing at least one kinase inhibitor-associated biomarker in the individual; (b) selecting (e.g., identifying or recommending) the individual for treatment based on the individual having the at least one kinase inhibitor-associated biomarker; and (c) administering to the individual i) an effective amount of a composition comprising nanoparticles comprising an mTOR inhibitor (such as a limus drug, e.g., sirolimus or a derivative thereof) and an albumin; and ii) an effective amount of a kinase inhibitor (such as a tyrosine kinase inhibitor). In some embodiments, the at least one kinase inhibitor-associated biomarker comprises a mutation of a kinase inhibitor-associated gene. In some embodiments, the at least one kinase inhibitor-associated biomarker comprises a copy number variation of a kinase inhibitor-associated gene. In some embodiments, the at least one kinase inhibitor-associated biomarker comprises an aberrant expression level of a kinase inhibitor-associated gene. In some embodiments, the at least one kinase inhibitor-associated biomarker comprises an aberrant activity level of a kinase inhibitor-associated gene. In some embodiments, the at least one kinase inhibitor-associated biomarker comprises an aberrant phosphorylation level of the protein encoded by the kinase inhibitor-associated gene. In some embodiments, the kinase inhibitor-associated gene is selected from the group consisting of ERK, MCL-1, and PIN1. In some embodiments, the kinase inhibitor is selected from the group consisting of nilotinib and sorafenib.

In some embodiments, there is provided a method of selecting (including identifying or recommending) an individual having a solid tumor (such as bladder cancer, renal cell carcinoma, or melanoma) for treatment with i) an effective amount of a composition comprising nanoparticles comprising an mTOR inhibitor (such as a limus drug, e.g., sirolimus or a derivative thereof) and an albumin; and ii) an effective amount of a kinase inhibitor (such as a tyrosine kinase inhibitor), wherein the method comprises (a) assessing at least one kinase inhibitor-associated biomarker in the individual; and (b) selecting or recommending the individual for treatment based on the individual having the at least one kinase inhibitor-associated biomarker. In some embodiments, the at least one kinase inhibitor-associated biomarker comprises a mutation of a kinase inhibitor-associated gene. In some embodiments, the at least one kinase inhibitor-associated biomarker comprises a copy number variation of a kinase inhibitor-associated gene. In some embodiments, the at least one kinase inhibitor-associated biomarker comprises an aberrant expression level of a kinase inhibitor-associated gene. In some embodiments, the at least one kinase inhibitor-associated biomarker comprises an aberrant activity level of a kinase inhibitor-associated gene. In some embodiments, the at least one kinase inhibitor-associated biomarker comprises an aberrant phosphorylation level of the protein encoded by the kinase inhibitor-associated gene. In some embodiments, the kinase inhibitor-associated gene is selected from the group consisting of ERK, MCL-1, and PIN1. In some embodiments, the kinase inhibitor is selected from the group consisting of nilotinib and sorafenib.

In some embodiments, there is provided a method of selecting (including identifying or recommending) and treating an individual having a solid tumor (such as bladder cancer, renal cell carcinoma, or melanoma), wherein the method comprises (a) assessing at least one kinase inhibitor-associated biomarker in the individual; (b) selecting or recommending the individual for treatment based on the individual having the at least one kinase inhibitor-associated biomarker; and (c) administering to the individual i) an effective amount of a composition comprising an mTOR inhibitor (such as a limus drug, e.g., sirolimus or a derivative thereof) and an albumin; and ii) an effective amount of a kinase inhibitor (such as a tyrosine kinase inhibitor). In some embodiments, the at least one kinase inhibitor-associated biomarker comprises a mutation of a kinase inhibitor-associated gene. In some embodiments, the at least one kinase inhibitor-associated biomarker comprises a copy number variation of a kinase inhibitor-associated gene. In some embodiments, the at least one kinase inhibitor-associated biomarker comprises an aberrant expression level of a kinase inhibitor-associated gene. In some embodiments, the at least one kinase inhibitor-associated biomarker comprises an aberrant activity level of a kinase inhibitor-associated gene. In some embodiments, the at least one kinase inhibitor-associated biomarker comprises an aberrant phosphorylation level of the protein encoded by the kinase inhibitor-associated gene. In some embodiments, the kinase inhibitor-associated gene is selected from the group consisting of ERK, MCL-1, and PIN1. In some embodiments, the kinase inhibitor is selected from the group consisting of nilotinib and sorafenib.

Also provided herein are methods of assessing whether an individual with a solid tumor (such as bladder cancer, renal cell carcinoma, or melanoma) is more likely to respond or less likely to respond to treatment with i) an effective amount of a composition comprising an mTOR inhibitor (such as a limus drug, e.g., sirolimus or a derivative thereof) and an albumin; and ii) an effective amount of a kinase inhibitor (such as a tyrosine kinase inhibitor), the method comprising assessing the at least one kinase inhibitor-associated biomarker in the individual. In some embodiments, the method further comprises administering to the individual who is determined to be likely to respond to the treatment i) an effective amount of a composition comprising an mTOR inhibitor (such as a limus drug, e.g., sirolimus or a derivative thereof) and an albumin; and ii) an effective amount of a kinase inhibitor. In some embodiments, the presence of the at least one kinase inhibitor-associated biomarker indicates that the individual is more likely to respond to the treatment, and the absence of the at least one kinase inhibitor-associated biomarker indicates that the individual is less likely to respond to the treatment. In some embodiments, the amount of the kinase inhibitor is determined based on the presence of the at least one kinase inhibitor-associated biomarker in the individual. In some embodiments, the kinase inhibitor is selected from the group consisting of nilotinib and sorafenib.

Also provided herein are methods of adjusting therapy treatment of an individual with a solid tumor (such as bladder cancer, renal cell carcinoma, or melanoma) receiving i) an effective amount of a composition comprising an mTOR inhibitor (such as a limus drug, e.g., sirolimus or a derivative thereof) and an albumin; and ii) an effective amount of a kinase inhibitor, the method comprising assessing at least one kinase inhibitor-associated biomarker in a sample isolated from the individual, and adjusting the therapy treatment based on the individual having the at least one kinase inhibitor-associated biomarker. In some embodiments, the amount of the kinase inhibitor is adjusted.

In some embodiments, there is provided a method of treating a solid tumor (such as bladder cancer, renal cell carcinoma, or melanoma) in an individual comprising administering to the individual a) an effective amount of a composition comprising nanoparticles comprising an mTOR inhibitor (such as a limus drug, e.g., sirolimus or a derivative thereof) and an albumin; and b) an effective amount of a cancer vaccine, wherein the individual is selected for treatment based on the individual having at least one biomarker indicative of favorable response to treatment with the cancer vaccine (hereinafter also referred to as a “cancer vaccine-associated biomarker”). In some embodiments, the cancer vaccine-associated biomarker comprises an aberration in a gene that affects the response to treatment of a solid tumor (such as bladder cancer, renal cell carcinoma, or melanoma) in an individual with a cancer vaccine (such as a gene encoding an antigen used in the preparation of the cancer vaccine, also referred to herein as a “cancer vaccine-associate gene”). In some embodiments, the at least one cancer vaccine-associated biomarker comprises a mutation of a cancer vaccine-associated gene, such as a mutation that results in a neo-antigen. In some embodiments, the at least one cancer vaccine-associated biomarker comprises a copy number variation of a cancer vaccine-associated gene. In some embodiments, the at least one cancer vaccine-associated biomarker comprises an aberrant expression level of a cancer vaccine-associated gene. In some embodiments, the at least one cancer vaccine-associated biomarker comprises an aberrant activity level of a cancer vaccine-associated gene. In some embodiments, the at least one cancer vaccine-associated biomarker comprises an aberrant phosphorylation level of the protein encoded by the cancer vaccine-associated gene. In some embodiments, the cancer vaccine-associated gene encodes a tumor-associated antigen (TAA), such as a neo-antigen. In some embodiments, the cancer vaccine is a vaccine prepared using autologous tumor cells. In some embodiments, the cancer vaccine is a vaccine prepared using allogeneic tumor cells. In some embodiments, the cancer vaccine is a vaccine prepared using a TAA.

In some embodiments, there is provided a method of treating a solid tumor (such as bladder cancer, renal cell carcinoma, or melanoma) in an individual comprising: (a) assessing at least one cancer vaccine-associated biomarker in the individual; and (b) administering to the individual i) an effective amount of a composition comprising nanoparticles comprising an mTOR inhibitor (such as a limus drug, e.g., sirolimus or a derivative thereof) and an albumin; and ii) an effective amount of a cancer vaccine, wherein the individual is selected for treatment based on having the at least one cancer vaccine-associated biomarker. In some embodiments, the at least one cancer vaccine-associated biomarker comprises a mutation of a cancer vaccine-associated gene, such as a mutation that results in a neo-antigen. In some embodiments, the at least one cancer vaccine-associated biomarker comprises a copy number variation of a cancer vaccine-associated gene. In some embodiments, the at least one cancer vaccine-associated biomarker comprises an aberrant expression level of a cancer vaccine-associated gene. In some embodiments, the at least one cancer vaccine-associated biomarker comprises an aberrant activity level of a cancer vaccine-associated gene. In some embodiments, the at least one cancer vaccine-associated biomarker comprises an aberrant phosphorylation level of the protein encoded by the cancer vaccine-associated gene. In some embodiments, the cancer vaccine-associated gene encodes a tumor-associated antigen (TAA), such as a neo-antigen. In some embodiments, the cancer vaccine is a vaccine prepared using autologous tumor cells. In some embodiments, the cancer vaccine is a vaccine prepared using allogeneic tumor cells. In some embodiments, the cancer vaccine is a vaccine prepared using a TAA.

In some embodiments, there is provided a method of treating a solid tumor (such as bladder cancer, renal cell carcinoma, or melanoma) in an individual comprising: (a) assessing at least one cancer vaccine-associated biomarker in the individual; (b) selecting (e.g., identifying or recommending) the individual for treatment based on the individual having the at least one cancer vaccine-associated biomarker; and (c) administering to the individual i) an effective amount of a composition comprising nanoparticles comprising an mTOR inhibitor (such as a limus drug, e.g., sirolimus or a derivative thereof) and an albumin; and ii) an effective amount of a cancer vaccine. In some embodiments, the at least one cancer vaccine-associated biomarker comprises a mutation of a cancer vaccine-associated gene. In some embodiments, the at least one cancer vaccine-associated biomarker comprises a copy number variation of a cancer vaccine-associated gene. In some embodiments, the at least one cancer vaccine-associated biomarker comprises an aberrant expression level of a cancer vaccine-associated gene. In some embodiments, the at least one cancer vaccine-associated biomarker comprises an aberrant activity level of a cancer vaccine-associated gene. In some embodiments, the at least one cancer vaccine-associated biomarker comprises an aberrant phosphorylation level of the protein encoded by the cancer vaccine-associated gene. In some embodiments, the cancer vaccine-associated gene encodes a tumor-associated antigen (TAA), such as a neo-antigen. In some embodiments, the cancer vaccine is a vaccine prepared using autologous tumor cells. In some embodiments, the cancer vaccine is a vaccine prepared using allogeneic tumor cells. In some embodiments, the cancer vaccine is a vaccine prepared using a TAA.

In some embodiments, there is provided a method of selecting (including identifying or recommending) an individual having a solid tumor (such as bladder cancer, renal cell carcinoma, or melanoma) for treatment with i) an effective amount of a composition comprising nanoparticles comprising an mTOR inhibitor (such as a limus drug, e.g., sirolimus or a derivative thereof) and an albumin; and ii) an effective amount of a cancer vaccine, wherein the method comprises (a) assessing at least one cancer vaccine-associated biomarker in the individual; and (b) selecting or recommending the individual for treatment based on the individual having the at least one cancer vaccine-associated biomarker. In some embodiments, the at least one cancer vaccine-associated biomarker comprises a mutation of a cancer vaccine-associated gene. In some embodiments, the at least one cancer vaccine-associated biomarker comprises a copy number variation of a cancer vaccine-associated gene. In some embodiments, the at least one cancer vaccine-associated biomarker comprises an aberrant expression level of a cancer vaccine-associated gene. In some embodiments, the at least one cancer vaccine-associated biomarker comprises an aberrant activity level of a cancer vaccine-associated gene. In some embodiments, the at least one cancer vaccine-associated biomarker comprises an aberrant phosphorylation level of the protein encoded by the cancer vaccine-associated gene. In some embodiments, the cancer vaccine-associated gene encodes a tumor-associated antigen (TAA), such as a neo-antigen. In some embodiments, the cancer vaccine is a vaccine prepared using autologous tumor cells. In some embodiments, the cancer vaccine is a vaccine prepared using allogeneic tumor cells. In some embodiments, the cancer vaccine is a vaccine prepared using a TAA.

In some embodiments, there is provided a method of selecting (including identifying or recommending) and treating an individual having a solid tumor (such as bladder cancer, renal cell carcinoma, or melanoma), wherein the method comprises (a) assessing at least one cancer vaccine-associated biomarker in the individual; (b) selecting or recommending the individual for treatment based on the individual having the at least one cancer vaccine-associated biomarker; and (c) administering to the individual i) an effective amount of a composition comprising an mTOR inhibitor (such as a limus drug, e.g., sirolimus or a derivative thereof) and an albumin; and ii) an effective amount of a cancer vaccine. In some embodiments, the at least one cancer vaccine-associated biomarker comprises a mutation of a cancer vaccine-associated gene. In some embodiments, the at least one cancer vaccine-associated biomarker comprises a copy number variation of a cancer vaccine-associated gene. In some embodiments, the at least one cancer vaccine-associated biomarker comprises an aberrant expression level of a cancer vaccine-associated gene. In some embodiments, the at least one cancer vaccine-associated biomarker comprises an aberrant activity level of a cancer vaccine-associated gene. In some embodiments, the at least one cancer vaccine-associated biomarker comprises an aberrant phosphorylation level of the protein encoded by the cancer vaccine-associated gene. In some embodiments, the cancer vaccine-associated gene encodes a tumor-associated antigen (TAA), such as a neo-antigen. In some embodiments, the cancer vaccine is a vaccine prepared using autologous tumor cells. In some embodiments, the cancer vaccine is a vaccine prepared using allogeneic tumor cells. In some embodiments, the cancer vaccine is a vaccine prepared using a TAA.

Also provided herein are methods of assessing whether an individual with a solid tumor (such as bladder cancer, renal cell carcinoma, or melanoma) is more likely to respond or less likely to respond to treatment with i) an effective amount of a composition comprising an mTOR inhibitor (such as a limus drug, e.g., sirolimus or a derivative thereof) and an albumin; and ii) an effective amount of a cancer vaccine, the method comprising assessing the at least one cancer vaccine-associated biomarker in the individual. In some embodiments, the method further comprises administering to the individual who is determined to be likely to respond to the treatment i) an effective amount of a composition comprising an mTOR inhibitor (such as a limus drug, e.g., sirolimus or a derivative thereof) and an albumin; and ii) an effective amount of a cancer vaccine. In some embodiments, the presence of the at least one cancer vaccine-associated biomarker indicates that the individual is more likely to respond to the treatment, and the absence of the at least one cancer vaccine-associated biomarker indicates that the individual is less likely to respond to the treatment. In some embodiments, the amount of the cancer vaccine is determined based on the presence of the at least one cancer vaccine-associated biomarker in the individual. In some embodiments, the cancer vaccine is selected from the group consisting of nilotinib and sorafenib.

Also provided herein are methods of adjusting therapy treatment of an individual with a solid tumor (such as bladder cancer, renal cell carcinoma, or melanoma) receiving i) an effective amount of a composition comprising an mTOR inhibitor (such as a limus drug, e.g., sirolimus or a derivative thereof) and an albumin; and ii) an effective amount of a cancer vaccine, the method comprising assessing at least one cancer vaccine-associated biomarker in a sample isolated from the individual, and adjusting the therapy treatment based on the individual having the at least one cancer vaccine-associated biomarker. In some embodiments, the amount of the cancer vaccine is adjusted.

Further contemplated are combinations of the methods described in this section, such that treatment of an individual may depend on the presence of an mTOR-activating aberration and any of the immunomodulator-, HDACi-, kinase inhibitor-, and cancer vaccine-associated biomarkers described herein.

mTOR-Activating Aberrations

The present application contemplates mTOR-activating aberrations in any one or more mTOR-associated genes described above, including deviations from the reference sequences (i.e. genetic aberrations), abnormal expression levels and/or abnormal activity levels of the one or more mTOR-associated genes. The present application encompasses treatments and methods based on the status of any one or more of the mTOR-activating aberrations disclosed herein.

The mTOR-activating aberrations described herein are associated with an increased (i.e. hyperactivated) mTOR signaling level or activity level. The mTOR signaling level or mTOR activity level described in the present application may include mTOR signaling in response to any one or any combination of the upstream signals described above, and may include mTOR signaling through mTORC1 and/or mTORC2, which may lead to measurable changes in any one or combinations of downstream molecular, cellular or physiological processes (such as protein synthesis, autophagy, metabolism, cell cycle arrest, apoptosis etc.). In some embodiments, the mTOR-activating aberration hyperactivates the mTOR activity by at least about any one of 10%, 20%, 30%, 40%, 60%, 70%, 80%, 90%, 100%, 200%, 500% or more above the level of mTOR activity without the mTOR-activating aberration. In some embodiments, the hyperactivated mTOR activity is mediated by mTORC1 only. In some embodiments, the hyperactivated mTOR activity is mediated by mTORC2 only. In some embodiments, the hyperactivated mTOR activity is mediated by both mTORC1 and mTORC2.

Methods of determining mTOR activity are known in the art. See, for example, Brian C G et al., Cancer Discovery, 2014, 4:554-563. The mTOR activity may be measured by quantifying any one of the downstream outputs (e.g. at the molecular, cellular, and/or physiological level) of the mTOR signaling pathway as described above. For example, the mTOR activity through mTORC1 may be measured by determining the level of phosphorylated 4EBP1 (e.g. P-S65-4EBP1), and/or the level of phosphorylated S6K1 (e.g. P-T389-S6K1), and/or the level of phosphorylated AKT1 (e.g. P-S473-AKT1). The mTOR activity through mTORC2 may be measured by determining the level of phosphorylated FoxO1 and/or FoxO3a. The level of a phosphorylated protein may be determined using any method known in the art, such as Western blot assays using antibodies that specifically recognize the phosphorylated protein of interest.

Candidate mTOR-activating aberrations may be identified through a variety of methods, for example, by literature search or by experimental methods known in the art, including, but not limited to, gene expression profiling experiments (e.g. RNA sequencing or microarray experiments), quantitative proteomics experiments, and gene sequencing experiments. For example, gene expression profiling experiments and quantitative proteomics experiments conducted on a sample collected from an individual having a solid tumor (such as bladder cancer, renal cell carcinoma, or melanoma) compared to a control sample may provide a list of genes and gene products (such as RNA, protein, and phosphorylated protein) that are present at aberrant levels. In some instances, gene sequencing (such as exome sequencing) experiments conducted on a sample collected from an individual having a solid tumor (such as bladder cancer, renal cell carcinoma, or melanoma) compared to a control sample may provide a list of genetic aberrations. Statistical association studies (such as genome-wide association studies) may be performed on experimental data collected from a population of individuals having a solid tumor to associate aberrations (such as aberrant levels or genetic aberrations) identified in the experiments with solid tumor. In some embodiments, targeted sequencing experiments (such as the ONCOPANEL™ test) are conducted to provide a list of genetic aberrations in an individual having a solid tumor (such as cancer, restenosis, or pulmonary hypertension).

The ONCOPANEL™ test can be used to survey exonic DNA sequences of cancer related genes and intronic regions for detection of genetic aberrations, including somatic mutations, copy number variations and structural rearrangements in DNA from various sources of samples (such as a tumor biopsy or blood sample), thereby providing a candidate list of genetic aberrations that may be mTOR-activating aberrations. In some embodiments, the mTOR-associated gene aberration is a genetic aberration or an aberrant level (such as expression level or activity level) in a gene selected from the ONCOPANEL™ test (CLIA certified). See, for example, Wagle N. et al. Cancer discovery 2.1 (2012): 82-93.

An exemplary version of ONCOPANEL™ test includes 300 cancer genes and 113 introns across 35 genes. The 300 genes included in the exemplary ONCOPANEL™ test are: ABL1, AKT1, AKT2, AKT3, ALK, ALOX12B, APC, AR, ARAF, ARID1A, ARIDIB, ARID2, ASXL1, ATM, ATRX, AURKA, AURKB, AXL, B2M, BAP1, BCL2, BCL2L1, BCL2L12, BCL6, BCOR, BCORL1, BLM, BMPR1A, BRAF, BRCA1, BRCA2, BRD4, BRIP1, BUB1B, CADM2, CARD11, CBL, CBLB, CCND1, CCND2, CCND3, CCNE1, CD274, CD58, CD79B, CDC73, CDH1, CDK1, CDK2, CDK4, CDK5, CDK6, CDK9, CDKNlA, CDKN1B, CDKN1C, CDKN2A, CDKN2B, CDKN2C, CEBPA, CHEK2, CIITA, CREBBP, CRKL, CRLF2, CRTC1, CRTC2, CSF1R, CSF3R, CTNNB1, CUX1, CYLD, DDB2, DDR2, DEPDC5, DICERI, DIS3, DMD, DNMT3A, EED, EGFR, EP300, EPHA3, EPHA5, EPHA7, ERBB2, ERBB3, ERBB4, ERCC2, ERCC3, ERCC4, ERCC5, ESRI, ETV1, ETV4, ETV5, ETV6, EWSR1, EXT1, EXT2, EZH2, FAM46C, FANCA, FANCC, FANCD2, FANCE, FANCF, FANCG, FAS, FBXW7, FGFR1, FGFR2, FGFR3, FGFR4, FH, FKBP9, FLCN, FLT1, FLT3, FLT4, FUS, GATA3, GATA4, GATA6, GLI1, GLI2, GLI3, GNA11, GNAQ, GNAS, GNB2L1, GPC3, GSTM5, H3F3A, HNF1A, HRAS, ID3, IDH1, IDH2, IGF1R, IKZF1, IKZF3, INSIGI, JAK2, JAK3, KCNIP1, KDM5C, KDM6A, KDM6B, KDR, KEAP1, KIT, KRAS, LINC00894, LMO1, LMO2, LMO3, MAP2K1, MAP2K4, MAP3K1, MAPK1, MCL1, MDM2, MDM4, MECOM, MEF2B, MEN1, MET, MITF, MLH1, MLL (KMT2A), MLL2 (KTM2D), MPL, MSH2, MSH6, MTOR, MUTYH, MYB, MYBL1, MYC, MYCL1 (MYCL), MYCN, MYD88, NBN, NEGRI, NF1, NF2, NFE2L2, NFKBIA, NFKBIZ, NKX2-1, NOTCH1, NOTCH2, NPM1, NPRL2, NPRL3, NRAS, NTRK1, NTRK2, NTRK3, PALB2, PARK2, PAX5, PBRM1, PDCD1LG2, PDGFRA, PDGFRB, PHF6, PHOX2B, PIK3C2B, PIK3CA, PIK3R1, PIM1, PMS1, PMS2, PNRC1, PRAME, PRDM1, PRF1, PRKAR1A, PRKCI, PRKCZ, PRKDC, PRPF40B, PRPF8, PSMD13, PTCH1, PTEN, PTK2, PTPN11, PTPRD, QKI, RAD21, RAF1, RARA, RB1, RBL2, RECQL4, REL, RET, RFWD2, RHEB, RHPN2, ROS1, RPL26, RUNX1, SBDS, SDHA, SDHAF2, SDHB, SDHC, SDHD, SETBP1, SETD2, SF1, SF3B1, SH2B3, SLITRK6, SMAD2, SMAD4, SMARCA4, SMARCB1, SMC1A, SMC3, SMO, SOCS1, SOX2, SOX9, SQSTM1, SRC, SRSF2, STAG1, STAG2, STAT3, STAT6, STK11, SUFU, SUZ12, SYK, TCF3, TCF7L1, TCF7L2, TERC, TERT, TET2, TLR4, TNFAIP3, TP53, TSC1, TSC2, U2AF1, VHL, WRN, WT1, XPA, XPC, XPO1, ZNF217, ZNF708, ZRSR2. The intronic regions surveyed in the exemplary ONCOPANEL™ test are tiled on specific introns of ABL1, AKT3, ALK, BCL2, BCL6, BRAF, CIITA, EGFR, ERG, ETV1, EWSR1, FGFR1, FGFR2, FGFR3, FUS, IGH, IGL, JAK2, MLL, MYC, NPM1, NTRK1, PAX5, PDGFRA, PDGFRB, PPARG, RAF1, RARA, RET, ROS1, SS18, TRA, TRB, TRG, TMPRSS2. mTOR-activating aberrations (such as genetic aberration and aberrant levels) of any of the genes included in any embodiment or version of the ONCOPANEL™ test, including, but not limited to the genes and intronic regions listed above, are contemplated by the present application to serve as a basis for selecting an individual for treatment with the mTOR inhibitor nanoparticle compositions.

Whether a candidate genetic aberration or aberrant level is an mTOR-activating aberration can be determined with methods known in the art. Genetic experiments in cells (such as cell lines) or animal models may be performed to ascertain that the solid tumor-associated aberrations identified from all aberrations observed in the experiments are mTOR-activating aberrations. For example, a genetic aberration may be cloned and engineered in a cell line or animal model, and the mTOR activity of the engineered cell line or animal model may be measured and compared with corresponding cell line or animal model that do not have the genetic aberration. An increase in the mTOR activity in such experiment may indicate that the genetic aberration is a candidate mTOR-activating aberration, which may be tested in a clinical study.

### Genetic Aberrations

Genetic aberrations of one or more mTOR-associated genes may comprise a change to the nucleic acid (such as DNA and RNA) or protein sequence (i.e. mutation) or an epigenetic feature associated with an mTOR-associated gene, including, but not limited to, coding, non-coding, regulatory, enhancer, silencer, promoter, intron, exon, and untranslated regions of the mTOR-associated gene.

The genetic aberration may be a germline mutation (including chromosomal rearrangement), or a somatic mutation (including chromosomal rearrangement). In some embodiments, the genetic aberration is present in all tissues, including normal tissue and the solid tumor tissue, of the individual. In some embodiments, the genetic aberration is present only in the solid tumor tissue of the individual. In some embodiments, the genetic aberration is present only in a fraction of the solid tumor tissue.

In some embodiments, the mTOR-activating aberration comprises a mutation of an mTOR-associated gene, including, but not limited to, deletion, frameshift, insertion, indel, missense mutation, nonsense mutation, point mutation, single nucleotide variation (SNV), silent mutation, splice site mutation, splice variant, and translocation. In some embodiments, the mutation may be a loss of function mutation for a negative regulator of the mTOR signaling pathway or a gain of function mutation of a positive regulator of the mTOR signaling pathway.

In some embodiments, the genetic aberration comprises a copy number variation of an mTOR-associated gene. Normally, there are two copies of each mTOR-associated gene per genome. In some embodiments, the copy number of the mTOR-associated gene is amplified by the genetic aberration, resulting in at least about any of 3, 4, 5, 6, 7, 8, or more copies of the mTOR-associated gene in the genome. In some embodiments, the genetic aberration of the mTOR-associated gene results in loss of one or both copies of the mTOR-associated gene in the genome. In some embodiments, the copy number variation of the mTOR-associated gene is loss of heterozygosity of the mTOR-associated gene. In some embodiments, the copy number variation of the mTOR-associated gene is deletion of the mTOR-associated gene. In some embodiments, the copy number variation of the mTOR-associated gene is caused by structural rearrangement of the genome, including deletions, duplications, inversion, and translocation of a chromosome or a fragment thereof.

In some embodiments, the genetic aberration comprises an aberrant epigenetic feature associated with an mTOR-associated gene, including, but not limited to, DNA methylation, hydroxymethylation, aberrant histone binding, chromatin remodeling, and the like. In some embodiments, the promotor of the mTOR-associated gene is hypermethylated in the individual, for example by at least about any of 10%, 20%, 30%, 40%, 50%, 60%, 70%, 80%, 90%, or more compared to a control level (such as a clinically accepted normal level in a standardized test).

In some embodiments, the mTOR-activating aberration is a genetic aberration (such as a mutation or a copy number variation) in any one of the mTOR-associated genes described above. In some embodiments, the mTOR-activating aberration is a mutation or a copy number variation in one or more genes selected from AKT1, MTOR, PIK3CA, PIK3CG, TSC1, TSC2, RHEB, STK11, NF1, NF2, PTEN, TP53, FGFR4, and BAP1.

Genetic aberrations in mTOR-associated genes have been identified in various human cancers, including hereditary cancers and sporadic cancers. For example, germline inactivating mutations in TSC1/2 cause tuberous sclerosis, and patients with this condition are present with lesions that include skin and brain hamartomas, renal angiomyolipomas, and renal cell carcinoma (RCC) (Krymskaya V P et al. 2011 FASEB Journal 25(6): 1922-1933). PTEN hamartoma tumor syndrome (PHTS) is linked to inactivating germline PTEN mutations and is associated with a spectrum of clinical manifestations, including breast cancer, endometrial cancer, follicular thyroid cancer, hamartomas, and RCC (Legendre C. et al. 2003 Transplantation proceedings 35(3 Suppl): 151S-153S). In addition, sporadic kidney cancer has also been shown to harbor somatic mutations in several genes in the PI3K-Akt-mTOR pathway (e.g. AKT1, MTOR, PIK3CA, PTEN, RHEB, TSC1, TSC2) (Power L A, 1990 Am. J Hosp. Pharm. 475.5: 1033-1049; Badesch D B et al. 2010 Chest 137(2): 376-3871; Kim J C & Steinberg G D, 2001, The Journal of urology, 165(3): 745-756; McKiernan J. et al. 2010, J. Urol. 183(Suppl 4)). Of the top 50 significantly mutated genes identified by the Cancer Genome Atlas in clear cell renal cell carcinoma, the mutation rate is about 17% for gene mutations that converge on mTORC1 activation (Cancer Genome Atlas Research Network. “Comprehensive molecular characterization of clear cell renal cell carcinoma.” 2013 Nature 499: 43-49). Genetic aberrations in mTOR-associated genes have been found to confer sensitivity in individuals having cancer to treatment with a limus drug. See, for example, Wagle et al., N. Eng. J. Med. 2014, 371:1426-33; Iyer et al., Science 2012, 338: 221; Wagle et al. Cancer Discovery 2014, 4:546-553; Grabiner et al., Cancer Discovery 2014, 4:554-563; Dickson et al. Int J. Cancer 2013, 132(7): 1711-1717, and Lim et al, J Clin. Oncol. 33, 2015 suppl; abstr 11010. Genetic aberrations of mTOR-associated genes described by the above references are incorporated herein. Exemplary genetic aberrations in some mTOR-associated genes are described below, and it is understood that the present application is not limited to the exemplary genetic aberrations described herein.

In some embodiments, the mTOR-activating aberration comprises a genetic aberration in MTOR. In some embodiments, the genetic aberration comprises an activating mutation of MTOR. In some embodiments, the activating mutation of MTOR is at one or more positions (such as about any one of 1, 2, 3, 4, 5, 6, or more positions) in the protein sequence of MTOR selected from the group consisting of N269, L1357, N1421, L1433, A1459, L1460, C1483, E1519, K1771, E1799, F1888, 11973, T1977, V2006, E2014, 12017, N2206, L2209, A2210, S2215, L2216, R2217, L2220, Q2223, A2226, E2419, L2431, 12500, R2505, and D2512. In some embodiments, the activating mutation of MTOR is one or more missense mutations (such as about any one of 1, 2, 3, 4, 5, 6, or more mutations) selected from the group consisting of N269S, L1357F, N1421D, L1433S, A1459P, L1460P, C1483F, C1483R, C1483W, C1483Y, E1519T, K1771R, E1799K, F1888I, F1888I L, I1973F, T1977R, T1977K, V2006I, E2014K, I2017T, N2206S, L2209V, A2210P, S2215Y, S2215F, S2215P, L2216P, R2217W, L2220F, Q2223K, A2226S, E2419K, L2431P, I2500M, R2505P, and D2512H. In some embodiments, the activating mutation of MTOR disrupts binding of MTOR with RHEB. In some embodiments, the activating mutation of MTOR disrupts binding of MTOR with DEPTOR.

In some embodiments, the mTOR-activating aberration comprises a genetic aberration in TSC1 or TSC2. In some embodiments, the genetic aberration comprises a loss of heterozygosity of TSC1 or TSC2. In some embodiments, the genetic aberration comprises a loss of function mutation in TSC1 or TSC2. In some embodiments, the loss of function mutation is a frameshift mutation or a nonsense mutation in TSC1 or TSC2. In some embodiments, the loss of function mutation is a frameshift mutation c.1907_1908del in TSC1. In some embodiments, the loss of function mutation is a splice variant of TSC1: c.1019+1G>A. In some embodiments, the loss of function mutation is the nonsense mutation c.1073G>A in TSC2, and/or p.Trp103* in TSC1. In some embodiments, the loss of function mutation comprises a missense mutation in TSC1 or in TSC2. In some embodiments, the missense mutation is in position A256 of TSC1, and/or position Y719 of TSC2. In some embodiments, the missense mutation comprises A256V in TSC1 or Y719H in TSC2.

In some embodiments, the mTOR-activating aberration comprises a genetic aberration in RHEB. In some embodiments, the genetic aberration comprises a loss of function mutation in RHEB. In some embodiments, the loss of function mutation is at one or more positions in the protein sequence of RHEB selected from Y35 and E139. In some embodiments, the loss of function mutation in RHEB is selected from Y35N, Y35C, Y35H and E139K.

In some embodiments, the mTOR-activating aberration comprises a genetic aberration in NF1. In some embodiments, the genetic aberration comprises a loss of function mutation in NF1. In some embodiments, the loss of function mutation in NF1 is a missense mutation at position D1644 in NF1. In some embodiments, the missense mutation is D1644A in NF1.

In some embodiments, the mTOR-activating aberration comprises a genetic aberration in NF2. In some embodiments, the genetic aberration comprises a loss of function mutation in NF2. In some embodiments, the loss of function mutation in NF2 is a nonsense mutation. In some embodiments, the nonsense mutation in NF2 is c.863C>G.

In some embodiments, the mTOR-activating aberration comprises a genetic aberration in PTEN. In some embodiments, the genetic aberration comprises a deletion of PTEN in the genome.

In some embodiments, the mTOR-activating aberration comprises a genetic aberration in PI3K. In some embodiments, the genetic aberration comprises a loss of function mutation in PIK3CA or PIK3CG. In some embodiments, the loss of function mutation comprises a missense mutation at a position in PIK3CA selected from the group consisting of E542, I844, and H1047. In some embodiments, the loss of function mutation comprises a missense in PIK3CA selected from the group consisting of E542K, I844V, and H1047R.

In some embodiments, the mTOR-activating aberration comprises a genetic aberration in AKT1. In some embodiments, the genetic aberration comprises an activating mutation in AKT1. In some embodiments, the activating mutation is a missense mutation in position H238 in AKT1. In some embodiments, the missense mutation is H238Y in AKT1.

In some embodiments, the mTOR-activating aberration comprises a genetic aberration in TP53. In some embodiments, the genetic aberration comprises a loss of function mutation in TP53. In some embodiments, the loss of function mutation is a frameshift mutation in TP53, such as A39fs*5.

The genetic aberrations of the mTOR-associated genes may be assessed based on a sample, such as a sample from the individual and/or reference sample. In some embodiments, the sample is a tissue sample or nucleic acids extracted from a tissue sample. In some embodiments, the sample is a cell sample (for example a CTC sample) or nucleic acids extracted from a cell sample. In some embodiments, the sample is a tumor biopsy. In some embodiments, the sample is a tumor sample or nucleic acids extracted from a tumor sample. In some embodiments, the sample is a biopsy sample or nucleic acids extracted from the biopsy sample. In some embodiments, the sample is a Formaldehyde Fixed-Paraffin Embedded (FFPE) sample or nucleic acids extracted from the FFPE sample. In some embodiments, the sample is a blood sample. In some embodiments, cell-free DNA is isolated from the blood sample. In some embodiments, the biological sample is a plasma sample or nucleic acids extracted from the plasma sample.

The genetic aberrations of the mTOR-associated gene may be determined by any method known in the art. See, for example, Dickson et al. Int. J Cancer, 2013, 132(7): 1711-1717; Wagle N. Cancer Discovery, 2014, 4:546-553; and Cancer Genome Atlas Research Network. Nature 2013, 499: 43-49. Exemplary methods include, but are not limited to, genomic DNA sequencing, bisulfite sequencing or other DNA sequencing-based methods using Sanger sequencing or next generation sequencing platforms; polymerase chain reaction assays; in situ hybridization assays; and DNA microarrays. The epigenetic features (such as DNA methylation, histone binding, or chromatin modifications) of one or more mTOR-associated genes from a sample isolated from the individual may be compared with the epigenetic features of the one or more mTOR-associated genes from a control sample. The nucleic acid molecules extracted from the sample can be sequenced or analyzed for the presence of the mTOR-activating genetic aberrations relative to a reference sequence, such as the wildtype sequences of AKT1, FLT-3, MTOR, PIK3CA, PIK3CG, TSC1, TSC2, RHEB, STK11, NF1, NF2, TP53, FGFR4, BAP1, KRAS, NRAS and PTEN.

In some embodiments, the genetic aberration of an mTOR-associated gene is assessed using cell-free DNA sequencing methods. In some embodiments, the genetic aberration of an mTOR-associated gene is assessed using next-generation sequencing. In some embodiments, the genetic aberration of an mTOR-associated gene isolated from a blood sample is assessed using next-generation sequencing. In some embodiments, the genetic aberration of an mTOR-associated gene is assessed using exome sequencing. In some embodiments, the genetic aberration of an mTOR-associated gene is assessed using fluorescence in-situ hybridization analysis. In some embodiments, the genetic aberration of an mTOR-associated gene is assessed prior to initiation of the methods of treatment described herein. In some embodiments, the genetic aberration of an mTOR-associated gene is assessed after initiation of the methods of treatment described herein. In some embodiments, the genetic aberration of an mTOR-associated gene is assessed prior to and after initiation of the methods of treatment described herein.

### Aberrant Levels

An aberrant level of an mTOR-associated gene may refer to an aberrant expression level or an aberrant activity level.

Aberrant expression level of an mTOR-associated gene comprises an increase or decrease in the level of a molecule encoded by the mTOR-associated gene compared to the control level. The molecule encoded by the mTOR-associated gene may include RNA transcript(s) (such as mRNA), protein isoform(s), phosphorylated and/or dephosphorylated states of the protein isoform(s), ubiquitinated and/or de-ubiquitinated states of the protein isoform(s), membrane localized (e.g. myristoylated, palmitoylated, and the like) states of the protein isoform(s), other post-translationally modified states of the protein isoform(s), or any combination thereof.

Aberrant activity level of an mTOR-associated gene comprises enhancement or repression of a molecule encoded by any downstream target gene of the mTOR-associated gene, including epigenetic regulation, transcriptional regulation, translational regulation, post-translational regulation, or any combination thereof of the downstream target gene. Additionally, activity of an mTOR-associated gene comprises downstream cellular and/or physiological effects in response to the mTOR-activating aberration, including, but not limited to, protein synthesis, cell growth, proliferation, signal transduction, mitochondria metabolism, mitochondria biogenesis, stress response, cell cycle arrest, autophagy, microtubule organization, and lipid metabolism.

In some embodiments, the mTOR-activating aberration (e.g. aberrant expression level) comprises an aberrant protein phosphorylation level. In some embodiments, the aberrant phosphorylation level is in a protein encoded by an mTOR-associated gene selected from the group consisting of AKT, TSC2, mTOR, PRAS40, S6K, S6, and 4EBPT. Exemplary phosphorylated species of mTOR-associated genes that may serve as relevant biomarkers include, but are not limited to, AKT S473 phosphorylation, PRAS40 T246 phosphorylation, mTOR S2448 phosphorylation, 4EBP1 T36 phosphorylation, S6K T389 phosphorylation, 4EBP1 T70 phosphorylation, and S6 S235 phosphorylation. In some embodiments, the individual is selected for treatment if the protein in the individual is phosphorylated. In some embodiments, the individual is selected for treatment if the protein in the individual is not phosphorylated. In some embodiments, the phosphorylation status of the protein is determined by immunohistochemistry.

Aberrant levels of mTOR-associates genes have been associated with cancer, such as solid tumors. For example, high levels (74%) of phosphorylated mTOR expression were found in human bladder cancer tissue array, and phosphorylated mTOR intensity was associated with reduced survival (Hansel D E et al, (2010) Am. J. Pathol. 176: 3062-3072). mTOR expression was shown to increase as a function of the disease stage in progression from superficial disease to invasive bladder cancer, as evident by activation of pS6-kinase, which was activated in 54 of 70 cases (77%) of T2 muscle-invasive bladder tumors (Seager C M et al, (2009) Cancer Prev. Res. (Phila) 2, 1008-1014). The mTOR signaling pathway is also known to be hyperactivated in pulmonary arterial hypertension.

The levels (such as expression levels and/or activity levels) of an mTOR-associated gene in an individual may be determined based on a sample (e.g., sample from the individual or reference sample). In some embodiments, the sample is from a tissue, organ, cell, or tumor. In some embodiments, the sample is a biological sample. In some embodiments, the biological sample is a biological fluid sample or a biological tissue sample. In further embodiments, the biological fluid sample is a bodily fluid. In some embodiments, the sample is a solid tumor tissue, normal tissue adjacent to said solid tumor tissue, normal tissue distal to said solid tumor tissue, blood sample, or other biological sample. In some embodiments, the sample is a fixed sample. Fixed samples include, but are not limited to, a formalin fixed sample, a paraffin-embedded sample, or a frozen sample. In some embodiments, the sample is a biopsy containing solid tumor cells. In a further embodiment, the biopsy is a fine needle aspiration of solid tumor cells. In a further embodiment, the biopsy is laparoscopy obtained solid tumor cells. In some embodiments, the biopsied cells are centrifuged into a pellet, fixed, and embedded in paraffin. In some embodiments, the biopsied cells are flash frozen. In some embodiments, the biopsied cells are mixed with an antibody that recognizes a molecule encoded by the mTOR-associated gene. In some embodiments, the at least one mTOR-associated gene comprises enhancement or repression of a molecule encoded by any downstream target gene of the mTOR-associated gene, including epigenetic regulation, transcriptional regulation, translational regulation, post-translational regulation, or any combination thereof of the downstream target gene. Additionally, activity of an mTOR-associated gene comprises downstream cellular and/or physiological effects in response to the mTOR-activating aberration, including, but not limited to, protein synthesis, cell growth, proliferation, signal transduction, mitochondria metabolism, mitochondria biogenesis, stress response, cell cycle arrest, autophagy, microtubule organization, and lipid metabolism.

In some embodiments, the mTOR-activating aberration (e.g. aberrant expression level) comprises an aberrant protein phosphorylation level. In some embodiments, the aberrant phosphorylation level is in a protein encoded by an mTOR-associated gene selected from the group consisting of AKT, TSC2, mTOR, PRAS40, S6K, S6, and 4EBP1. Exemplary phosphorylated species of mTOR-associated genes that may serve as relevant biomarkers include, but are not limited to, AKT S473 phosphorylation, PRAS40 T246 phosphorylation, mTOR S2448 phosphorylation, 4EBP1 T36 phosphorylation, S6K T389 phosphorylation, 4EBP1 T70 phosphorylation, and S6 S235 phosphorylation. In some embodiments, the individual is selected for treatment if the protein in the individual is phosphorylated. In some embodiments, the individual is selected for treatment if the protein in the individual is not phosphorylated. In some embodiments, the phosphorylation status of the protein is determined by immunohistochemistry.

Aberrant levels of mTOR-associates genes have been associated with cancer, such as solid tumors. For example, high levels (74%) of phosphorylated mTOR expression were found in human bladder cancer tissue array, and phosphorylated mTOR intensity was associated with reduced survival (Hansel D E et al, (2010) Am. J. Pathol. 176: 3062-3072). mTOR expression was shown to increase as a function of the disease stage in progression from superficial disease to invasive bladder cancer, as evident by activation of pS6-kinase, which was activated in 54 of 70 cases (77%) of T2 muscle-invasive bladder tumors (Seager C M et al, (2009) Cancer Prev. Res. (Phila) 2, 1008-1014). The mTOR signaling pathway is also known to be hyperactivated in pulmonary arterial hypertension.

The levels (such as expression levels and/or activity levels) of an mTOR-associated gene in an individual may be determined based on a sample (e.g., sample from the individual or reference sample). In some embodiments, the sample is from a tissue, organ, cell, or tumor. In some embodiments, the sample is a biological sample. In some embodiments, the biological sample is a biological fluid sample or a biological tissue sample. In further embodiments, the biological fluid sample is a bodily fluid. In some embodiments, the sample is a solid tumor tissue, normal tissue adjacent to said solid tumor tissue, normal tissue distal to said solid tumor tissue, blood sample, or other biological sample. In some embodiments, the sample is a fixed sample. Fixed samples include, but are not limited to, a formalin fixed sample, a paraffin-embedded sample, or a frozen sample. In some embodiments, the sample is a biopsy containing solid tumor cells. In a further embodiment, the biopsy is a fine needle aspiration of solid tumor cells. In a further embodiment, the biopsy is laparoscopy obtained solid tumor cells. In some embodiments, the biopsied cells are centrifuged into a pellet, fixed, and embedded in paraffin. In some embodiments, the biopsied cells are flash frozen. In some embodiments, the biopsied cells are mixed with an antibody that recognizes a molecule encoded by the mTOR-associated biomarker comprises an aberrant phosphorylation level of the protein encoded by the mTOR-associated gene comprises enhancement or repression of a molecule encoded by any downstream target gene of the mTOR-associated gene, including epigenetic regulation, transcriptional regulation, translational regulation, post-translational regulation, or any combination thereof of the downstream target gene. Additionally, activity of an mTOR-associated gene comprises downstream cellular and/or physiological effects in response to the mTOR-activating aberration, including, but not limited to, protein synthesis, cell growth, proliferation, signal transduction, mitochondria metabolism, mitochondria biogenesis, stress response, cell cycle arrest, autophagy, microtubule organization, and lipid metabolism.

In some embodiments, the mTOR-activating aberration (e.g. aberrant expression level) comprises an aberrant protein phosphorylation level. In some embodiments, the aberrant phosphorylation level is in a protein encoded by an mTOR-associated gene selected from the group consisting of AKT, TSC2, mTOR, PRAS40, S6K, S6, and 4EBP1. Exemplary phosphorylated species of mTOR-associated genes that may serve as relevant biomarkers include, but are not limited to, AKT S473 phosphorylation, PRAS40 T246 phosphorylation, mTOR S2448 phosphorylation, 4EBP1 T36 phosphorylation, S6K T389 phosphorylation, 4EBP1 T70 phosphorylation, and S6 S235 phosphorylation. In some embodiments, the individual is selected for treatment if the protein in the individual is phosphorylated. In some embodiments, the individual is selected for treatment if the protein in the individual is not phosphorylated. In some embodiments, the phosphorylation status of the protein is determined by immunohistochemistry.

Aberrant levels of mTOR-associates genes have been associated with cancer, such as solid tumors. For example, high levels (74%) of phosphorylated mTOR expression were found in human bladder cancer tissue array, and phosphorylated mTOR intensity was associated with reduced survival (Hansel D E et al, (2010) Am. J Pathol. 176: 3062-3072). mTOR expression was shown to increase as a function of the disease stage in progression from superficial disease to invasive bladder cancer, as evident by activation of pS6-kinase, which was activated in 54 of 70 cases (77%) of T2 muscle-invasive bladder tumors (Seager C M et al, (2009) Cancer Prev. Res. (Phila) 2, 1008-1014). The mTOR signaling pathway is also known to be hyperactivated in pulmonary arterial hypertension.

The levels (such as expression levels and/or activity levels) of an mTOR-associated gene in an individual may be determined based on a sample (e.g., sample from the individual or reference sample). In some embodiments, the sample is from a tissue, organ, cell, or tumor. In some embodiments, the sample is a biological sample. In some embodiments, the biological sample is a biological fluid sample or a biological tissue sample. In further embodiments, the biological fluid sample is a bodily fluid. In some embodiments, the sample is a solid tumor tissue, normal tissue adjacent to said solid tumor tissue, normal tissue distal to said solid tumor tissue, blood sample, or other biological sample. In some embodiments, the sample is a fixed sample. Fixed samples include, but are not limited to, a formalin fixed sample, a paraffin-embedded sample, or a frozen sample. In some embodiments, the sample is a biopsy containing solid tumor cells. In a further embodiment, the biopsy is a fine needle aspiration of solid tumor cells. In a further embodiment, the biopsy is laparoscopy obtained solid tumor cells. In some embodiments, the biopsied cells are centrifuged into a pellet, fixed, and embedded in paraffin. In some embodiments, the biopsied cells are flash frozen. In some embodiments, the biopsied cells are mixed with an antibody that recognizes a molecule encoded by the mTOR-associated gene. In some embodiments, a biopsy is taken to determine whether an individual has a solid tumor (such as bladder cancer, renal cell carcinoma, or melanoma) and is then used as a sample. In some embodiments, the sample comprises surgically obtained solid tumor cells. In some embodiments, samples may be obtained at different times than when the determining of expression levels of mTOR-associated gene occurs. In some embodiments, the sample comprises a circulating metastatic cancer cell. In some embodiments, the sample is obtained by sorting circulating tumor cells (CTCs) from blood. In a further embodiment, the CTCs have detached from a primary tumor and circulate in a bodily fluid. In yet a further embodiment, the CTCs have detached from a primary tumor and circulate in the bloodstream. In a further embodiment, the CTCs are an indication of metastasis.

In some embodiments, the level of a protein encoded by an mTOR-associated gene is determined to assess the aberrant expression level of the mTOR-associated gene. In some embodiments, the level of a protein encoded by a downstream target gene of an mTOR-associated gene is determined to assess the aberrant activity level of the mTOR-associated gene. In some embodiments, protein level is determined using one or more antibodies specific for one or more epitopes of the individual protein or proteolytic fragments thereof. Detection methodologies suitable for use in the practice of the invention include, but are not limited to, immunohistochemistry, enzyme linked immunosorbent assays (ELISAs), Western blotting, mass spectroscopy, and immuno-PCR. In some embodiments, levels of protein(s) encoded by the mTOR-associated gene and/or downstream target gene(s) thereof in a sample are normalized (such as divided) by the level of a housekeeping protein (such as glyceraldehyde 3-phosphate dehydrogenase, or GAPDH) in the same sample.

In some embodiments, the level of an mRNA encoded by an mTOR-associated gene is determined to assess the aberrant expression level of the mTOR-associated gene. In some embodiments, the level of an mRNA encoded by a downstream target gene of an mTOR-associated gene is determined to assess the aberrant activity level of the mTOR-associated gene. In some embodiments, a reverse-transcription (RT) polymerase chain reaction (PCR) assay (including a quantitative RT-PCR assay) is used to determine the mRNA levels. In some embodiments, a gene chip or next-generation sequencing methods (such as RNA (cDNA) sequencing or exome sequencing) are used to determine the levels of RNA (such as mRNA) encoded by the mTOR-associated gene and/or downstream target genes thereof. In some embodiments, an mRNA level of the mTOR-associated gene and/or downstream target genes thereof in a sample are normalized (such as divided) by the mRNA level of a housekeeping gene (such as GAPDH) in the same sample.

The levels of an mTOR-associated gene may be a high level or a low level as compared to a control or reference. In some embodiments, wherein the mTOR-associated gene is a positive regulator of the mTOR activity (such as mTORC1 and/or mTORC2 activity), the aberrant level of the mTOR associated gene is a high level compared to the control. In some embodiments, wherein the mTOR-associated gene is a negative regulator of the mTOR activity (such as mTORC1 and/or mTORC2 activity), the aberrant level of the mTOR associated gene is a low level compared to the control.

In some embodiments, the level of the mTOR-associated gene in an individual is compared to the level of the mTOR-associated gene in a control sample. In some embodiments, the level of the mTOR-associated gene in an individual is compared to the level of the mTOR-associated gene in multiple control samples. In some embodiments, multiple control samples are used to generate a statistic that is used to classify the level of the mTOR-associated gene in an individual with a solid tumor (such as bladder cancer, renal cell carcinoma, or melanoma).

The classification or ranking of the level (i.e., high or low) of the mTOR-associated gene may be determined relative to a statistical distribution of control levels. In some embodiments, the classification or ranking is relative to a control sample, such as a normal tissue (e.g. peripheral blood mononuclear cells), or a normal epithelial cell sample (e.g. a buccal swap or a skin punch) obtained from the individual. In some embodiments, the level of the mTOR-associated gene is classified or ranked relative to a statistical distribution of control levels. In some embodiments, the level of the mTOR-associated gene is classified or ranked relative to the level from a control sample obtained from the individual.

Control samples can be obtained using the same sources and methods as non-control samples. In some embodiments, the control sample is obtained from a different individual (for example an individual not having the solid tumor (such as bladder cancer, renal cell carcinoma, or melanoma); an individual having a benign or less advanced form of a disease corresponding to the solid tumor; and/or an individual sharing similar ethnic, age, and gender). In some embodiments when the sample is a tumor tissue sample, the control sample may be a non-cancerous sample from the same individual. In some embodiments, multiple control samples (for example from different individuals) are used to determine a range of levels of the mTOR-associated genes in a particular tissue, organ, or cell population.

In some embodiments, the control sample is a cultured tissue or cell that has been determined to be a proper control. In some embodiments, the control is a cell that does not have the mTOR-activating aberration. In some embodiments, a clinically accepted normal level in a standardized test is used as a control level for determining the aberrant level of the mTOR-associated gene. In some embodiments, the level of the mTOR-associated gene or downstream target genes thereof in the individual is classified as high, medium or low according to a scoring system, such as an immunohistochemistry-based scoring system.

In some embodiments, the level of the mTOR-associated gene is determined by measuring the level of the mTOR-associated gene in an individual and comparing to a control or reference (e.g., the median level for the given patient population or level of a second individual). For example, if the level of the mTOR-associated gene for the single individual is determined to be above the median level of the patient population, that individual is determined to have high expression level of the mTOR-associated gene. Alternatively, if the level of the mTOR-associated gene for the single individual is determined to be below the median level of the patient population, that individual is determined to have low expression level of the mTOR-associated gene. In some embodiments, the individual is compared to a second individual and/or a patient population which is responsive to the treatment. In some embodiments, the individual is compared to a second individual and/or a patient population which is not responsive to the treatment. In some embodiments, the levels are determined by measuring the level of a nucleic acid encoded by the mTOR-associated gene and/or a downstream target gene thereof. For example, if the level of a molecule (such as an mRNA or a protein) encoded by the mTOR-associated gene for the single individual is determined to be above the median level of the patient population, that individual is determined to have a high level of the molecule (such as mRNA or protein) encoded by the mTOR-associated gene. Alternatively, if the level of a molecule (such as an mRNA or a protein) encoded by the mTOR-associated gene for the single individual is determined to be below the median level of the patient population, that individual is determined to have a low level of the molecule (such as mRNA or protein) encoded by the mTOR-associated gene.

In some embodiments, the control level of an mTOR-associated gene is determined by obtaining a statistical distribution of the levels of mTOR-associated gene. In some embodiments, the level of the mTOR-associated gene is classified or ranked relative to control levels or a statistical distribution of control levels.

In some embodiments, bioinformatics methods are used for the determination and classification of the levels of the mTOR-associated gene, including the levels of downstream target genes of the mTOR-associated gene as a measure of the activity level of the mTOR-associated gene. Numerous bioinformatics approaches have been developed to assess gene set expression profiles using gene expression profiling data. Methods include but are not limited to those described in Segal, E. et al. Nat. Genet. 34:66-176 (2003); Segal, E. et al. Nat. Genet. 36:1090-1098 (2004); Barry, W. T. et al. Bioinformatics 21:1943-1949 (2005); Tian, L. et al. Proc Nat'l Acad Sci USA 102:13544-13549 (2005); Novak B A and Jain A N. Bioinformatics 22:233-41 (2006); Maglietta R et al. Bioinformatics 23:2063-72 (2007); Bussemaker H J, BMC Bioinformatics 8 Suppl 6:S6 (2007).

In some embodiments, the control level is a pre-determined threshold level. In some embodiments, mRNA level is determined, and a low level is an mRNA level less than about any of 1, 0.9, 0.8, 0.7, 0.6, 0.5, 0.4, 0.3, 0.2, 0.1, 0.05, 0.02, 0.01, 0.005, 0.002, 0.001 or less time that of what is considered as clinically normal or of the level obtained from a control. In some embodiments, a high level is an mRNA level more than about 1.1, 1.2, 1.3, 1.5, 1.7, 2, 2.2, 2.5, 2.7, 3, 5, 7, 10, 20, 50, 70, 100, 200, 500, 1000 times or more than 1000 times that of what is considered as clinically normal or of the level obtained from a control.

In some embodiments, protein expression level is determined, for example by Western blot or an enzyme-linked immunosorbent assay (ELISA). For example, the criteria for low or high levels can be made based on the total intensity of a band on a protein gel corresponding to the protein encoded by the mTOR-associated gene that is blotted by an antibody that specifically recognizes the protein encoded by the mTOR-associated gene, and normalized (such as divided) by a band on the same protein gel of the same sample corresponding to a housekeeping protein (such as GAPDH) that is blotted by an antibody that specifically recognizes the housekeeping protein (such as GAPDH). In some embodiments, the protein level is low if the protein level is less than about any of 1, 0.9, 0.8, 0.7, 0.6, 0.5, 0.4, 0.3, 0.2, 0.1, 0.05, 0.02, 0.01, 0.005, 0.002, 0.001 or less time of what is considered as clinically normal or of the level obtained from a control. In some embodiments, the protein level is high if the protein level is more than about any of 1.1, 1.2, 1.3, 1.5, 1.7, 2, 2.2, 2.5, 2.7, 3, 5, 7, 10, 20, 50, or 100 times or more than 100 times of what is considered as clinically normal or of the level obtained from a control.

In some embodiments, protein expression level is determined, for example by immunohistochemistry. For example, the criteria for low or high levels can be made based on the number of positive staining cells and/or the intensity of the staining, for example by using an antibody that specifically recognizes the protein encoded by the mTOR-associated gene. In some embodiments, the level is low if less than about 1%, 5%, 10%, 15%, 20%, 25%, 30%, 35%, 40%, 45%, or 50% cells have positive staining. In some embodiments, the level is low if the staining is 1%, 5%, 10%, 15%, 20%, 25%, 30%, 35%, 40%, 45%, or 50% less intense than a positive control staining. In some embodiments, the level is high if more than about 40%, 45%, 50%, 55%, 60%, 65%, 70%, 75%, 80%, 85%, or 90%, cells have positive staining. In some embodiments, the level is high if the staining is as intense as positive control staining. In some embodiments, the level is high if the staining is 80%, 85%, or 90% as intense as positive control staining.

In some embodiments, the scoring is based on an “H-score” as described in US Pat. Pub. No. 2013/0005678. An H-score is obtained by the formula: 3×percentage of strongly staining cells+2×percentage of moderately staining cells+percentage of weakly staining cells, giving a range of 0 to 300.

In some embodiments, strong staining, moderate staining, and weak staining are calibrated levels of staining, wherein a range is established and the intensity of staining is binned within the range. In some embodiments, strong staining is staining above the 75th percentile of the intensity range, moderate staining is staining from the 25th to the 75th percentile of the intensity range, and low staining is staining is staining below the 25th percentile of the intensity range. In some aspects one skilled in the art, and familiar with a particular staining technique, adjusts the bin size and defines the staining categories.

In some embodiments, the label high staining is assigned where greater than 50% of the cells stained exhibited strong reactivity, the label no staining is assigned where no staining was observed in less than 50% of the cells stained, and the label low staining is assigned for all of other cases.

In some embodiments, the assessment and/or scoring of the genetic aberration or the level of the mTOR-associated gene in a sample, patient, etc., is performed by one or more experienced clinicians, i.e., those who are experienced with the mTOR-associated gene expression and the mTOR-associated gene product staining patterns. For example, in some embodiments, the clinician(s) is blinded to clinical characteristics and outcome for the samples, patients, etc. being assessed and scored.

In some embodiments, level of protein phosphorylation is determined. The phosphorylation status of a protein may be assessed from a variety of sample sources. In some embodiments, the sample is a tumor biopsy. The phosphorylation status of a protein may be assessed via a variety of methods. In some embodiments, the phosphorylation status is assessed using immunohistochemistry. The phosphorylation status of a protein may be site specific. The phosphorylation status of a protein may be compared to a control sample. In some embodiments, the phosphorylation status is assessed prior to initiation of the methods of treatment described herein. In some embodiments, the phosphorylation status is assessed after initiation of the methods of treatment described herein. In some embodiments, the phosphorylation status is assessed prior to and after initiation of the methods of treatment described herein.

Further provided herein are methods of directing treatment of a solid tumor (such as bladder cancer, renal cell carcinoma, or melanoma) by delivering a sample to a diagnostic lab for determination of the level of an mTOR-associated gene; providing a control sample with a known level of the mTOR-associated gene; providing an antibody to a molecule encoded by the mTOR-associated gene or an antibody to a molecule encoded by a downstream target gene of the mTOR-associated gene; individually contacting the sample and control sample with the antibody, and/or detecting a relative amount of antibody binding, wherein the level of the sample is used to provide a conclusion that a patient should receive a treatment with any one of the methods described herein.

Also provided herein are methods of directing treatment of a solid tumor (such as bladder cancer, renal cell carcinoma, or melanoma), further comprising reviewing or analyzing data relating to the status (such as presence/absence or level) of an mTOR-activating aberration in a sample; and providing a conclusion to an individual, such as a health care provider or a health care manager, about the likelihood or suitability of the individual to respond to a treatment, the conclusion being based on the review or analysis of data. In one aspect of the invention a conclusion is the transmission of the data over a network.

### Resistance Biomarkers

Genetic aberrations and aberrant levels of certain genes may be associated with resistance to the treatment methods described herein. In some embodiments, the individual having an aberration (such as genetic aberration or aberrant level) in a resistance biomarker is excluded from the methods of treatment using the mTOR inhibitor nanoparticles as described herein. In some embodiments, the status of the resistance biomarkers combined with the status of one or more of the mTOR-activating aberrations are used as the basis for selecting an individual for any one of the methods of treatment using mTOR inhibitor nanoparticles as described herein.

For example, TFE3, also known as transcription factor binding to IGHM enhancer 3, TFEA, RCCP2, RCCX1, or bHLHe33, is a transcription factor that specifically recognizes and binds MUE3-type E-box sequences in the promoters of genes. TFE3 promotes expression of genes downstream of transforming growth factor beta (TGF-beta) signaling. Translocation of TFE3 has been associated with renal cell carcinomas and other cancers. In some embodiments, the nucleic acid sequence of a wildtype TFE3 gene is identified by the Genbank accession number NC_000023.11 from nucleotide 49028726 to nucleotide 49043517 of the complement strand of chromosome X according to the GRCh38.p2 assembly of the human genome. Exemplary translocations of TFE3 that may be associated with resistance to treatment using the mTOR inhibitor nanoparticles as described herein include, but are not limited to, Xp11 translocation, such as t(X; 1)(p11.2; q21), t(X; 1)(p11.2; p34), (X; 17)(p11.2; q25.3), and inv(X)(p11.2; q12). Translocation of the TFE3 locus can be assessed using immunohistochemical methods or fluorescence in situ hybridization (FISH).

### Dosing and Method of Administering

The dose of the mTOR inhibitor nanoparticle composition (such as sirolimus/albumin nanoparticle composition) administered to an individual (e.g., a human) in combination therapy may vary with the particular composition, the method of administration, and the particular stage of solid tumor being treated. The amount should be sufficient to produce a desirable response, such as a therapeutic or prophylactic response against solid tumor. In some embodiments, the amount of mTOR inhibitor (such as a limus drug, e.g., sirolimus or a derivative thereof) in the composition is below the level that induces a toxicological effect (e.g., an effect above a clinically acceptable level of toxicity) or is at a level where a potential side effect can be controlled or tolerated when the mTOR inhibitor nanoparticle composition is administered to the individual.

In some embodiments, the mTOR inhibitor nanoparticle composition (such as sirolimus/albumin nanoparticle composition) is administered to the individual simultaneously with the second therapeutic agent. For example, the mTOR inhibitor nanoparticle compositions and the second therapeutic agent are administered with a time separation of no more than about 15 minutes, such as no more than about any of 10, 5, or 1 minutes. In one example, wherein the compounds are in solution, simultaneous administration can be achieved by administering a solution containing the combination of compounds. In another example, simultaneous administration of separate solutions, one of which contains the mTOR inhibitor nanoparticle composition (such as sirolimus/albumin nanoparticle composition) and the other of which contains the second therapeutic agent, can be employed. In one example, simultaneous administration can be achieved by administering a composition containing the combination of compounds. In another example, simultaneous administration can be achieved by administering two separate compositions, one comprising the mTOR inhibitor nanoparticle composition (such as sirolimus/albumin nanoparticle composition) and the other comprising the second therapeutic agent. In some embodiments, simultaneous administration of the mTOR inhibitor (such as a limus drug, e.g., sirolimus or a derivative thereof) in the nanoparticle composition and the second therapeutic agent can be combined with supplemental doses of the mTOR inhibitor and/or the second therapeutic agent.

In other embodiments, the mTOR inhibitor nanoparticle composition (such as sirolimus/albumin nanoparticle composition) and the second therapeutic agent are not administered simultaneously. In some embodiments, the mTOR inhibitor nanoparticle composition (such as sirolimus/albumin nanoparticle composition) is administered before the second therapeutic agent. In other embodiments, the second therapeutic agent is administered before the mTOR inhibitor nanoparticle composition (such as sirolimus/albumin nanoparticle composition). The time difference in non-simultaneous administrations can be greater than 1 minute, five minutes, 10 minutes, 15 minutes, 30 minutes, 45 minutes, 60 minutes, two hours, three hours, six hours, nine hours, 12 hours, 24 hours, 36 hours, or 48 hours. In other embodiments, the first administered compound is provided time to take effect on the patient before the second administered compound is administered. In some embodiments, the difference in time does not extend beyond the time for the first administered compound to complete its effect in the patient, or beyond the time the first administered compound is completely or substantially eliminated or deactivated in the patient.

In some embodiments, the administration of the mTOR inhibitor nanoparticle composition (such as sirolimus/albumin nanoparticle composition) and the second therapeutic agent are concurrent, i.e., the administration period of the mTOR inhibitor nanoparticle composition and that of the second therapeutic agent overlap with each other. In some embodiments, the mTOR inhibitor nanoparticle composition (such as sirolimus/albumin nanoparticle composition) is administered for at least one cycle (for example, at least any of 2, 3, or 4 cycles) prior to the administration of the second therapeutic agent. In some embodiments, the second therapeutic agent is administered for at least any of one, two, three, or four weeks. In some embodiments, the administrations of the mTOR inhibitor nanoparticle composition (such as sirolimus/albumin nanoparticle composition) and the second therapeutic agent are initiated at about the same time (for example, within any one of 1, 2, 3, 4, 5, 6, or 7 days). In some embodiments, the administrations of the mTOR inhibitor nanoparticle composition (such as sirolimus/albumin nanoparticle composition) and the second therapeutic agent are terminated at about the same time (for example, within any one of 1, 2, 3, 4, 5, 6, or 7 days). In some embodiments, the administration of the second therapeutic agent continues (for example for about any one of 1, 2, 3, 4, 5, 6, 7, 8, 9, 10, 11, or 12 months) after the termination of the administration of the mTOR inhibitor nanoparticle composition (such as sirolimus/albumin nanoparticle composition). In some embodiments, the administration of the second therapeutic agent is initiated after (for example after about any one of 1, 2, 3, 4, 5, 6, 7, 8, 9, 10, 11, or 12 months) the initiation of the administration of the mTOR inhibitor nanoparticle composition (such as sirolimus/albumin nanoparticle composition). In some embodiments, the administrations of the mTOR inhibitor nanoparticle composition (such as sirolimus/albumin nanoparticle composition) and the second therapeutic agent are initiated and terminated at about the same time. In some embodiments, the administrations of the mTOR inhibitor nanoparticle composition (such as sirolimus/albumin nanoparticle composition) and the second therapeutic agent are initiated at about the same time and the administration of the second therapeutic agent continues (for example for about any one of 1, 2, 3, 4, 5, 6, 7, 8, 9, 10, 11, or 12 months) after the termination of the administration of the mTOR inhibitor nanoparticle composition. In some embodiments, the administration of the mTOR inhibitor nanoparticle composition (such as sirolimus/albumin nanoparticle composition) and the second therapeutic agent stop at about the same time and the administration of the second therapeutic agent is initiated after (for example after about any one of 1, 2, 3, 4, 5, 6, 7, 8, 9, 10, 11, or 12 months) the initiation of the administration of the mTOR inhibitor nanoparticle composition.

In some embodiments, the administration of the mTOR inhibitor nanoparticle composition (such as sirolimus/albumin nanoparticle composition) and the second therapeutic agent are non-concurrent. For example, in some embodiments, the administration of the mTOR inhibitor nanoparticle composition (such as sirolimus/albumin nanoparticle composition) is terminated before the second therapeutic agent is administered. In some embodiments, the administration of the second therapeutic agent is terminated before the mTOR inhibitor nanoparticle composition (such as sirolimus/albumin nanoparticle composition) is administered. The time period between these two non-concurrent administrations can range from about two to eight weeks, such as about four weeks.

The dosing frequency of the mTOR inhibitor nanoparticle composition (such as sirolimus/albumin nanoparticle composition) and the second therapeutic agent may be adjusted over the course of the treatment, based on the judgment of the administering physician. When administered separately, the mTOR inhibitor nanoparticle composition (such as sirolimus/albumin nanoparticle composition) and the second therapeutic agent can be administered at different dosing frequency or intervals. For example, the mTOR inhibitor nanoparticle composition (such as sirolimus/albumin nanoparticle composition) can be administered weekly, while a second therapeutic agent can be administered more or less frequently. In some embodiments, sustained continuous release formulation of the nanoparticle and/or second therapeutic agent may be used. Various formulations and devices for achieving sustained release are known in the art. A combination of the administration configurations described herein can also be used.

The mTOR inhibitor nanoparticle composition (such as sirolimus/albumin nanoparticle composition) and the second therapeutic agent can be administered using the same route of administration or different routes of administration. In some embodiments (for both simultaneous and sequential administrations), the mTOR inhibitor (such as a limus drug, e.g., sirolimus or a derivative thereof) in the mTOR inhibitor nanoparticle composition and the second therapeutic agent are administered at a predetermined ratio. For example, in some embodiments, the ratio by weight of the mTOR inhibitor (such as a limus drug, e.g., sirolimus or a derivative thereof) in the mTOR inhibitor nanoparticle composition and the second therapeutic agent is about 1 to 1. In some embodiments, the weight ratio may be between about 0.001 to about 1 and about 1000 to about 1, or between about 0.01 to about 1 and 100 to about 1. In some embodiments, the ratio by weight of the mTOR inhibitor (such as a limus drug, e.g., sirolimus or a derivative thereof) in the mTOR inhibitor nanoparticle composition and the second therapeutic agent is less than about any of 100:1, 50:1, 30:1, 10:1, 9:1, 8:1, 7:1, 6:1, 5:1, 4:1, 3:1, 2:1, and 1:1 In some embodiments, the ratio by weight of the mTOR inhibitor (such as a limus drug, e.g., sirolimus or a derivative thereof) in the mTOR inhibitor nanoparticle composition and the second therapeutic agent is more than about any of 1:1, 2:1, 3:1, 4:1, 5:1, 6:1, 7:1, 8:1, 9:1, 30:1, 50:1, 100:1. Other ratios are contemplated.

The doses required for the mTOR inhibitor (such as a limus drug, e.g., sirolimus or a derivative thereof) in the mTOR inhibitor nanoparticle composition and/or the second therapeutic agent may (but not necessarily) be the same or lower than what is normally required when each agent is administered alone. Thus, in some embodiments, a subtherapeutic amount of the mTOR inhibitor (such as a limus drug, e.g., sirolimus or a derivative thereof) in the mTOR inhibitor nanoparticle composition and/or the second therapeutic agent is administered. “Subtherapeutic amount” or “subtherapeutic level” refer to an amount that is less than the therapeutic amount, that is, less than the amount normally used when the mTOR inhibitor nanoparticle composition (such as sirolimus/albumin nanoparticle composition) and/or the second therapeutic agent are administered alone. The reduction may be reflected in terms of the amount administered at a given administration and/or the amount administered over a given period of time (reduced frequency).

In some embodiments, enough second therapeutic agent is administered so as to allow reduction of the normal dose of the mTOR inhibitor (such as a limus drug, e.g., sirolimus or a derivative thereof) in the mTOR inhibitor nanoparticle composition required to effect the same degree of treatment by at least about any of 5%, 10%, 20%, 30%, 50%, 60%, 70%, 80%, 90%, or more. In some embodiments, enough mTOR inhibitor (such as a limus drug, e.g., sirolimus or a derivative thereof) in the mTOR inhibitor nanoparticle composition is administered so as to allow reduction of the normal dose of the second therapeutic agent required to effect the same degree of treatment by at least about any of 5%, 10%, 20%, 30%, 50%, 60%, 70%, 80%, 90%, or more.

In some embodiments, the dose of both the mTOR inhibitor (such as a limus drug, e.g., sirolimus or a derivative thereof) in the mTOR inhibitor nanoparticle composition and the second therapeutic agent are reduced as compared to the corresponding normal dose of each when administered alone. In some embodiments, both the mTOR inhibitor (such as a limus drug, e.g., sirolimus or a derivative thereof) in the mTOR inhibitor nanoparticle composition and the second therapeutic agent are administered at a subtherapeutic, i.e., reduced, level. In some embodiments, the dose of the mTOR inhibitor (such as a limus drug, e.g., sirolimus or a derivative thereof) in the mTOR inhibitor nanoparticle composition and/or the second therapeutic agent is substantially less than the established maximum toxic dose (MTD). For example, the dose of the mTOR inhibitor nanoparticle composition (such as sirolimus/albumin nanoparticle composition) and/or the second therapeutic agent is less than about 50%, 40%, 30%, 20%, or 10% of the MTD.

A combination of the administration configurations described herein can be used. The combination therapy methods described herein may be performed alone or in conjunction with another therapy, such as surgery, radiation, gene therapy, immunotherapy, bone marrow transplantation, stem cell transplantation, hormone therapy, targeted therapy, cryotherapy, ultrasound therapy, photodynamic therapy, and/or chemotherapy and the like. Additionally, a person having a greater risk of developing the solid tumor may receive treatments to inhibit and/or delay the development of the disease.

As will be understood by those of ordinary skill in the art, the appropriate doses of second chemotherapeutic agents will be approximately those already employed in clinical therapies wherein the second therapeutic agent is administered alone or in combination with other chemotherapeutic agents. Variation in dosage will likely occur depending on the condition being treated. As described above, in some embodiments, the second chemotherapeutic agent may be administered at a reduced level.

Thus, in some embodiments, according to any of the methods described herein where the second therapeutic agent is pomalidomide, the pomalidomide is administered as a daily oral dose of about 1 to about 4 mg (including for example about any of 1, 1.5, 2, 2.5, 3, 3.5, or 4 mg, including any range between these values) on days 1-21 of a 28-day cycle for at least one (such as at least any of 2, 3, 4, 5, 6, 7, 8, 9, 10 or more) cycle. In some embodiments, the pomalidomide is administered as a daily oral dose of no more than about 4 (such as no more than about any of 4, 3.5, 3, 2.5, 2, 1.5, 1 or less) mg on days 1-21 of a 28-day cycle for at least one (such as at least any of 2, 3, 4, 5, 6, 7, 8, 9, 10 or more) cycle. In some embodiments, the pomalidomide is administered as a daily oral dose of about 4 mg on days 1-21 of a 28-day cycle for at least one (such as at least any of 2, 3, 4, 5, 6, 7, 8, 9, 10 or more) cycle. In some embodiments, the pomalidomide is administered until progression of the hematological malignancy. In some embodiments, the method further comprises administering dexamethasone to the individual. In some embodiments, the dexamethasone is administered as a daily dose (such as an oral dose) of about 20 to about 40 mg (including for example about any of 20, 25, 30, 35, or 40 mg, including any range between these values) on days 1, 8, 15, and 22 of a 28-day cycle for at least one (such as at least any of 2, 3, 4, 5, 6, 7, 8, 9, 10 or more) cycle. In some embodiments, the dexamethasone is administered as a daily dose (such as an oral dose) of about 40 mg on days 1, 8, 15, and 22 of a 28-day cycle for at least one (such as at least any of 2, 3, 4, 5, 6, 7, 8, 9, 10 or more) cycle. The dose of pomalidomide may be discontinued or interrupted, with or without dose reduction, to manage adverse drug reactions. In some embodiments, the pomalidomide is administered according to the prescribing information of an approved brand of pomalidomide.

In some embodiments, according to any of the methods described herein where the second therapeutic agent is lenalidomide, the lenalidomide is administered as a daily oral dose of about 15 to about 25 mg (including for example about any of 15, 16, 17, 18, 19, 20, 21, 22, 23, 24, or 25 mg, including any range between these values) on days 1-21 of a 28-day cycle for at least one (such as at least any of 2, 3, 4, 5, 6, 7, 8, 9, 10 or more) cycle. In some embodiments, the lenalidomide is administered as a daily oral dose of no more than about 25 (such as no more than about any of 25, 22.5, 20, 17.5, 15, 12.5, 10, or less) mg on days 1-21 of a 28-day cycle for at least one (such as at least any of 2, 3, 4, 5, 6, 7, 8, 9, 10 or more) cycle. In some embodiments, the lenalidomide is administered as a daily oral dose of about 25 mg on days 1-21 of a 28-day cycle for at least one (such as at least any of 2, 3, 4, 5, 6, 7, 8, 9, 10 or more) cycle. In some embodiments, the lenalidomide is administered until progression of the hematological malignancy. In some embodiments, the method further comprises administering dexamethasone to the individual. In some embodiments, the dexamethasone is administered as a daily dose (such as an oral dose) of about 20 to about 40 mg (including for example about any of 20, 25, 30, 35, or 40 mg, including any range between these values) on days 1, 8, 15, and 22 of a 28-day cycle for at least one (such as at least any of 2, 3, 4, 5, 6, 7, 8, 9, 10 or more) cycle. In some embodiments, the dexamethasone is administered as a daily dose (such as an oral dose) of about 40 mg on days 1, 8, 15, and 22 of a 28-day cycle for at least one (such as at least any of 2, 3, 4, 5, 6, 7, 8, 9, 10 or more) cycle. The dose of lenalidomide may be discontinued or interrupted, with or without dose reduction, to manage adverse drug reactions. In some embodiments, the lenalidomide is administered according to the prescribing information of an approved brand of lenalidomide.

In some embodiments, according to any of the methods described herein where the second therapeutic agent is romidepsin, the romidepsin is administered as an IV dose of about 5 to about 14 mg/m² (including for example about any of 5, 6, 7, 8, 9, 10, 11, 12, 13, or 14 mg/m², including any range between these values) on days 1, 8, and 15 of a 28-day cycle for at least one (such as at least any of 2, 3, 4, 5, 6, 7, 8, 9, 10 or more) cycle. In some embodiments, the romidepsin is administered as an IV dose of no more than about 14 (such as no more than about any of 14, 12, 10, 8, 6, 4, 2 or less) mg/m² on days 1, 8, and 15 of a 28-day cycle for at least one (such as at least any of 2, 3, 4, 5, 6, 7, 8, 9, 10 or more) cycle. In some embodiments, the romidepsin is administered as an IV dose of about 14 mg/m² on days 1, 8, and 15 of a 28-day cycle for at least one (such as at least any of 2, 3, 4, 5, 6, 7, 8, 9, 10 or more) cycle. The dose of romidepsin may be discontinued or interrupted, with or without dose reduction, to manage adverse drug reactions. In some embodiments, the romidepsin is administered according to the prescribing information of an approved brand of romidepsin.

In some embodiments, according to any of the methods described herein where the second therapeutic agent is nilotinib, the nilotinib is administered as a bi-daily oral dose of about 200 to about 400 mg (including for example about any of 200, 220, 240, 260, 280, 300, 320, 340, 360, 380, or 400 mg, including any range between these values) on days 1-28 of a 28-day cycle for at least one (such as at least any of 2, 3, 4, 5, 6, 7, 8, 9, 10 or more) cycle. In some embodiments, the nilotinib is administered as a bi-daily oral dose of no more than about 400 (such as no more than about any of 400, 350, 300, 250, 200, 150 or less) mg on days 1-28 of a 28-day cycle for at least one (such as at least any of 2, 3, 4, 5, 6, 7, 8, 9, 10 or more) cycle. In some embodiments, the nilotinib is administered as a bi-daily oral dose of about 300 mg on days 1-28 of a 28-day cycle for at least one (such as at least any of 2, 3, 4, 5, 6, 7, 8, 9, 10 or more) cycle. In some embodiments, the nilotinib is administered as a bi-daily oral dose of about 400 mg on days 1-28 of a 28-day cycle for at least one (such as at least any of 2, 3, 4, 5, 6, 7, 8, 9, 10 or more) cycle. In some embodiments, the two daily doses of nilotinib are administered approximately 12 hours apart. The dose of nilotinib may be discontinued or interrupted, with or without dose reduction, to manage adverse drug reactions. In some embodiments, the nilotinib is administered according to the prescribing information of an approved brand of nilotinib.

In some embodiments, according to any of the methods described herein where the second therapeutic agent is sorafenib, the sorafenib is administered as a bi-daily oral dose of about 250 to about 400 mg (including for example about any of 250, 275, 300, 325, 350, 375, or 400 mg, including any range between these values) on days 1-28 of a 28-day cycle for at least one (such as at least any of 2, 3, 4, 5, 6, 7, 8, 9, 10 or more) cycle. In some embodiments, the sorafenib is administered as a bi-daily oral dose of no more than about 400 (such as no more than about any of 400, 375, 350, 325, 300, 275, 250 or less) mg on days 1-28 of a 28-day cycle for at least one (such as at least any of 2, 3, 4, 5, 6, 7, 8, 9, 10 or more) cycle. In some embodiments, the sorafenib is administered as a bi-daily oral dose of about 400 mg on days 1-28 of a 28-day cycle for at least one (such as at least any of 2, 3, 4, 5, 6, 7, 8, 9, 10 or more) cycle. The dose of sorafenib may be discontinued or interrupted, with or without dose reduction, to manage adverse drug reactions. In some embodiments, the sorafenib is administered according to the prescribing information of an approved brand of sorafenib.

Whether administered in therapeutic or sub-therapeutic amounts, the combination of the mTOR inhibitor nanoparticle composition (such as sirolimus/albumin nanoparticle composition) and the second therapeutic agent should be effective in treating a solid tumor. For example, a sub-therapeutic amount of an mTOR inhibitor nanoparticle composition (such as sirolimus/albumin nanoparticle composition) can be an effective amount if, when combined with a second therapeutic agent, the combination is effective in the treatment of the solid tumor, and vice versa.

The dose of the mTOR inhibitor nanoparticle composition (such as sirolimus/albumin nanoparticle composition) and the dose of the second therapeutic agent administered to an individual (such as a human) may vary with the particular composition, the mode of administration, and the type of disease being treated. In some embodiments, the doses are effective to result in an objective response (such as a partial response or a complete response). In some embodiments, the doses are sufficient to result in a complete response in the individual. In some embodiments, the doses are sufficient to result in a partial response in the individual. In some embodiments, the doses administered are sufficient to produce an overall response rate of more than about any of 20%, 25%, 30%, 35%, 40%, 45%, 50%, 55%, 60%, 64%, 65%, 70%, 75%, 80%, 85%, or 90% among a population of individuals treated with the mTOR inhibitor nanoparticle composition (such as sirolimus/albumin nanoparticle composition) and the second therapeutic agent. Responses of an individual to the treatment of the methods described herein can be determined, for example, based on RECIST levels.

In some embodiments, the amounts of the mTOR inhibitor nanoparticle composition (such as sirolimus/albumin nanoparticle composition) and the second therapeutic agent are sufficient to prolong progress-free survival of the individual. In some embodiments, the amounts of the mTOR inhibitor nanoparticle composition (such as sirolimus/albumin nanoparticle composition) and the second therapeutic agent are sufficient to prolong overall survival of the individual. In some embodiments, the amounts of the mTOR inhibitor nanoparticle composition (such as sirolimus/albumin nanoparticle composition) and the second therapeutic agent are sufficient to produce clinical benefit of more than about any of 50%, 60%, 70%, or 77% among a population of individuals treated with the mTOR inhibitor nanoparticle composition and the second therapeutic agent.

In some embodiments, the amounts of the mTOR inhibitor nanoparticle composition (such as sirolimus/albumin nanoparticle composition) and the second therapeutic agent are sufficient to decrease the size of a tumor, decrease the number of cancer cells, or decrease the growth rate of a tumor by at least about any of 10%, 20%, 30%, 40%, 50%, 60%, 70%, 80%, 90%, 95% or 100% compared to the corresponding tumor size, number of cancer cells, or tumor growth rate in the same individual prior to treatment or compared to the corresponding activity in other individuals not receiving the treatment. Standard methods can be used to measure the magnitude of this effect, such as in vitro assays with purified enzyme, cell-based assays, animal models, or human testing.

In some embodiments, the amounts of the mTOR inhibitor nanoparticle composition (such as sirolimus/albumin nanoparticle composition) and the second therapeutic agent are below the levels that induce a toxicological effect (i.e., an effect above a clinically acceptable level of toxicity) or are at a level where a potential side effect can be controlled or tolerated when the mTOR inhibitor nanoparticle composition and the second therapeutic agent are administered to the individual.

In some embodiments, the amount of the mTOR inhibitor nanoparticle composition (such as sirolimus/albumin nanoparticle composition) is close to a maximum tolerated dose (MTD) of the composition following the same dosing regimen when administered with the second therapeutic agent. In some embodiments, the amount of the mTOR inhibitor nanoparticle composition (such as sirolimus/albumin nanoparticle composition) is more than about any of 80%, 90%, 95%, or 98% of the MTD when administered with the second therapeutic agent.

In some embodiments, the amount of an mTOR inhibitor (such as a limus drug, e.g., sirolimus) in the mTOR inhibitor nanoparticle composition is included in any of the following ranges: about 0.1 mg to about 1000 mg, about 0.1 mg to about 2.5 mg, about 0.5 mg to about 5 mg, about 5 mg to about 10 mg, about 10 mg to about 15 mg, about 15 mg to about 20 mg, about 20 mg to about 25 mg, about 20 mg to about 50 mg, about 25 mg to about 50 mg, about 50 mg to about 75 mg, about 50 mg to about 100 mg, about 75 mg to about 100 mg, about 100 mg to about 125 mg, about 125 mg to about 150 mg, about 150 mg to about 175 mg, about 175 mg to about 200 mg, about 200 mg to about 225 mg, about 225 mg to about 250 mg, about 250 mg to about 300 mg, about 300 mg to about 350 mg, about 350 mg to about 400 mg, about 400 mg to about 450 mg, or about 450 mg to about 500 mg, about 500 mg to about 600 mg, about 600 mg to about 700 mg, about 700 mg to about 800 mg, about 800 mg to about 900 mg, or about 900 mg to about 1000 mg, including any range between these values. In some embodiments, the amount of an mTOR inhibitor (such as a limus drug, e.g., sirolimus) in the effective amount of the composition (e.g., a unit dosage form) is in the range of about 5 mg to about 500 mg, such as about 30 to about 400 mg, 30 mg to about 300 mg, or about 50 mg to about 200 mg. In some embodiments, the amount of an mTOR inhibitor (such as a limus drug, e.g., sirolimus) in the effective amount of the mTOR inhibitor nanoparticle composition (e.g., a unit dosage form) is in the range of about 150 mg to about 500 mg, including for example, about 150 mg, about 225 mg, about 250 mg, about 300 mg, about 325 mg, about 350 mg, about 375 mg, about 400 mg, about 425 mg, about 450 mg, about 475 mg, or about 500 mg. In some embodiments, the concentration of the mTOR inhibitor (such as a limus drug, e.g., sirolimus) in the mTOR inhibitor nanoparticle composition is dilute (about 0.1 mg/ml) or concentrated (about 100 mg/ml), including for example about any of 0.1 mg/ml to about 50 mg/ml, about 0.1 mg/ml to about 20 mg/ml, about 1 mg/ml to about 10 mg/ml, about 2 mg/ml to about 8 mg/ml, about 4 mg/ml to about 6 mg/ml, or about 5 mg/ml. In some embodiments, the concentration of the mTOR inhibitor (such as a limus drug, e.g., sirolimus) in the mTOR inhibitor nanoparticle composition is at least about any of 0.5 mg/ml, 1.3 mg/ml, 1.5 mg/ml, 2 mg/ml, 3 mg/ml, 4 mg/ml, 5 mg/ml, 6 mg/ml, 7 mg/ml, 8 mg/ml, 9 mg/ml, 10 mg/ml, 15 mg/ml, 20 mg/ml, 25 mg/ml, 30 mg/ml, 40 mg/ml, or 50 mg/ml.

In some embodiments of any of the above aspects, the amount of an mTOR inhibitor (such as a limus drug, e.g., sirolimus) in the mTOR inhibitor nanoparticle composition is at least about any of 1 mg/kg, 2.5 mg/kg, 3.5 mg/kg, 5 mg/kg, 6.5 mg/kg, 7.5 mg/kg, 10 mg/kg, 15 mg/kg, 20 mg/kg, 25 mg/kg, 30 mg/kg, 35 mg/kg, 40 mg/kg, 45 mg/kg, 50 mg/kg, 55 mg/kg, or 60 mg/kg. In some embodiments, the effective amount of an mTOR inhibitor (such as a limus drug, e.g., sirolimus) in the mTOR inhibitor nanoparticle composition is less than about any of 350 mg/kg, 300 mg/kg, 250 mg/kg, 200 mg/kg, 150 mg/kg, 100 mg/kg, 50 mg/kg, 25 mg/kg, 20 mg/kg, 10 mg/kg, 7.5 mg/kg, 6.5 mg/kg, 5 mg/kg, 3.5 mg/kg, 2.5 mg/kg, or 1 mg/kg.

In some embodiments of any of the above aspects, the amount of an mTOR inhibitor (such as a limus drug, e.g., sirolimus) in the mTOR inhibitor nanoparticle composition is about any of 25 mg/m², 30 mg/m², 50 mg/m², 60 mg/m², 75 mg/m², 80 mg/m², 90 mg/m², 100 mg/m², 120 mg/m², 160 mg/m², 175 mg/m², 180 mg/m², 200 mg/m², 210 mg/m², 220 mg/m², 250 mg/m², 260 mg/m², 300 mg/m², 350 mg/m², 400 mg/m², 500 mg/m², 540 mg/m², 750 mg/m², 1000 mg/m², or 1080 mg/m² mTOR inhibitor. In some embodiments, the mTOR inhibitor nanoparticle composition includes less than about any of 350 mg/m², 300 mg/m², 250 mg/m², 200 mg/m², 150 mg/m², 120 mg/m², 100 mg/m², 90 mg/m², 50 mg/m², or 30 mg/m² mTOR inhibitor (such as a limus drug, e.g., sirolimus). In some embodiments, the amount of the mTOR inhibitor (such as a limus drug, e.g., sirolimus) per administration is less than about any of 25 mg/m², 22 mg/m², 20 mg/m², 18 mg/m², 15 mg/m², 14 mg/m², 13 mg/m², 12 mg/m², 11 mg/m², 10 mg/m², 9 mg/m², 8 mg/m², 7 mg/m², 6 mg/m², 5 mg/m², 4 mg/m², 3 mg/m², 2 mg/m², or 1 mg/m². In some embodiments, the effective amount of mTOR inhibitor (such as a limus drug, e.g., sirolimus) in the mTOR inhibitor nanoparticle composition is included in any of the following ranges: about 1 to about 5 mg/m², about 5 to about 10 mg/m², about 10 to about 25 mg/m², about 25 to about 50 mg/m², about 50 to about 75 mg/m², about 75 to about 100 mg/m², about 100 to about 125 mg/m², about 125 to about 150 mg/m², about 150 to about 175 mg/m², about 175 to about 200 mg/m², about 200 to about 225 mg/m², about 225 to about 250 mg/m², about 250 to about 300 mg/m², about 300 to about 350 mg/m², or about 350 to about 400 mg/m². In some embodiments, the effective amount of mTOR inhibitor (such as a limus drug, e.g., sirolimus) in the mTOR inhibitor nanoparticle composition is about 30 to about 300 mg/m², such as about 100 to about 150 mg/m², about 120 mg/m², about 130 mg/m², or about 140 mg/m².

In some embodiments, the combination of compounds exhibits a synergistic effect (i.e., greater than additive effect) in the treatment of the solid tumor. The term “synergistic effect” refers to the action of two agents, such as an mTOR inhibitor nanoparticle composition (such as sirolimus/albumin nanoparticle composition) and a second therapeutic agent, producing an effect, for example, slowing the symptomatic progression of cancer or symptoms thereof, which is greater than the simple addition of the effects of each drug administered by themselves. A synergistic effect can be calculated, for example, using suitable methods such as the Sigmoid-Emax equation (Holford, N. H. G. and Schemer, L. B., Clin. Pharmacokinet. 6: 429-453 (1981)), the equation of Loewe additivity (Loewe, S. and Muischnek, H., Arch. Exp. Pathol Pharmacol. 114: 313-326 (1926)) and the median-effect equation (Chou, T. C. and Talalay, P., Adv. Enzyme Regul. 22: 27-55 (1984)). Each equation referred to above can be applied to experimental data to generate a corresponding graph to aid in assessing the effects of the drug combination. The corresponding graphs associated with the equations referred to above are the concentration-effect curve, isobologram curve and combination index curve, respectively.

In different embodiments, depending on the combination and the effective amounts used, the combination of compounds can inhibit cancer growth, achieve cancer stasis, or even achieve substantial or complete cancer regression.

While the amounts of an mTOR inhibitor nanoparticle composition (such as sirolimus/albumin nanoparticle composition) and a second therapeutic agent should result in the effective treatment of a solid tumor, the amounts, when combined, are preferably not excessively toxic to the individual (i.e., the amounts are preferably within toxicity limits as established by medical guidelines). In some embodiments, either to prevent excessive toxicity and/or provide a more efficacious treatment of a solid tumor, a limitation on the total administered dosage is provided.

Different dosage regimens may be used to treat a solid tumor. In some embodiments, a daily dosage, such as any of the exemplary dosages described above, is administered once, twice, three times, or four times a day for three, four, five, six, seven, eight, nine, or ten days. Depending on the stage and severity of the cancer, a shorter treatment time (e.g., up to five days) may be employed along with a high dosage, or a longer treatment time (e.g., ten or more days, or weeks, or a month, or longer) may be employed along with a low dosage. In some embodiments, a once- or twice-daily dosage is administered every other day.

In some embodiments, the dosing frequencies for the administration of the mTOR inhibitor nanoparticle composition (such as sirolimus/albumin nanoparticle composition) include, but are not limited to, daily, every two days, every three days, every four days, every five days, every six days, weekly without break, three out of four weeks (such as on days 1, 8, and 15 of a 28-day cycle), once every three weeks, once every two weeks, or two out of three weeks. In some embodiments, the mTOR inhibitor nanoparticle composition (such as sirolimus/albumin nanoparticle composition) is administered about once every 2 weeks, once every 3 weeks, once every 4 weeks, once every 6 weeks, or once every 8 weeks. In some embodiments, the mTOR inhibitor nanoparticle composition (such as sirolimus/albumin nanoparticle composition) is administered at least about any of 1×, 2×, 3×, 4×, 5×, 6×, or 7× (i.e., daily) a week. In some embodiments, the intervals between each administration are less than about any of 6 months, 3 months, 1 month, 20 days, 15, days, 14 days, 13 days, 12 days, 11 days, 10 days, 9 days, 8 days, 7 days, 6 days, 5 days, 4 days, 3 days, 2 days, or 1 day. In some embodiments, the intervals between each administration are more than about any of 1 month, 2 months, 3 months, 4 months, 5 months, 6 months, 8 months, or 12 months. In some embodiments, there is no break in the dosing schedule. In some embodiments, the interval between each administration is no more than about a week.

In some embodiments, the dosing frequency is once every two days for one time, two times, three times, four times, five times, six times, seven times, eight times, nine times, ten times, or eleven times. In some embodiments, the dosing frequency is once every two days for five times. In some embodiments, the mTOR inhibitor (such as a limus drug, e.g., sirolimus or a derivative thereof) is administered over a period of at least ten days, wherein the interval between each administration is no more than about two days, and wherein the dose of the mTOR inhibitor at each administration is about 0.25 mg/m² to about 250 mg/m², about 0.25 mg/m² to about 150 mg/m², about 0.25 mg/m² to about 75 mg/m², such as about 0.25 mg/m² to about 25 mg/m², or about 25 mg/m² to about 50 mg/m².

The administration of the mTOR inhibitor nanoparticle composition (such as sirolimus/albumin nanoparticle composition) can be extended over an extended period of time, such as from about a month up to about seven years. In some embodiments, the mTOR inhibitor nanoparticle composition (such as sirolimus/albumin nanoparticle composition) is administered over a period of at least about any of 2, 3, 4, 5, 6, 7, 8, 9, 10, 11, 12, 18, 24, 30, 36, 48, 60, 72, or 84 months.

In some embodiments, the dosage of an mTOR inhibitor (such as a limus drug, e.g., sirolimus or a derivative thereof) in a nanoparticle composition can be in the range of 5-400 mg/m² when given on a 3 week schedule, or 5-250 mg/m² (such as 80-150 mg/m², for example 100-120 mg/m²) when given on a weekly schedule. For example, the amount of an mTOR inhibitor (such as a limus drug, e.g., sirolimus or a derivative thereof) is about 60 to about 300 mg/m² (e.g., about 260 mg/m²) on a three week schedule.

In some embodiments, the exemplary dosing schedules for the administration of the mTOR inhibitor nanoparticle composition (such as sirolimus/albumin nanoparticle composition) include, but are not limited to, 100 mg/m², weekly, without break; 10 mg/m² weekly, 3 out of four weeks (such as on days 1, 8, and 15 of a 28-day cycle); 45 mg/m² weekly, 3 out of four weeks (such as on days 1, 8, and 15 of a 28-day cycle); 75 mg/m² weekly, 3 out of four weeks (such as on days 1, 8, and 15 of a 28-day cycle); 100 mg/m², weekly, 3 out of 4 weeks; 125 mg/m², weekly, 3 out of 4 weeks; 125 mg/m², weekly, 2 out of 3 weeks; 130 mg/m², weekly, without break; 175 mg/m², once every 2 weeks; 260 mg/m², once every 2 weeks; 260 mg/m², once every 3 weeks; 180-300 mg/m², every three weeks; 60-175 mg/m², weekly, without break; 20-150 mg/m² twice a week; and 150-250 mg/m² twice a week. The dosing frequency of the mTOR inhibitor nanoparticle composition (such as sirolimus/albumin nanoparticle composition) may be adjusted over the course of the treatment based on the judgment of the administering physician.

In some embodiments, the individual is treated for at least about any of one, two, three, four, five, six, seven, eight, nine, or ten treatment cycles.

The mTOR inhibitor nanoparticle composition (such as sirolimus/albumin nanoparticle composition) described herein allow infusion of the mTOR inhibitor nanoparticle composition to an individual over an infusion time that is shorter than about 24 hours. For example, in some embodiments, the mTOR inhibitor nanoparticle composition (such as sirolimus/albumin nanoparticle composition) is administered over an infusion period of less than about any of 24 hours, 12 hours, 8 hours, 5 hours, 3 hours, 2 hours, 1 hour, 30 minutes, 20 minutes, or 10 minutes. In some embodiments, the mTOR inhibitor nanoparticle composition (such as sirolimus/albumin nanoparticle composition) is administered over an infusion period of about 30 minutes.

In some embodiments, the exemplary dose of the mTOR inhibitor (in some embodiments a limus drug, e.g., sirolimus) in the mTOR inhibitor nanoparticle composition includes, but is not limited to, about any of 50 mg/m², 60 mg/m², 75 mg/m², 80 mg/m², 90 mg/m², 100 mg/m², 120 mg/m², 160 mg/m², 175 mg/m², 200 mg/m², 210 mg/m², 220 mg/m², 260 mg/m², and 300 mg/m². For example, the dosage of an mTOR inhibitor (such as a limus drug, e.g., sirolimus or a derivative thereof) in a nanoparticle composition can be in the range of about 100-400 mg/m² when given on a 3 week schedule, or about 10-250 mg/m² when given on a weekly schedule.

In some embodiments, the dosage of an mTOR inhibitor (such as a limus drug, e.g., sirolimus) is about 100 mg to about 400 mg, for example about 100 mg, about 200 mg, about 300 mg, or about 400 mg. In some embodiments, the limus drug is administered at about 100 mg weekly, about 200 mg weekly, about 300 mg weekly, about 100 mg twice weekly, or about 200 mg twice weekly. In some embodiments, the administration is further followed by a monthly maintenance dose (which can be the same or different from the weekly doses).

In some embodiments when the limus nanoparticle composition is administered intravenously, the dosage of an mTOR inhibitor (such as a limus drug, e.g., sirolimus) in a nanoparticle composition can be in the range of about 30 mg to about 400 mg. The mTOR inhibitor nanoparticle composition (such as sirolimus/albumin nanoparticle composition) described herein allow infusion of the mTOR inhibitor nanoparticle composition to an individual over an infusion time that is shorter than about 24 hours. For example, in some embodiments, the mTOR inhibitor nanoparticle composition (such as sirolimus/albumin nanoparticle composition) is administered over an infusion period of less than about any of 24 hours, 12 hours, 8 hours, 5 hours, 3 hours, 2 hours, 1 hour, 30 minutes, 20 minutes, or 10 minutes. In some embodiments, the mTOR inhibitor nanoparticle composition (such as sirolimus/albumin nanoparticle composition) is administered over an infusion period of about 30 minutes to about 40 minutes.

In some embodiments, each dosage contains both an mTOR inhibitor nanoparticle composition (such as sirolimus/albumin nanoparticle composition) and a second therapeutic agent to be delivered as a single dosage, while in other embodiments, each dosage contains either the mTOR inhibitor nanoparticle composition or the second therapeutic agent to be delivered as separate dosages.

An mTOR inhibitor nanoparticle composition (such as sirolimus/albumin nanoparticle composition) and a second therapeutic agent, in pure form or in an appropriate pharmaceutical composition, can be administered via any of the accepted modes of administration or agents known in the art. The compositions and/or agents can be administered, for example, orally, nasally, parenterally (such as intravenous, intramuscular, or subcutaneous), topically, transdermally, intravaginally, intravesically, intracistemally, or rectally. The dosage form can be, for example, a solid, semi-solid, lyophilized powder, or liquid dosage form, such as tablets, pills, soft elastic or hard gelatin capsules, powders, solutions, suspensions, suppositories, aerosols, or the like, preferably in unit dosage forms suitable for simple administration of precise dosages.

As discussed above, the mTOR inhibitor nanoparticle composition (such as sirolimus/albumin nanoparticle composition) and the second therapeutic agent can be administered in a single unit dose or separate dosage forms. Accordingly, the phrase “pharmaceutical combination” includes a combination of two drugs in either a single dosage form or a separate dosage forms, i.e., the pharmaceutically acceptable carriers and excipients described throughout the application can be combined with an mTOR inhibitor nanoparticle composition (such as sirolimus/albumin nanoparticle composition) and a second therapeutic agent in a single unit dose, as well as individually combined with an mTOR inhibitor nanoparticle composition and a second therapeutic agent when these compounds are administered separately.

Auxiliary and adjuvant agents may include, for example, preserving, wetting, suspending, sweetening, flavoring, perfuming, emulsifying, and dispensing agents. Prevention of the action of microorganisms is generally provided by various antibacterial and antifungal agents, such as, parabens, chlorobutanol, phenol, sorbic acid, and the like. Isotonic agents, such as sugars, sodium chloride, and the like, may also be included. Prolonged absorption of an injectable pharmaceutical form can be brought about by the use of agents delaying absorption, for example, aluminum monostearate and gelatin. The auxiliary agents also can include wetting agents, emulsifying agents, pH buffering agents, and antioxidants, such as citric acid, sorbitan monolaurate, triethanolamine oleate, butylated hydroxytoluene, and the like.

Solid dosage forms can be prepared with coatings and shells, such as enteric coatings and others well-known in the art. They can contain pacifying agents and can be of such composition that they release the active compound or compounds in a certain part of the intestinal tract in a delayed manner. Examples of embedded compositions that can be used are polymeric substances and waxes. The active compounds also can be in microencapsulated form, if appropriate, with one or more of the above-mentioned excipients.

Liquid dosage forms for oral administration include pharmaceutically acceptable emulsions, solutions, suspensions, syrups, and elixirs. Such dosage forms are prepared, for example, by dissolving, dispersing, etc., the mTOR inhibitor nanoparticle composition (such as sirolimus/albumin nanoparticle composition) or second therapeutic agents described herein, or a pharmaceutically acceptable salt thereof, and optional pharmaceutical adjuvants in a carrier, such as, for example, water, saline, aqueous dextrose, glycerol, ethanol and the like; solubilizing agents and emulsifiers, such as ethyl alcohol, isopropyl alcohol, ethyl carbonate, ethyl acetate, benzyl alcohol, benzyl benzoate, propyleneglycol, 1,3-butyleneglycol, dimethyl formamide; oils, in particular, cottonseed oil, groundnut oil, corn germ oil, olive oil, castor oil and sesame oil, glycerol, tetrahydrofurfuryl alcohol, polyethyleneglycols and fatty acid esters of sorbitan; or mixtures of these substances, and the like, to thereby form a solution or suspension.

In some embodiments, depending on the intended mode of administration, the pharmaceutically acceptable compositions will contain about 1% to about 99% by weight of the compounds described herein, or a pharmaceutically acceptable salt thereof, and 99% to 1% by weight of a pharmaceutically acceptable excipient. In one example, the composition will be between about 5% and about 75% by weight of a compound described herein, or a pharmaceutically acceptable salt thereof, with the rest being suitable pharmaceutical excipients.

Actual methods of preparing such dosage forms are known, or will be apparent, to those skilled in this art. Reference is made, for example, to Remington's Pharmaceutical Sciences, 18th Ed., (Mack Publishing Company, Easton, Pa., 1990).

The mTOR inhibitor nanoparticle composition (such as sirolimus/albumin nanoparticle composition) can be administered to an individual (such as a human) via various routes, including, for example, intravenous, intra-arterial, intraperitoneal, intrapulmonary, oral, inhalation, intravesicular, intramuscular, intra-tracheal, subcutaneous, intraocular, intrathecal, transmucosal, and transdermal. In some embodiments, sustained continuous release formulation of the composition may be used. In some embodiments, the composition is administered intravenously. In some embodiments, the composition is administered intraportally. In some embodiments, the composition is administered intraarterially. In some embodiments, the composition is administered intraperitoneally.

### Nanoparticle Compositions

The mTOR inhibitor nanoparticle compositions described herein comprise nanoparticles comprising (in various embodiments consisting essentially of or consisting of) an mTOR inhibitor (such as a limus drug, e.g., sirolimus or a derivative thereof) and an albumin (such as human serum albumin). Nanoparticles of poorly water soluble drugs (such as macrolides) have been disclosed in, for example, U.S. Pat. Nos. 5,916,596; 6,506,405; 6,749,868, 6,537,579, 7,820,788, and 8,911,786, and also in U.S. Pat. Pub. Nos. 2006/0263434, and 2007/0082838; PCT Patent Application WO08/137148, each of which is incorporated herein by reference in their entirety.

In some embodiments, the composition comprises nanoparticles with an average or mean diameter of no greater than about 1000 nanometers (nm), such as no greater than about any of 900, 800, 700, 600, 500, 400, 300, 200, and 100 nm. In some embodiments, the average or mean diameters of the nanoparticles is no greater than about 200 nm. In some embodiments, the average or mean diameters of the nanoparticles is no greater than about 150 nm. In some embodiments, the average or mean diameters of the nanoparticles is no greater than about 100 nm. In some embodiments, the average or mean diameter of the nanoparticles is about 10 to about 400 nm. In some embodiments, the average or mean diameter of the nanoparticles is about 10 to about 150 nm. In some embodiments, the average or mean diameter of the nanoparticles is about 40 to about 120 nm. In some embodiments, the nanoparticles are no less than about 50 nm. In some embodiments, the nanoparticles are sterile-filterable.

In some embodiments, the nanoparticles in the composition described herein have an average diameter of no greater than about 200 nm, including for example no greater than about any one of 190, 180, 170, 160, 150, 140, 130, 120, 110, 100, 90, 80, 70, or 60 nm. In some embodiments, at least about 50% (for example at least about any one of 60%, 70%, 80%, 90%, 95%, or 99%) of the nanoparticles in the composition have a diameter of no greater than about 200 nm, including for example no greater than about any one of 190, 180, 170, 160, 150, 140, 130, 120, 110, 100, 90, 80, 70, or 60 nm. In some embodiments, at least about 50% (for example at least any one of 60%, 70%, 80%, 90%, 95%, or 99%) of the nanoparticles in the composition fall within the range of about 10 nm to about 400 nm, including for example about 10 nm to about 200 nm, about 20 nm to about 200 nm, about 30 nm to about 180 nm, about 40 nm to about 150 nm, about 40 nm to about 120 nm, and about 60 nm to about 100 nm.

In some embodiments, the albumin has sulfhydryl groups that can form disulfide bonds. In some embodiments, at least about 5% (including for example at least about any one of 10%, 15%, 20%, 25%, 30%, 40%, 50%, 60%, 70%, 80%, or 90%) of the albumin in the nanoparticle portion of the composition are crosslinked (for example crosslinked through one or more disulfide bonds).

In some embodiments, the nanoparticles comprising the mTOR inhibitor (such as a limus drug, e.g., sirolimus or a derivative thereof) are associated (e.g., coated) with an albumin (such as human albumin or human serum albumin). In some embodiments, the composition comprises an mTOR inhibitor (such as a limus drug, e.g., sirolimus or a derivative thereof) in both nanoparticle and non-nanoparticle forms (e.g., in the form of solutions or in the form of soluble albumin/nanoparticle complexes), wherein at least about any one of 50%, 60%, 70%, 80%, 90%, 95%, or 99% of the mTOR inhibitor in the composition are in nanoparticle form. In some embodiments, the mTOR inhibitor (such as a limus drug, e.g., sirolimus or a derivative thereof) in the nanoparticles constitutes more than about any one of 50%, 60%, 70%, 80%, 90%, 95%, or 99% of the nanoparticles by weight. In some embodiments, the nanoparticles have a non-polymeric matrix. In some embodiments, the nanoparticles comprise a core of an mTOR inhibitor (such as a limus drug, e.g., sirolimus or a derivative thereof) that is substantially free of polymeric materials (such as polymeric matrix).

In some embodiments, the composition comprises an albumin in both nanoparticle and non-nanoparticle portions of the composition, wherein at least about any one of 50%, 60%, 70%, 80%, 90%, 95%, or 99% of the albumin in the composition are in non-nanoparticle portion of the composition.

In some embodiments, the weight ratio of an albumin (such as human albumin or human serum albumin) and a mTOR inhibitor (such as a limus drug, e.g., sirolimus or a derivative thereof) in the mTOR inhibitor nanoparticle composition is about 18:1 or less, such as about 15:1 or less, for example about 10:1 or less. In some embodiments, the weight ratio of an albumin (such as human albumin or human serum albumin) and an mTOR inhibitor (such as a limus drug, e.g., sirolimus or a derivative thereof) in the composition falls within the range of any one of about 1:1 to about 18:1, about 2:1 to about 15:1, about 3:1 to about 13:1, about 4:1 to about 12:1, about 5:1 to about 10:1. In some embodiments, the weight ratio of an albumin and an mTOR inhibitor (such as a limus drug, e.g., sirolimus or a derivative thereof) in the nanoparticle portion of the composition is about any one of 1:2, 1:3, 1:4, 1:5, 1:9, 1:10, 1:15, or less. In some embodiments, the weight ratio of the albumin (such as human albumin or human serum albumin) and the mTOR inhibitor (such as a limus drug, e.g., sirolimus or a derivative thereof) in the composition is any one of the following: about 1:1 to about 18:1, about 1:1 to about 15:1, about 1:1 to about 12:1, about 1:1 to about 10:1, about 1:1 to about 9:1, about 1:1 to about 8:1, about 1:1 to about 7:1, about 1:1 to about 6:1, about 1:1 to about 5:1, about 1:1 to about 4:1, about 1:1 to about 3:1, about 1:1 to about 2:1, about 1:1 to about 1:1.

In some embodiments, the mTOR inhibitor nanoparticle composition (such as sirolimus/albumin nanoparticle composition) comprises one or more of the above characteristics.

The nanoparticles described herein may be present in a dry formulation (such as lyophilized composition) or suspended in a biocompatible medium. Suitable biocompatible media include, but are not limited to, water, buffered aqueous media, saline, buffered saline, optionally buffered solutions of amino acids, optionally buffered solutions of proteins, optionally buffered solutions of sugars, optionally buffered solutions of vitamins, optionally buffered solutions of synthetic polymers, lipid-containing emulsions, and the like.

In some embodiments, the pharmaceutically acceptable carrier comprises an albumin (such as human albumin or human serum albumin). The albumin may either be natural in origin or synthetically prepared. In some embodiments, the albumin is human albumin or human serum albumin. In some embodiments, the albumin is a recombinant albumin.

Human serum albumin (HSA) is a highly soluble globular protein of Mᵣ 65K and consists of 585 amino acids. HSA is the most abundant protein in the plasma and accounts for 70-80% of the colloid osmotic pressure of human plasma. The amino acid sequence of HSA contains a total of 17 disulfide bridges, one free thiol (Cys 34), and a single tryptophan (Trp 214). Intravenous use of HSA solution has been indicated for the prevention and treatment of hypovolumic shock (see, e.g., Tullis, JAMA, 237: 355-360, 460-463, (1977)) and Houser et al., Surgery, Gynecology and Obstetrics, 150: 811-816 (1980)) and in conjunction with exchange transfusion in the treatment of neonatal hyperbilirubinemia (see, e.g., Finlayson, Seminars in Thrombosis and Hemostasis, 6, 85-120, (1980)). Other albumins are contemplated, such as bovine serum albumin. Use of such non-human albumins could be appropriate, for example, in the context of use of these compositions in non-human mammals, such as the veterinary (including domestic pets and agricultural context). Human serum albumin (HSA) has multiple hydrophobic binding sites (a total of eight for fatty acids, an endogenous ligand of HSA) and binds a diverse set of drugs, especially neutral and negatively charged hydrophobic compounds (Goodman et al., The Pharmacological Basis of Therapeutics, 9th ed, McGraw-Hill New York (1996)). Two high affinity binding sites have been proposed in subdomains IIA and IIIA of HSA, which are highly elongated hydrophobic pockets with charged lysine and arginine residues near the surface which function as attachment points for polar ligand features (see, e.g., Fehske et al., Biochem. Pharmcol., 30, 687-92 (198a), Vorum, Dan. Med. Bull., 46, 379-99 (1999), Kragh-Hansen, Dan. Med. Bull., 1441, 131-40 (1990), Curry et al., Nat. Struct. Biol., 5, 827-35 (1998), Sugio et al., Protein. Eng., 12, 439-46 (1999), He et al., Nature, 358, 209-15 (199b), and Carter et al., Adv. Protein. Chem., 45, 153-203 (1994)). Rapamycin and propofol have been shown to bind HSA (see, e.g., Paal et al., Eur. J Biochem., 268(7), 2187-91 (200a), Purcell et al., Biochim. Biophys. Acta, 1478(a), 61-8 (2000), Altmayer et al., Arzneimittelforschung, 45, 1053-6 (1995), and Garrido et al., Rev. Esp. Anestestiol. Reanim., 41, 308-12 (1994)). In addition, docetaxel has been shown to bind to human plasma proteins (see, e.g., Urien et al., Invest. New Drugs, 14(b), 147-51 (1996)).

The albumin (such as human albumin or human serum albumin) in the composition generally serves as a carrier for the mTOR inhibitor, i.e., the albumin in the composition makes the mTOR inhibitor (such as a limus drug, e.g., sirolimus or a derivative thereof) more readily suspendable in an aqueous medium or helps maintain the suspension as compared to compositions not comprising an albumin. This can avoid the use of toxic solvents (or surfactants) for solubilizing the mTOR inhibitor, and thereby can reduce one or more side effects of administration of the mTOR inhibitor (such as a limus drug, e.g., sirolimus or a derivative thereof) into an individual (such as a human). Thus, in some embodiments, the composition described herein is substantially free (such as free) of surfactants, such as Cremophor (or polyoxyethylated castor oil, including Cremophor EL® (BASF)). In some embodiments, the mTOR inhibitor nanoparticle composition (such as sirolimus/albumin nanoparticle composition) is substantially free (such as free) of surfactants. A composition is “substantially free of Cremophor” or “substantially free of surfactant” if the amount of Cremophor or surfactant in the composition is not sufficient to cause one or more side effect(s) in an individual when the mTOR inhibitor nanoparticle composition (such as sirolimus/albumin nanoparticle composition) is administered to the individual. In some embodiments, the mTOR inhibitor nanoparticle composition (such as sirolimus/albumin nanoparticle composition) contains less than about any one of 20%, 15%, 10%, 7.5%, 5%, 2.5%, or 1% organic solvent or surfactant. In some embodiments, the albumin is human albumin or human serum albumin. In some embodiments, the albumin is recombinant albumin.

The amount of an albumin in the composition described herein will vary depending on other components in the composition. In some embodiments, the composition comprises an albumin in an amount that is sufficient to stabilize the mTOR inhibitor (such as a limus drug, e.g., sirolimus or a derivative thereof) in an aqueous suspension, for example, in the form of a stable colloidal suspension (such as a stable suspension of nanoparticles). In some embodiments, the albumin is in an amount that reduces the sedimentation rate of the mTOR inhibitor (such as a limus drug, e.g., sirolimus or a derivative thereof) in an aqueous medium. For particle-containing compositions, the amount of the albumin also depends on the size and density of nanoparticles of the mTOR inhibitor.

An mTOR inhibitor (such as a limus drug, e.g., sirolimus or a derivative thereof) is “stabilized” in an aqueous suspension if it remains suspended in an aqueous medium (such as without visible precipitation or sedimentation) for an extended period of time, such as for at least about any of 0.1, 0.2, 0.25, 0.5, 1, 2, 3, 4, 5, 6, 7, 8, 9, 10, 11, 12, 24, 36, 48, 60, or 72 hours. The suspension is generally, but not necessarily, suitable for administration to an individual (such as a human). Stability of the suspension is generally (but not necessarily) evaluated at a storage temperature (such as room temperature (such as 20-25° C.) or refrigerated conditions (such as 4° C.)). For example, a suspension is stable at a storage temperature if it exhibits no flocculation or particle agglomeration visible to the naked eye or when viewed under the optical microscope at 1000 times, at about fifteen minutes after preparation of the suspension. Stability can also be evaluated under accelerated testing conditions, such as at a temperature that is higher than about 40° C.

In some embodiments, the albumin is present in an amount that is sufficient to stabilize the mTOR inhibitor (such as a limus drug, e.g., sirolimus or a derivative thereof) in an aqueous suspension at a certain concentration. For example, the concentration of the mTOR inhibitor (such as a limus drug, e.g., sirolimus or a derivative thereof) in the composition is about 0.1 to about 100 mg/ml, including for example about any of 0.1 to about 50 mg/ml, about 0.1 to about 20 mg/ml, about 1 to about 10 mg/ml, about 2 mg/ml to about 8 mg/ml, about 4 to about 6 mg/ml, or about 5 mg/ml. In some embodiments, the concentration of the mTOR inhibitor (such as a limus drug, e.g., sirolimus or a derivative thereof) is at least about any of 1.3 mg/ml, 1.5 mg/ml, 2 mg/ml, 3 mg/ml, 4 mg/ml, 5 mg/ml, 6 mg/ml, 7 mg/ml, 8 mg/ml, 9 mg/ml, 10 mg/ml, 15 mg/ml, 20 mg/ml, 25 mg/ml, 30 mg/ml, 40 mg/ml, and 50 mg/ml. In some embodiments, the albumin is present in an amount that avoids use of surfactants (such as Cremophor), so that the composition is free or substantially free of surfactant (such as Cremophor).

In some embodiments, the composition, in liquid form, comprises from about 0.1% to about 50% (w/v) (e.g., about 0.5% (w/v), about 5% (w/v), about 10% (w/v), about 15% (w/v), about 20% (w/v), about 30% (w/v), about 40% (w/v), or about 50% (w/v)) of an albumin. In some embodiments, the composition, in liquid form, comprises about 0.5% to about 5% (w/v) of albumin.

In some embodiments, the weight ratio of the albumin to the mTOR inhibitor (such as a limus drug, e.g., sirolimus or a derivative thereof) in the mTOR inhibitor nanoparticle composition is such that a sufficient amount of mTOR inhibitor binds to, or is transported by, the cell. While the weight ratio of an albumin to an mTOR inhibitor (such as a limus drug, e.g., sirolimus or a derivative thereof) will have to be optimized for different albumin and mTOR inhibitor combinations, generally the weight ratio of an albumin to an mTOR inhibitor (such as a limus drug, e.g., sirolimus or a derivative thereof) (w/w) is about 0.01:1 to about 100:1, about 0.02:1 to about 50:1, about 0.05:1 to about 20:1, about 0.1:1 to about 20:1, about 1:1 to about 18:1, about 2:1 to about 15:1, about 3:1 to about 12:1, about 4:1 to about 10:1, about 5:1 to about 9:1, or about 9:1. In some embodiments, the albumin to mTOR inhibitor (such as a limus drug, e.g., sirolimus or a derivative thereof) weight ratio is about any of 18:1 or less, 15:1 or less, 14:1 or less, 13:1 or less, 12:1 or less, 11:1 or less, 10:1 or less, 9:1 or less, 8:1 or less, 7:1 or less, 6:1 or less, 5:1 or less, 4:1 or less, and 3:1 or less. In some embodiments, the weight ratio of the albumin (such as human albumin or human serum albumin) to the mTOR inhibitor (such as a limus drug, e.g., sirolimus or a derivative thereof) in the composition is any one of the following: about 1:1 to about 18:1, about 1:1 to about 15:1, about 1:1 to about 12:1, about 1:1 to about 10:1, about 1:1 to about 9:1, about 1:1 to about 8:1, about 1:1 to about 7:1, about 1:1 to about 6:1, about 1:1 to about 5:1, about 1:1 to about 4:1, about 1:1 to about 3:1, about 1:1 to about 2:1, about 1:1 to about 1:1.

In some embodiments, the albumin allows the composition to be administered to an individual (such as a human) without significant side effects. In some embodiments, the albumin (such as human serum albumin or human albumin) is in an amount that is effective to reduce one or more side effects of administration of the mTOR inhibitor (such as a limus drug, e.g., sirolimus or a derivative thereof) to a human. The term “reducing one or more side effects” of administration of the mTOR inhibitor (such as a limus drug, e.g., sirolimus or a derivative thereof) refers to reduction, alleviation, elimination, or avoidance of one or more undesirable effects caused by the mTOR inhibitor, as well as side effects caused by delivery vehicles (such as solvents that render the limus drugs suitable for injection) used to deliver the mTOR inhibitor. Such side effects include, for example, myelosuppression, neurotoxicity, hypersensitivity, inflammation, venous irritation, phlebitis, pain, skin irritation, peripheral neuropathy, neutropenic fever, anaphylactic reaction, venous thrombosis, extravasation, and combinations thereof. These side effects, however, are merely exemplary and other side effects, or combination of side effects, associated with limus drugs (such as a limus drug, e.g., sirolimus or a derivative thereof) can be reduced.

In some embodiments, the mTOR inhibitor nanoparticle compositions described herein comprise nanoparticles comprising an mTOR inhibitor (such as a limus drug, e.g., sirolimus or a derivative thereof) and an albumin (such as human albumin or human serum albumin), wherein the nanoparticles have an average diameter of no greater than about 200 nm. In some embodiments, the mTOR inhibitor nanoparticle compositions described herein comprise nanoparticles comprising an mTOR inhibitor (such as a limus drug, e.g., sirolimus or a derivative thereof) and an albumin (such as human albumin or human serum albumin), wherein the nanoparticles have an average diameter of no greater than about 150 nm. In some embodiments, the mTOR inhibitor nanoparticle compositions described herein comprise nanoparticles comprising an mTOR inhibitor (such as a limus drug, e.g., sirolimus or a derivative thereof) and an albumin (such as human albumin or human serum albumin), wherein the nanoparticles have an average diameter of no greater than about 150 nm (for example about 100 nm). In some embodiments, the mTOR inhibitor nanoparticle compositions described herein comprise nanoparticles comprising sirolimus and human albumin (such as human serum albumin), wherein the nanoparticles have an average diameter of no greater than about 150 nm (for example about 100 nm). In some embodiments, the mTOR inhibitor nanoparticle compositions described herein comprise nanoparticles comprising sirolimus and human albumin (such as human serum albumin), wherein the average or mean diameter of the nanoparticles is about 10 to about 150 nm. In some embodiments, the mTOR inhibitor nanoparticle compositions described herein comprise nanoparticles comprising sirolimus and human albumin (such as human serum albumin), wherein the average or mean diameter of the nanoparticles is about 40 to about 120 nm.

In some embodiments, the mTOR inhibitor nanoparticle compositions described herein comprise nanoparticles comprising an mTOR inhibitor (such as a limus drug, e.g., sirolimus or a derivative thereof) and an albumin (such as human albumin or human serum albumin), wherein the nanoparticles have an average diameter of no greater than about 200 nm, wherein the weight ratio of the albumin and the mTOR inhibitor in the composition is no greater than about 9:1 (such as about 9:1 or about 8:1). In some embodiments, the mTOR inhibitor nanoparticle compositions described herein comprise nanoparticles comprising an mTOR inhibitor (such as a limus drug, e.g., sirolimus or a derivative thereof) and an albumin (such as human albumin or human serum albumin), wherein the nanoparticles have an average diameter of no greater than about 150 nm, wherein the weight ratio of the albumin and the mTOR inhibitor in the composition is no greater than about 9:1 (such as about 9:1 or about 8:1). In some embodiments, the mTOR inhibitor nanoparticle compositions described herein comprise nanoparticles comprising an mTOR inhibitor (such as a limus drug, e.g., sirolimus or a derivative thereof) and an albumin (such as human albumin or human serum albumin), wherein the nanoparticles have an average diameter of about 150 nm, wherein the weight ratio of the albumin and the mTOR inhibitor in the composition is no greater than about 9:1 (such as about 9:1 or about 8:1). In some embodiments, the mTOR inhibitor nanoparticle compositions described herein comprise nanoparticles comprising sirolimus and human albumin (such as human serum albumin), wherein the nanoparticles have an average diameter of no greater than about 150 nm (for example about 100 nm), wherein the weight ratio of albumin and mTOR inhibitor in the composition is about 9:1 or about 8:1. In some embodiments, the average or mean diameter of the nanoparticles is about 10 nm to about 150 nm. In some embodiments, the average or mean diameter of the nanoparticles is about 40 nm to about 120 nm.

In some embodiments, the mTOR inhibitor nanoparticle compositions described herein comprise nanoparticles comprising an mTOR inhibitor (such as a limus drug, e.g., sirolimus or a derivative thereof) associated (e.g., coated) with an albumin (such as human albumin or human serum albumin). In some embodiments, the mTOR inhibitor nanoparticle compositions described herein comprise nanoparticles comprising an mTOR inhibitor (such as a limus drug, e.g., sirolimus or a derivative thereof) associated (e.g., coated) with an albumin (such as human albumin or human serum albumin), wherein the nanoparticles have an average diameter of no greater than about 200 nm. In some embodiments, the mTOR inhibitor nanoparticle compositions described herein comprise nanoparticles comprising an mTOR inhibitor (such as a limus drug, e.g., sirolimus or a derivative thereof) associated (e.g., coated) with an albumin (such as human albumin or human serum albumin), wherein the nanoparticles have an average diameter of no greater than about 150 nm. In some embodiments, the mTOR inhibitor nanoparticle compositions described herein comprise nanoparticles comprising an mTOR inhibitor (such as a limus drug, e.g., sirolimus or a derivative thereof) associated (e.g., coated) with an albumin (such as human albumin or human serum albumin), wherein the nanoparticles have an average diameter of about 10 nm to about 150 nm. In some embodiments, the mTOR inhibitor nanoparticle compositions described herein comprise nanoparticles comprising an mTOR inhibitor (such as a limus drug, e.g., sirolimus or a derivative thereof) associated (e.g., coated) with an albumin (such as human albumin or human serum albumin), wherein the nanoparticles have an average diameter of about 40 nm to about 120 nm. In some embodiments, the mTOR inhibitor nanoparticle compositions described herein comprise nanoparticles comprising sirolimus associated (e.g., coated) with human albumin (such as human serum albumin), wherein the nanoparticles have an average diameter of no greater than about 150 nm (for example about 100 nm). In some embodiments, the mTOR inhibitor nanoparticle compositions described herein comprise nanoparticles comprising sirolimus associated (e.g., coated) with human albumin (such as human serum albumin), wherein the nanoparticles have an average diameter of about 10 nm to about 150 nm. In some embodiments, the mTOR inhibitor nanoparticle compositions described herein comprise nanoparticles comprising sirolimus associated (e.g., coated) with human albumin (such as human serum albumin), wherein the nanoparticles have an average diameter of about 40 nm to about 120 nm.

In some embodiments, the mTOR inhibitor nanoparticle compositions described herein comprise nanoparticles comprising an mTOR inhibitor (such as a limus drug, e.g., sirolimus or a derivative thereof) associated (e.g., coated) with an albumin (such as human albumin or human serum albumin), wherein the weight ratio of the albumin and the mTOR inhibitor in the composition is no greater than about 9:1 (such as about 9:1 or about 8:1). In some embodiments, the mTOR inhibitor nanoparticle compositions described herein comprise nanoparticles comprising an mTOR inhibitor (such as a limus drug, e.g., sirolimus or a derivative thereof) associated (e.g., coated) with an albumin (such as human albumin or human serum albumin), wherein the nanoparticles have an average diameter of no greater than about 200 nm, wherein the weight ratio of the albumin and the mTOR inhibitor in the composition is no greater than about 9:1 (such as about 9:1 or about 8:1). In some embodiments, the mTOR inhibitor nanoparticle compositions described herein comprise nanoparticles comprising an mTOR inhibitor (such as a limus drug, e.g., sirolimus or a derivative thereof) associated (e.g., coated) with an albumin (such as human albumin or human serum albumin), wherein the nanoparticles have an average diameter of no greater than about 150 nm, wherein the weight ratio of the albumin and the mTOR inhibitor in the composition is no greater than about 9:1 (such as about 9:1 or about 8:1). In some embodiments, the mTOR inhibitor nanoparticle compositions described herein comprise nanoparticles comprising an mTOR inhibitor (such as a limus drug, e.g., sirolimus or a derivative thereof) associated (e.g., coated) with an albumin (such as human albumin or human serum albumin), wherein the nanoparticles have an average diameter of about 150 nm, wherein the weight ratio of the albumin and the mTOR inhibitor in the composition is no greater than about 9:1 (such as about 9:1 or about 8:1). In some embodiments, the mTOR inhibitor nanoparticle compositions described herein comprise nanoparticles comprising sirolimus associated (e.g., coated) with human albumin (such as human serum albumin), wherein the nanoparticles have an average diameter of no greater than about 150 nm (for example about 100 nm), wherein the weight ratio of albumin and the sirolimus in the composition is about 9:1 or about 8:1. In some embodiments, the average or mean diameter of the nanoparticles is about 10 nm to about 150 nm. In some embodiments, the average or mean diameter of the nanoparticles is about 40 nm to about 120 nm.

In some embodiments, the mTOR inhibitor nanoparticle compositions described herein comprise nanoparticles comprising an mTOR inhibitor (such as a limus drug, e.g., sirolimus or a derivative thereof) stabilized by an albumin (such as human albumin or human serum albumin). In some embodiments, the mTOR inhibitor nanoparticle compositions described herein comprise nanoparticles comprising an mTOR inhibitor (such as a limus drug, e.g., sirolimus or a derivative thereof) stabilized by an albumin (such as human albumin or human serum albumin), wherein the nanoparticles have an average diameter of no greater than about 200 nm. In some embodiments, the mTOR inhibitor nanoparticle compositions described herein comprise nanoparticles comprising an mTOR inhibitor (such as a limus drug, e.g., sirolimus or a derivative thereof) stabilized by an albumin (such as human albumin or human serum albumin), wherein the nanoparticles have an average diameter of no greater than about 150 nm. In some embodiments, the mTOR inhibitor nanoparticle compositions described herein comprise nanoparticles comprising an mTOR inhibitor (such as a limus drug, e.g., sirolimus or a derivative thereof) stabilized by an albumin (such as human albumin or human serum albumin), wherein the nanoparticles have an average diameter of no greater than about 150 nm (for example about 100 nm). In some embodiments, the mTOR inhibitor nanoparticle compositions described herein comprise nanoparticles comprising sirolimus stabilized by human albumin (such as human serum albumin), wherein the nanoparticles have an average diameter of no greater than about 150 nm (for example about 100 nm). In some embodiments, the average or mean diameter of the nanoparticles is about 10 nm to about 150 nm. In some embodiments, the average or mean diameter of the nanoparticles is about 40 nm to about 120 nm.

In some embodiments, the mTOR inhibitor nanoparticle compositions described herein comprise nanoparticles comprising an mTOR inhibitor (such as a limus drug, e.g., sirolimus or a derivative thereof) stabilized by an albumin (such as human albumin or human serum albumin), wherein the weight ratio of the albumin and the mTOR inhibitor in the composition is no greater than about 9:1 (such as about 9:1 or about 8:1). In some embodiments, the mTOR inhibitor nanoparticle compositions described herein comprise nanoparticles comprising an mTOR inhibitor (such as a limus drug, e.g., sirolimus or a derivative thereof) stabilized by an albumin (such as human albumin or human serum albumin), wherein the nanoparticles have an average diameter of no greater than about 200 nm, wherein the weight ratio of the albumin and the mTOR inhibitor in the composition is no greater than about 9:1 (such as about 9:1 or about 8:1). In some embodiments, the mTOR inhibitor nanoparticle compositions described herein comprise nanoparticles comprising an mTOR inhibitor (such as a limus drug, e.g., sirolimus or a derivative thereof) stabilized by an albumin (such as human albumin or human serum albumin), wherein the nanoparticles have an average diameter of no greater than about 150 nm, wherein the weight ratio of the albumin and the mTOR inhibitor in the composition is no greater than about 9:1 (such as about 9:1 or about 8:1). In some embodiments, the mTOR inhibitor nanoparticle compositions described herein comprise nanoparticles comprising an mTOR inhibitor (such as a limus drug, e.g., sirolimus or a derivative thereof) stabilized by an albumin (such as human albumin or human serum albumin), wherein the nanoparticles have an average diameter of about 150 nm, wherein the weight ratio of the albumin and the mTOR inhibitor in the composition is no greater than about 9:1 (such as about 9:1 or about 8:1). In some embodiments, the mTOR inhibitor nanoparticle compositions described herein comprise nanoparticles comprising sirolimus stabilized by human albumin (such as human serum albumin), wherein the nanoparticles have an average diameter of no greater than about 150 nm (for example about 100 nm), wherein the weight ratio of albumin and the sirolimus in the composition is about 9:1 or about 8:1. In some embodiments, the average or mean diameter of the nanoparticles is about 10 nm to about 150 nm. In some embodiments, the average or mean diameter of the nanoparticles is about 40 nm to about 120 nm.

In some embodiments, the mTOR inhibitor nanoparticle composition comprises nab-sirolimus. In some embodiments, the mTOR inhibitor nanoparticle composition is nab-sirolimus. nab-sirolimus is a formulation of sirolimus stabilized by human albumin USP, which can be dispersed in directly injectable physiological solution. The weight ratio of human albumin and sirolimus is about 8:1 to about 9:1. When dispersed in a suitable aqueous medium such as 0.9% sodium chloride injection or 5% dextrose injection, nab-sirolimus forms a stable colloidal suspension of sirolimus. The mean particle size of the nanoparticles in the colloidal suspension is about 100 nanometers. Since HSA is freely soluble in water, nab-sirolimus can be reconstituted in a wide range of concentrations ranging from dilute (0.1 mg/ml sirolimus or a derivative thereof) to concentrated (20 mg/ml sirolimus or a derivative thereof), including for example about 2 mg/ml to about 8 mg/ml, or about 5 mg/ml.

Methods of making nanoparticle compositions are known in the art. For example, nanoparticles containing an mTOR inhibitor (such as a limus drug, e.g., sirolimus or a derivative thereof) and an albumin (such as human serum albumin or human albumin) can be prepared under conditions of high shear forces (e.g., sonication, high pressure homogenization, or the like). These methods are disclosed in, for example, U.S. Pat. Nos. 5,916,596; 6,506,405; 6,749,868, 6,537,579, 7,820,788, and 8,911,786, and also in U.S. Pat. Pub. Nos. 2007/0082838, 2006/0263434 and PCT Application WO08/137148.

Briefly, the mTOR inhibitor (such as a limus drug, e.g., sirolimus or a derivative thereof) is dissolved in an organic solvent, and the solution can be added to an albumin solution. The mixture is subjected to high pressure homogenization. The organic solvent can then be removed by evaporation. The dispersion obtained can be further lyophilized. Suitable organic solvent include, for example, ketones, esters, ethers, chlorinated solvents, and other solvents known in the art. For example, the organic solvent can be methylene chloride or chloroform/ethanol (for example with a ratio of 1:9, 1:8, 1:7, 1:6, 1:5, 1:4, 1:3, 1:2, 1:1, 2:1, 3:1, 4:1, 5:1, 6:1, 7:1, 8:1, or 9:1).

mTOR Inhibitor

The methods described herein in some embodiments comprise administration of nanoparticle compositions of mTOR inhibitors. “mTOR inhibitor” used herein refers to an inhibitor of mTOR. mTOR is a serine/threonine-specific protein kinase downstream of the phosphatidylinositol 3-kinase (PI3K)/Akt (protein kinase B) pathway, and a key regulator of cell survival, proliferation, stress, and metabolism. mTOR pathway dysregulation has been found in many human carcinomas, and mTOR inhibition produced substantial inhibitory effects on tumor progression.

The mammalian target of rapamycin (mTOR) (also known as mechanistic target of rapamycin or FK506 binding protein 12-rapamycin associated protein 1 (FRAP1)) is an atypical serine/threonine protein kinase that is present in two distinct complexes, mTOR Complex 1 (mTORC1) and mTOR Complex 2 (mTORC2). mTORC1 is composed of mTOR, regulatory-associated protein of mTOR (Raptor), mammalian lethal with SEC13 protein 8 (MLST8), PRAS40 and DEPTOR (Kim et al. (2002). Cell 110: 163-75; Fang et al. (2001). Science 294 (5548): 1942-5). mTORC1 integrates four major signal inputs: nutrients (such as amino acids and phosphatidic acid), growth factors (insulin), energy and stress (such as hypoxia and DNA damage). Amino acid availability is signaled to mTORC1 via a pathway involving the Rag and Ragulator (LAMTOR1-3) Growth factors and hormones (e.g., insulin) signal to mTORC1 via Akt, which inactivates TSC2 to prevent inhibition of mTORC1. Alternatively, low ATP levels lead to the AMPK-dependent activation of TSC2 and phosphorylation of raptor to reduce mTORC1 signaling proteins.

Active mTORC1 has a number of downstream biological effects including translation of mRNA via the phosphorylation of downstream targets (4E-BP1 and p70 S6 Kinase), suppression of autophagy (Atg13, ULK1), ribosome biogenesis, and activation of transcription leading to mitochondrial metabolism or adipogenesis. Accordingly, mTORC1 activity promotes either cellular growth when conditions are favorable or catabolic processes during stress or when conditions are unfavorable.

mTORC2 is composed of mTOR, rapamycin-insensitive companion of mTOR (RICTOR), GβL, and mammalian stress-activated protein kinase interacting protein 1 (mSIN1). In contrast to mTORC1, for which many upstream signals and cellular functions have been defined (see above), relatively little is known about mTORC2 biology. mTORC2 regulates cytoskeletal organization through its stimulation of F-actin stress fibers, paxillin, RhoA, Rac1, Cdc42, and protein kinase C α (PKCα). It had been observed that knocking down mTORC2 components affects actin polymerization and perturbs cell morphology (Jacinto et al. (2004). Nat. Cell Biol. 6, 1122-1128; Sarbassov et al. (2004). Curr. Biol. 14, 1296-1302). This suggests that mTORC2 controls the actin cytoskeleton by promoting protein kinase Cα (PKCα) phosphorylation, phosphorylation of paxillin and its relocalization to focal adhesions, and the GTP loading of RhoA and Rac1. The molecular mechanism by which mTORC2 regulates these processes has not been determined.

In some embodiments, the mTOR inhibitor (such as a limus drug, e.g., sirolimus or a derivative thereof) is an inhibitor of mTORC1. In some embodiments, the mTOR inhibitor (such as a limus drug, e.g., sirolimus or a derivative thereof) is an inhibitor of mTORC2. In some embodiments, the mTOR inhibitor (such as a limus drug, e.g., sirolimus or a derivative thereof) is an inhibitor of both mTORC1 and mTORC2.

In some embodiments, the mTOR inhibitor is a limus drug, which includes sirolimus and its analogs. Examples of limus drugs include, but are not limited to, temsirolimus (CCI-779), everolimus (RAD001), ridaforolimus (AP-23573), deforolimus (MK-8669), zotarolimus (ABT-578), pimecrolimus, and tacrolimus (FK-506). In some embodiments, the limus drug is selected from the group consisting of temsirolimus (CCI-779), everolimus (RAD001), ridaforolimus (AP-23573), deforolimus (MK-8669), zotarolimus (ABT-578), pimecrolimus, and tacrolimus (FK-506). In some embodiments, the mTOR inhibitor is an mTOR kinase inhibitor, such as CC-115 or CC-223.

In some embodiments, the mTOR inhibitor is sirolimus. Sirolimus is macrolide antibiotic that complexes with FKBP-12 and inhibits the mTOR pathway by binding mTORC1.

In some embodiments, the mTOR inhibitor is selected from the group consisting of sirolimus (rapamycin), BEZ235 (NVP-BEZ235), everolimus (also known as RAD001, Zortress, Certican, and Afinitor), AZD8055, temsirolimus (also known as CCI-779 and Torisel), CC-115, CC-223, PI-103, Ku-0063794, INK 128, AZD2014, NVP-BGT226, PF-04691502, CH5132799, GDC-0980 (RG7422), Torin 1, WAY-600, WYE-125132, WYE-687, GSK2126458, PF-05212384 (PKI-587), PP-121, OSI-027, Palomid 529, PP242, XL765, GSK1059615, WYE-354, and ridaforolimus (also known as deforolimus).

BEZ235 (NVP-BEZ235) is an imidazoquilonine derivative that is an mTORC1 catalytic inhibitor (Roper J, et al. PLoS One, 2011, 6(9), e25132). Everolimus is the 40-O-(2-hydroxyethyl) derivative of sirolimus and binds the cyclophilin FKBP-12, and this complex also mTORC1. AZD8055 is a small molecule that inhibits the phosphorylation of mTORC1 (p70S6K and 4E-BP1). Temsirolimus is a small molecule that forms a complex with the FK506-binding protein and prohibits the activation of mTOR when it resides in the mTORC1 complex. PI-103 is a small molecule that inhibits the activation of the rapamycin-sensitive (mTORC1) complex (Knight et al. (2006) Cell. 125: 733-47). KU-0063794 is a small molecule that inhibits the phosphorylation of mTORC1 at Ser2448 in a dose-dependent and time-dependent manner. INK 128, AZD2014, NVP-BGT226, CH5132799, WYE-687, and are each small molecule inhibitors of mTORC1. PF-04691502 inhibits mTORC1 activity. GDC-0980 is an orally bioavailable small molecule that inhibits Class I PI3 Kinase and TORC1. Torin 1 is a potent small molecule inhibitor of mTOR. WAY-600 is a potent, ATP-competitive and selective inhibitor of mTOR. WYE-125132 is an ATP-competitive small molecule inhibitor of mTORC1. GSK2126458 is an inhibitor of mTORC1. PKI-587 is a highly potent dual inhibitor of PI3Kα, PI3Kγ and mTOR. PP-121 is a multi-target inhibitor of PDGFR, Hck, mTOR, VEGFR2, Src and Abl. OSI-027 is a selective and potent dual inhibitor of mTORC1 and mTORC2 with IC50 of 22 nM and 65 nM, respectively. Palomid 529 is a small molecule inhibitor of mTORC1 that lacks affinity for ABCB1/ABCG2 and has good brain penetration (Lin et al. (2013) Int J Cancer DOI: 10.1002/ijc. 28126 (e-published ahead of print). PP242 is a selective mTOR inhibitor. XL765 is a dual inhibitor of mTOR/PI3k for mTOR, p110α, p110β, p110γ and p110δ. GSK1059615 is a novel and dual inhibitor of PI3Kα, PI3Kβ, PI3Kδ, PI3Kγ and mTOR. WYE-354 inhibits mTORC1 in HEK293 cells (0.2 μM-5 μM) and in HUVEC cells (10 nM-1p M). WYE-354 is a potent, specific and ATP-competitive inhibitor of mTOR. Deforolimus (Ridaforolimus, AP23573, MK-8669) is a selective mTOR inhibitor.

Other Components in the mTOR Inhibitor Nanoparticle Compositions

The nanoparticles described herein can be present in a composition that include other agents, excipients, or stabilizers. For example, to increase stability by increasing the negative zeta potential of nanoparticles, certain negatively charged components may be added. Such negatively charged components include, but are not limited to bile salts of bile acids consisting of glycocholic acid, cholic acid, chenodeoxycholic acid, taurocholic acid, glycochenodeoxycholic acid, taurochenodeoxycholic acid, litocholic acid, ursodeoxycholic acid, dehydrocholic acid and others; phospholipids including lecithin (egg yolk) based phospholipids which include the following phosphatidylcholines: palmitoyloleoylphosphatidylcholine, palmitoyllinoleoylphosphatidylcholine, stearoyllinoleoylphosphatidylcholine stearoyloleoylphosphatidylcholine, stearoylarachidoylphosphatidylcholine, and dipalmitoylphosphatidylcholine. Other phospholipids including L-α-dimyristoylphosphatidylcholine (DMPC), dioleoylphosphatidylcholine (DOPC), distearyolphosphatidylcholine (DSPC), hydrogenated soy phosphatidylcholine (HSPC), and other related compounds. Negatively charged surfactants or emulsifiers are also suitable as additives, e.g., sodium cholesteryl sulfate and the like.

In some embodiments, the composition is suitable for administration to a human. In some embodiments, the composition is suitable for administration to a mammal such as, in the veterinary context, domestic pets and agricultural animals. There are a wide variety of suitable formulations of the mTOR inhibitor nanoparticle composition (such as sirolimus/albumin nanoparticle composition) (see, e.g., U.S. Pat. Nos. 5,916,596 and 6,096,331). The following formulations and methods are merely exemplary and are in no way limiting. Formulations suitable for oral administration can consist of (a) liquid solutions, such as an effective amount of the compound dissolved in diluents, such as water, saline, or orange juice, (b) capsules, sachets or tablets, each containing a predetermined amount of the active ingredient, as solids or granules, (c) suspensions in an appropriate liquid, and (d) suitable emulsions. Tablet forms can include one or more of lactose, mannitol, corn starch, potato starch, microcrystalline cellulose, acacia, gelatin, colloidal silicon dioxide, croscarmellose sodium, talc, magnesium stearate, stearic acid, and other excipients, colorants, diluents, buffering agents, moistening agents, preservatives, flavoring agents, and pharmacologically compatible excipients. Lozenge forms can comprise the active ingredient in a flavor, usually sucrose and acacia or tragacanth, as well as pastilles comprising the active ingredient in an inert base, such as gelatin and glycerin, or sucrose and acacia, emulsions, gels, and the like containing, in addition to the active ingredient, such excipients as are known in the art.

Examples of suitable carriers, excipients, and diluents include, but are not limited to, lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water, saline solution, syrup, methylcellulose, methyl- and propylhydroxybenzoates, talc, magnesium stearate, and mineral oil. The formulations can additionally include lubricating agents, wetting agents, emulsifying and suspending agents, preserving agents, sweetening agents or flavoring agents.

Formulations suitable for parenteral administration include aqueous and non-aqueous, isotonic sterile injection solutions, which can contain anti-oxidants, buffers, bacteriostats, and solutes that render the formulation compatible with the blood of the intended recipient, and aqueous and non-aqueous sterile suspensions that can include suspending agents, solubilizers, thickening agents, stabilizers, and preservatives. The formulations can be presented in unit-dose or multi-dose sealed containers, such as ampules and vials, and can be stored in a freeze-dried (lyophilized) condition requiring only the addition of the sterile liquid excipient, for example, water, for injections, immediately prior to use. Extemporaneous injection solutions and suspensions can be prepared from sterile powders, granules, and tablets of the kind previously described. Injectable formulations are preferred.

In some embodiments, the composition is formulated to have a pH range of about 4.5 to about 9.0, including for example pH ranges of about any of 5.0 to about 8.0, about 6.5 to about 7.5, and about 6.5 to about 7.0. In some embodiments, the pH of the composition is formulated to no less than about 6, including for example no less than about any of 6.5, 7, or 8 (such as about 8). The composition can also be made to be isotonic with blood by the addition of a suitable tonicity modifier, such as glycerol.

### Immunomodulators

The methods described herein in some embodiments comprise administration of nanoparticle compositions of mTOR inhibitors in combination with an immunomodulator. “Immunomodulator” used herein refers to a therapeutic agent that when present, alters, suppresses or stimulates the body's immune system. Immunomodulators can include compositions or formulations that activate the immune system (e.g., adjuvants or activators), or downregulate the immune system. Adjuvants can include aluminum-based compositions, as well as compositions that include bacterial or mycobacterial cell wall components. Activators can include molecules that activate antigen presenting cells to stimulate the cellular immune response. For example, activators can be immunostimulant peptides. Activators can include, but are not limited to, agonists of toll-like receptors TLR-2, 3, 4, 6, 7, 8, or 9, granulocyte macrophage colony stimulating factor (GM-CSF); TNF; CD40L; CD28; FLT-3 ligand; or cytokines such as IL-1, IL-2, IL-4, IL-7, IL-12, IL-15, or IL-21. Activators can include agonists of activating receptors (including co-stimulatory receptors) on T cells, such as an agonist (e.g., agonistic antibody) of CD28, OX40, GITR, 4-1BB, ICOS, CD27, CD40, or HVEM. Activators can also include compounds that inhibit the activity of an immune suppressor, such as an inhibitor of the immune suppressors IL-10, FasL, IL-35, TGF-β, indoleamine-2,3 dioxygenase (IDO), or cyclophosphamide, or inhibit the activity of an immune checkpoint such as an antagonist (e.g., antagonistic antibody) of CTLA4, PD-1, PD-L1, PD-L2, LAG3, B7-1, B7-H3, B7-H4, BTLA, VISTA, KIR, A2aR, or TIM3. Activators can also include costimulatory molecules such as CD40, CD80, or CD86. Immunomodulators can also include agents that downregulate the immune system such as antibodies against IL-12p70, antagonists of toll-like receptors TLR-2, 3, 4, 5, 6, 8, or 9, or general suppressors of immune function such as cyclophosphamide, cyclosporin A or FK506. Other antibodies of interest include those directed to tumor cell targets, including for example anti-GD2 antibody (such as dinutuximab). These agents (e.g., adjuvants, activators, or downregulators) can be combined to shape an optimal immune response.

The indoleamine-2,3 dioxygenase (IDO) enzyme catalyzes the breakdown of the essential amino acid tryptophan, and has emerged as a key target in cancer immunotherapy because of its role in enabling cancers to evade the immune system. IDO activity leads to a tryptophan deficit, which starves cytotoxic T-cells within the tumor microenvironment. Additionally, the resulting tryptophan metabolites activate regulatory T-cells, which further suppresses the immune response to the tumor. IDO is overexpressed by antigen presenting cells in many cancers, and high IDO expression appears to correlate with poor outcome in a number of cancers, including ovarian cancer, AML, endometrial carcinoma, colon cancer, and melanoma. Blocking IDO enhances immune response against tumors. IDO inhibitors include, but are not limited to, small molecule or antibody-based inhibitors, such as 1-methyl-[D]-tryptophan (D-1MT, NSC-721782), epacadostat (INCB24360), norharmane (β-Carboline), rosmarinic acid, and COX-2 inhibitors.

As used herein, the term “immune checkpoint inhibitors,” “checkpoint inhibitors,” and the like refers to compounds that inhibit the activity of control mechanisms of the immune system. Immune system checkpoints, or immune checkpoints, are inhibitory pathways in the immune system that generally act to maintain self-tolerance or modulate the duration and amplitude of physiological immune responses to minimize collateral tissue damage. Checkpoint inhibitors can inhibit an immune system checkpoint by inhibiting the activity of a protein in the pathway. Immune system checkpoint proteins include, but are not limited to, cytotoxic T-lymphocyte antigen 4 (CTLA4), programmed cell death 1 protein (PD-1), programmed cell death 1 ligand 1 (PD-L1), programmed cell death ligand 2 (PD-L2), lymphocyte activation gene 3 (LAG3), B7-1, B7-H3, B7-H4, T cell membrane protein 3 (TIM3), B- and T-lymphocyte attenuator (BTLA), V-domain immunoglobulin (Ig)-containing suppressor of T-cell activation (VISTA), Killer-cell immunoglobulin-like receptor (KIR), and A2A adenosine receptor (A2aR). As such, checkpoint inhibitors include antagonists of CTLA4, PD-1, PD-L1, PD-L2, LAG3, B7-1, B7-H3, B7-H4, BTLA, VISTA, KIR, A2aR, or TIM3. For example, antibodies that bind to CTLA4, PD-1, PD-L1, PD-L2, LAG3, B7-1, B7-H3, B7-H4, BTLA, VISTA, KIR, A2aR, or TIM3 and antagonize their function are checkpoint inhibitors. Moreover, any molecule (e.g., peptide, nucleic acid, small molecule, etc.) that inhibits the inhibitory function of an immune system checkpoint is a checkpoint inhibitor.

Sirolimus, derivatives thereof, and other mTOR inhibitors are generally regarded as immunosuppressive agents and therefore there has been no interest in combining immuno-oncology antibody drugs (for example, anti-PD-1 or anti-PD-L1) with mTOR inhibitors, since the main goal of those therapies is to activate the immune system against the target cells or disease. We propose, however, that the use of mTOR inhibitors, specifically ABI-009 (albumin-bound nanoparticles of sirolimus) may activate the immune system, including for example T cells, such as CD8⁺ T cells or memory T cells, to further improve the activity of these immune-oncology agents against the disease.

CTLA-4 is an immune checkpoint molecule, which is up-regulated on activated T-cells. An anti-CTLA4 mAb can block the interaction of CTLA-4 with CD80/86 and switch off the mechanism of immune suppression and enable continuous stimulation of T-cells by DCs. Two IgG mAb directed against CTLA-4, ipilimumab and tremelimumab, have been tested in clinical trials for a number of indications. Ipilimumab is approved by the FDA for the treatment of melanoma.

PD-1 is a part of the B7/CD28 family of co-stimulatory molecules that regulate T-cell activation and tolerance, and thus antagonistic anti-PD-1 antibodies can be useful for overcoming tolerance. Engagement of the PD-1/PD-L1 pathway results in inhibition of T-cell effector function, cytokine secretion and proliferation. (Turnis et al., OncoImmunology 1(7):1172-1174, 2012). High levels of PD-1 are associated with exhausted or chronically stimulated T cells. Moreover, increased PD-1 expression correlates with reduced survival in cancer patients. Nivolumab is a human mAb to PD-1 that is FDA approved for the treatment of unresectable or metastatic melanoma, as well as squamous non-small cell lung cancer.

In some embodiments, according to any of the methods described above, the immunomodulator enhances an immune response in the individual and may include, but is not limited to, a cytokine, a chemokine, a stem cell growth factor, a lymphotoxin, an hematopoietic factor, a colony stimulating factor (CSF), erythropoietin, thrombopoietin, tumor necrosis factor-alpha (TNF), TNF-beta, granulocyte-colony stimulating factor (G-CSF), granulocyte macrophage-colony stimulating factor (GM-CSF), interferon-alpha, interferon-beta, interferon-gamma, interferon-lambda, stem cell growth factor designated “S1 factor”, human growth hormone, N-methionyl human growth hormone, bovine growth hormone, parathyroid hormone, thyroxine, insulin, proinsulin, relaxin, prorelaxin, follicle stimulating hormone (FSH), thyroid stimulating hormone (TSH), luteinizing hormone (LH), hepatic growth factor, prostaglandin, fibroblast growth factor, prolactin, placental lactogen, OB protein, mullerian-inhibiting substance, mouse gonadotropin-associated peptide, inhibin, activin, vascular endothelial growth factor, integrin, NGF-beta, platelet-growth factor, TGF-alpha, TGF-beta, insulin-like growth factor-I, insulin-like growth factor-II, macrophage-CSF (M-CSF), IL-1, IL-1a, IL-2, IL-3, IL-4, IL-5, IL-6, IL-7, IL-8, IL-9, IL-10, IL-11, IL-12, IL-13, IL-14, IL-15, IL-16, IL-17, IL-18, IL-21, IL-25, LIF, FLT-3, angiostatin, thrombospondin, endostatin, lymphotoxin, thalidomide, lenalidomide, or pomalidomide. In some embodiments, the immunomodulator is pomalidomide or an enantiomer or a mixture of enantiomers thereof, or a pharmaceutically acceptable salt, solvate, hydrate, co-crystal, clathrate, or polymorph thereof. In some embodiments, the immunomodulator is lenalidomide or an enantiomer or a mixture of enantiomers thereof, or a pharmaceutically acceptable salt, solvate, hydrate, co-crystal, clathrate, or polymorph thereof.

In some embodiments, according to any of the methods described above, the immunomodulator enhances an immune response in the individual and may include, but is not limited to, an antagonistic antibody selected from the group consisting of anti-CTLA4 (such as Ipilimumab and Tremelimumab), anti-PD-1 (such as Nivolumab, Pidilizumab, and Pembrolizumab), anti-PD-L1 (such as MPDL3280A, BMS-936559, MED14736, and Avelumab), anti-PD-L2, anti-LAG3 (such as BMS-986016 or C9B7W), anti-B7-1, anti-B7-H3 (such as MGA271), anti-B7-H4, anti-TIM3, anti-BTLA, anti-VISTA, anti-KIR (such as Lirilumab and IPH2101), anti-A2aR, anti-CD52 (such as alemtuzumab), anti-IL-10, anti-FasL, anti-IL-35, and anti-TGF-0 (such as Fresolumimab). In some embodiments, the antibody is an antagonistic antibody. In some embodiments, the antibody is a monoclonal antibody. In some embodiments, the antibody is human or humanized.

In some embodiments, according to any of the methods described above, the immunomodulator enhances an immune response in the individual and may include, but is not limited to, an antibody selected from the group consisting of anti-CD28, anti-OX40 (such as MED16469), anti-GITR (such as TRX518), anti-4-1BB (such as BMS-663513 and PF-05082566), anti-ICOS (such as JTX-2011, Jounce Therapeutics), anti-CD27 (such as Varlilumab and hCD27.15), anti-CD40 (such as CP870,893), and anti-HVEM. In some embodiments, the antibody is an agonistic antibody. In some embodiments, the antibody is a monoclonal antibody. In some embodiments, the antibody is human or humanized.

Thus, in some embodiments, there is provided a method of treating a solid tumor (such as bladder cancer, renal cell carcinoma, or melanoma) in an individual (such as a human) comprising administering to the individual a) an effective amount of a composition comprising nanoparticles comprising an mTOR inhibitor (such as a limus drug, e.g., sirolimus or a derivative thereof) and an albumin; and b) an effective amount of an immunomodulator. In some embodiments, the immunomodulator is an immunostimulator. In some embodiments, the immunostimulator directly stimulates the immune system of an individual. In some embodiments, the immunomodulator is an IMiDs® compound (Celgene). IMiDs® compounds are proprietary small molecule, orally available compounds that modulate the immune system and other biological targets through multiple mechanisms of action, such as lenalidomide and pomalidomide. In some embodiments, the immunomodulator is small molecule or antibody-based IDO inhibitor. In some embodiments, the immunomodulator is selected from the group consisting of a cytokine, a chemokine, a stem cell growth factor, a lymphotoxin, an hematopoietic factor, a colony stimulating factor (CSF), erythropoietin, thrombopoietin, tumor necrosis factor-alpha (TNF), TNF-beta, granulocyte-colony stimulating factor (G-CSF), granulocyte macrophage-colony stimulating factor (GM-CSF), interferon-alpha, interferon-beta, interferon-gamma, interferon-lambda, stem cell growth factor designated “S1 factor”, human growth hormone, N-methionyl human growth hormone, bovine growth hormone, parathyroid hormone, thyroxine, insulin, proinsulin, relaxin, prorelaxin, follicle stimulating hormone (FSH), thyroid stimulating hormone (TSH), luteinizing hormone (LH), hepatic growth factor, prostaglandin, fibroblast growth factor, prolactin, placental lactogen, OB protein, mullerian-inhibiting substance, mouse gonadotropin-associated peptide, inhibin, activin, vascular endothelial growth factor, integrin, NGF-beta, platelet-growth factor, TGF-alpha, TGF-beta, insulin-like growth factor-I, insulin-like growth factor-II, macrophage-CSF (M-CSF), IL-1, IL-1a, IL-2, IL-3, IL-4, IL-5, IL-6, IL-7, IL-8, IL-9, IL-10, IL-11, IL-12, IL-13, IL-14, IL-15, IL-16, IL-17, IL-18, IL-21, IL-25, LIF, FLT-3, angiostatin, thrombospondin, endostatin, lymphotoxin, thalidomide, lenalidomide, and pomalidomide. In some embodiments, the immunomodulator is lenalidomide or an enantiomer or a mixture of enantiomers thereof, or a pharmaceutically acceptable salt, solvate, hydrate, co-crystal, clathrate, or polymorph thereof. In some embodiments, the immunomodulator is pomalidomide or an enantiomer or a mixture of enantiomers thereof, or a pharmaceutically acceptable salt, solvate, hydrate, co-crystal, clathrate, or polymorph thereof. In some embodiments, the immunomodulator is an agonistic antibody that targets an activating receptor (including co-stimulatory receptors) on an immune cell (such as a T cell). In some embodiments, the immunomodulator is an agonistic antibody selected from the group consisting of anti-CD28, anti-OX40 (such as MEDI6469), anti-GITR (such as TRX518), anti-4-1BB (such as BMS-663513 and PF-05082566), anti-ICOS (such as JTX-2011, Jounce Therapeutics), anti-CD27 (such as Varlilumab and hCD27.15), anti-CD40 (such as CP870,893), and anti-HVEM. In some embodiments, the immunomodulator is an immune checkpoint inhibitor. In some embodiments, the immune checkpoint inhibitor is an antagonistic antibody that targets an immune checkpoint protein. In some embodiments, the immunomodulator is an antagonistic antibody selected from the group consisting of anti-CTLA4 (such as Ipilimumab and Tremelimumab), anti-PD-1 (such as Nivolumab, Pidilizumab, and Pembrolizumab), anti-PD-L1 (such as MPDL3280A, BMS-936559, MEDI4736, and Avelumab), anti-PD-L2, anti-LAG3 (such as BMS-986016 or C9B7W), anti-B7-1, anti-B7-H3 (such as MGA271), anti-B7-H4, anti-TIM3, anti-BTLA, anti-VISTA, anti-KIR (such as Lirilumab and IPH2101), anti-A2aR, anti-CD52 (such as alemtuzumab), anti-IL-10, anti-FasL, anti-IL-35, and anti-TGF-0 (such as Fresolumimab).

In some embodiments, the immunomodulator is an immunostimulator. In some embodiments, the immunomodulator is an immunostimulator that directly stimulates the immune system of an individual. In some embodiments, the immunomodulator is an agonistic antibody that targets an activating receptor on an immune cell (such as a T cell). In some embodiments, the immunomodulator is an immune checkpoint inhibitor. In some embodiments, the immune checkpoint inhibitor is an antagonistic antibody that targets an immune checkpoint protein. In some embodiments, the immunomodulator is selected from the group consisting of a cytokine, a chemokine, a stem cell growth factor, a lymphotoxin, an hematopoietic factor, a colony stimulating factor (CSF), erythropoietin, thrombopoietin, tumor necrosis factor-alpha (TNF), TNF-beta, granulocyte-colony stimulating factor (G-CSF), granulocyte macrophage-colony stimulating factor (GM-CSF), interferon-alpha, interferon-beta, interferon-gamma, interferon-lambda, stem cell growth factor designated “S1 factor”, human growth hormone, N-methionyl human growth hormone, bovine growth hormone, parathyroid hormone, thyroxine, insulin, proinsulin, relaxin, prorelaxin, follicle stimulating hormone (FSH), thyroid stimulating hormone (TSH), luteinizing hormone (LH), hepatic growth factor, prostaglandin, fibroblast growth factor, prolactin, placental lactogen, OB protein, mullerian-inhibiting substance, mouse gonadotropin-associated peptide, inhibin, activin, vascular endothelial growth factor, integrin, NGF-beta, platelet-growth factor, TGF-alpha, TGF-beta, insulin-like growth factor-I, insulin-like growth factor-II, macrophage-CSF (M-CSF), IL-1, IL-1a, IL-2, IL-3, IL-4, IL-5, IL-6, IL-7, IL-8, IL-9, IL-10, IL-11, IL-12, IL-13, IL-14, IL-15, IL-16, IL-17, IL-18, IL-21, IL-25, LIF, FLT-3, angiostatin, thrombospondin, endostatin, lymphotoxin, thalidomide, lenalidomide, and pomalidomide. In some embodiments, the immunomodulator is pomalidomide or an enantiomer or a mixture of enantiomers thereof, or a pharmaceutically acceptable salt, solvate, hydrate, co-crystal, clathrate, or polymorph thereof. In some embodiments, the immunomodulator is lenalidomide or an enantiomer or a mixture of enantiomers thereof, or a pharmaceutically acceptable salt, solvate, hydrate, co-crystal, clathrate, or polymorph thereof. In some embodiments, the immunomodulator is an antagonistic antibody selected from the group consisting of anti-CTLA4 (such as Ipilimumab and Tremelimumab), anti-PD-1 (such as Nivolumab, Pidilizumab, and Pembrolizumab), anti-PD-L1 (such as MPDL3280A, BMS-936559, MED14736, and Avelumab), anti-PD-L2, anti-LAG3 (such as BMS-986016 or C9B7W), anti-B7-1, anti-B7-H3 (such as MGA271), anti-B7-H4, anti-TIM3, anti-BTLA, anti-VISTA, anti-KIR (such as Lirilumab and IPH2101), anti-A2aR, anti-CD52 (such as alemtuzumab), anti-IL-10, anti-FasL, anti-IL-35, and anti-TGF-0 (such as Fresolumimab).

### Histone Deacetylase Inhibitors

The methods described herein in some embodiments comprise administration of nanoparticle compositions of mTOR inhibitors in combination with a histone deacetylase inhibitor. Histone deacetylase (HDAC) inhibitors have demonstrated significant clinical benefit as single agents in cutaneous and peripheral T cell lymphomas, and have received FDA approval for these indications.

Histone deacetylases are divided into 4 classes: class-I (HDAC1, 2, 3, 8), class-IIa (HDAC4, 5, 7, 9), class-IIb (HDAC6, 10), class-III (SIRT1-7), and class-IV (HDAC11). These classes differ in their subcellular localization (class-I HDACs are present in nucleus and class-II enzymes are cytoplasmic) and their intracellular targets. Although HDACs are typically associated with target histone proteins, recent studies reveal at least 3,600 acetylation sites on 1,750 non-histone proteins in cancer cells associated with various functions including gene expression, DNA replication and repair, cdl cycle progression, cytoskeletal reorganization, and protein chaperone activity. Clinical trials with non-selective HDAC inhibitors (HDACi) have shown efficacy, but are limited due to side effects, such as fatigue, diarrhea, and thrombocytopenia.

HDAC inhibitors include, but are not limited to, vorinostat (SAHA), panobinostat (LBH589), belinostat (PXD101, CAS 414864-00-9), tacedinaline (N-acetyldinaline, CI-994), givinostat (gavinostat, ITF2357), FRM-0334 (EVP-0334), resveratrol (SRT501), CUDC-101, quisinostat (JNJ-26481585), abexinostat (PCI-24781), dacinostat (LAQ824, NVP-LAQ824), valproic acid, 4-(dimethylamino)N-[6-(hydroxyamino)-6-oxohexyl]-benzamide (HDAC1 inhibitor), 4-Iodo suberoylanilide hydroxamic acid (HDAC1 and HDAC6 inhibitor), romidepsin (a cyclic tetrapeptide with HDAC inhibitory activity primarily towards class-I HDACs), 1-naphthohydroxamic acid (HDAC1 and HDAC6 inhibitor), HDAC inhibitors based on amino-benzamide biasing elements (e.g., mocetinostat (MGCD103) and entinostat (MS275), which are highly selective for HDAC1, 2 and 3), AN-9 (CAS 122110-53-6), APHA Compound 8 (CAS 676599-90-9), apicidin (CAS 183506-66-3), BML-210 (CAS 537034-17-6), salermide (CAS 1105698-15-4), suberoyl bis-hydroxamic acid (CAS 38937-66-5) (HDAC1 and HDAC3 inhibitor), butyrylhydroxamic acid (CAS 4312-91-8), CAY10603 (CAS 1045792-66-2) (HDAC6 inhibitor), CBHA (CAS 174664-65-4), ricolinostat (ACY1215, rocilinostat), trichostatin-A, WT-161, tubacin, and Merck60.

In some embodiments, the HDAC inhibitor is a nucleotide based or protein/peptide based inhibitor of an HDAC. For example, nucleotide based inhibitors of an HDAC can include, but are not limited to, short hairpin RNA (shRNA), RNA interference (RNAi), short interfering RNA (siRNA), microRNA (miRNA), locked nucleic acids (LNA), DNA, peptide-nucleic acids (PNA), morpholinos, and aptamers. In some embodiments, nucleotide based inhibitors are composed of at least one modified base. In some embodiments, nucleotide based inhibitors bind to the mRNA of an HDAC and decrease or inhibit its translation, or increase its degradation. In some embodiments, nucleotide based inhibitors decrease the expression (e.g., at the mRNA transcript and/or protein level) of an HDAC in cells and/or in a subject. In some embodiments, nucleotide based inhibitors bind to an HDAC and decrease its enzymatic activity

Protein or peptide based inhibitors of an HDAC can include but are not limited to peptides, recombinant proteins, and antibodies or fragments thereof. Protein or peptide based inhibitors can be composed of at least one non-natural amino acid. In some embodiments protein or peptide based inhibitors decrease the expression (e.g., at the mRNA transcript and/or protein level) of an HDAC in cells and/or in a subject. In some embodiments, protein or peptide based inhibitors bind to an HDAC and decrease its enzymatic activity.

Methods for identifying and/or generating nucleotide based or protein/peptide based inhibitors for a protein described herein are commonly known in the art.

In some embodiments, according to any of the methods described above, the histone deacetylase inhibitor may include, but is not limited to, vorinostat (SAHA), panobinostat (LBH589), belinostat (PXD101, CAS 414864-00-9), tacedinaline (N-acetyldinaline, CI-994), givinostat (gavinostat, ITF2357), FRM-0334 (EVP-0334), resveratrol (SRT501), CUDC-101, quisinostat (JNJ-26481585), abexinostat (PCI-24781), dacinostat (LAQ824, NVP-LAQ824), valproic acid, 4-(dimethylamino)N-[6-(hydroxyamino)-6-oxohexyl]-benzamide (HDAC1 inhibitor), 4-Iodo suberoylanilide hydroxamic acid (HDAC1 and HDAC6 inhibitor), romidepsin (a cyclic tetrapeptide with HDAC inhibitory activity primarily towards class-I HDACs), 1-naphthohydroxamic acid (HDAC1 and HDAC6 inhibitor), HDAC inhibitors based on amino-benzamide biasing elements (e.g., mocetinostat (MGCD103) and entinostat (MS275), which are highly selective for HDAC1, 2 and 3), AN-9 (CAS 122110-53-6), APHA Compound 8 (CAS 676599-90-9), apicidin (CAS 183506-66-3), BML-210 (CAS 537034-17-6), salermide (CAS 1105698-15-4), suberoyl bis-hydroxamic acid (CAS 38937-66-5) (HDAC1 and HDAC3 inhibitor), butyrylhydroxamic acid (CAS 4312-91-8), CAY10603 (CAS 1045792-66-2) (HDAC6 inhibitor), CBHA (CAS 174664-65-4), ricolinostat (ACY1215, rocilinostat), trichostatin-A, WT-161, tubacin, and Merck60.

Thus, in some embodiments, there is provided a method of treating a solid tumor (such as bladder cancer, renal cell carcinoma, or melanoma) in an individual (such as a human) comprising administering to the individual a) an effective amount of a composition comprising nanoparticles comprising an mTOR inhibitor (such as a limus drug, e.g., sirolimus or a derivative thereof) and an albumin; and b) an effective amount of a histone deacetylase inhibitor. In some embodiments, the histone deacetylase inhibitor is specific to only one HDAC. In some embodiments, the histone deacetylase inhibitor is specific to only one class of HDAC. In some embodiments, the histone deacetylase inhibitor is specific to two or more HDACs or two or more classes of HDACs. In some embodiments, the histone deacetylase inhibitor is specific to class I and II HDACs. In some embodiments, the histone deacetylase inhibitor is specific to class III HDACs. In some embodiments, the histone deacetylase inhibitor is selected from the group consisting of vorinostat (SAHA), panobinostat (LBH589), belinostat (PXD101, CAS 414864-00-9), tacedinaline (N-acetyldinaline, CI-994), givinostat (gavinostat, ITF2357), FRM-0334 (EVP-0334), resveratrol (SRT501), CUDC-101, quisinostat (JNJ-26481585), abexinostat (PCI-24781), dacinostat (LAQ824, NVP-LAQ824), valproic acid, 4-(dimethylamino)N-[6-(hydroxyamino)-6-oxohexyl]-benzamide (HDAC1 inhibitor), 4-Iodo suberoylanilide hydroxamic acid (HDAC1 and HDAC6 inhibitor), romidepsin (a cyclic tetrapeptide with HDAC inhibitory activity primarily towards class-I HDACs), 1-naphthohydroxamic acid (HDAC1 and HDAC6 inhibitor), HDAC inhibitors based on amino-benzamide biasing elements (e.g., mocetinostat (MGCD103) and entinostat (MS275), which are highly selective for HDAC1, 2 and 3), AN-9 (CAS 122110-53-6), APHA Compound 8 (CAS 676599-90-9), apicidin (CAS 183506-66-3), BML-210 (CAS 537034-17-6), salermide (CAS 1105698-15-4), suberoyl bis-hydroxamic acid (CAS 38937-66-5) (HDAC1 and HDAC3 inhibitor), butyrylhydroxamic acid (CAS 4312-91-8), CAY10603 (CAS 1045792-66-2) (HDAC6 inhibitor), CBHA (CAS 174664-65-4), ricolinostat (ACY1215, rocilinostat), trichostatin-A, WT-161, tubacin, and Merck60. In some embodiments, the histone deacetylase inhibitor is romidepsin.

### Kinase Inhibitors

The methods described herein in some embodiments comprise administration of nanoparticle compositions of mTOR inhibitors in combination with a kinase inhibitor (such as a tyrosine kinase inhibitor). Kinase inhibitors have demonstrated significant clinical benefit as single agents for several indications, including non-small cell lung cancer, renal cell carcinoma, and chronic myeloid leukemia, and have received FDA approval for these indications.

A kinase is an enzyme that catalyzes the transfer of phosphate groups from high-energy, phosphate-donating molecules to specific substrates. Kinases are part of the larger family of phosphotransferases. The phosphorylation state of a molecule, whether it be a protein, lipid, or carbohydrate, can affect its activity, reactivity, and/or its ability to bind other molecules. Therefore, kinases are critical in metabolism, cell signaling, protein regulation, cellular transport, secretory processes, and many other cellular pathways.

Protein kinases act on proteins, phosphorylating them on serine, threonine, tyrosine, and/or histidine residues. Phosphorylation can modify the function of a protein in many ways. It can increase or decrease a protein's activity, stabilize it or mark it for destruction, localize it within a specific cellular compartment, and it can initiate or disrupt its interaction with other proteins. The protein kinases make up the majority of all kinases and are widely studied. These kinases, in conjunction with phosphatases, play a major role in protein and enzyme regulation as well as signaling in the cell.

“Kinase inhibitors,” as used herein, refer to molecules and pharmaceuticals, the administration of which to a subject results in the inhibition of a kinase. Examples of tyrosine kinase inhibitors include, but are not limited to, apatinib, cabozantinib, canertinib, crenolanib, crizotinib, dasatinib, erlotinib, foretinib, fostamatinib, ibrutinib, idelalisib, imatinib, lapatinib, linifanib, motesanib, mubritinib, nilotinib, nintedanib, radotinib, sorafenib, sunitinib, vatalanib, and vemurafenib.

In some embodiments, according to any of the methods described above, the kinase inhibitor may include, but is not limited to, apatinib, cabozantinib, canertinib, crenolanib, crizotinib, dasatinib, erlotinib, foretinib, fostamatinib, ibrutinib, idelalisib, imatinib, lapatinib, linifanib, motesanib, mubritinib, nilotinib, nintedanib, radotinib, sorafenib, sunitinib, vatalanib, and vemurafenib. In some embodiments, the kinase inhibitor is a tyrosine kinase inhibitor. In some embodiments, the kinase inhibitor is a serine/threonine kinase inhibitor. In some embodiments, the kinase inhibitor is a Raf kinase inhibitor. In some embodiments, the kinase inhibitor inhibits more than one class of kinase (e.g., an inhibitor of more than one of a tyrosine kinase, a Raf kinase, and a serine/threonine kinase). In some embodiments, the kinase inhibitor is nilotinib. In some embodiments, the kinase inhibitor is sorafenib.

Suitable tyrosine kinase inhibitors include, for example, imatinib (Gleevec®), nilotinim, gefitinib (Iressa®; ZD-1839), erlotinib (Tarceva®; OSI-774), sunitinib malate (Sutent®), sorafenib (Nexavar®), and Lapatinib (GW562016; Tykerb). In some embodiments, the tyrosine kinase inhibitor is a multiple reversible ErbB1 family tyrosine kinase inhibitor (e.g., laptinib). In some embodiments, the tyrosine kinase inhibitor is a single reversible EGFR tyrosine kinase inhibitor (e.g., gefitinib or erlotinib). In some embodiments, the tyrosine kinase inhibitor is erlotinib. In some embodiments, the tyrosine kinase inhibitor is gefitinib. In some embodiments, the tyrosine kinase inhibitor is a single irreversible EGFR tyrosine kinase inhibitor (e.g., EKB-569 or CL-387,785). In some embodiments, the tyrosine kinase inhibitor is a multiple irreversible ErbB family tyrosine kinase inhibitor (e.g. canertinib (CL-1033; PD183805), HKI-272, BIBW 2992, or HKI-357). In some embodiments, the tyrosine kinase inhibitor is a multiple reversible tyrosine kinase inhibitor (e.g., ZD-6474, ZD-6464, AEE 788, or XL647). In some embodiments, the tyrosine kinase inhibitor inhibits ErbB family heterodimerization (e.g., BMS-599626). In some embodiments, the tyrosine kinase inhibitor inhibits protein folding by affecting HSP90 (e.g., benzoquinone ansamycin, IPI-504, or 17-AAG). In some embodiments, the tyrosine kinase inhibitor is an inhibitor of BCR-AbI. In some embodiments, the tyrosine kinase inhibitor is an inhibitor of IGF-IR.

Thus, in some embodiments, there is provided a method of treating a solid tumor (such as bladder cancer, renal cell carcinoma, or melanoma) in an individual (such as a human) comprising administering to the individual a) an effective amount of a composition comprising nanoparticles comprising an mTOR inhibitor (such as a limus drug, e.g., sirolimus or a derivative thereof) and an albumin; and b) an effective amount of a kinase inhibitor. In some embodiments, the kinase inhibitor is a tyrosine kinase inhibitor. In some embodiments, the kinase inhibitor is a serine/threonine kinase inhibitor. In some embodiments, the kinase inhibitor is a Raf kinase inhibitor. In some embodiments, the kinase inhibitor inhibits more than one class of kinase (e.g., an inhibitor of more than one of a tyrosine kinase, a Raf kinase, and a serine/threonine kinase). In some embodiments, the kinase inhibitor is selected from the group consisting of apatinib, cabozantinib, canertinib, crenolanib, crizotinib, dasatinib, erlotinib, foretinib, fostamatinib, ibrutinib, idelalisib, imatinib, lapatinib, linifanib, motesanib, mubritinib, nilotinib, nintedanib, radotinib, sorafenib, sunitinib, vatalanib, and vemurafenib. In some embodiments, the kinase inhibitor is nilotinib. In some embodiments, the kinase inhibitor is sorafenib.

### Cancer Vaccines

The methods described herein in some embodiments comprise administration of nanoparticle compositions of mTOR inhibitors in combination with a cancer vaccine (such as a vaccine prepared using autologous or allogeneic tumor cells or a TAA). Cancer vaccines have demonstrated significant clinical benefit in therapies for several solid tumor indications, including prostate cancer, breast cancer, lung cancer, melanoma, pancreatic cancer, colorectal cancer, and renal cell carcinoma, and have received FDA approval for treatment of asymptomatic and minimally symptomatic metastatic castration-resistant prostate cancer (mCRPC).

A cancer vaccine is a form of active immunotherapy that increases the ability of an individual's immune system to respond to a TAA and mount an immune response to eliminate malignant cells (Melero, I. et al. (2014). Nature reviews Clinical oncology, 11(9), 509-524). Cancer vaccines may be designed to target multiple, undefined antigens, or to specifically target a given antigen or group of antigens. Polyvalent vaccines can be prepared from autologous or allogeneic cells, such as from whole tumor cells or from dendritic cells that have been fused with tumor cells, transfected with DNA or RNA derived from a tumor, or loaded with lysate from tumor cells. Antigen-specific vaccines can be prepared from a single antigen, including short peptides with narrow epitope specificity or long peptides having multiple epitopes, or from a mixture of several different antigens.

The immunogenicity of antigens in a cancer vaccine can be increased in several ways, such as by combining the antigen with one or more adjuvants. Adjuvants can be selected to elicit a desired immune response for cancer immunotherapy, such as activation of type 1 T helper cells (T_{H}1) and cytotoxic T lymphocytes (CTLs). Adjuvants useful for cancer vaccines include, for example, alum (such as aluminum hydroxide or phosphate), microbes and microbial derivatives (such as the bacterium Bacillus Calmette-Guerin, CpG, Detox B, monophosphoryl lipid A, and poly I.C), keyhole limpet hemocyanin (KLH), oil emulsions or surfactants (such as AS02, AS03, MF59, Montanide ISA-51™, and QS21), particulates (such as AS04, polylactide co-glycolide, and virosomes), viral vectors (such as adenovirus, vaccinia, and fowlpox), delta innulin based synthetic polysaccharide, imidzaquinolines, saponins, flagellin, and natural or synthetic cytokines (such as IL-2, IL-12, IFN-α, and GM-CSF). See, for example, Banday, A. H. et al. (2015). Immunopharmacology and immunotoxicology, 37(1), 1-11 and Melero, I. et al., supra. Antigens and adjuvants can also be packaged in immunogenic delivery vehicles to increase cancer vaccine potency. Such delivery vehicles include, but are not limited to, liposomal microspheres, recombinant viral vectors, and cultured mature dendritic cells. Immunogenicity can also be increased by using a prime/boost strategy, where the immune system is primed with a first cancer vaccine targeting an antigen then boosted with a second cancer vaccine targeting the same antigen but in a different vector.

A cancer vaccine may include any molecules and pharmaceuticals, the administration of which to a subject results in an increase in the ability of the subject's immune system to mount an immune response against at least one tumor-associated antigen. Examples of cancer vaccines include, but are not limited to, polyvalent vaccines prepared from autologous tumor cells, polyvalent vaccines prepared from allogeneic tumor cells, and antigen-specific vaccines prepared from at least one tumor-associated antigen. Antigen-specific vaccines can comprise the at least one tumor-associated antigen, fragments thereof, or nucleic acids (such as recombinant viral vectors) encoding the at least one tumor-associated antigen or fragments thereof.

In some embodiments, according to any of the methods described above, the cancer vaccine may include, but is not limited to, a vaccine prepared using autologous tumor cells, a vaccine prepared using allogeneic tumor cells, and a vaccine prepared using at least one tumor-associated antigen (TAA). In some embodiments, the TAA is selected, for example, from the group consisting of heat shock proteins, melanocyte antigen gp100, MAGE antigens, BAGE, GAGE, NY-ESO-1, Melan-A, PSA, HER2, hTERT, p53, survivin, KRAS, WT1, alphafetoprotein (AFP), carcinoembryonic antigen (CEA), CA-125, GM2, MUC-1, epithelial tumor antigen (ETA), tyrosinase, and Trp-2. In some embodiments, the TAA is a neo-antigen, such as bcr-abl or a mutated form of a protein selected from the group consisting of β-catenin, HSP70-2, CDK4, MUM1, CTNNB1, CDC27, TRAPPC1, TPI, ASCC3, HHAT, FN1, OS-9, PTPRK, CDKN2A, HLA-A11, GAS7, GAPDH, SIRT2, GPNMB, SNRP116, RBAF600, SNRPD1, Prdx5, CLPP, PPP1R3B, EF2, ACTN4, ME1, NF-YC, HLA-A2, HSP70-2, KIAA1440, and CASP8 (for examples of identifying neoantigens see Gubin, M. M. et al. (2015). The Journal ofclinical investigation, 125(9), 3413-3421; Lu, Y. C., & Robbins, P. F. (2016, February). Seminars in immunology. 28(1): 22-27; and Schumacher, T. N., & Schreiber, R. D. (2015). Science, 348(6230), 69-74). In some embodiments, the TAA is a polypeptide derived from a virus implicated in human cancer, such as Human Papilloma Viruses (HPV), Hepatitis Viruses (HBV and HCV), Human T-Lymphotropic Virus (HTLV), Merkel cell polyomavirus, Epstein-Barr Virus (EBV), and Kaposi's Sarcoma-associated Herpesvirus (KSHV).

Suitable cancer vaccines include, for example, GVAX, ADXS11-001, ADXS31-001, ADXS31-164, ALVAC-CEA vaccine, BiovaxID, Prostvac, CDX110, CDX1307, CDX1401, CimaVax-EGF, CV9104, Lapuleucel-T, NeuVax, GRNVAC1, GI-6207, GI-6301, GI-4000, Tecemotide, CBLI, Cvac, and SCIBI.

### Articles of Manufacture and Kits

In some embodiments of the invention, there is provided an article of manufacture containing materials useful for the treatment of a solid tumor comprising an mTOR inhibitor nanoparticle composition (such as sirolimus/albumin nanoparticle composition) and a second therapeutic agent. The article of manufacture can comprise a container and a label or package insert on or associated with the container. Suitable containers include, for example, bottles, vials, syringes, etc. The containers may be formed from a variety of materials such as glass or plastic. Generally, the container holds a composition which is effective for treating a disease or disorder described herein, and may have a sterile access port (for example the container may be an intravenous solution bag or a vial having a stopper pierceable by a hypodermic injection needle). At least one active agent in the composition is a) a nanoparticle formulation of an mTOR inhibitor; or b) a second therapeutic agent. The label or package insert indicates that the composition is used for treating the particular condition in an individual. The label or package insert will further comprise instructions for administering the composition to the individual. Articles of manufacture and kits comprising combination therapies described herein are also contemplated.

Package insert refers to instructions customarily included in commercial packages of therapeutic products that contain information about the indications, usage, dosage, administration, contraindications and/or warnings concerning the use of such therapeutic products. In some embodiments, the package insert indicates that the composition is used for treating a solid tumor (such as bladder cancer, renal cell carcinoma, or melanoma).

Additionally, the article of manufacture may further comprise a second container comprising a pharmaceutically-acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

Kits are also provided that are useful for various purposes, e.g., for treatment of a solid tumor (such as bladder cancer, renal cell carcinoma, or melanoma). Kits of the invention include one or more containers comprising an mTOR inhibitor nanoparticle composition (such as sirolimus/albumin nanoparticle composition) (or unit dosage form and/or article of manufacture), and in some embodiments, further comprise a second therapeutic agent (such as the agents described herein) and/or instructions for use in accordance with any of the methods described herein. The kit may further comprise a description of selection of individuals suitable for treatment. Instructions supplied in the kits of the invention are typically written instructions on a label or package insert (e.g., a paper sheet included in the kit), but machine-readable instructions (e.g., instructions carried on a magnetic or optical storage disk) are also acceptable.

For example, in some embodiments, the kit comprises a composition comprising an mTOR inhibitor nanoparticle composition (such as sirolimus/albumin nanoparticle composition). In some embodiments, the kit comprises a) a composition comprising an mTOR inhibitor nanoparticle composition (such as sirolimus/albumin nanoparticle composition), and b) a second therapeutic agent. In some embodiments, the kit comprises a) a composition comprising an mTOR inhibitor nanoparticle composition (such as sirolimus/albumin nanoparticle composition), and b) instructions for administering the mTOR inhibitor nanoparticle composition in combination with a second therapeutic agent to an individual for treatment of a solid tumor, such as bladder cancer, renal cell carcinoma, or melanoma. In some embodiments, the kit comprises a) a composition comprising an mTOR inhibitor nanoparticle composition (such as sirolimus/albumin nanoparticle composition), b) a second therapeutic agent, and c) instructions for administering the mTOR inhibitor nanoparticle composition and the second therapeutic agent to an individual for treatment of a solid tumor, such as bladder cancer, renal cell carcinoma, or melanoma. The mTOR inhibitor nanoparticle composition (such as sirolimus/albumin nanoparticle composition) and the second therapeutic agent can be present in separate containers or in a single container. For example, the kit may comprise one distinct composition or two or more compositions wherein one composition comprises an mTOR inhibitor nanoparticle composition (such as sirolimus/albumin nanoparticle composition) and another composition comprises the second therapeutic agent.

The kits of the invention are in suitable packaging. Suitable packaging includes, but is not limited to, vials, bottles, jars, flexible packaging (e.g., sealed Mylar or plastic bags), and the like. Kits may optionally provide additional components such as buffers and interpretative information. The present application thus also provides articles of manufacture, which include vials (such as sealed vials), bottles, jars, flexible packaging, and the like.

The instructions relating to the use of the mTOR inhibitor nanoparticle composition (such as sirolimus/albumin nanoparticle composition) and the second therapeutic agent generally include information as to dosage, dosing schedule, and route of administration for the intended treatment. The containers may be unit doses, bulk packages (e.g., multi-dose packages) or sub-unit doses. For example, kits may be provided that contain sufficient dosages of an mTOR inhibitor nanoparticle composition (such as sirolimus/albumin nanoparticle composition) and a second therapeutic agent as disclosed herein to provide effective treatment of an individual for an extended period, such as any of a week, 8 days, 9 days, 10 days, 11 days, 12 days, 13 days, 2 weeks, 3 weeks, 4 weeks, 6 weeks, 8 weeks, 3 months, 4 months, 5 months, 7 months, 8 months, 9 months, or more. Kits may also include multiple unit doses of the mTOR inhibitor nanoparticle composition (such as sirolimus/albumin nanoparticle composition) and the second therapeutic agent and instructions for use, packaged in quantities sufficient for storage and use in pharmacies, for example, hospital pharmacies and compounding pharmacies.

Those skilled in the art will recognize that several embodiments are possible within the scope and spirit of this invention. The invention will now be described in greater detail by reference to the following non-limiting examples. The following examples further illustrate the invention but, of course, should not be construed as in any way limiting its scope.

### Exemplary Embodiments

Embodiment 1. In some embodiments, there is provided a method of treating a solid tumor in an individual, comprising administering to the individual: a) an effective amount of a composition comprising nanoparticles comprising an mTOR inhibitor and an albumin, and b) an effective amount of a second therapeutic agent, wherein the second therapeutic agent is selected from the group consisting of an immunomodulator, a histone deacetylase inhibitor, and a kinase inhibitor.

Embodiment 2. In some further embodiments of embodiment 1, the solid tumor is bladder cancer, renal cell carcinoma, or melanoma.

Embodiment 3. In some further embodiments of embodiment 1 or 2, the solid tumor is relapsed or refractory to a standard therapy for the solid tumor.

Embodiment 4. In some further embodiments of any one of embodiments 1-3, the amount of the mTOR inhibitor in the mTOR inhibitor nanoparticle composition is from about 10 mg/m² to about 150 mg/m².

Embodiment 5. In some further embodiments of embodiment 4, the amount of the mTOR inhibitor in the mTOR inhibitor nanoparticle composition is about 45 mg/m² to about 100 mg/m².

Embodiment 6. In some further embodiments of embodiment 4, the amount of the mTOR inhibitor in the mTOR inhibitor nanoparticle composition is about 75 mg/m² to about 100 mg/m².

Embodiment 7. In some further embodiments of any one of embodiments 1-6, the mTOR inhibitor nanoparticle composition is administered weekly.

Embodiment 8. In some further embodiments of any one of embodiments 1-6, the mTOR inhibitor nanoparticle composition is administered 3 out of every 4 weeks.

Embodiment 9. In some further embodiments of any one of embodiments 1-8, the mTOR inhibitor nanoparticle composition and the second therapeutic agent are administered sequentially to the individual.

Embodiment 10. In some further embodiments of any one of embodiments 1-8, the mTOR inhibitor nanoparticle composition and the second therapeutic agent are administered simultaneously to the individual.

Embodiment 11. In some further embodiments of any one of embodiments 1-10, the mTOR inhibitor is a limus drug.

Embodiment 12. In some further embodiments of embodiment 11, the limus drug is sirolimus.

Embodiment 13. In some further embodiments of any one of embodiments 1-12, the average diameter of the nanoparticles in the composition is no greater than about 150 nm.

Embodiment 14. In some further embodiments of embodiment 13, the average diameter of the nanoparticles in the composition is no greater than about 120 nm.

Embodiment 15. In some further embodiments of any one of embodiments 1-14, the weight ratio of the albumin to the mTOR inhibitor in the nanoparticle composition is no greater than about 9:1.

Embodiment 16. In some further embodiments of any one of embodiments 1-15, the nanoparticles comprise the mTOR inhibitor associated with the albumin.

Embodiment 17. In some further embodiments of embodiment 16, the nanoparticles comprise the mTOR inhibitor coated with the albumin.

Embodiment 18. In some further embodiments of any one of embodiments 1-17, the mTOR inhibitor nanoparticle composition is administered intravenously, intraarterially, intraperitoneally, intravesicularlly, subcutaneously, intrathecally, intrapulmonarily, intramuscularly, intratracheally, intraocularly, transdermally, orally, or by inhalation.

Embodiment 19. In some further embodiments of embodiment 18, the mTOR inhibitor nanoparticle composition is administered intravenously.

Embodiment 20. In some further embodiments of any one of embodiments 1-19, the individual is human.

Embodiment 21. In some further embodiments of any one of embodiments 1-20, the method further comprises selecting the individual for treatment based on the presence of at least one mTOR-activating aberration.

Embodiment 22. In some further embodiments of embodiment 21, the mTOR-activating aberration comprises a mutation in an mTOR-associated gene.

Embodiment 23. In some further embodiments of embodiment 21 or 22, the mTOR-activating aberration is in at least one mTOR-associated gene selected from the group consisting of AKT1, FLT-3, MTOR, PIK3CA, PIK3CG, TSC1, TSC2, RHEB, STK11, NF1, NF2, TP53, FGFR4, BAP1, KRAS, NRAS and PTEN.

Embodiment 24. In some further embodiments of any one of embodiments 1-23, the second therapeutic agent is an immunomodulator.

Embodiment 25. In some further embodiments of embodiment 24, the immunomodulator is an IMiD® compound.

Embodiment 26. In some further embodiments of embodiment 24, the immunomodulator is an immune checkpoint inhibitor.

Embodiment 27. In some further embodiments of embodiment 24, the immunomodulator is selected from the group consisting of pomalidomide and lenalidomide.

Embodiment 28. In some further embodiments of any one of embodiments 24-27, the method further comprises selecting the individual for treatment based on the presence of at least one biomarker indicative of favorable response to treatment with an immunomodulator.

Embodiment 29. In some further embodiments of embodiment 28, the at least one biomarker comprises a mutation in an immunomodulator-associated gene.

Embodiment 30. In some further embodiments of any one of embodiments 1-23, the second therapeutic agent is a histone deacetylase inhibitor.

Embodiment 31. In some further embodiments of embodiment 30, the histone deacetylase inhibitor is selected from the group consisting of romidepsin, panobinostat, ricolinostat, and belinostat.

Embodiment 32. In some further embodiments of embodiment 30 or 31, the method further comprises selecting the individual for treatment based on the presence of at least one biomarker indicative of favorable response to treatment with a histone deacetylase inhibitor (HDACi).

Embodiment 33. In some further embodiments of embodiment 32, the at least one biomarker comprises a mutation in an HDACi-associated gene.

Embodiment 34. In some further embodiments of any one of embodiments 1-23, the second therapeutic agent is a kinase inhibitor.

Embodiment 35. In some further embodiments of embodiment 34, the kinase inhibitor is selected from the group consisting of nilotinib and sorafenib.

Embodiment 36. In some further embodiments of embodiment 34 or 35, the method further comprises selecting the individual for treatment based on the presence of at least one biomarker indicative of favorable response to treatment with a kinase inhibitor.

Embodiment 37. In some further embodiments of embodiment 36, the at least one biomarker comprises a mutation in a kinase inhibitor-associated gene.

Embodiment 38. In some further embodiments of any one of embodiments 1-23, the second therapeutic agent is a cancer vaccine.

Embodiment 39. In some further embodiments of embodiment 38, the cancer vaccine is selected from the group consisting of a vaccine prepared from autologous tumor cells, a vaccine prepared from allogeneic tumor cells, and a vaccine prepared from at least one tumor-associated antigen.

Embodiment 40. In some further embodiments of embodiment 38 or 39, the method further comprises selecting the individual for treatment based on the presence of at least one biomarker indicative of favorable response to treatment with a cancer vaccine.

Embodiment 41. In some further embodiments of embodiment 36, the at least one biomarker comprises a mutation in a cancer vaccine-associated gene.

Embodiment 42. In some further embodiments of any one of embodiments 1-41, the solid tumor is bladder cancer.

Embodiment 43. In some further embodiments of any one of embodiments 1-41, the solid tumor is renal cell carcinoma.

Embodiment 44. In some further embodiments of any one of embodiments 1-41, the solid tumor is melanoma.

### EXAMPLES

### Example 1: Evaluation of Drugs in Combination with Nab-Sirolimus for Anti-Tumor Activity in a UMUC3 (Human Bladder Cancer) Cell Line Mouse Xenograft Model

The anti-tumor efficacy of a panel of drugs, including mitomycin C, cisplatin, gemcitabine, valrubicin, docetaxel, and immune checkpoint inhibitors (ICI) in combination with nab-sirolimus are evaluated and compared in a UMUC3 cell xenograft model in athymic nude mice. ICIs include antagonistic antibodies targeting an immune checkpoint protein, such as anti-CTLA4 (such as Ipilimumab and Tremelimumab), anti-PD-1 (such as Nivolumab, Pidilizumab, and Pembrolizumab), anti-PD-L1 (such as MPDL3280A, BMS-936559, MED14736, and Avelumab), anti-PD-L2, anti-LAG3 (such as BMS-986016 or C9B7W), anti-B7-1, anti-B7-H3 (such as MGA271), anti-B7-H4, anti-TIM3, anti-BTLA, anti-VISTA, anti-KIR (such as Lirilumab and IPH2101), or anti-A2aR.

The human bladder cancer (adenocarcinoma) cell line UMUC3 is prepared as follows. A frozen (liquid nitrogen) aliquot of the UMUC3 cell line (ATCC) is thawed out, dispersed into a 75 cm² flask containing DMEM media supplemented with 10% fetal bovine calf serum (FBS) and incubated at 37° C. in humidified atmosphere of 5% CO₂. As cells become 80% confluent, the cultures are expanded to 150 cm² flasks. The cultures are further expanded until sufficient cells are available for injection into mice (10×10⁶ cells per mouse).

Tumors are established from the UMUC3 cells are follows. Female athymic nude mice are obtained and housed in filter-topped cages supplied with autoclaved bedding. Animal handling procedures are under laminar flow hood. Each mouse is ear tagged for individual identification, and the body weight of each mouse is recorded. UMUC3 cells (10×10⁶ cells per flank in 0.1 mL PBS with 20% Matrigel) are injected subcutaneously into the right flank of each mouse to implant the tumor. Tumor measurements are recorded three times per week (such as on Mondays, Wednesdays and Fridays) until tumors become approximately 60 to 160 mm³ total.

Prior to the treatment, body weights and tumor measurements of all mice are recorded. The mice are sorted into treatment groups, such as 7 treatment groups of 8 mice each, based upon tumor size. The mice are treated with the drugs according to the dosing regimen as described in Table 1 below. The treatment comprises dosing for 3 weeks.

TABLE 1
Group treatments.

| Group | # Mice | Test material | ROA | Frequency*** |
|---|---|---|---|---|
| 1 | 8 | Saline | IV* | Twice weekly/3 weeks |
| 2 | 8 | nab-sirolimus | IV | Twice weekly/3 weeks |
| | | (nab-S) | | |
| 3 | 8 | MMC + nab-S^{#} | IP**/IV | Twice weekly/3 weeks |
| 4 | 8 | Cis + nab-S^{#} | IP/IV | Twice weekly/3 weeks |
| 5 | 8 | GEM + nab-S^{#} | IP/IV | Twice weekly/3 weeks |
| 6 | 8 | Val + nab-S^{#} | IP/IV | Twice weekly/3 weeks |
| 7 | 8 | Doc + nab-S^{#} | IP/IV | Twice weekly/3 weeks |
| 8 | 8 | ICI + nab-S^{#} | IP/IV | Twice weekly/3 weeks |

*IV = intravenous injection
**IP = intraperitoneal injection
***Dose twice weekly for 3 weeks: total 6 doses
^{#}In each combination of drugs being administered comprising nab-sirolimus and a second drug (such as MMC, Cis, GEM, Val, Doc, and ICI), the second drug is administered immediately before nab-sirolimus.

The mice are monitored during the course of the treatment by recording body weights three times per week (such as on Mondays, Wednesdays and Fridays), recording signs of distress daily, and recording tumor measurements three times per week (such as on Mondays, Wednesdays and Fridays). Measurements of tumor sizes and body weights are continued for 2 weeks following completion of the dosing regimen, or until the mouse is sacrificed when the tumor size of the mouse is more than 2000 mm³.

### Example 2: Phase I Clinical Study of ABI-009 (Nab-Sirolimus) in Pediatric Patients with Recurrent or Refractory Solid Tumors, Including CNS Tumors as a Single Agent and in Combination with Temozolomide and Irinotecan

A single-arm non-randomized Phase 1 dose escalation study is designed to determine the toxicity profile, maximum tolerated dose, recommended Phase 2 dose, as well as pharmacokinetic and pharmacodynamic parameters of ABI-009 as single agent or in combination with temozolomide and irinotecan for treating recurrent or refractory solid tumors, including central nervous system (CNS) tumors, in pediatric patients. Efficacy of AB-009 in combination with irinotecan and temozolomide in treating solid tumors of the pediatric patients is assessed within the confines of the Phase 1 study. Furthermore, expression of biomarkers, such as SK61 and 4E-BP1 are determined in patients before the treatment. Exemplary solid tumors to be investigated include neuroblastoma (NB), osteosarcoma (OS), Ewing sarcoma (EWS), rhabdomyosarcoma (RMS), medulloblastoma (MB), gliomas, renal tumors, and hepatic tumors (such as hepatoblastoma and hepatocellular carcinoma).

Primary objectives of the clinical study include: 1) to estimate the maximum tolerated dose (MTD) and/or recommended Phase 2 dose (RP2D) of ABI-009 administered as an intravenous dose over 30 minutes on Days 1 and 8 of a 21-day cycle, in combination with temozolomide and irinotecan (administered on Days 1-5) to pediatric patients with recurrent/refractory solid tumors, including CNS tumors; 2) to define and describe the toxicities of single agent ABI-009 administered as an intravenous dose over 30 minutes on Days 1 and 8 of a 21-day cycle in pediatric patients with recurrent or refractory cancer; 3) to define and describe the toxicities of ABI-009 administered as an intravenous dose over 30 minutes on Days 1 and 8 of a 21-day cycle in combination with temozolomide and irinotecan (administered on Days 1-5) in pediatric patients with recurrent or refractory cancer; and 4) to characterize the pharmacokinetics of ABI-009 in pediatric patients with recurrent or refractory cancer. Secondary objective of the study is to preliminarily define the antitumor activity of ABI-009 in combination with temozolomide and irinotecan within the confines of a Phase 1 study. Exploratory objective of the study is to examine SK61 and 4E-BP1 expression status in archival tumor tissue from solid tumor pediatric patients using immunohistochemistry.

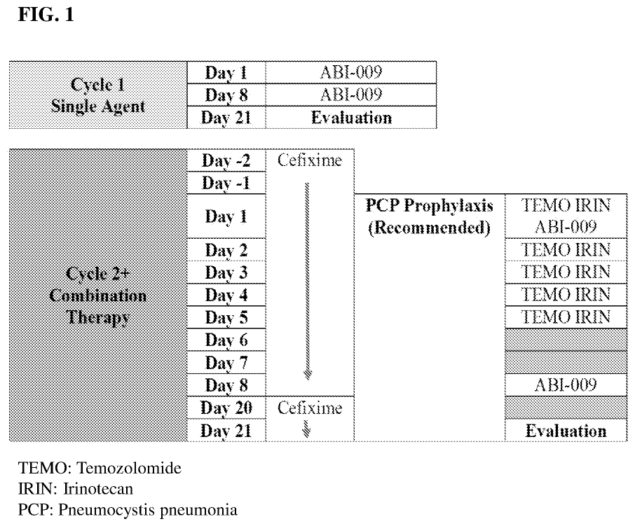

FIG. 1 shows the experimental design schema. ABI-009 is given intravenously over 30 minutes on Days 1 and 8 of each 21-day cycle. During Cycle 2+, ABI-009 is given 1 hour after irinotecan administration during Cycle 2. For subsequent cycles, ABI-009 is given within 8 hours after temozolomide and irinotecan. Temozolomide is administered orally, once daily on Days 1-5 of each 21-day cycle from Cycle 2+. Irinotecan is administered orally, once daily on Days 1-5 one hour after temozolomide of each 21-day cycle from Cycle 2+. Cefixime or an equivalent antibiotic is used as diarrheal prophylaxis and administered 2 days prior to the first dose of irinotecan, during irinotecan administration, and 3 days after the last does of irinotecan of each cycle. A cycle of therapy is considered 21 days. A cycle may be repeated for a total of 35 cycles, up to a total duration of therapy of approximately 24 months.

The dose escalation schema is shown in Table 1 below. Dose level 1 is the starting dose level, which is determined based on the recommended Phase 2 dose of ABI-009, irinotecan and temozolomide in previous clinical studies. If the MTD has been exceeded at Dose Level 1, then the subsequent cohort of patients will be treated at Dose Level −1. If Dose Level −1 is not well tolerated the study will be closed to accrual.

TABLE 1
Dosing schema.

| | Cycle 1 | Cycle 2+ | | |
|---|---|---|---|---|
| Dose | ABI-009 | ABI-009 | Irinotecan | Temozolomide |
| Level | (mg/m²) | (mg/m²) | (mg/m²) | (mg/m²) |
| −1 | 20 | 20 | 90 | 125 |
| 1 | 35 | 35 | 90 | 125 |
| 2 | 45 | 45 | 90 | 125 |
| 3 | 55 | 55 | 90 | 125 |

The rolling six design is utilized for dose escalation and patient accrual. See, for example, Skolnik J M, Barrett J S, Jayaraman B, et al: “Shortening the timeline of pediatric phase I trials: the rolling six design.” J Clin Oncol 26:190-5, 2008. Briefly, two to six patients can be concurrently enrolled onto a dose level, dependent upon (1) the number of patients enrolled at the current dose level, (2) the number of patients who have experienced dose-limiting toxicity (DLT) at the current dose level, and (3) the number of patients entered but with tolerability data pending at the current dose level. For example, when three participants are enrolled onto a dose cohort, if toxicity data is available for all three when the fourth participant entered and there are no DLTs, the dose is escalated and the fourth participant is enrolled to the subsequent dose level. If data is not yet available for one or more of the first three participants and no DLT has been observed, or if one DLT has been observed, the new participant is entered at the same dose level. Lastly, if two or more DLTs have been observed, the dose level is de-escalated. This process is repeated for participants five and six. In place of suspending accrual after every three participants, accrual is only suspended when a cohort of six is filled. When participants are inevaluable for toxicity, they are replaced with the next available participant if escalation or de-escalation rules have not been fulfilled at the time the next available participant is enrolled onto the study.

If two or more of a cohort of up to six patients experience DLT at a given dose level, then the MTD has been exceeded and dose escalation will be stopped. In the unlikely event that two DLTs observed out of 6 evaluable patients are of different classes of Adverse Effects (e.g., hepatotoxicity and myelosuppression), expansion of the cohort to 12 patients will be considered (if one of the DLTs does not appear to be dose-related, the Adverse Effects are readily reversible, AND study chair/DVL leadership/IND sponsor all agree that expansion of the cohort is acceptable). Once the MTD or RP2D has been defined, up to 6 additional patients with relapsed/refractory solid tumors may be enrolled to acquire PK data in a representative number of young patients (i.e., 6 patients <12 years old and 6 patients ≥12 years old).

Patients from Cycle 1 continue onto Cycle 2 if they do not experience a dose-limiting toxicity (DLT) and have again met laboratory parameters as defined in the eligibility section except for the following repeat cycle modified starting criteria: cholesterol ≤400 mg/dL OR ≤500 mg/dL and on lipid lowering medication, and triglycerides ≤300 mg/dL OR ≤500 mg/dL and on lipid lowering medication. Patients with progressive disease after Cycle 1 therapy with ABI-009 alone may remain on study provided they do not meet other exclusion criteria.

For Cycles 2+ part of the study, a cycle may be repeated every 21 days if the patient has at least stable disease and has again met laboratory parameters as defined in the eligibility section except for the following repeat cycle modified starting criteria: cholesterol ≤400 mg/dL OR ≤500 mg/dL and on lipid lowering medication, and triglycerides ≤300 mg/dL OR ≤500 mg/dL and on lipid lowering medication.

Maximum Tolerated Dose (MTD) for combination therapy is determined as the maximum dose at which ≤33% of patients experience DLT during Cycle 2 of therapy. Recommended Phase 2 Dose for combination therapy is determined as the MTD defined in Cycle 2 or in the absence of DLT, or Dose Level 3 (55 mg/m² ABI-009, 90 mg/m² irinotecan, and 125 mg/m² temozolomide).

The DLT observation period is the first two cycles of therapy. DLTs observed during Cycle 1 are counted towards Cycle 2 combination therapy MTD determination. CTCAE v 4 or current version will be used for grading toxicities. Any patient who receives at least one dose of the study drug(s) is considered evaluable for adverse events. In addition, for the dose-escalation portion, patients must receive at least 100% of the prescribed dose during Cycle 1 and 100% of the prescribed dose during Cycle 2 per protocol guidelines and must have the appropriate toxicity monitoring studies performed during Cycle 1 and Cycle 2 to be considered evaluable for DLT. Patients who are not evaluable for toxicity at a given dose level during either Cycle 1 or Cycle 2 will be replaced.

DLT is defined differently for hematological and non-hematological toxicities. Non-hematological DLT is defined as any Grade 3 or greater non-hematological toxicity attributable to the investigational drug with the specific exclusion of: Grade 3 nausea and vomiting <3 days duration; Grade 3 liver enzyme elevation, including ALT/AST/GGT, that returns to Grade ≤1 or baseline prior to the time for the next treatment cycle. Note: For the purposes of this study the ULN for ALT is defined as 45 U/L; Grade 3 fever; Grade 3 infection; Grade 3 hypophosphatemia, hypokalemia, hypocalcemia or hypomagnesemia responsive to oral supplementation; Grade 3 or 4 hypertriglyceridemia that returns to Grade ≤2 prior to the start of the next treatment cycle. The severity (grade) of hypertriglyceridemia is based upon fasting levels. If Grade 3 or 4 triglycerides are detected when routine (non-fasting) laboratory studies are performed, the test should be repeated within 3 days in the fasting state to permit accurate grading; Grade 3 hyperglycemia that returns to ≤Grade 2 or baseline (with or without the use of insulin or oral diabetic agents) prior to the start of the next treatment cycle. The severity (grade) of hyperglycemia is based upon fasting levels. If Grade 3 hyperglycemia is detected when routine (non-fasting) laboratory studies are performed, the test should be repeated within 3 days in the fasting state to permit accurate grading; Grade 3 or 4 hypercholesterolemia that returns to ≤Grade 2 after initiation of lipid lowering medication prior to the next treatment cycle. The severity (grade) of hypercholesterolemia is based upon fasting levels. If Grade 3 or 4 hypercholesterolemia is detected when routine (non-fasting) laboratory studies are performed, the test should be repeated within 3 days in the fasting state to permit accurate grading. Non-hematological toxicity also includes a delay of 14 days between treatment cycles. Allergic reactions that necessitate discontinuation of study drug are not be considered a dose-limiting toxicity.

Hematological DLT is defined as: Grade 4 neutropenia for >7 days; Platelet count <20,000/mm³ on 2 separate days, or requiring a platelet transfusion on 2 separate days, within a 7 day period; Myelosuppression that causes a delay of >14 days between treatment cycles; and Grade 3 or 4 thromboembolic event. Grade 3 or 4 febrile neutropenia is not be considered a dose-limiting toxicity.

Dose modification for elevated fasting triglycerides is as shown in Table 2 below.

TABLE 2

| Grade | Action |
|---|---|
| Grade 2 | Continue temsirolimus; if triglycerides are between 301 and |
| | 400 mg/dL consider treatment with an HMG-COA reductase |
| | inhibitor depending upon recommendations of institutional |
| | hyperlipidemia consultants. HMG-COA reductase inhibitor is |
| | recommended if triglycerides are between 401 and 500 mg/dL |
| Grade | Hold temsirolimus until recovery to ≤Grade 2 |
| 3-4 | An HMG-COA reductase inhibitor should be started, and |
| | dosages should be adjusted based upon recommendations from |
| | institutional hyperlipidemia consultants |
| | Upon retreatment at the same dose level, if Grade 3 or 4 |
| | toxicity recurs, lipid lowering medication should be adjusted |
| | in consultation with institutional hyperlipidemia consultants. |
| | Temsirolimus should be held until recovery to ≤Grade 2. |
| | Upon retreatment with temsirolimus concurrent with an HMG- |
| | CoA reductase inhibitor, if Grade 3 or 4 elevations recur, |
| | temsirolimus should be held until recovery to ≤Grade 2. |
| | Further lipid lowering medication options should be discussed |
| | with institutional hyperlipidemia consultants. Upon recovery to |
| | ≤Grade 2, temsirolimus should be restarted at the next lower |
| | dose level. If the patient is being treated on the lowest dose |
| | level, protocol therapy should be discontinued. |

Disease evaluations are performed at the end of Cycle 1, at the end of Cycle 2, then every other cycle for 2 cycles, then every 3 cycles. Disease response is assessed using the Response Evaluation Criteria in Solid Tumors (RECIST) guideline (version 1.1).

In additional to monitoring toxicity and response, pharmacokinetic and pharmacodynamic studies are performed. ABI-009 pharmacokinetics (PK) are determined using validated LC-MS/MS assays. Irinotecan and temozolomide pharmacokinetics are determined using a validated HPLC assay with fluorescence detection. For ABI-009 PK studies, plasma samples (2 mL per time point) are obtained from patients at the following time points during Cycle 1 (single agent) and Cycle 2 (Combination therapy) of the study: Day 1: pre-dose, end of infusion, and then 1, 2, 4, and 8 hrs after beginning of infusion; Day 2: 24 hours post-D1 ABI-009 dose; Day 4 (±1 day): 72 hours (±24 hours) post-D1 ABI-009 dose; and Day 8: Pre-ABI-009 dose. Optionally, CSF collection at any time point post-infusion can be obtained and analyzed to determine PK of ABI-009. For irinotecan and temozolomide PK studies, plasma samples (2 mL per time point) are obtained from patients at the following time points during Cycle 2 (Combination Therapy) of the study: Day 1: Pre-dose, and then 10 min, 1 hr, 3 hrs, and 6 hrs post-irinotecan dose; and Day 2: Pre-Day 2 irinotecan dose (24 hours after Day 1 irinotecan dose). Pharmacokinetics parameters (Tₘₐₓ, Cₘₐₓ, t_{1/2}, AUC, Cl/F) are calculated using standard non-compartmental or compartmental methods, as needed.

Additionally, tumor tissue samples are analyzed by immunohistochemistry to evaluate SK61 and 4E-BP1 expression in pediatric solid tumors prior to treatment with ABI-009. Paraffin embedded tissue block or unstained slides are required prior to enrollment. The analysis is performed during Cycle 1 of the study.

### Eligibility

Eligible individuals must meet all of the following inclusion criteria:

(1) Patients must be ≥12 months and ≤21 years of age;

(2) Patients must be diagnosed with recurrent or refractory solid tumors, including CNS tumors;

(3) Patients must have the following performance status: Karnofsky ≥50% for patients >16 year of age and Lansky ≥50% for patients ≤16 years of age. Neurologic deficits in patients with CNS tumors must have been relatively stable for at least 7 days prior to study enrollment. Patients who are unable to walk because of paralysis, but who are up in a wheelchair, will be considered ambulatory for the purpose of assessing the performance score.

(4) Patients must have fully recovered from the acute toxic effects of all prior anti-cancer chemotherapy and must meet at least the following duration from prior anti-cancer directed therapy prior to enrollment. If after the required timeframe, the numerical eligibility criteria are met, e.g. blood count criteria, the patient is considered to have recovered adequately: (i) Cytotoxic chemotherapy or other chemotherapy known to be myelosuppressive: ≥21 days after the last dose of cytotoxic or myelosuppressive chemotherapy (42 days if prior nitrosourea); (ii) Anti-cancer agents not known to be myelosuppressive (e.g. not associated with reduced platelet or ANC counts): ≥7 days after the last dose of agent; (iii) Antibodies: ≥3 half-lives for the antibody or ≥30 days must have elapsed after the last dose, whichever is shorter, and must have recovered from all acute toxicities; (iv) Hematopoietic growth factors: ≥14 days after the last dose of a long-acting growth factor (e.g. Neulasta) or 7 days for short-acting growth factor. For agents that have known adverse events occurring beyond 7 days after administration, this period must be extended beyond the time during which adverse events are known to occur; (v) Immunotherapy or Immune Modulatory Drugs: ≥42 days after the completion of any type of immunotherapy, immune modulatory drugs (e.g. cytokines, adjuvants, etc.) except steroids, or tumor directed vaccines; (vi) Stem cell Infusions (with or without TBI): Allogeneic (non-autologous) bone marrow or stem cell transplant, or any stem cell infusion including DLI or boost infusion: ≥84 days after infusion and no evidence of GVHD; autologous stem cell infusion including boost infusion: ≥42 days; g) Cellular Therapy: ≥42 days after the completion of any type of cellular therapy (e.g. modified T cells, NK cells, dendritic cells, etc.); (vii) XRT/External Beam Irradiation including Protons: ≥14 days after local XRT; ≥150 days after TBI, craniospinal XRT or if radiation to 50% of the pelvis; ≥42 days if other substantial BM radiation; i) Radiopharmaceutical therapy (e.g., radiolabeled antibody, 131I-MIBG): ≥42 days after systemically administered radiopharmaceutical therapy; (viii) Irinotecan, temozolomide and mTOR inhibitor exposure: Patients who have received prior single agent therapy with irinotecan, temozolomide, or an mTOR inhibitor, excluding ABI-009, are eligible; Patients who have received prior combination therapy with two of the three agents, excluding ABI-009 are eligible; Patients who have received prior therapy with all three agents in combination (i.e. irinotecan, temozolomide, and a mTOR inhibitor) are not eligible; Patients who have previously received irinotecan and temozolomide and progressed or had significant toxicity with these two drugs are not eligible; and

(5) Patients must meet organ function criteria described below:

(i) Adequate Bone Marrow Function Defined as: For patients with solid tumors without known bone marrow involvement: Peripheral absolute neutrophil count (ANC) ≥1000/mm³; Platelet count ≥100,000/mm³ (transfusion independent, defined as not receiving platelet transfusions for at least 7 days prior to enrollment); Hemoglobin ≥8.0 g/dL at baseline (may receive RBC transfusions). For patients with known bone marrow metastatic disease: Peripheral absolute neutrophil count (ANC) ≥1000/mm³; Platelet count ≥100,000/mm³ (transfusion independent, defined as not receiving platelet transfusions for at least 7 days prior to enrollment); May receive transfusions provided they are not known to be refractory to red cell or platelet transfusions. These patients will not be evaluable for hematologic toxicity. At least 5 of every cohort of 6 patients with a solid tumor must be evaluable for hematologic toxicity, for the dose-escalation part of the study. If dose-limiting hematologic toxicity is observed, all subsequent patients enrolled must be evaluable for hematologic toxicity.

(ii) Adequate Renal Function Defined as: Creatinine clearance or radioisotope GFR ≥70 ml/min/1.73 m² or a serum creatinine based on age/gender as shown in Table 3 below.

TABLE 3

| | Maximum Serum | |
|---|---|---|
| | Creatinine (mg/dL) | |
| Age | Male | Female |
| 1 to < 2 years | 0.6 | 0.6 |
| 2 to < 6 years | 0.8 | 0.8 |
| 6 to < 10 years | 1 | 1 |
| 10 to < 13 years | 1.2 | 1.2 |
| 13 to < 16 years | 1.5 | 1.4 |
| ≥16 years | 1.7 | 1.4 |

* The threshold creatinine values in this Table were derived from the Schwartz formula for estimating GFR utilizing child length and stature data published by the CDC.

(iii) Adequate Liver Function Defined as: Bilirubin (sum of conjugated+unconjugated)≤1.5×upper limit of normal (ULN) for age; SGPT (ALT)≤110 U/L. For the purpose of this study, the ULN for SGPT is 45 U/L; Serum albumin ≥2 g/dL.

(iv) Adequate Pulmonary Function Defined as: Pulse oximetry >94% on room air if there is clinical indication for determination (e.g. dyspnea at rest).

(v) Adequate Neurologic Function Defined as: Patients with seizure disorder may be enrolled if on non-enzyme inducing anticonvulsants and well controlled; Nervous system disorders (CTCAE v4) resulting from prior therapy must be Grade 2.

(vi) Adequate Metabolic Function Defined as: Serum triglyceride level ≤300 mg/dL; Serum cholesterol level ≤300 mg/dL; Random or fasting blood glucose within the upper normal limits for age. If the initial blood glucose is a random sample that is outside of the normal limits, than follow-up fasting blood glucose can be obtained and must be within the upper normal limits for age.

(vii) Adequate Blood Pressure Control Defined as: A blood pressure (BP) ≤the 95th percentile for age, height, and gender and not receiving medication for treatment of hypertension.

(viii) Adequate Coagulation Defined as: Not actively on any anticoagulants and INR ≤1.5.

Patients meeting any one or more of the following exclusion criteria are not eligible for enrolling in the study: (1) Patients with interstitial lung disease and/or pneumonitis are not eligible; (2) Patients must not be receiving any strong CYP3A4 inducers or inhibitors within 7 days prior to enrollment; (3) Patient with a history of allergic reactions attributed to compounds of similar composition temsirolimus/other mTOR inhibitors, temozolomide or irinotecan are not eligible; (4) Patients with hypersensitivity to albumin are not eligible; (5) Patients have a BSA of <0.2 m² at the time of study enrollment are not eligible; (6) Patients with current or recent deep vein thrombosis are not eligible; and (5) Patients who have had or are planning to have the following invasive procedures are not eligible: Major surgical procedure, laparoscopic procedure, open biopsy or significant traumatic injury within 28 days prior to enrollment; Subcutaneous port placement or central line placement is not considered major surgery. External central lines must be placed at least 3 days prior to enrollment and subcutaneous ports must be placed at least 7 days prior to enrollment; Core biopsy within 7 days prior to enrollment; or Fine needle aspirate within 7 days prior to enrollment. For purposes of this study, bone marrow aspirate and biopsy are not considered surgical procedures and therefore are permitted within 14 days prior to start of protocol therapy.

### Example 3: Phase II Trial of ABI-009 in Combination with Vinorelbine (V) and Cyclophosphamide (C) for First Relapse/Disease Progression of Rhabdomyosarcoma (RMS)

Patients with RMS have a poor prognosis at first relapse/disease progression. VC has activity in RMS, however it would be desirable to improve the outcome for these patients.

Patients with, for example, biopsy-proven RMS, <30 years of age and unfavorable prognosis at first relapse/progression are eligible. Entry criteria, for example: life expectancy >8 weeks, performance status <2, adequate organ function and written informed consent. Patients are randomized between two regimens administered, for example, every 3 weeks for a maximum of 12 cycles:

Regimen A—V 25 mg/m² intravenously (IV) days 1 and 8; C 1.2 g/m² IV day 1 of a 3 week cycle;

Regimen B—VC identical to regimen A; ABI-009 15 mg/m²-45 mg/m² IV days 1, 8 and 15 or days 1 and 8 of a 3 week cycle.

Primary endpoint is, for example, event-free survival (EFS) at 6 months. Disease response at week 6 is assessed, for example, using RECIST. The study is powered, for example, to detect a 15% difference in EFS between the two regimens (α=0.2, 1−β=0.8, 2-sided test). Interim analysis is planned, for example, when 30%, 50% and 75% of the expected events occur.

### Example 4: Treatment of Solid Tumors with the Combination of Nab-Sirolimus and Anti-PD-1 Antibody

Immunocompetent mice bearing syngeneic tumors are treated with the combination of ABI-009 and anti-PD-1 antibody (such as clone RMP1-14 from Bio X Cell, West Lebanon, N.H., USA). A solid tumor cell line, such as mouse melanoma cell line B16-F10, is cultured, for example, in DMEM media supplemented with 10% fetal bovine calf serum (FBS) and incubated at 37° C. in humidified atmosphere of 5% CO₂. Mice, such as female C57BL/6 mice (5-6 weeks old), are injected, for example, subcutaneously with 1×10⁴ B16 cancer cells in 0.1 ml PBS with 20% Matrigel per flank.

Treatment starts when tumors grow, for example, to an average volume of 100 mm³. Mice are divided, for example, into at least one experimental group treated with the combination of ABI-009 and anti-PD-1 antibody, and one control group that receives no treatment or mock treatment. ABI-009 is administered, for example, intravenously (IV) at 5 mg/kg 3 times a week. Anti-PD1 antibody is administered, for example, intraperitoneally (IP) at 250 μg 3 times a week. For the combination treatment, ABI-009 is administered, for example, concurrently with, 1 week prior to, or 1 week following the administration of anti-PD-1 antibody. The animals in each group are monitored, for example, for tumor volume, adverse response, histopathology of tumor, body weight and general health condition (eating, walking, daily activities).

### Example 5: Treatment of Solid Tumors with the Combination of Nab-Sirolimus and Cancer Vaccines

Immunocompetent mice bearing syngeneic tumors are treated with the combination of ABI-009 and a cancer vaccine. A solid tumor cell line, such as mouse melanoma cell line B16 is transduced with a tumor-associated antigen, such as the human gp100 gene, to generate the B16-gp100 cell line, which is cultured, for example, in DMEM media supplemented with 10% fetal bovine calf serum (FBS) and incubate at 37° C. in humidified atmosphere of 5% CO₂. On Day 0, for example, mice, such as female C57BL/6 mice (6-8 weeks old), are injected, for example, intradermally with 2×10⁵ B16-gp100 cancer cells.

The cancer vaccine contains recombinant tumor-associated antigen, such as protein gp100, with an adjuvant, such as recombinant heat shock protein (HSP; hsp110). The adjuvant-based vaccine, such as HSP-based anti-tumor gp100 vaccine, is generated, for example, by incubating and non-covalently complexing gp100 and hsp110 recombinant proteins at an equal molar ratio.

Treatment starts, for example, on Day 10. Mice are divided, for example, into at least one experimental group treated, for example, on Day 10 and Day 17 with the combination of ABI-009 and cancer vaccine, such as gp100 cancer vaccine, and one control group that receives no treatment or mock treatment. ABI-009 is administered, for example, IV at 5 mg/kg. The cancer vaccine, such as gp100 vaccine, is administered, for example, intradermally at 25 μg. The animals in each group are monitored, for example, for tumor volume, adverse response, histopathology of tumor, body weight and general health condition (eating, walking, daily activities).

1: A method of treating a solid tumor in an individual, comprising administering to the individual: a) an effective amount of a composition comprising nanoparticles comprising an mTOR inhibitor and an albumin, and b) an effective amount of a second therapeutic agent, wherein the second therapeutic agent is selected from the group consisting of an immunomodulator, a histone deacetylase inhibitor, and a kinase inhibitor.

2: The method of claim 1, wherein the solid tumor is bladder cancer, renal cell carcinoma, soft-tissue sarcoma, or melanoma.

3: The method of claim 1, wherein the solid tumor is relapsed or refractory to a standard therapy for the solid tumor.

4: The method of claim 1, wherein the amount of the mTOR inhibitor in the mTOR inhibitor nanoparticle composition is from about 10 mg/m² to about 150 mg/m².

5-6. (canceled)

7: The method of claim 1, wherein the mTOR inhibitor nanoparticle composition is administered weekly.

8. (canceled)

9: The method of claim 1, wherein the mTOR inhibitor nanoparticle composition and the second therapeutic agent are administered sequentially to the individual.

10: The method of claim 1, wherein the mTOR inhibitor nanoparticle composition and the second therapeutic agent are administered simultaneously to the individual.

11: The method of claim 1, wherein the mTOR inhibitor is a limus drug.

12: The method of claim 11, wherein the limus drug is sirolimus.

13: The method of claim 1, wherein the average diameter of the nanoparticles in the composition is no greater than about 150 nm.

14. (canceled)

15: The method of claim 1, wherein the weight ratio of the albumin to the mTOR inhibitor in the nanoparticle composition is no greater than about 9:1.

16: The method of claim 1, wherein the nanoparticles comprise the mTOR inhibitor associated with the albumin.

17-18. (canceled)

19: The method of claim 1, wherein the mTOR inhibitor nanoparticle composition is administered intravenously.

20: The method of claim 1, wherein the individual is human.

21: The method of claim 1, further comprising selecting the individual for treatment based on the presence of at least one mTOR-activating aberration.

22-23. (canceled)

24: The method of claim 1, wherein the second therapeutic agent is an immunomodulator.

25. (canceled)

26: The method of claim 24, wherein the immunomodulator is an immune checkpoint inhibitor or an anti-PD-1 antibody.

27-29. (canceled)

30: The method of claim 1, wherein the second therapeutic agent is a histone deacetylase inhibitor.

31-33. (canceled)

34: The method of claim 1, wherein the second therapeutic agent is a kinase inhibitor.

35: The method of claim 34, wherein the kinase inhibitor is a tyrosine kinase inhibitor.

36-44. (canceled)
